(19) United States
(12) Patent Application Publication
(10) Pub. No.: US 2023/0255942 A1
(43) Pub. Date: Aug. 17, 2023

(54) CRF1 RECEPTOR ANTAGONIST FOR THE TREATMENT OF CONGENITAL ADRENAL HYPERPLASIA

(71) Applicants: Neurocrine Biosciences, Inc., San Diego, CA (US); Sanofi, Paris (FR)

(72) Inventors: Robert H. Farber, San Diego, CA (US); Jean L. Chan, San Diego, CA (US); Eiry Roberts, San Diego, CA (US); Gordon B. Cutler, JR., Deltaville, VA (US); Arline Nakanishi, San Diego, CA (US); Anne Charlier, Paris (FR); Gordon Raphael Loewen, San Diego, CA (US); Xiaoping Zhang, San Diego, CA (US); Nagdeep Giri, San Diego, CA (US); Scott Stirn, San Diego, CA (US); Brian Sayers, San Diego, CA (US); Graeme Taylor, San Diego, CA (US); Christina Marie Costa, San Diego, CA (US); Stacy Parks, Bend, OR (US); Anthony D. Vickery, Bend, OR (US); Kristie M. Downing, Bend, OR (US); Kingsley Iyoha, Nottingham (GB); Ayanda Ngwenya-Jones, Nottingham (GB); Gurvinder Mehton, Nottingham (GB)

(21) Appl. No.: 18/009,537

(22) PCT Filed: Jun. 9, 2021
(86) PCT No.: PCT/US2021/036668

§ 371 Date: Dec. 9, 2022

(30) Foreign Application Priority Data
Jun. 10, 2020 (FR) FR2006039

(51) Int. Cl. A61K 31/426; A61K 45/06; A61K 31/573; A61P 5/38; A61K 9/00; A61K 9/08

(52) U.S. Cl. CPC A61K 31/426; A61K 45/06; A61K 31/573; A61P 5/38; A61K 9/0053; A61K 9/08

(57) ABSTRACT

Provided are methods related to treating congenital adrenal hyperplasia in a subject in need thereof comprising administering to the subject a compound of Formula (I): [INSERT FORMULA] (I), or a pharmaceutically acceptable salt thereof.

### TECHNICAL FIELD

The present disclosure relates to 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, (i.e., a compound of Formula (I), or a pharmaceutically acceptable salt thereof, also referred to herein as crinecerfont) for the treatment of congenital adrenal hyperplasia (CAH).

### BACKGROUND

The compound of Formula (I)

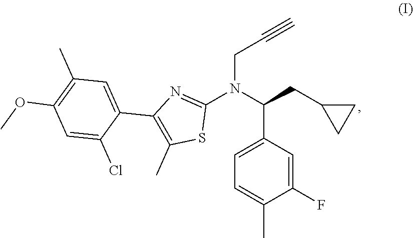 [SMILES: C#CCN(c1nc(-c2cc(C)c(OC)cc2Cl)c(C)s1)[C@@H](CC1CC1)c1ccc(C)c(F)c1]

4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, is a selective corticotropin-releasing hormone receptor 1 (CRF1) receptor antagonist that is being developed for the treatment of congenital adrenal hyperplasia associated with high adrenocorticotropin and adrenal steroid insufficiency. The compound of Formula (I) can be prepared according to the methods described in U.S. Pat. Nos. 6,586,456 and 8,314,249, each of which is hereby incorporated by reference in its entirety.

One clinical manifestation of the absence of cortisol that occurs in congenital adrenal hyperplasia (CAH) is the lack of feedback inhibition of pituitary adrenocorticotropic hormone (ACTH) secretion. Increased ACTH levels cause adrenal hyperplasia and the enzyme block causes a shunting of cortisol precursor steroids to alternate pathways. Most notably, the shunting to androgens leads to virilization and other developmental complications in females, and the elevated ACTH levels are associated with the formation of testicular adrenal rest tumors in males. In addition, since the same enzyme (21-hydroxylase) is used in the pathway for the biosynthesis of the mineralocorticoids, a number of these patients suffer from aldosterone deficiency which can result in dehydration and death due to salt-wasting.

While survival is properly ensured through steroid replacement strategies based on physiologic dosing of glucocorticoids (e.g., hydrocortisone) and mineralocorticoids (e.g., fludrocortisone), these doses are often inadequate to suppress the overproduction of ACTH, progestogens, and androgens (e.g., 17-hydroxyprogesterone [17-OHP], androstenedione, and testosterone). The uncontrolled symptoms of androgen excess, indeed, have a substantial impact on the day-to-day functioning and development of these patients. The glucocorticoid doses required to treat the androgen excess are typically well above the normal physiologic doses used for cortisol replacement alone (as in patients with Addison's disease). This increased exposure to glucocorticoids can lead to iatrogenic Cushing's syndrome, increased cardiovascular risk factors, glucose intolerance, and decreased bone mineral density in CAH patients (Elnecave et al., J Pediatr Endocrinol Metab. 2008 December; 21(12):1155-62; King et al., J Clin Endocrinol Metab. 2006 March; 91(3):865-9; Migeon and Wisniewski, Endocrinol Metab Clin North Am. 2001 March; 30(1):193-206).

Corticotropin-releasing factor is a hypothalamic hormone released directly into the hypophysial portal vasculature and acts on specific CRF₁ receptors on corticotropes in the anterior pituitary to stimulate the release of ACTH. Blockade of these receptors has been shown to decrease the release of ACTH in both animals and humans. Therefore, compounds that block CRF₁ receptors have the potential to directly inhibit the excessive ACTH release that occurs in CAH and thereby allow for normalization of androgen production while using lower, more physiologic doses of hydrocortisone. The compound of Formula (I), or a pharmaceutically acceptable salt thereof, may provide an important therapeutic approach to treat patients with CAH.

### SUMMARY

Provided herein are compounds, pharmaceutical compositions, and methods related to treating congenital adrenal hyperplasia in a subject.

Provided herein is a compound of Formula (I):

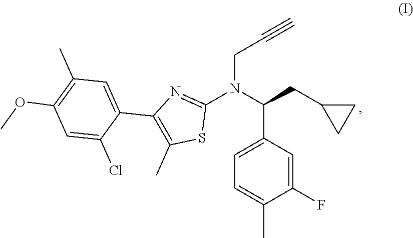 [SMILES: C#CCN(c1nc(-c2cc(C)c(OC)cc2Cl)c(C)s1)[C@@H](CC1CC1)c1ccc(C)c(F)c1]

or a pharmaceutically acceptable salt thereof;

for use in a method of treating congenital adrenal hyperplasia in a subject,

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a compound of Formula (I), or a pharmaceutically acceptable salt thereof for use in a method of treating congenital adrenal hyperplasia in a subject,

wherein the compound of Formula (I), or a pharmaceutically acceptable salt thereof, is administered at a frequency of not less than twice daily; and

wherein the amount of the compound of Formula (I), or a pharmaceutically acceptable salt thereof, administered daily is greater than 200 mg based on the weight of the free base.

Provided herein is a compound of Formula (I), or a pharmaceutically acceptable salt thereof, for use in a method of reducing the severity of one or more symptoms selected from hirsutism, precocious puberty, fertility problems, acne, and growth impairment in a subject having classic congenital adrenal hyperplasia,

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a compound of Formula (I), or a pharmaceutically acceptable salt thereof, for use in method of reducing the level of one or more biomarkers of congenital adrenal hyperplasia in a subject having congenital adrenal hyperplasia,

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a compound of Formula (I), or a pharmaceutically acceptable salt thereof, for use in a method of reducing the dosage of corticosteroid administered to a subject having congenital adrenal hyperplasia for controlling congenital adrenal hyperplasia,

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a compound of Formula (I), or a pharmaceutically acceptable salt thereof, for use in a method of reducing the severity of one or more side effects of glucocorticoid treatment in a subject having congenital adrenal hyperplasia,

wherein the compound of Formula (I), or a pharmaceutically acceptable salt thereof, is administered at a frequency of not less than twice daily;

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations; and

wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

Provided herein is a compound of Formula (I), or a pharmaceutically acceptable salt thereof, for use in a method of treating congenital adrenal hyperplasia in a subject, the method comprising:

(a) selecting a subject who has a glucocorticoid dose of greater than 11 mg/m²/day after a time period of being administered a compound of Formula (I), or a pharmaceutically acceptable thereof, at an amount of about 100 mg twice daily based on the weight of the free base;

and

(b) administering to the subject the compound of Formula (I), or a pharmaceutically acceptable salt thereof, at a frequency of twice daily;

wherein the amount of the compound of Formula (I), or a pharmaceutically acceptable salt thereof, in the first administration is less than the amount of the compound of Formula (I), or a pharmaceutically acceptable salt thereof, in the second administration;

and wherein the amount of the compound of Formula (I), or a pharmaceutically acceptable salt thereof, administered daily is greater than or equal to about 200 mg based on the weight of the free base.

Provided herein is a method of treating congenital adrenal hyperplasia in a subject in need thereof comprising administering to the subject a compound of Formula (I):

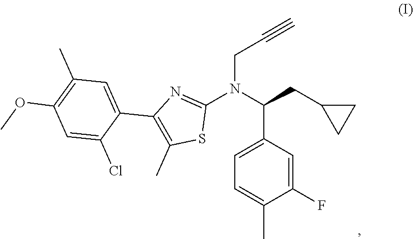 [SMILES: C#CCN(c1nc(-c2cc(C)c(OC)cc2Cl)c(C)s1)[C@@H](CC1CC1)c1ccc(C)c(F)c1]

or a pharmaceutically acceptable salt thereof;

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a method of treating congenital adrenal hyperplasia in a subject in need thereof comprising administering to the subject a compound of Formula (I), or a pharmaceutically acceptable salt thereof;

wherein the compound of Formula (I), or a pharmaceutically acceptable salt thereof, is administered at a frequency of not less than twice daily; and

wherein the amount of the compound of Formula (I), or a pharmaceutically acceptable salt thereof, administered daily is greater than 200 mg based on the weight of the free base.

Provided herein is a method for reducing the severity of one or more symptoms selected from hirsutism, precocious puberty, fertility problems, acne, and growth impairment in a subject having classic congenital adrenal hyperplasia,

comprising administering to the subject a compound of Formula (I), or a pharmaceutically acceptable salt thereof;

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

A method of reducing the level of one or more biomarkers of congenital adrenal hyperplasia in a subject having congenital adrenal hyperplasia comprising administering to the subject a compound of Formula (I), or a pharmaceutically acceptable salt thereof;

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a method of reducing the dosage of corticosteroid administered to a subject having congenital adrenal hyperplasia for controlling congenital adrenal hyperplasia comprising administering to the subject a compound of Formula (I), or a pharmaceutically acceptable salt thereof;

wherein the compound of Formula (I), or a pharmaceutically acceptable salt thereof, is administered at a frequency of not less than twice daily; and

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a method of reducing the severity of one or more side effects of glucocorticoid treatment in a subject having congenital adrenal hyperplasia comprising administering to the subject a compound of Formula (I), or a pharmaceutically acceptable salt thereof,

wherein the compound of Formula (I), or a pharmaceutically acceptable salt thereof, is administered at a frequency of not less than twice daily;

wherein the amount of the compound of Formula (I), or a pharmaceutically acceptable salt thereof, in the first administration is less than the amount of the compound of Formula (I), or a pharmaceutically acceptable salt thereof, in the second and any subsequent administrations; and

wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

Provided herein is a method of treating congenital adrenal hyperplasia in a subject, the method comprising:

(a) selecting a subject who has a glucocorticoid dose of greater than 11 mg/m²/day after a time period of being administered a compound of Formula (I), or a pharmaceutically acceptable thereof, at an amount of about 100 mg twice daily based on the weight of the free base; and

(b) administering to the subject the compound of Formula (I), or a pharmaceutically acceptable salt thereof, at a frequency of twice daily;

wherein the amount of the compound of Formula (I), or a pharmaceutically acceptable salt thereof, in the first administration is less than the amount of the compound of Formula (I), or a pharmaceutically acceptable salt thereof, in the second administration; and

wherein the amount of the compound of Formula (I), or a pharmaceutically acceptable salt thereof, administered daily is greater than or equal to about 200 mg based on the weight of the free base.

Also provided herein are pharmaceutical compositions comprising the compound of Formula (I) for use in any of the methods disclosed herein.

Other features and advantages of the methods, processes, formulations, and uses provided herein will be apparent from the following detailed description and figures, and from the claims.

### DESCRIPTION OF DRAWINGS

FIG. 1 shows the dissolution performance of several spray-dried dispersion formulations in 0.5 wt % simulated intestinal fluid (SIF) in phosphate buffered saline (PBS), pH 6.5.

FIG. 2 shows the vertical membrane flux cell integrated in the μDiss Profiler™ used for the membrane flux assay.

FIG. 3 shows non-sink dissolution data for several spray-dried dispersion formulations and the compound of Formula (I) in 0.5 wt % SIF in PBS, pH 6.5.

FIG. 4 is a graph showing membrane flux of 1 mg/mL GB/IB 0.5 wt % SIF doses of the compound of Formula (I) and various spray-dried dispersion formulations over time. The solid lines indicate flux (μg min⁻¹ cm⁻²) and the broken lines indicate concentration (μg/mL) in 0.5% SIF.

FIG. 5 is a flow diagram of the spray drying manufacturing process used to prepare a 1000 g batch of a SDD containing 25% of the compound of Formula (I) and 75% PVP/VA 64.

FIGS. 6A and 6B are line graphs showing the pharmacokinetic results of a bioavailability and food effect study in dogs. FIG. 6A shows the results from Cohort 1 and FIG. 6B shows the results from Cohort 2.

FIG. 7 is a flow chart showing the study design of a Phase 1 study of the pharmacokinetics and food effect of the compound of Formula (I) in healthy adult subjects.

FIGS. 8A and 8B are line graphs showing the mean plasma concentration versus time profiles for the compound of Formula (I) under fasted and fed conditions, respectively, in healthy adult subjects.

FIGS. 9A-9C are spaghetti plots of the pharmacokinetics of the compound of Formula (I) in healthy adult subjects under fasted and fed conditions. FIG. 9A shows the AUC₀₋ₜₗₐₛₜ values. FIG. 9B shows the AUC_{0-∞} values. FIG. 9C shows the Cₘₐₓ values.

FIG. 10 is a flow chart showing the study design of a Phase 1 study of the bioavailability, pharmacokinetics and food effect of the compound of Formula (I) in healthy adult subjects.

FIG. 11 shows the study design of a Phase 2 study of the compound of Formula (I) in adult subjects with congenital adrenal hyperplasia.

FIGS. 12A and 12B show the arithmetic mean values for adrenocorticotropic hormone (ACTH) (FIG. 12A) and 17-hydroxyprogesterone (17-OHP) (FIG. 12B) for all 8 Cohort 1 subjects plotted at each time point for pre-treatment baseline (circles), day 1 (squares), and day 14 (triangles).

FIGS. 13A and 13B show arithmetic mean values for androstenedione (FIG. 13A) and testosterone (FIG. 13B) for all 8 Cohort 1 subjects were plotted at each timepoint for pre-treatment baseline (circles), day 1 (squares), and day 14 (triangles).

FIGS. 14A and 14B show the reduction of ACTH at timepoints 8-, 10-, and 12-hours postdose. FIG. 14A shows the values for each time point as compared to baseline. FIG. 14B shows the mean values across all three timepoints.

FIGS. 15A and 15B show the reduction of 17-OHP at timepoints 8-, 10-, and 12-hours postdose. FIG. 15A shows the values for each time point as compared to baseline. FIG. 15B shows the mean values across all three timepoints.

FIGS. 16A and 16B show the reduction of androstenedione at timepoints 8-, 10-, and 12-hours postdose. FIG. 16A shows the values for each time point as compared to baseline.

FIG. 16B shows the mean values across all three timepoints.

FIG. 17A shows the plasma ACTH Mean Blood Concentrations following the compound of Formula (I) 50 mg Dose qhs (Cohort 1; n=8). Error bars represent the standard error of the mean for each morning window timepoint. ACTH normal ranges: Female 6 to 58 pg/mL; Male 7 to 69 pg/mL.

FIG. 17B shows the serum 17-OHP Mean Blood Concentrations following the compound of Formula (I) 50 mg Dose qhs (Cohort 1; n=8). Error bars represent the standard error of the mean for each morning window timepoint. 17-OHP normal ranges: Female <207 ng/dL; Male <139 ng/dL.

FIG. 17C: shows the serum Androstenedione Mean Blood Concentrations following the compound of Formula (I) 50 mg Dose qhs (Cohort 1; n=8). Error bars represent the standard error of the mean for each morning window timepoint. Androstenedione normal ranges: Female 26 to 214 ng/mL; Male 33 to 134 ng/mL.

FIG. 18A shows the plasma ACTH Mean Blood Concentrations following the compound of Formula (I) 100 mg Dose qhs (Cohort 2; n=4). Error bars represent the standard error of the mean for each morning window timepoint. ACTH normal ranges: Female 6 to 58 pg/mL; Male 7 to 69 pg/mL.

FIG. 18B shows the serum 17-OHP Mean Blood Concentrations following the compound of Formula (I) 100 mg Dose qhs (Cohort 2; n=4). Error bars represent the standard error of the mean for each morning window timepoint. 17-OHP normal ranges: Female <207 ng/dL; Male <139 ng/dL.

FIG. 18C shows the serum Androstenedione Mean Blood Concentrations following the compound of Formula (I) 100 mg Dose qhs (Cohort 2; n=4). Error bars represent the standard error of the mean for each morning window timepoint. Androstenedione normal ranges: Female 26 to 214 ng/mL; Male 33 to 134 ng/mL.

FIG. 19A shows the plasma ACTH Mean Blood Concentrations following the compound of Formula (I) 100 mg Dose with Evening Meal (Cohort 3). Error bars represent the standard error of the mean for each morning window timepoint. ACTH normal ranges: Female 6 to 58 pg/mL; Male 7 to 69 pg/mL.

FIG. 19B shows the serum 17-OHP Mean Blood Concentrations following the compound of Formula (I) 100 mg Dose with Evening Meal (Cohort 3). Error bars represent the standard error of the mean for each morning window timepoint. 17-OHP normal ranges: Female <207 ng/dL; Male <139 ng/dL.

FIG. 19C shows the serum Androstenedione Mean Blood Concentrations following the compound of Formula (I) 100 mg Dose with Evening Meal (Cohort 3). Error bars represent the standard error of the mean for each morning window timepoint. Androstenedione normal ranges: Female 26 to 214 ng/mL; Male 33 to 134 ng/mL.

FIG. 20 is a scheme showing the manufacturing process for forming 50 mg capsules of the compound of Formula (I).

FIG. 21 is an alternative scheme showing the manufacturing process for forming 50 mg capsules of the compound of Formula (I).

FIGS. 22A and 22B show a scheme showing the manufacturing process for forming SDD granules of the compound of Formula (I).

FIG. 23 is a scheme showing the manufacturing process for forming 50 mg/nL liquid formulation 1 of the compound of Formula (I).

FIG. 24 is a scheme showing the manufacturing process for forming 50 mg/nL liquid formulation 2 of the compound of Formula (I).

FIG. 25 is an XRPD spectrum of the compound of Formula (I) free base crystalline form I.

FIG. 26 is a DSC spectrum of the compound of Formula (I) free base crystalline form 1.

FIG. 27 is an XRPD spectrum of the compound of Formula (I) tosylate crystalline form 1.

FIG. 28 is a DSC and TGA spectrum of the compound of Formula (I) tosylate crystalline form 1.

### DETAILED DESCRIPTION

As described herein, 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine having the Formula (I):

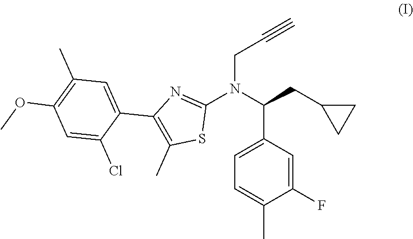 [SMILES: C#CCN(c1nc(-c2cc(C)c(OC)cc2Cl)c(C)s1)[C@@H](CC1CC1)c1ccc(C)c(F)c1]

or a pharmaceutically acceptable salt thereof, is a selective CRF1 receptor antagonist that has been found to be effective treating congenital adrenal hyperplasia. Specifically, the compound of Formula (I) has been found to effectively reduce several biomarkers associated with congenital adrenal hyperplasia. As used herein, the term “crinecerfont” refers to the compound of Formula (I) and includes any pharmaceutically acceptable salts and/or polymorphs thereof. In addition to the chemical name disclosed above, crinecerfont may also be named 4-(2-chloro-4-methoxy-5-methylphenyl)-N-[(1S)-2-cyclopropyl-1-(3-fluoro-4-methylphenyl)ethyl]-5-methyl-N-(prop-2-yn-1-yl)-1,3-thiazol-2-amine (see International Nonproprietary Names for Pharmaceutical Substances (INN), WHO Drug Information, Vol. 32, No. 4, 2018). Crinecerfont has an assigned CAS No. of 752253-39-7 with a CAS name of 2-Thiazolamine, 4-(2-chloro-4-methoxy-5-methylphenyl)-N-[(1S)-2-cyclopropyl-1-(3-fluoro-4-methylphenyl)ethyl]-5-methyl-N-2-propyn-1-yl- (CA INDEX NAME). Crinecerfont has also been referred to in the art as “SSR125543” and “NBI-74788”.

Newborn screening for CAH is performed by immunoassay to measure 17-OHP levels in heel-stick capillary blood specimens obtained within the first 72 hours of life. The blood sample is analyzed for 17-OHP by commercially available dissociation-enhanced lanthanide fluoroimmunoassay (DELFIA; PerkinElmer, Waltham Mass.) (White et al., J. Pediatr. 163:10-12 (2013)). Second-tier screening tests utilizing biochemical and molecular genetic testing methods, performed between 8 and 14 days of life, are employed by nine states in the United States and strongly recommended by an additional 5 states. The biochemical method includes immunoassay with organic solvent extraction or liquid chromatography followed by tandem mass spectrometry to measure steroid ratios of 17-OHP, androstenedione, and 21-deoxycortisol to cortisol (see, e.g., Speiser et al., Int. J. Pediatr. Endocrinol. 2010:494173, 2010). The genetic screen looks for CYP21A2 mutations that are associated with CAH. While not widely employed in the U.S., the addition of a second screening could potentially improve the sensitivity of the overall screening process, where sensitivity of the first screen alone is approximately 72%.

In absence of results from the newborn screening, female infants with classical CAH are typically identified due to the presence of ambiguous genitalia. Males have normal genitalia at birth and therefore are not diagnosed unless newborn screening is conducted or other medical complications come to attention. Infants who are not initially diagnosed with CAH and suffer from the salt-wasting form of the disease are later diagnosed in the setting of poor weight gain, vomiting, hyperkalemia and hyponatremia within the first few weeks of life.

Treatment of CAH is based on normalization of hormone and steroid levels using a variety of medications from diagnosis in infancy through adulthood. Glucocorticoids are the current standard treatment in CAH and are used both to correct the endogenous cortisol deficiency and for reducing the elevated ACTH levels from the pituitary, which drives increased androgen production. Unlike the treatment of Addison's disease (adrenal insufficiency), in which cortisol replacement is sufficient, the treatment of CAH must also reduce ACTH production, to control the subsequent androgen excess as well. Thus, the goals of glucocorticoid treatment include cortisol replacement and suppression of ACTH to prevent virilization and menstrual disturbances in women and to inhibit testicular adrenal rest tumors in men. Mineralocorticoid replacement is needed to achieve normal plasma renin activity for maintenance of regular blood pressure, electrolyte balance, and volume status in those patients with the salt-wasting form of CAH.

The regimen of glucocorticoid treatment must support normal physiology and also ensure that sufficient cortisol is available during events that may elicit a strong stress response (e.g., intercurrent illness, exercise, hypotension). Careful monitoring is also necessary to avoid the development of iatrogenic Cushing's syndrome due to glucocorticoid overtreatment in an effort to adequately suppress androgen production, or Addisonian syndrome due to under-treatment.

Overtreatment with mineralocorticoids may cause hypertension while under-treatment may lead to low blood pressure, salt loss, fatigue and increased requirements for glucocorticoids. Typical laboratory tests for monitoring treatment efficacy include measurement of plasma concentrations of 17-OHP, androstenedione, testosterone, renin activity, and electrolytes.

Adult patients with CAH have an increased prevalence of risk factors for cardiovascular disease including obesity, hypertension, and insulin resistance (see, e.g., Kim et al., Semin. Reprod. Med. 27(4):316-21 (2009)). A study of a large cohort of pediatric and adult CAH patients (n=244) demonstrated that patients are prescribed a variety of glucocorticoid treatment regimens yet frequently suffer from poor hormonal control and the aforementioned adverse outcomes (see, e.g., Finkielstain et al., J. Clin. Endocrinol Metab. 97(12):4429-38 (2012)).

Treatment of CAH includes efforts to normalize the cortisol deficiency with glucocorticoids (usually hydrocortisone in children but often more potent agents with narrow therapeutic indices, such as dexamethasone, in adults) and, if necessary for salt-wasting, mineralocorticoids (usually fludrocortisone). The glucocorticoid doses required to achieve sufficient suppression of excess androgens, however, are usually well above the normal physiologic dose used for cortisol replacement alone as in patients with Addison's disease.

This increased exposure to glucocorticoids can lead to iatrogenic Cushing's syndrome, increased cardiovascular risk factors, glucose intolerance, and decreased bone mineral density in CAH patients (see, e.g., Elnecave et al., J. Pediatr. Endocrinol. Metab. 21:1155-62 (2008); King et al., J. Clin. Endocrinol. Metab. 91(3):8656-59 (2006); Migeon et al., Endocrinol. Metab. Clin. North Am. 30:193-206 (2001)). Recently, best practices for the clinical management of congenital adrenal hyperplasia were published in the Journal of Clinical Endocrinology and Metabolism (Speiser, P. W., et al. J. Clin. Endocrinol. Metab. November 2018, 103(11): 1-46). This article is incorporated by reference in its entirety.

Corticotropin-releasing factor (CRF) was isolated from ovine hypothalami and identified as a 41-amino acid peptide. CRF has been found to produce profound alterations in endocrine, nervous, and immune system function. CRF is believed to be the major physiological regulator of the basal and stress-induced release of adrenocorticotropic hormone (“ACTH”), 8-endorphin, and other pro-opiomelanocortin (“POMC”)-derived peptides from the anterior pituitary (see, e.g., Vale et al., Science 213:1394-1397, 1981). Secretion of CRY causes release of ACTH from corticotrophs in the anterior pituitary via binding to the CRF₁ receptor, a member of the class B family of G-protein coupled receptors.

Due to the physiological significance of CRF, the development of biologically-active small molecules having significant CRF₁ receptor binding activity and which are capable of antagonizing the CRF₁ receptor remains a desirable goal and has been the subject of ongoing research and development for the treatment of anxiety, depression, irritable bowel syndrome, post-traumatic stress disorder, and substance abuse.

The pituitary hormone ACTH, under the control of hypothalamic corticotropin-releasing factor (CRF), stimulates uptake of cholesterol and drives the synthesis of pregnenolone initiating steroidogenesis in the adrenal gland. The adrenal cortex is comprised of three zones, which produce distinct classes of hormones many of which are driven by ACTH mobilizing cholesterol through this pathway. Deficiencies in these enzymes as a result of mutation or deletion cause the substrate concentrations to increase. In the most common form of CAH resulting from mutations or deletions in the 21-hydroxylase gene (CYP21A2), potent androgens are produced by the adrenal because of the accumulation of the steroid precursors, progesterone and 17-hydroxyprogesterone (17-OHP). Plasma levels of 17-OHP can reach 10-1000 times the normal concentration in these cases. These increases result in the overproduction of androgens, specifically androstenedione, testosterone, and dihydroxytestosterone causing virilization in females. In addition, 21-hydroxylase deficiency in CAH causes insufficient biosynthesis of glucocorticoids and mineralocorticoids, specifically cortisol and aldosterone. Cortisol is a critical negative feedback regulator of hypothalamic CRY secretion and pituitary ACTH release. The lack of glucocorticoid synthesis and release eliminates the restraint on the hypothalamus and pituitary, which causes ACTH levels to increase. The excessive ACTH stimulation causes hypertrophy of the zona fasciculata and zona reticularis resulting in adrenal hyperplasia.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “about” preceding a value for DSC, TGA, or T_{g}, which are reported as degrees Celsius, have an allowable variability of ±5° C. In all other instances, unless otherwise specified, the term “about” preceding a stated value includes the stated value and also includes ±20% of the stated value, and includes more specifically values of 10%, ±5%, ±2%, and ±1% of the stated value.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

“Room temperature” or “RT” refers to the ambient temperature of a typical laboratory, which is typically around 25° C.

“Spray-drying” refers to the method of producing a dry powder from a solution or slurry. The solution or slurry is atomized or rapidly dried with a hot gas, e.g., air or nitrogen, that causes the solvent to evaporate quickly and uniformly. A “spray-dried dispersion” refers to the powder obtained from the spray-drying process.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

“Subject,” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate. In some embodiments, the subject is a human.

In some embodiments, the subject has experienced and/or exhibited at least one symptom of the disease or disorder to be treated and/or prevented. In some embodiments, the subject has been identified or diagnosed as having congenital adrenal hyperplasia (CAH). In some embodiments, the subject is suspected of having CAH. In some embodiments, the subject has a clinical record indicating that the subject has CAH (and optionally the clinical record indicates that the subject should be treated with any of the compositions provided herein). In some embodiments, the subject is a pediatric subject.

The term “pediatric subject” as used herein refers to a subject under the age of 21 years at the time of diagnosis or treatment. The term “pediatric” can be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman et al., Textbook of Pediatrics, 15th Ed. Philadelphia: W.B. Saunders Company, 1996; Rudolph et al., Rudolph's Pediatrics, 21st Ed. New York: McGraw-Hill, 2002; and Avery et al., Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “preventing,” as used herein, means the prevention of the onset, recurrence or spread, in whole or in part, of the disease or condition as described herein, or a symptom thereof.

The term “administration” or “administering” refers to a method of giving a dosage of a compound or pharmaceutical formulation to a vertebrate or invertebrate, including a mammal, a bird, a fish, or an amphibian. The preferred method of administration can vary depending on various factors, e.g., the components of the pharmaceutical formulation, the site of the disease, and the severity of the disease.

As used herein, “therapeutically effective amount” is an amount of the compound of Formula (I), or a pharmaceutically acceptable salt thereof, or an amount of a pharmaceutical composition comprising the compound of Formula (I), which is sufficient to achieve the desired effect and can vary according to the nature and severity of the disease condition, and the potency of the compound. A therapeutic effect is the relief, to some extent, of one or more of the symptoms of the disease, and can include curing a disease. “Curing” means that the symptoms of active disease are eliminated. However, certain long-term or permanent effects of the disease can exist even after a cure is obtained (such as, e.g., extensive tissue damage).

The term “amorphous” means a solid in a solid state that is a non-crystalline state. Amorphous solids are disordered arrangements of molecules and therefore possess no distinguishable crystal lattice or unit cell and consequently have no definable long range ordering. The solid state form of a solid may be determined by polarized light microscopy, X-ray powder diffraction (XRPD), differential scanning calorimetry (DSC), or other standard techniques known to those of skill in the art.

As used herein, “time of day window” refers to a period of time defined by a window start time and a window stop time. These times all refer to local times where a sample was taken. The phrase “same time of day window” when referring to samples taken from the subject mean, e.g., that a sample taken at 8:15 a.m. and a sample taken at 9:15 a.m. are considered to be taken in the same time of day window of, e.g., 2 a.m. to 10 a.m. or 6 a.m. to 10 a.m.

### Methods

The present disclosure relates to methods of treating congenital adrenal hyperplasia (CAH). The methods include administering to a subject a therapeutically effective amount of a compound of Formula (I), or pharmaceutically acceptable salt thereof.

In some embodiments, the compound of Formula (I), or a pharmaceutically acceptable salt thereof, is administered at a frequency of not less than twice daily; and the amount of the compound of Formula (I), or a pharmaceutically acceptable salt thereof, in the first administration is less than the amount of the compound of Formula (I), or a pharmaceutically acceptable salt thereof, in the second and any subsequent administrations.

In some embodiments, the compound of Formula (I), or a pharmaceutically acceptable salt thereof, is administered at a frequency of not less than twice daily; and the amount of the compound of Formula (I), or a pharmaceutically acceptable salt thereof, administered daily is greater than 200 mg based on the weight of the free base.

Provided herein is a method of treating congenital adrenal hyperplasia (CAH) comprising administering a compound of Formula (I), or a pharmaceutically acceptable salt thereof to normalize or partially normalize levels of biomarkers associated with congenital adrenal hyperplasia. In some embodiments, normalizing or partially normalizing levels of biomarkers comprises reducing levels of elevated biomarkers or increasing levels of depressed biomarkers as compared to subject without CAH.

Provided herein is a method of treating congenital adrenal hyperplasia in a subject in need thereof comprising administering a compound of Formula (I), or a pharmaceutically acceptable salt thereof, in an amount sufficient to reduce the level of one or more biomarkers associated with congenital adrenal hyperplasia. In some embodiments, the biomarkers are selected from (a) 17-hydroxyprogesterone (17-OHP); (b) adrenocorticotropic hormone (ACTH); and (c) androstenedione in the subject.

In some embodiments, the reduction in level of any of the biomarkers (e.g., any of 17-OHP, ACTH, and androstenedione) is determined by comparing the level of the biomarker as measured during the circadian release on a day prior to administering the compound of Formula (I), or a pharmaceutically acceptable salt thereof and the level of the biomarker as measured during the circadian release on the day after administering the compound of Formula (I), or a pharmaceutically acceptable salt thereof. A day prior to administering the compound of Formula (I) applies to a subject that has not previously been administered the compound of Formula (I) within at least the past 24 hours.

In some embodiments, the circadian release of biomarkers associated with CAH occurs between the hours of 2 a.m. and 10 a.m. In other embodiments, the circadian release of biomarkers associated with CAH occurs between the hours of 6 a.m. and 10 a.m.

In some embodiments of any of the methods disclosed herein, the compound of Formula (I), or a pharmaceutically acceptable salt, is administered to the subject at nighttime or administration prior to sleep (i.e., bedtime administration). In some embodiments, the compound of Formula (I), or a pharmaceutically acceptable salt thereof, is administered three to eight hours prior to the circadian release of the biomarker. In some embodiments, the compound of Formula (I), or a pharmaceutically acceptable salt thereof, is administered six to eight hours prior to the circadian release of the biomarker. Administration prior to the circadian release may be adapted for shift workers (e.g., those who work at night and sleep during the day), in which case administration will not necessarily occur at nighttime. Administration is therefore dependent upon the expected circadian release of the biomarker, and can vary depending upon the individual's (i.e., subject, patient) particular work and sleep patterns.

In some embodiments of the methods provided herein, the level of 17-hydroxyprogesterone is reduced by at least 10%, at least 15%, at least 20%, at least 25%, at least 30%, at lease 35%, at least 40%, at least 50%, at least 55% or at least 60% from pre-administration levels. In some embodiments, the level of 17-hydroxyprogesterone is reduced by at least 25%. In some embodiments, the level of 17-hydroxyprogesterone is reduced by at least 50%. In some embodiments of the methods provided herein, the level of 17-hydroxyprogesterone is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some embodiments, the level of 17-hydroxyprogesterone is reduced to a level within the range of 17-hydroxyprogesterone expected for a subject without CAH, i.e., less than 1,000 ng/dL or less than 200 ng/dL.

In some embodiments of the methods provided herein, the level of adrenocorticotropic hormone is reduced by at least 10%, at least 15%, at least 20%, at least 25%, at least 30%, at lease 35%, at least 40%, at least 50%, at least 55% or at least 60% from pre-administration levels. In some embodiments, the level of adrenocorticotropic hormone is reduced by at least 25%. In some embodiments, the level of adrenocorticotropic hormone is reduced by at least 40%. In some embodiments, the level of adrenocorticotropic hormone is reduced by at least 50%.

In some embodiments of the methods provided herein, the level of adrenocorticotropic hormone is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some embodiments, the level of adrenocorticotropic hormone is reduced to a level within the range of adrenocorticotropic hormone expected for a subject without CAH.

In some embodiments of the methods provided herein, the level of androstenedione is reduced by at least 10%, at least 15%, at least 20%, at least 25%, at least 30%, at lease 35%, at least 40%, at least 50%, at least 55% or at least 60% from pre-administration levels. In some embodiments, the level of androstenedione is reduced by at least 25%. In some embodiments, the level of androstenedione is reduced by at least 30%. In some embodiments, the level of androstenedione is reduced by at least 50%.

In some embodiments of the methods provided herein, the level of androstenedione is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some embodiments, the level of androstenedione is reduced to a level within the range of androstenedione expected for a subject without CAH, i.e., less than 200 ng/dL.

Also provided herein is a method for reducing the severity of one or more symptoms selected from hirsutism, precocious puberty, fertility problems, acne, and growth impairment in a subject having classic congenital adrenal hyperplasia, comprising administering a compound of Formula (I), or a pharmaceutically acceptable salt thereof, in an amount sufficient to reduce one or more biomarker of CAH in a subject, e.g., reduce the androstenedione in the subject. Growth impairment can refer to, e.g., accelerated height velocity, accelerated weight velocity, and/or accelerated bone age.

Provided herein is a method for reducing the level of one or more biomarkers of congenital adrenal hyperplasia in a subject having congenital adrenal hyperplasia comprising administering to the subject a compound of Formula (I), or a pharmaceutically acceptable salt thereof. In some embodiments, the one or more biomarkers of congenital adrenal hyperplasia are selected from (a) 17-hydroxyprogesterone (17-OHP); (b) adrenocorticotropic hormone (ACTH); and (c) androstenedione.

Provided herein is a method for reducing the dosage of corticosteroid administered to a subject having congenital adrenal hyperplasia for controlling congenital adrenal hyperplasia comprising administering to the subject a compound of Formula (I), or a pharmaceutically acceptable salt thereof. In some embodiments, the corticosteroid is a glucocorticoid.

Also provided herein is a method of reducing the severity of one or more side effects of glucocorticoid treatment in a subject having congenital adrenal hyperplasia comprising administering to the subject a compound of Formula (I), or a pharmaceutically acceptable salt thereof. The long-term effects of glucocorticoid treatment are well documented in the art (see, e.g., Oray, M. et al. (2016): Long-term effect of glucocorticoids, Expert Opinion on Drug Safety. DOI: 10.1517/14740338.2016.1140743). Such side effects are associated with every biological system, e.g., musculoskeletal (e.g., osteoporosis, avascular necrosis of bone, and myopathy), endocrine and metabolic (e.g., hyperglycemia, diabetes mellitus, dyslipidemia, weight gain, Cushing syndrome, Cushingoid features, growth suppression, adrenal suppression), gastrointestinal (e.g., gastritis, peptic ulcer, gastrointestinal bleeding, visceral perforation, hepatic steatosis, pancreatitis), cardiovascular (e.g., hypertension, coronary heart disease, ischemic heart disease, heart failure), dermatologic (e.g., dermatoprosis, skin atrophy, ecchymosis, purpura, erosions, striae, delayed wound healing, easy bruising, acne, hirsutism, and hair loss), neuropsychiatric (e.g., mood changes, depression, euphoria, mood lability, irritability, akathisia, anxiety, cognitive impairment, psychosis, dementia, and delirium), ophthalmologic (e.g., cataract, glaucoma, ptosis, mydriasis, opportunistic ocular infections, and central serous chorioretinopathy), and immunologic (e.g., suppression of cell-mediated immunity, predisposition to infections, and reactivation of latent infections).

Accordingly, in some embodiments, the side effects of glucocorticoid treatment are selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections, and any combination thereof.

Provided herein is a method of treating congenital adrenal hyperplasia in a subject comprising
(i) measuring the level of one or more biomarkers selected from (a) 17-hydroxyprogesterone (17-OHP); (b) adrenocorticotropic hormone (ACTH); and (c) androstenedione in a biological sample obtained from the subject;
(ii) analyzing the level of the one or more biomarkers to determine if the level of the one or more biomarkers is elevated compared to a healthy subject not having congenital adrenal hyperplasia; and
(iii) administering to the subject a compound of Formula (I), or a pharmaceutically acceptable salt thereof if the subject is determined to have elevated levels of the one or more biomarkers.

In some embodiments, the method further comprises (iv) measuring the level of the one or more biomarkers after administering a compound of Formula (I), or a pharmaceutically acceptable salt thereof, in a biological sample obtained from the subject to determine whether the subject has reduced levels of the one or more biomarkers as compared with the measurement of step (i). In some embodiments, the method further comprises (v) continuing the administration of the compound of Formula (I), or a pharmaceutically acceptable salt thereof if the subject has reduced levels of the one or more biomarkers.

In some embodiments, steps (i) and (iv) are performed on biological samples taken from the subject in a similar manner and within a same time of day window. In some embodiments, steps (i) and (iv) are performed on biological samples taken from the subject within the time of day window from 2 a.m. to 10 a.m. In some embodiments, steps (i) and (iv) are performed on biological samples taken from the subject within the time of day window from 6 a.m. to 10 a.m.

In some embodiments, steps (i) and (iv) comprise measuring the levels of at least two biomarkers selected from (a) 17-hydroxyprogesterone (17-OHP); (b) adrenocorticotropic hormone (ACTH); and (c) androstenedione.

In some embodiments, steps (i) and (iv) comprise measuring the levels of (a) 17-hydroxyprogesterone (17-OHP); (b) adrenocorticotropic hormone (ACTH); and (c) androstenedione.

In some embodiments, step (i) comprises measuring the level of 17-hydroxyprogesterone (17-OHP), wherein the level of 17-hydroxyprogesterone (17-OHP) is elevated when it is greater than or equal to 1,000 ng/dL.

In some embodiments, step (i) comprises measuring the level of androstenedione, wherein the level of androstenedione is elevated when it is greater than 200 ng/dL.

In some embodiments of the methods of the present disclosure, the compound of Formula (I) is administered at an amount equivalent to from about 25 mg to about 150 mg of the compound of Formula (I) free base. In some embodiments, compound of Formula (I), or a pharmaceutically acceptable salt thereof, is administered at an amount equivalent to about 50 mg or about 100 mg of the compound of Formula (I) free base.

In some embodiments of the methods disclosed herein, compound of Formula (I) is administered in the free base form.

In some embodiments of the methods disclosed herein, the compound of Formula (I) is administered twice daily (i.e., as a first and second administration). In some embodiments, the amount of the compound of Formula (I), or a pharmaceutically acceptable salt in the first administration is less than the amount of the compound of Formula (I), or a pharmaceutically acceptable salt thereof, in the second administration.

Also provided herein is method of treating congenital adrenal hyperplasia in a subject, the method comprising:

(a) selecting a subject who has a glucocorticoid dose of greater than 11 mg/m²/day after a time period of being administered a compound of Formula (I), or a pharmaceutically acceptable thereof, at an amount of about 100 mg twice daily based on the weight of the free base; and

(b) administering to the subject the compound of Formula (I), or a pharmaceutically acceptable salt thereof, at a frequency of twice daily;

wherein the amount of the compound of Formula (I), or a pharmaceutically acceptable salt thereof, in the first administration is less than the amount of the compound of Formula (I), or a pharmaceutically acceptable salt thereof, in the second administration.

In some embodiments, the amount of the compound of Formula (I), or a pharmaceutically acceptable salt thereof, administered daily in step (b) is greater than or equal to about 200 mg based on the weight of the free base.

In some embodiments, the glucocorticoid dose of step (a) is measured in hydrocortisone equivalents (which may be adjusted for body surface area (BSA)).

In some embodiments, the time period of administration in step (a) is at least about 4 weeks. In some embodiments, the time period of administration in step (a) is at least about 24 weeks. In some embodiments, the time period of administration in step (a) is at least about 6 months. In some embodiments, the time period of administration in step (a) is at least about one year.

Also provided herein is a method of treating CAH in a pediatric subject. The methods include administering to a pediatric subject a therapeutically effective amount of a compound of Formula (I), or pharmaceutically acceptable salt thereof. In some embodiments, the pediatric subject weighs greater than or equal to about 55 kg and a therapeutically effective amount of the compound of Formula (I), or a pharmaceutically acceptable salt thereof is about 100 mg administered twice daily (i.e., a total daily amount of about 200 mg based on the free base). In some embodiments, the pediatric subject weighs from about 10 kg to about 20 kg and a therapeutically effective amount of the compound of Formula (I), or a pharmaceutically acceptable salt thereof is about 25 mg administered twice daily (i.e., a total daily amount of about 50 mg based on the free base). In some embodiments, the pediatric subject weighs from about 20 kg to about 55 kg and a therapeutically effective amount of the compound of Formula (I), or a pharmaceutically acceptable salt thereof is about 50 mg administered twice daily (i.e., a total daily amount of about 100 mg, based on the free base). In some embodiments, the method includes administering to a pediatric subject a therapeutically effective amount of a SDD of the present disclosure that includes a polymer and a compound of Formula (I), or a pharmaceutically acceptable salt thereof. In some embodiments, the method includes administering to a pediatric subject a therapeutically effective amount of pharmaceutical composition of the present disclosure that contains a SDD that includes a polymer and a compound of Formula (I), or a pharmaceutically acceptable salt thereof. In some embodiments, the pediatric subject is a neonate. In some embodiments, the pediatric subject is an infant. In some embodiments, the pediatric subject is a child. In some embodiments, the pediatric subject is an adolescent.

In some embodiments of the methods of the present disclosure, the compound of Formula (I), or a pharmaceutically acceptable salt thereof, is administered to the subject in a fed state. The term “fed state,” as used herein, refers to administration of the compound of Formula (I), or a pharmaceutically acceptable salt thereof from about 1 hour before consumption of food or a nutritional composition to about 1 hour after consumption of food or a nutritional composition. The term “fasted state,” as used herein, refers to a gap of at least two hours between consumption of food or a nutritional composition and administration of the compound of Formula (I), or a pharmaceutically acceptable salt thereof. In some embodiments, the compound of Formula (I), or a pharmaceutically acceptable salt thereof is administered to the subject with food or a nutritional composition, such as a nutritional supplement or formula, a meal replacement beverage, a liquid dietary supplement, or a high caloric liquid meal. In some embodiments, the compound of Formula (I), or a pharmaceutically acceptable salt thereof is administered to the subject within about 1 hour before the subject has consumed food or a nutritional composition. In some embodiments, the compound of Formula (I), or a pharmaceutically acceptable salt thereof is administered to the subject within about 1 hour after the subject has consumed food or a nutritional composition. Examples of suitable nutritional compositions include, but are not limited to, infant formulas, dietary supplements, dietary substitutes, and rehydration compositions. In some embodiments, the food is a product containing concentrated calories and protein. In some embodiments, the nutritional composition is a composition utilized for enteral and parenteral supplementation for infants, specialty infant formulas, supplements for the elderly, and supplements for those with gastrointestinal difficulties and/or malabsorption. Adult and pediatric nutritional formulas are well known in the art and are commercially available (e.g., Similac®, Ensure®, Jevity® and Alimentum® from Ross Products Division, Abbott Laboratories, Columbus, Ohio).

In some embodiments, the nutritional composition is in liquid form. The energy density of the nutritional compositions, when in liquid form, can range from about 0.6 Kcal to about 3 Kcal per mL. In some embodiments, the nutritional composition is in solid or powdered form. When in solid or powdered form, the nutritional supplements can contain from about 1.2 to more than 9 Kcals per gram, such as about 3 to 7 Kcals per gram.

In some embodiments, the nutritional composition is a meal replacement bar. Examples include PowerBar®, Glucerna® bars, Choice DM® bars, Ensure® bars, and Boost® bars. In some embodiments, the nutritional composition is a nutrition shake or meal replacement beverage. Commercially available examples include the Ensure® branded adult products (such as Ensure® Original, Ensure® Plus, Ensure® Enlive, Ensure® High Protein, Ensure® Clear, and Ensure® Light), Glucerna®, Choice DM®, Slim Fast®, Pediasure®, Glytrol®, and Resource®. In some embodiments, the nutritional composition is Ensure® Plus. In some embodiments, the nutritional composition is vanilla-flavored Ensure® Plus. Ensure Plus® is a high calorie liquid dietary supplement that contains 1500 calories per liter with a caloric distribution of 14.7% protein, 32% fat and 53.3% carbohydrate.

In some embodiments of the disclosed methods, the compound of Formula (I), or a pharmaceutically acceptable salt thereof is administered to the subject with 8 fluid ounces (237 mL) of Ensure® Plus. In some embodiments, the Ensure® Plus is vanilla-flavored.

In some embodiments of the methods, the compound of Formula (I), or a pharmaceutically acceptable salt thereof is administered to the subject after administration of the nutritional composition. In some embodiments, the compound of Formula (I), or a pharmaceutically acceptable salt thereof is administered to the subject before administration of the nutritional composition. In some embodiments, the compound of Formula (I), or a pharmaceutically acceptable salt thereof, is administered to the subject at the same time as administration of the nutritional composition.

In some embodiments, the compound of Formula (I), or a pharmaceutically acceptable salt thereof is administered to the subject, followed by administration of the nutritional composition. In some embodiments, the nutritional composition is administered about 1 minute, about 5 minutes, about 10 minutes, about 15 minutes, about 20 minutes, about 25 minutes, about 30 minutes, about 35 minutes, about 40 minutes, about 45 minutes, or about 60 minutes, or within a range defined by any of the preceding values after administration of the compound of Formula (I), or a pharmaceutically acceptable salt thereof. In some embodiments, the nutritional composition is administered 1 minute, 5 minutes, 10 minutes, 15 minutes, 20 minutes, 25 minutes, 30 minutes, 35 minutes, 40 minutes, 45 minutes, or 60 minutes, or within a range defined by any of the preceding values after administration of the compound of Formula (I), or a pharmaceutically acceptable salt thereof. In some embodiments, the nutritional composition is administered within 30 minutes of administering the compound of Formula (I), or a pharmaceutically acceptable salt thereof. In some embodiments of the disclosed methods, the compound of Formula (I), or a pharmaceutically acceptable salt thereof, is administered to the subject with 8 fluid ounces (237 mL) of Ensure® Plus. In some embodiments, the Ensure® Plus is vanilla-flavored.

In some embodiments, the nutritional composition is administered to the subject, followed by administration of the compound of Formula (I), or a pharmaceutically acceptable salt thereof. In some embodiments, compound of Formula (I), or a pharmaceutically acceptable salt thereof is administered about 1 minute, about 5 minutes, about 10 minutes, about 15 minutes, about 20 minutes, about 25 minutes, about 30 minutes, about 35 minutes, about 40 minutes, about 45 minutes, or about 60 minutes, or within a range defined by any of the preceding values after administration of the nutritional composition. In some embodiments, the compound of Formula (I), or a pharmaceutically acceptable salt thereof, is administered 1 minute, 5 minutes, 10 minutes, 15 minutes, 20 minutes, 25 minutes, 30 minutes, 35 minutes, 40 minutes, 45 minutes, or 60 minutes, or within a range defined by any of the preceding values after administration of the nutritional composition. In some embodiments, the compound of Formula (I), or a pharmaceutically acceptable salt thereof, is administered within 30 minutes of administering the nutritional composition. In some embodiments of the disclosed methods, the compound of Formula (I), or a pharmaceutically acceptable salt thereof, is administered to the subject with 8 fluid ounces (237 mL) of Ensure® Plus. In some embodiments, the Ensure® Plus is vanilla-flavored.

In some embodiments of the methods, a food effect is observed between administration of the compound of Formula (I), or a pharmaceutically acceptable salt thereof in a fed state versus a fasted state. The term “food effect,” as used herein, refers to the relative difference in AUC (area under the curve AUC₍₀₋ₜ₎ and/or AUC_{(0-∞)}) or Cₘₐₓ (maximum plasma concentration or peak plasma concentration) of an active substance, when the compound of Formula (I), or a pharmaceutically acceptable salt thereof, is administered orally to a subject, concomitantly with food or in a fed state as compared to the same values when the same compound of Formula (I), or a pharmaceutically acceptable salt thereof is administered in a fasted state. The food effect (F) is calculated as:

F %=[(X_{fasted}−X_{fed})/X_{fasted}]×100

where X_{fed} and X_{fasted} are the values of AUC (AUC₍₀₋ₜ₎ and/or AUC_{(0-∞)}) or Cₘₐₓ in the fed and fasted state, respectively. In some embodiments, an increased, or positive, food effect is observed when the compound of Formula (I), or a pharmaceutically acceptable salt thereof is administered to a subject in a fed state. In some embodiments, administration of the compound of Formula (I), or a pharmaceutically acceptable salt thereof, results in an increased, or positive, food effect, whereby an increased Cₘₐₓ and/or AUC are observed when administered orally in the fed state as compared to the fasting state.

In some embodiments of the methods, the ratio of the AUC in the fed state to the AUC in the fasted state is about 5 to about 10, such as about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 8 to about 10. In some embodiments, the ratio of the AUC in the fed state to the AUC in the fasted state is about 5, about 6, about 7, about 8, about 9, or about 10, or within a range defined by any of the preceding values. In some embodiments of the methods, the ratio of the AUC in the fed state to the AUC in the fasted state is about 10 to about 20.

In some embodiments of the methods, the ratio of the AUC in the fed state to the AUC in the fasted state is 5 to 10, such as 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 8 to 10. In some embodiments, the ratio of the AUC in the fed state to the AUC in the fasted state is 5, 6, 7, 8, 9, or 10, or within a range defined by any of the preceding values.

In some embodiments of the methods, the ratio of the Cₘₐₓ in the fed state to the Cₘₐₓ in the fasted state is about 5 to about 10, such as about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 8 to about 10. In some embodiments, the ratio of the Cₘₐₓ in the fed state to the Cₘₐₓ in the fasted state is about 5, about 6, about 7, about 8, about 9, or about 10, or within a range defined by any of the preceding values. In some embodiments, the mean Cₘₐₓ of the compound of Formula (I), or pharmaceutically acceptable salt thereof, is about 1.5 to about 3 times higher in the fed stated compared to the fasted state.

In some embodiments of the methods, the ratio of the Cₘₐₓ in the fed state to the Cₘₐₓ in the fasted state is 5 to 10, such as 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 8 to 10. In some embodiments, the ratio of the Cₘₐₓ in the fed state to the Cₘₐₓ in the fasted state is 5, 6, 7, 8, 9, or 10, or within a range defined by any of the preceding values. In some embodiments, the mean Cₘₐₓ of the compound of Formula (I), or pharmaceutically acceptable salt thereof, is 1.5 to 3 times higher in the fed stated compared to the fasted state. In some embodiments, the mean Cₘₐₓ of the compound of Formula (I), or pharmaceutically acceptable salt thereof, is about 2 times higher in the fed stated compared to the fasted state. In some embodiments of the methods, the ratio of the Cₘₐₓ in the fed state to the Cₘₐₓ in the fasted state is about 10 to about 20.

In some embodiments, the compound of Formula (I), or a pharmaceutically acceptable salt thereof, is administered to the subject with a meal. In some embodiments, the meal is a high fat, high caloric meal. In some embodiments, the meal is a low fat, low caloric meal. In some embodiments, the compound of Formula (I), or a pharmaceutically acceptable salt thereof, is administered within approximately 5 minutes after the start of the meal. In some embodiments, the meal is an evening meal. In some embodiments, the meal is a morning meal. In some embodiments, the meal is completed within about 30 minutes after administration of the compound of Formula (I), or a pharmaceutically acceptable salt thereof.

In some embodiments of the methods disclosed herein (e.g., when the compound of Formula (I) is administered at a frequency of twice daily), the first administration of the compound of Formula (I), or a pharmaceutically acceptable salt thereof is with a morning meal. In some embodiments of the methods disclosed herein, the second administration of the compound of Formula (I), or a pharmaceutically acceptable salt thereof is with an evening meal. In some embodiments of the methods disclosed herein, the first administration of the compound of Formula (I), or a pharmaceutically acceptable salt thereof, is with a morning meal and the second administration of the compound of Formula (I), or a pharmaceutically acceptable salt thereof is with an evening meal. In some embodiments of the methods disclosed herein (e.g., when the compound of Formula (I) is administered at a frequency of twice daily), there are about 6 to about 14 hours between the morning and evening meals. In some embodiments, there are about 8 to about 14 hours between the morning and evening meals. In some embodiments, there are about 11 to about 13 hours between the morning and evening meals. In some embodiments, there are about 12 hours between the morning and evening meals.

In some embodiments, the fed state is with a high fat meal. In some embodiments, the fed state is with a low fat meal. The FDA has provided draft guidelines regarding high fat and low fat meals (“Assessing the Effects of Food on Drugs in INDs and NDAs—Clinical Pharmacology Considerations Guidance for Industry,” U.S. Department of Health and Human Services, Food and Drug Administration, Center for Drug Evaluation and Research (CDER), February 2019, Clinical Pharmacology). Table 1 shows test meal definitions provided by the FDA guidance.

TABLE 1

| | | Fat | | |
|---|---|---|---|---|
| Meal Type | Total Kcal | Kcal | Grams | Percent |
| High-Fat | 800-1000 | 500-600 | 55-65 | 50 |
| Low-Fat | 400-500 | 100-125 | 11-14 | 25 |

The composition of a high fat meal provided by the FDA guidance is depicted in Table 2.

TABLE 2
Composition of a High Fat Meal*

| | |
|---|---|
| Total Calories | 800-1000 |
| Calories from Protein | 150 |
| Calories from Carbohydrates | 250 |
| Calories from Fat | 500-600 |
| An Example of a High Fat | Two eggs fried in butter |
| Breakfast | Two strips of bacon |
| | Two slices of toast with butter |
| | Four ounces of hash brown potatoes |
| | Eight ounces of whole milk |

*50 percent of calories are derived from fat. Substitutions can be made to this meal, if the content, volume, and viscosity are maintained.

The composition of a low fat meal provided by the FDA guidance is depicted in Table 3.

TABLE 3
Composition of a Low Fat Meal

| | |
|---|---|
| Total Calories | 400-500 |
| Fat (g) | 250 |
| Percent Calories from Fat | 25 |
| An Example of a Low Fat | Eight ounces milk (1 percent fat) |
| Breakfast* | One boiled egg |
| | One packet flavored instant oatmeal |
| | made with water |

*This low-fat breakfast contains 387 calories and has 10 grams of fat.

In some embodiments, a high fat meal contains 800-1000 total Kcal and 500-600 fat Kcal. In some embodiments, a low fat meal contains 400-500 total Kcal and 100-125 fat Kcal.

Also provided herein is a method of improving gastrointestinal absorption of a compound of Formula (I), or pharmaceutically acceptable salt thereof, in a subject. The method includes orally administering to the subject a pharmaceutical composition of the present disclosure, wherein the improvement is relative to oral administration of the compound of Formula (I), or pharmaceutically acceptable salt thereof, which has not been prepared as a spray-dried dispersion. In some embodiments, the subject is a pediatric subject.

Also provided herein is a method of improving oral bioavailability of a compound of Formula (I), or pharmaceutically acceptable salt thereof, in a subject. The method includes orally administering to the subject a pharmaceutical composition of the present disclosure, wherein the improvement is relative to oral administration of the compound of Formula (I), or pharmaceutically acceptable salt thereof, which has not been prepared as a spray-dried dispersion.

In some embodiments of the methods provided herein, the subject is a pediatric subject.

Also provided herein is a method of treating congenital adrenal hyperplasia (CAH), in a subject in need thereof, comprising administering to the subject a pharmaceutical composition of the present disclosure, wherein the pharmaceutical composition comprises a therapeutically effective amount of the compound of Formula (I), or a pharmaceutically acceptable salt thereof. In some embodiments, the pharmaceutical composition is a lipidic semi-solid formulation. In some embodiments, the pharmaceutical composition is a liquid formulation. In some embodiments, the pharmaceutical composition is administered to the subject in a fed state.

Also provided herein is a pharmaceutical composition of the present disclosure for use in a method of treating congenital adrenal hyperplasia (CAH) in a subject. In some embodiments, the subject is in a fed state.

In some embodiments, the pharmaceutical composition is administered to the subject with a nutritional composition. In some embodiments, the nutritional composition is a liquid dietary supplement comprising about 1000 to about 2000 calories per liter with a fat content greater than about 30%. In some embodiments, the nutritional composition is a liquid dietary supplement comprising 1500 calories per liter with a caloric distribution of 14.7% protein, 32% fat and 53.3% carbohydrate. In some embodiments, the nutritional composition is administered in an amount of about 6 to about 12 fluid ounces. In some embodiments, the nutritional composition is administered in an amount of about 8 fluid ounces. In some embodiments, the nutritional composition is administered within 30 minutes of administration of the pharmaceutical composition.

In some embodiments, the pharmaceutical composition exhibits a positive food effect. In some embodiments, the positive food effect is measured in terms of Cₘₐₓ, AUC, or a combination thereof of a compound of Formula (I) when comparing oral administration of the pharmaceutical composition in the fed and fasting states. In some embodiments, the ratio of the AUC of the compound of Formula (I) in the fed state to the AUC of the compound of Formula (I) in the fasted state is about 5 to about 10. In some embodiments, the ratio of the Cₘₐₓ of the compound of Formula (I) in the fed state to the Cₘₐₓ of the compound of Formula (I) in the fasted state is about 5 to about 10. In some embodiments, the ratio of the AUC of the compound of Formula (I) in the fed state to the AUC of the compound of Formula (I) in the fasted state is about 10 to about 20. In some embodiments, the ratio of the Cₘₐₓ of the compound of Formula (I) in the fed state to the Cₘₐₓ of the compound of Formula (I) in the fasted state is about 10 to about 20. In some embodiments, the ratio of the AUC of the compound of Formula (I) in the fed state to the AUC of the compound of Formula (I) in the fasted state is about 1 to about 4 or about 5 to about 10. In some embodiments, the ratio of the Cₘₐₓ of the compound of Formula (I) in the fed state to the Cₘₐₓ of the compound of Formula (I) in the fasted state is about 1 to about 4 or about 5 to about 10. In some embodiments, the ratio of the AUC of the compound of Formula (I) in the fed state to the AUC of the compound of Formula (I) in the fasted state is about 1 to about 4. In some embodiments, the ratio of the Cₘₐₓ of the compound of Formula (I) in the fed state to the Cₘₐₓ of the compound of Formula (I) in the fasted state is about 1 to about 4. In some embodiments, the ratio of the AUC of the compound of Formula (I) in the fed state to the AUC of the compound of Formula (I) in the fasted state is about 1.5 to about 3. In some embodiments, the ratio of the Cₘₐₓ of the compound of Formula (I) in the fed state to the Cₘₐₓ of the compound of Formula (I) in the fasted state is about 1.5 to about 3. In some embodiments, the ratio of the AUC of the compound of Formula (I) in the fed state to the AUC of the compound of Formula (I) in the fasted state is 1 to 4 or 5 to 10. In some embodiments, the ratio of the Cₘₐₓ of the compound of Formula (I) in the fed state to the Cₘₐₓ of the compound of Formula (I) in the fasted state is 1 to 4 or 5 to 10. In some embodiments, the ratio of the AUC of the compound of Formula (I) in the fed state to the AUC of the compound of Formula (I) in the fasted state is 1 to 4. In some embodiments, the ratio of the Cₘₐₓ of the compound of Formula (I) in the fed state to the Cₘₐₓ of the compound of Formula (I) in the fasted state is 1 to 4. In some embodiments, the ratio of the AUC of the compound of Formula (I) in the fed state to the AUC of the compound of Formula (I) in the fasted state is 1.5 to 3. In some embodiments, the ratio of the Cₘₐₓ of the compound of Formula (I) in the fed state to the Cₘₐₓ of the compound of Formula (I) in the fasted state is 1.5 to 3.

In some embodiments, the subject is a pediatric subject.

In some embodiments, the pharmaceutical composition is formulated for oral administration and exhibits a positive food effect when administered orally. In some embodiments, the compound of Formula (I) has a ratio of the AUC in the fed state to the AUC in the fasted state of about 5 to about 10. In some embodiments, the compound of Formula (I) has a ratio of the Cₘₐₓ in the fed state to the Cₘₐₓ in the fasted state of about 5 to about 10. In some embodiments, the compound of Formula (I) has a ratio of the AUC in the fed state to the AUC in the fasted state of about 10 to about 20. In some embodiments, the compound of Formula (I) has a ratio of the Cₘₐₓ in the fed state to the Cₘₐₓ in the fasted state of about 10 to about 20. In some embodiments, the compound of Formula (I) has a ratio of the AUC in the fed state to the AUC in the fasted state of about 1 to about 4 or about 5 to about 10. In some embodiments, the compound of Formula (I) has a ratio of the Cₘₐₓ in the fed state to the Cₘₐₓ in the fasted state of about 1 to about 4 or about 5 to about 10. In some embodiments, the compound of Formula (I) has a ratio of the AUC in the fed state to the AUC in the fasted state of about 1 to about 4. In some embodiments, the compound of Formula (I) has a ratio of the Cₘₐₓ in the fed state to the Cₘₐₓ in the fasted state of about 1 to about 4. In some embodiments, the compound of Formula (I) has a ratio of the AUC in the fed state to the AUC in the fasted state of about 1.5 to about 3. In some embodiments, the compound of Formula (I) has a ratio of the Cₘₐₓ in the fed state to the Cₘₐₓ in the fasted state of about 1.5 to about 3. In some embodiments, the compound of Formula (I) has a ratio of the AUC in the fed state to the AUC in the fasted state of 1 to 4 or 5 to 10. In some embodiments, the compound of Formula (I) has a ratio of the Cₘₐₓ in the fed state to the Cₘₐₓ in the fasted state of 1 to 4 or 5 to 10. In some embodiments, the compound of Formula (I) has a ratio of the AUC in the fed state to the AUC in the fasted state of 1 to 4. In some embodiments, the compound of Formula (I) has a ratio of the Cₘₐₓ in the fed state to the Cₘₐₓ in the fasted state of 1 to 4. In some embodiments, the compound of Formula (I) has a ratio of the AUC in the fed state to the AUC in the fasted state of 1.5 to 3. In some embodiments, the compound of Formula (I) has a ratio of the Cₘₐₓ in the fed state to the Cₘₐₓ in the fasted state of 1.5 to 3.

In some embodiments, the pharmaceutical composition is administered to the subject with a meal. In some embodiments, the meal is a high fat meal. In some embodiments, the meal is a low fat meal. In some embodiments, the pharmaceutical composition is administered within about 5 minutes after the start of the meal. In some embodiments, the meal is an evening meal. In some embodiments, the meal is a morning meal.

In some embodiments, administering the pharmaceutical composition exhibits a positive food effect. In some embodiments, the positive food effect is measured in terms of Cₘₐₓ, AUC, or combinations thereof of the compound of Formula (I) when comparing oral administration of the pharmaceutical composition in the fed and fasting states. In some embodiments, the ratio of the AUC of the compound of Formula (I) in the fed state to the AUC of the compound of Formula (I) in the fasted state is about 5 to about 10. In some embodiments, the ratio of the Cₘₐₓ of the compound of Formula (I) in the fed state to the Cₘₐₓ of the compound of Formula (I) in the fasted state is about 5 to about 10. In some embodiments, the ratio of the AUC of the compound of Formula (I) in the fed state to the AUC of the compound of Formula (I) in the fasted state is about 10 to about 20. In some embodiments, the ratio of the Cₘₐₓ of the compound of Formula (I) in the fed state to the Cₘₐₓ of the compound of Formula (I) in the fasted state is about 10 to about 20. In some embodiments, the ratio of the AUC of the compound of Formula (I) in the fed state to the AUC of the compound of Formula (I) in the fasted state is about 1 to about 4 or about 5 to about 10. In some embodiments, the ratio of the Cₘₐₓ of the compound of Formula (I) in the fed state to the Cₘₐₓ of the compound of Formula (I) in the fasted state is about 1 to about 4 or about 5 to about 10. In some embodiments, the ratio of the AUC of the compound of Formula (I) in the fed state to the AUC of the compound of Formula (I) in the fasted state is about 1 to about 4. In some embodiments, the ratio of the Cₘₐₓ of the compound of Formula (I) in the fed state to the Cₘₐₓ of the compound of Formula (I) in the fasted state is about 1 to about 4. In some embodiments, the ratio of the AUC of the compound of Formula (I) in the fed state to the AUC of the compound of Formula (I) in the fasted state is about 1.5 to about 3. In some embodiments, the ratio of the Cₘₐₓ of the compound of Formula (I) in the fed state to the Cₘₐₓ of the compound of Formula (I) in the fasted state is about 1.5 to about 3. In some embodiments, the ratio of the AUC of the compound of Formula (I) in the fed state to the AUC of the compound of Formula (I) in the fasted state is 1 to 4 or 5 to 10. In some embodiments, the ratio of the Cₘₐₓ of the compound of Formula (I) in the fed state to the Cₘₐₓ of the compound of Formula (I) in the fasted state is 1 to 4 or 5 to 10. In some embodiments, the ratio of the AUC of the compound of Formula (I) in the fed state to the AUC of the compound of Formula (I) in the fasted state is 1 to 4. In some embodiments, the ratio of the Cₘₐₓ of the compound of Formula (I) in the fed state to the Cₘₐₓ of the compound of Formula (I) in the fasted state is 1 to 4. In some embodiments, the ratio of the AUC of the compound of Formula (I) in the fed state to the AUC of the compound of Formula (I) in the fasted state is 1.5 to 3. In some embodiments, the ratio of the Cₘₐₓ of the compound of Formula (I) in the fed state to the Cₘₐₓ of the compound of Formula (I) in the fasted state is 1.5 to 3.

For the avoidance of doubt, also provided herein is the corresponding compound of Formula (I), or a pharmaceutically acceptable salt thereof, or corresponding pharmaceutical composition comprising a compound of Formula (I), or a pharmaceutically acceptable salt thereof, for use in the corresponding methods, as described herein.

For the avoidance of doubt, also provided herein is use of the corresponding compound of Formula (I), or a pharmaceutically acceptable salt thereof, in the manufacture of a medicament for use in the corresponding methods, as described herein.

For the avoidance of doubt, also provided herein is use of the corresponding pharmaceutical composition comprising a compound of Formula (I), or a pharmaceutically acceptable salt thereof, in the manufacture of a medicament for use in the corresponding methods, as described herein.

### Reduction in Glucocorticoid Burden, Adrenal Androgens and Precursors

Glucocorticoids are a class of corticosteroids, which are a class of steroid hormones. Glucocorticoids are corticosteroids that bind to the glucocorticoid receptor that is present in almost every vertebrate animal cell. In some embodiments, the subject is concurrently receiving a dose of a glucocorticoid. In some embodiments, the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate. In some embodiments, the glucocorticoid is cortisol (hydrocortisone). In some embodiments, the glucocorticoid is cortisone. In some embodiments, the glucocorticoid is prednisone. In some embodiments, the glucocorticoid is dexamethasone.

In some embodiments, the glucocorticoid dose is measured in hydrocortisone equivalents. In some embodiments, the glucocorticoid dose is measured as a multiple of the upper limit of normal of physiologic dosing in hydrocortisone equivalents. Any glucocorticoid can be given in a dose that provides approximately the same glucocorticoid effects as normal cortisol production; this is referred to as physiologic, replacement, or maintenance dosing.

In some embodiments, the glucocorticoid dose is a physiologic dose as measured after a time period of administration of the compound of Formula (I), or a pharmaceutically acceptable salt thereof. In some embodiments, the glucocorticoid dose is a physiologic dose of about 4 to about 12 mg/m²/day as measured after a time period of administration of the compound of Formula (I), or a pharmaceutically acceptable salt thereof. In some embodiments, the glucocorticoid dose is a physiologic dose of about 4 to about 9 mg/m²/day as measured after a time period of administration of the compound of Formula (I), or a pharmaceutically acceptable salt thereof. In some embodiments, the glucocorticoid dose is a physiologic dose that is less than about 8 mg/m²/day as measured after a time period of administration of the compound of Formula (I), or a pharmaceutically acceptable salt thereof.

In some embodiments, the glucocorticoid dose is a physiologic dose as measured after a time period of administration of the pharmaceutical composition comprising the compound of Formula (I), or a pharmaceutically acceptable salt thereof. In some embodiments, the glucocorticoid dose is a physiologic dose of about 4 to about 12 mg/m²/day as measured after a time period of administration of the pharmaceutical composition comprising the compound of Formula (I), or a pharmaceutically acceptable salt thereof. In some embodiments, the glucocorticoid dose is a physiologic dose of about 4 to about 9 mg/m²/day as measured after a time period of administration of the pharmaceutical composition comprising the compound of Formula (I), or a pharmaceutically acceptable salt thereof. In some embodiments, the glucocorticoid dose is a physiologic dose that is less than about 8 mg/m²/day as measured after a time period of administration of the pharmaceutical composition comprising the compound of Formula (I), or a pharmaceutically acceptable salt thereof.

In some embodiments, the glucocorticoid dose concurrently given to the subject is a normal physiological dose of hydrocortisone equivalents. In some embodiments, the glucocorticoid dose concurrently given to the subject is determined after a time period of administration of the compound of Formula (I), or a pharmaceutically acceptable salt thereof. In some embodiments, the glucocorticoid dose concurrently given to the subject is determined after a time period of administration of the pharmaceutical composition comprising the compound of Formula (I), or a pharmaceutically acceptable salt thereof. In some embodiments, a normal physiological dose of hydrocortisone equivalents is about 2 to about 16 mg/m²/day. In some embodiments, a normal physiological dose of hydrocortisone equivalents is about 4 to about 12 mg/m²/day. In some embodiments, a normal physiological dose of hydrocortisone equivalents is about 5 to about 11 mg/m²/day. In some embodiments, a normal physiological dose of hydrocortisone equivalents is about 6 to about 10 mg/m²/day. In some embodiments, a normal physiological dose of hydrocortisone equivalents is about 7 to about 9 mg/m²/day. In some embodiments, a normal physiological dose of hydrocortisone equivalents is about 4 to about 9 mg/m²/day. In some embodiments, a normal physiological dose of hydrocortisone equivalents is about 8 mg/m²/day. In some embodiments, a normal physiological dose of hydrocortisone equivalents is about 12 mg/m²/day. In some embodiments, a normal physiological dose of hydrocortisone equivalents is less than about 8 mg/m²/day. In some embodiments, a normal physiological dose of hydrocortisone equivalents is about 2, about 3, about 4, about 5, about 6, about 7, about 8, about 9, about 10, about 11, about 12, about 13, about 14, about 15 or about 16 mg/m²/day, or within a range defined by any of the preceding values.

In some embodiments, the glucocorticoid dose concurrently given to the subject is at the upper limit of normal of a normal physiological dose of hydrocortisone equivalents. In some embodiments, the glucocorticoid dose concurrently given to the subject is determined after a time period of administration of the compound of Formula (I), or a pharmaceutically acceptable salt thereof. In some embodiments, the glucocorticoid dose concurrently given to the subject is determined after a time period of administration of the pharmaceutical composition comprising the compound of Formula (I), or a pharmaceutically acceptable salt thereof. In some embodiments, the upper limit of normal is 1.5 times the normal physiological dose. In some embodiments, the upper limit of normal is about 1.5 times the normal physiological dose. In some embodiments, the upper limit of normal is about 1.5 times the normal physiological dose. In some embodiments, the upper limit of normal is about 2 times the normal physiological dose. In some embodiments, the upper limit of normal is about 2.5 times the normal physiological dose. In some embodiments, the upper limit of normal is about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 times the normal physiological dose, or within a range defined by any of the preceding values.

In some embodiments, the glucocorticoid dose of the subject is reduced by about 10% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 30% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 40% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 60% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 70% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by less than about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 20% to about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by greater than about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

In some embodiments, the glucocorticoid dose of the subject is reduced by about 10% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 20% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 30% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 40% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 50% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 60% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 70% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by less than about 20% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 20% to about 50% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by greater than about 50% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof.

In some embodiments, the glucocorticoid dose of the subject is reduced within a range defined by any of the preceding values.

In some embodiments, the level of 17-hydroxyprogesterone is reduced by at least 25% after a time period of administration of the compound of Formula (I), or a pharmaceutically acceptable salt thereof, wherein the reduction of the level of 17-hydroxyprogesterone is relative to the level of 17-hydroxyprogesterone prior to administration of the compound of Formula (I), or a pharmaceutically acceptable salt thereof. In some embodiments, the level of 17-hydroxyprogesterone is reduced by at least 50% after a time period of administration of the compound of Formula (I), or a pharmaceutically acceptable salt thereof, wherein the reduction of the level of 17-hydroxyprogesterone is relative to the level of 17-hydroxyprogesterone prior to administration of the compound of Formula (I), or a pharmaceutically acceptable salt thereof. In some embodiments, the level of 17-hydroxyprogesterone is less than 1.5 times the upper limit of normal after a time period of administration of the compound of Formula (I), or a pharmaceutically acceptable salt thereof. In some embodiments, the level of 17-hydroxyprogesterone is within normal limits after a time period of administration of the compound of Formula (I), or a pharmaceutically acceptable salt thereof.

In some embodiments, the level of 17-hydroxyprogesterone is reduced by at least about 25% after a time period of administration of the pharmaceutical composition comprising the compound of Formula (I), or a pharmaceutically acceptable salt thereof, wherein the reduction of the level of 17-hydroxyprogesterone is relative to the level of 17-hydroxyprogesterone prior to administration of the pharmaceutical composition comprising the compound of Formula (I), or a pharmaceutically acceptable salt thereof. In some embodiments, the level of 17-hydroxyprogesterone is reduced by at least about 50% after a time period of administration of the pharmaceutical composition comprising the compound of Formula (I), or a pharmaceutically acceptable salt thereof, wherein the reduction of the level of 17-hydroxyprogesterone is relative to the level of 17-hydroxyprogesterone prior to administration of the pharmaceutical composition comprising the compound of Formula (I), or a pharmaceutically acceptable salt thereof. In some embodiments, the level of 17-hydroxyprogesterone is less than about 1.5 times the upper limit of normal after a time period of administration of the pharmaceutical composition comprising the compound of Formula (I), or a pharmaceutically acceptable salt thereof. In some embodiments, the level of 17-hydroxyprogesterone is within normal limits after a time period of administration of the pharmaceutical composition comprising the compound of Formula (I), or a pharmaceutically acceptable salt thereof.

In some embodiments, the level of 17-hydroxyprogesterone of the subject is reduced within a range defined by any of the preceding values.

In some embodiments, the level of adrenocorticotropic hormone is reduced by at least 25% after a time period of administration of the compound of Formula (I), or a pharmaceutically acceptable salt thereof, wherein the reduction of the level of adrenocorticotropic hormone is relative to the level of adrenocorticotropic hormone prior to administration of the compound of Formula (I), or a pharmaceutically acceptable salt thereof. In some embodiments, the level of adrenocorticotropic hormone is reduced by at least 40% after a time period of administration of the compound of Formula (I), or a pharmaceutically acceptable salt thereof, wherein the reduction of the level of adrenocorticotropic hormone is relative to the level of adrenocorticotropic hormone prior to administration of the compound of Formula (I), or a pharmaceutically acceptable salt thereof. In some embodiments, the level of adrenocorticotropic hormone is reduced by at least 50% after a time period of administration of the compound of Formula (I), or a pharmaceutically acceptable salt thereof, wherein the reduction of the level of adrenocorticotropic hormone is relative to the level of adrenocorticotropic hormone prior to administration of the compound of Formula (I), or a pharmaceutically acceptable salt thereof. In some embodiments, the level of adrenocorticotropic hormone is less than 1.5 times the upper limit of normal after a time period of administration of the compound of Formula (I), or a pharmaceutically acceptable salt thereof. In some embodiments, the level of adrenocorticotropic hormone is within normal limits after a time period of administration of the compound of Formula (I), or a pharmaceutically acceptable salt thereof.

In some embodiments, the level of adrenocorticotropic hormone is reduced by at least about 25% after a time period of administration of the pharmaceutical composition comprising the compound of Formula (I), or a pharmaceutically acceptable salt thereof, wherein the reduction of the level of adrenocorticotropic hormone is relative to the level of adrenocorticotropic hormone prior to administration of the pharmaceutical composition comprising the compound of Formula (I), or a pharmaceutically acceptable salt thereof. In some embodiments, the level of adrenocorticotropic hormone is reduced by at least about 40% after a time period of administration of the pharmaceutical composition comprising the compound of Formula (I), or a pharmaceutically acceptable salt thereof, wherein the reduction of the level of adrenocorticotropic hormone is relative to the level of adrenocorticotropic hormone prior to administration of the pharmaceutical composition comprising the compound of Formula (I), or a pharmaceutically acceptable salt thereof. In some embodiments, the level of adrenocorticotropic hormone is reduced by at least about 50% after a time period of administration of the pharmaceutical composition comprising the compound of Formula (I), or a pharmaceutically acceptable salt thereof, wherein the reduction of the level of adrenocorticotropic hormone is relative to the level of adrenocorticotropic hormone prior to administration of the pharmaceutical composition comprising the compound of Formula (I), or a pharmaceutically acceptable salt thereof. In some embodiments, the level of adrenocorticotropic hormone is less than about 1.5 times the upper limit of normal after a time period of administration of the pharmaceutical composition comprising the compound of Formula (I), or a pharmaceutically acceptable salt thereof. In some embodiments, the level of adrenocorticotropic hormone is within normal limits after a time period of administration of the pharmaceutical composition comprising the compound of Formula (I), or a pharmaceutically acceptable salt thereof.

In some embodiments, the level of adrenocorticotropic hormone of the subject is reduced within a range defined by any of the preceding values.

In some embodiments, the level of androstenedione is reduced by at least 25% after a time period of administration of the compound of Formula (I), or a pharmaceutically acceptable salt thereof, wherein the reduction of the level of androstenedione is relative to the level of androstenedione prior to administration of the compound of Formula (I), or a pharmaceutically acceptable salt thereof. In some embodiments, the level of androstenedione is reduced by at least 30% after a time period of administration of the compound of Formula (I), or a pharmaceutically acceptable salt thereof, wherein the reduction of the level of androstenedione is relative to the level of androstenedione prior to administration of the compound of Formula (I), or a pharmaceutically acceptable salt thereof. In some embodiments, the level of androstenedione is reduced by at least 50% after a time period of administration of the compound of Formula (I), or a pharmaceutically acceptable salt thereof, wherein the reduction of the level of androstenedione is relative to the level of androstenedione prior to administration of the compound of Formula (I), or a pharmaceutically acceptable salt thereof. In some embodiments, the level of androstenedione is less than 1.5 times the upper limit of normal after a time period of administration of the compound of Formula (I), or a pharmaceutically acceptable salt thereof. In some embodiments, the level of androstenedione is within normal limits after a time period of administration of the compound of Formula (I), or a pharmaceutically acceptable salt thereof.

In some embodiments, the level of androstenedione is reduced by at least about 25% after a time period of administration of the pharmaceutical composition comprising the compound of Formula (I), or a pharmaceutically acceptable salt thereof, wherein the reduction of the level of androstenedione is relative to the level of androstenedione prior to administration of the pharmaceutical composition comprising the compound of Formula (I), or a pharmaceutically acceptable salt thereof. In some embodiments, the level of androstenedione is reduced by at least about 30% after a time period of administration of the pharmaceutical composition comprising the compound of Formula (I), or a pharmaceutically acceptable salt thereof, wherein the reduction of the level of androstenedione is relative to the level of androstenedione prior to administration of the pharmaceutical composition comprising the compound of Formula (I), or a pharmaceutically acceptable salt thereof. In some embodiments, the level of androstenedione is reduced by at least about 50% after a time period of administration of the pharmaceutical composition comprising the compound of Formula (I), or a pharmaceutically acceptable salt thereof, wherein the reduction of the level of androstenedione is relative to the level of androstenedione prior to administration of the pharmaceutical composition comprising the compound of Formula (I), or a pharmaceutically acceptable salt thereof. In some embodiments, the level of androstenedione is less than about 1.5 times the upper limit of normal after a time period of administration of the pharmaceutical composition comprising the compound of Formula (I), or a pharmaceutically acceptable salt thereof. In some embodiments, the level of androstenedione is within normal limits after a time period of administration of the pharmaceutical composition comprising the compound of Formula (I), or a pharmaceutically acceptable salt thereof.

In some embodiments, the level of androstenedione of the subject is reduced within a range defined by any of the preceding values.

In some embodiments, the level of testosterone is reduced by at least 25% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof. In some embodiments, the level of testosterone is reduced by at least 30% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof. In some embodiments, the level of testosterone is reduced by at least 50% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof. In some embodiments, the level of testosterone is less than 1.5 times the upper limit of normal after a time period of administration of the compound of Formula (I), or a pharmaceutically acceptable salt thereof. In some embodiments, the level of testosterone is within normal limits after a time period of administration of the compound of Formula (I), or a pharmaceutically acceptable salt thereof.

In some embodiments, the level of testosterone is reduced by at least about 25% after a time period of administration of the pharmaceutical composition comprising the compound of Formula (I), or a pharmaceutically acceptable salt thereof, wherein the reduction of the level of testosterone is relative to the level of testosterone prior to administration of the pharmaceutical composition comprising the compound of Formula (I), or a pharmaceutically acceptable salt thereof. In some embodiments, the level of testosterone is reduced by at least about 30% after a time period of administration of the pharmaceutical composition comprising the compound of Formula (I), or a pharmaceutically acceptable salt thereof, wherein the reduction of the level of testosterone is relative to the level of testosterone prior to administration of the pharmaceutical composition comprising the compound of Formula (I), or a pharmaceutically acceptable salt thereof. In some embodiments, the level of testosterone is reduced by at least about 50% after a time period of administration of the pharmaceutical composition comprising the compound of Formula (I), or a pharmaceutically acceptable salt thereof, wherein the reduction of the level of testosterone is relative to the level of testosterone prior to administration of the pharmaceutical composition comprising the compound of Formula (I), or a pharmaceutically acceptable salt thereof. In some embodiments, the level of testosterone is less than about 1.5 times the upper limit of normal after a time period of administration of the pharmaceutical composition comprising the compound of Formula (I), or a pharmaceutically acceptable salt thereof. In some embodiments, the level of testosterone is within normal limits after a time period of administration of the pharmaceutical composition comprising the compound of Formula (I), or a pharmaceutically acceptable salt thereof.

In some embodiments, the level of testosterone of the subject is reduced within a range defined by any of the preceding values.

In some embodiments, the level of 17-hydroxyprogesterone is reduced by at least 50% and the level of androstenedione is reduced by at least 50% after a time period of administration of the compound of Formula (I), or a pharmaceutically acceptable salt thereof, wherein the reduction of the level of 17-hydroxyprogesterone and the level of androstenedione is relative to the level of 17-hydroxyprogesterone and the level of androstenedione prior to administration of the compound of Formula (I), or a pharmaceutically acceptable salt thereof. In some embodiments, the level of 17-hydroxyprogesterone is less than 1.5 times the upper limit of normal and the level of androstenedione is less than 1.5 times the upper limit of normal after a time period of administration of the compound of Formula (I), or a pharmaceutically acceptable salt thereof. In some embodiments, the level of 17-hydroxyprogesterone is within normal limits and the level of androstenedione is within normal limits after a time period of administration of the compound of Formula (I), or a pharmaceutically acceptable salt thereof.

In some embodiments, the level of 17-hydroxyprogesterone is reduced by at least about 50% and the level of androstenedione is reduced by at least about 50% after a time period of administration of the pharmaceutical composition comprising the compound of Formula (I), or a pharmaceutically acceptable salt thereof, wherein the reduction of the level of 17-hydroxyprogesterone and the level of androstenedione is relative to the level of 17-hydroxyprogesterone and the level of androstenedione prior to administration of the pharmaceutical composition comprising the compound of Formula (I), or a pharmaceutically acceptable salt thereof. In some embodiments, the level of 17-hydroxyprogesterone is less than about 1.5 times the upper limit of normal and the level of androstenedione is less than about 1.5 times the upper limit of normal after a time period of administration of the pharmaceutical composition comprising the compound of Formula (I), or a pharmaceutically acceptable salt thereof. In some embodiments, the level of 17-hydroxyprogesterone is within normal limits and the level of androstenedione is within normal limits after a time period of administration of the pharmaceutical composition comprising the compound of Formula (I), or a pharmaceutically acceptable salt thereof.

In some embodiments, the level of 17-hydroxyprogesterone and androstenedione of the subject is reduced within a range defined by any of the preceding values.

In some embodiments, the subject exhibits a decrease in glucocorticoid burden after a time period of administration of the compound of Formula (I), or a pharmaceutically acceptable salt thereof, wherein the decrease in glucocorticoid burden is relative to the glucocorticoid burden prior to administration of the compound of Formula (I), or a pharmaceutically acceptable salt thereof. In some embodiments, one or more symptoms selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor (TART), control of ovarian adrenal rest tumors (OART) and fertility, is improved after a time period of administration of the compound of Formula (I), or a pharmaceutically acceptable salt thereof, wherein the improvement in the one or more symptoms is relative to the status of the one or more symptoms prior to administration of the compound of Formula (I), or a pharmaceutically acceptable salt thereof.

In some embodiments, the subject exhibits a decrease in glucocorticoid burden after a time period of administration of the pharmaceutical composition comprising the compound of Formula (I), or a pharmaceutically acceptable salt thereof, wherein the decrease in glucocorticoid burden is relative to the glucocorticoid burden prior to administration of the pharmaceutical composition comprising the compound of Formula (I), or a pharmaceutically acceptable salt thereof. In some embodiments, one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor (TART), control of ovarian adrenal rest tumor (OART), and fertility, is improved after a time period of administration of the pharmaceutical composition comprising the compound of Formula (I), or a pharmaceutically acceptable salt thereof, wherein the improvement in the one or more symptoms is relative to the status of the one or more symptoms prior to administration of the pharmaceutical composition comprising the compound of Formula (I), or a pharmaceutically acceptable salt thereof.

In some embodiments, the level of one or more adrenal steroids, or a precursor thereof, is reduced by at least 25% after a time period of administration of the compound of Formula (I), or a pharmaceutically acceptable salt thereof, wherein the reduction of the level of the adrenal steroid, or a precursor thereof, is relative to the level of adrenal steroid, or a precursor thereof, prior to administration of the compound of Formula (I), or a pharmaceutically acceptable salt thereof. In some embodiments, the level of adrenal steroid, or a precursor thereof, is reduced by at least 50% after a time period of administration of the compound of Formula (I), or a pharmaceutically acceptable salt thereof, wherein the reduction of the level of adrenal steroid, or a precursor thereof, is relative to the level of adrenal steroid, or a precursor thereof, prior to administration of the compound of Formula (I), or a pharmaceutically acceptable salt thereof. In some embodiments, the level of the adrenal steroid, or a precursor thereof, is less than 1.5 times the upper limit of normal after a time period of administration of the compound of Formula (I), or a pharmaceutically acceptable salt thereof. In some embodiments, the level of the adrenal steroid, or a precursor thereof, is within normal limits after a time period of administration of the compound of Formula (I), or a pharmaceutically acceptable salt thereof.

In some embodiments, the quality of life as measured by the EuroQol 5 Dimensions 5 Levels (EQ-5D-5L) in the subject is improved after a time period of administration of the compound of Formula (I), or a pharmaceutically acceptable salt thereof, wherein the improvement in the EuroQol 5 Dimensions 5 Levels (EQ-5D-5L) is relative to the EuroQol 5 Dimensions 5 Levels (EQ-5D-5L) results prior to administration of the compound of Formula (I), or a pharmaceutically acceptable salt thereof.

In some embodiments, fatigue is reduced in the subject after a time period of administration of the compound of Formula (I), or a pharmaceutically acceptable salt thereof, wherein the reduction in fatigue is relative to the fatigue prior to administration of the compound of Formula (I), or a pharmaceutically acceptable salt thereof.

In some embodiments, sleep is improved in the subject after a time period of administration of the compound of Formula (I), or a pharmaceutically acceptable salt thereof, wherein the improvement in sleep is relative to the sleep prior to administration of the compound of Formula (I), or a pharmaceutically acceptable salt thereof. An improvement in sleep can comprise one or more of reduction in latency to sleep onset, increase in total sleep time, and/or an improvement in sleep quality.

In some embodiments, insulin resistance is reduced in the subject after a time period of administration of the compound of Formula (I), or a pharmaceutically acceptable salt thereof, wherein the reduction of insulin resistance is relative to the insulin resistance prior to administration of the compound of Formula (I), or a pharmaceutically acceptable salt thereof.

In some embodiments, glucose tolerance (e.g., an impaired glucose tolerance) is improved in the subject after a time period of administration of the compound of Formula (I), or a pharmaceutically acceptable salt thereof, wherein the improvement in glucose tolerance is relative to the glucose tolerance prior to administration of the compound of Formula (I), or a pharmaceutically acceptable salt thereof.

In some embodiments, glucose control is increased in the subject after a time period of administration of the compound of Formula (I), or a pharmaceutically acceptable salt thereof, wherein the increase in glucose control is relative to the glucose control prior to administration of the compound of Formula (I), or a pharmaceutically acceptable salt thereof.

In some embodiments, lipid levels reflecting dyslipidemia are improved (e.g., reduced) in the subject after a time period of administration of the compound of Formula (I), or a pharmaceutically acceptable salt thereof, wherein the improvement in lipid levels is relative to the lipid levels prior to administration of the compound of Formula (I), or a pharmaceutically acceptable salt thereof.

In some embodiments, lipid levels reflecting hyperlipidemia are reduced in the subject after a time period of administration of the compound of Formula (I), or a pharmaceutically acceptable salt thereof, wherein the reduction in lipid levels is relative to the lipid levels prior to administration of the compound of Formula (I), or a pharmaceutically acceptable salt thereof.

In some embodiments, bone mineral density is increased in the subject after a time period of administration of the compound of Formula (I), or a pharmaceutically acceptable salt thereof, wherein the increase in bone mineral density is relative to the bone mineral density prior to administration of the compound of Formula (I), or a pharmaceutically acceptable salt thereof.

In some embodiments, bone turnover is improved (e.g., an increase in bone turnover markers consistent with a decrease in bone loss) in the subject after a time period of administration of the compound of Formula (I), or a pharmaceutically acceptable salt thereof, wherein the improvement in bone turnover is relative to the bone turnover prior to administration of the compound of Formula (I), or a pharmaceutically acceptable salt thereof.

In some embodiments, fat mass is decreased in the subject after a time period of administration of the compound of Formula (I), or a pharmaceutically acceptable salt thereof, wherein the decrease in fat mass is relative to the fat mass prior to administration of the compound of Formula (I), or a pharmaceutically acceptable salt thereof.

In some embodiments, body weight is decreased in the subject (e.g., in a subject who is overweight, obese, and/or exhibits central obesity) after a time period of administration of the compound of Formula (I), or a pharmaceutically acceptable salt thereof, wherein the decrease in body weight is relative to the body weight prior to administration of the compound of Formula (I), or a pharmaceutically acceptable salt thereof.

In some embodiments, central obesity is decreased in the subject after a time period of administration of the compound of Formula (I), or a pharmaceutically acceptable salt thereof, wherein the decrease in central obesity is relative to the central obesity prior to administration of the compound of Formula (I), or a pharmaceutically acceptable salt thereof.

In some embodiments, blood pressure is improved in the subject (e.g., a decrease in blood pressure in a subject with hypertension) after a time period of administration of the compound of Formula (I), or a pharmaceutically acceptable salt thereof, wherein the improvement in blood pressure is relative to the blood pressure prior to administration of the compound of Formula (I), or a pharmaceutically acceptable salt thereof.

In some embodiments, the severity of hirsutism is decreased in the subject after a time period of administration of the compound of Formula (I), or a pharmaceutically acceptable salt thereof, wherein the decrease in the severity of hirsutism is relative to the severity of hirsutism prior to administration of the compound of Formula (I), or a pharmaceutically acceptable salt thereof.

In some embodiments, menstrual regularity is improved or restored in the subject after a time period of administration of the compound of Formula (I), or a pharmaceutically acceptable salt thereof, wherein the improvement or restoration of menstrual regularity is relative to the menstrual cycle to administration of the compound of Formula (I), or a pharmaceutically acceptable salt thereof. In some embodiments, an ovulatory menstrual cycle is restored in the subject after a time period of administration of the compound of Formula (I), or a pharmaceutically acceptable salt thereof.

In some embodiments, control of testicular adrenal rest tumor (TART) is improved in the subject after a time period of administration of the compound of Formula (I), or a pharmaceutically acceptable salt thereof, wherein the improvement in control of testicular adrenal rest tumor is relative to the control of testicular adrenal rest tumor prior to administration of the compound of Formula (I), or a pharmaceutically acceptable salt thereof.

In some embodiments, the incidence and/or severity of testicular adrenal rest tumor (TART) is reduced in the subject after a time period of administration of the compound of Formula (I), or a pharmaceutically acceptable salt thereof.

In some embodiments, control of ovarian adrenal rest tumor (OART) is improved in the subject after a time period of administration of the compound of Formula (I), or a pharmaceutically acceptable salt thereof, wherein the improved control of ovarian adrenal rest tumor is relative to the control of ovarian adrenal rest tumor prior to administration of the compound of Formula (I), or a pharmaceutically acceptable salt thereof.

In some embodiments, the incidence and/or severity of ovarian adrenal rest tumor (OART) is reduced in the subject after a time period of administration of the compound of Formula (I), or a pharmaceutically acceptable salt thereof.

In some embodiments, fertility is improved and/or restored in the subject after a time period of administration of the compound of Formula (I), or a pharmaceutically acceptable salt thereof, wherein the improved and/or restored in fertility is relative to the fertility prior to administration of the compound of Formula (I), or a pharmaceutically acceptable salt thereof.

In some embodiments, gonadotropin levels (including, e.g., LH and FSH) are improved and/or normalized in the subject after a time period of administration of the compound of Formula (I), or a pharmaceutically acceptable salt thereof, wherein the improvement and/or normalization in gonadotropin levels is relative to the gonadotropin levels prior to administration of the compound of Formula (I), or a pharmaceutically acceptable salt thereof.

In some embodiments, progesterone levels are decreased in the subject after a time period of administration of the compound of Formula (I), or a pharmaceutically acceptable salt thereof, wherein the decrease in progesterone levels is relative to the progesterone levels prior to administration of the compound of Formula (I), or a pharmaceutically acceptable salt thereof.

In some embodiments, semen quality (e.g., sperm concentration, morphology, motility, vitality, and volume) is improved in the subject after a time period of administration of the compound of Formula (I), or a pharmaceutically acceptable salt thereof, wherein the improvement in semen quality is relative to the semen quality prior to administration of the compound of Formula (I), or a pharmaceutically acceptable salt thereof.

In some embodiments, LH (luteinizing hormone) levels are increased in the subject after a time period of administration of the compound of Formula (I), or a pharmaceutically acceptable salt thereof, wherein the increase in LH levels are relative to the LH levels prior to administration of the compound of Formula (I), or a pharmaceutically acceptable salt thereof.

In some embodiments, the time period of administration is at least about 4 weeks. In some embodiments, the time period of administration is at least about 24 weeks. In some embodiments, the time period of administration is at least about one year. In some embodiments, the time period of administration is at least 4 weeks. In some embodiments, the time period of administration is at least 24 weeks. In some embodiments, the time period of administration is at least one year. In some embodiments, the time period of administration is less than about 1 day. In some embodiments, the time period of administration is about 1, 2, 3, 4, 5, 6 or 7 days, or within a range of any of the preceding values. In some embodiments, the time period of administration is about 1, 2, 3, 4, 5, 6, 7, 8, 9, 10, 11, 12, 13, 14, 15, 16, 17, 18, 19, 20, 21, 22, 23 or 24 weeks, or within a range of any of the preceding values. In some embodiments, the time period of administration is about 1, 2, 3, 4, 5, 6, 7, 8, 9, 10, 11, or 12 months, or within a range of any of the preceding values. It is understood that comparative measurements occur preferably during the morning.

In some embodiments, the subject is a pediatric subject. In some embodiments, the pediatric subject is less than or equal to six years old. In some embodiments, the pediatric subject is greater than six years old and less than eleven years old. In some embodiments, the pediatric subject is greater than ten years old and less than fifteen years old. In some embodiments, the pediatric subject is greater than fourteen years old and less than nineteen years old. In some embodiments, the pediatric subject weighs less than 55 kg. In some embodiments, the pediatric subject weighs from about 20 kg to about 55 kg. In some embodiments, the pediatric subject weighs from about 10 kg to about 20 kg.

In some embodiments, the subject is an adult subject. In some embodiments, the subject is over eighteen years old. In some embodiments, the subject is female. In some embodiments, the subject is male.

In some embodiments, the compound of Formula (I), or a pharmaceutically acceptable salt thereof, is administered as a pharmaceutical composition described herein. In some embodiments, the compound of Formula (I), or a pharmaceutically acceptable salt thereof, is administered as a pharmaceutical composition described in Example 9. In some embodiments, the compound of Formula (I), or a pharmaceutically acceptable salt thereof, is administered as a pharmaceutical composition described in Example 11. In some embodiments, the compound of Formula (I), or a pharmaceutically acceptable salt thereof, is administered as a pharmaceutical composition described in Example 12. In some embodiments, the compound of Formula (I), or a pharmaceutically acceptable salt thereof, is administered as a pharmaceutical composition described in Example 13. In some embodiments, the compound of Formula (I), or a pharmaceutically acceptable salt thereof, is administered as a hydrochloric acid salt or p-toluenesulfonic acid salt.

In some embodiments, the compound of Formula (I), or a pharmaceutically acceptable salt thereof, is administered as a p-toluenesulfonic acid salt described herein.

For the avoidance of doubt, also provided herein is the corresponding compound of Formula (I), or a pharmaceutically acceptable salt thereof, or corresponding pharmaceutical composition comprising a compound of Formula (I), or a pharmaceutically acceptable salt thereof, for use in the corresponding methods, as described herein.

For the avoidance of doubt, also provided herein is use of the corresponding compound of Formula (I), or a pharmaceutically acceptable salt thereof, in the manufacture of a medicament for use in the corresponding methods, as described herein.

For the avoidance of doubt, also provided herein is use of the corresponding pharmaceutical composition comprising a compound of Formula (I), or a pharmaceutically acceptable salt thereof, in the manufacture of a medicament for use in the corresponding methods, as described herein.

p-Toluenesulfonic Acid Salt

In some embodiments of any of the methods or uses provided herein, the compound of Formula (I) or a pharmaceutically acceptable salt thereof is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, p-toluenesulfonic acid salt.

In some embodiments, the 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, p-toluenesulfonic acid salt is a crystalline salt. In some embodiments, the p-toluenesulfonic acid crystalline salt has Form 1.

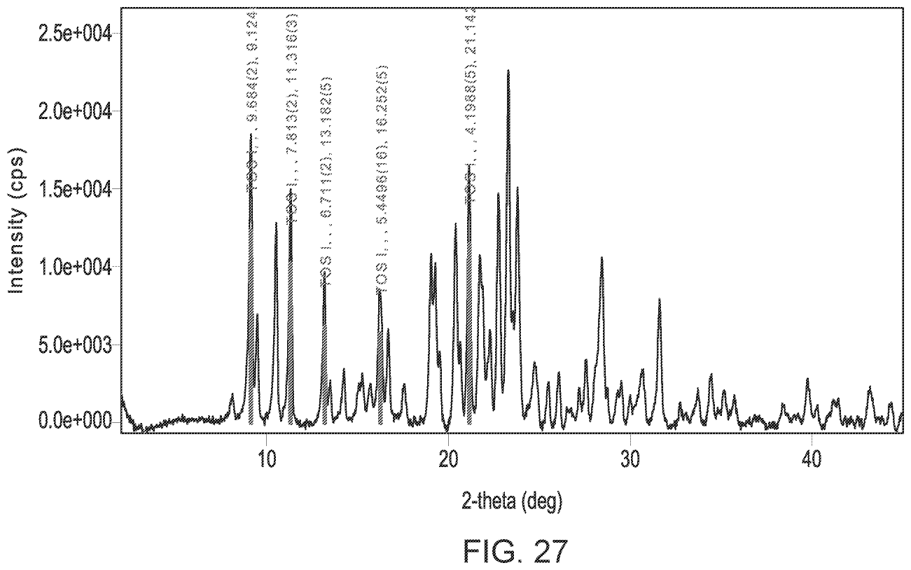

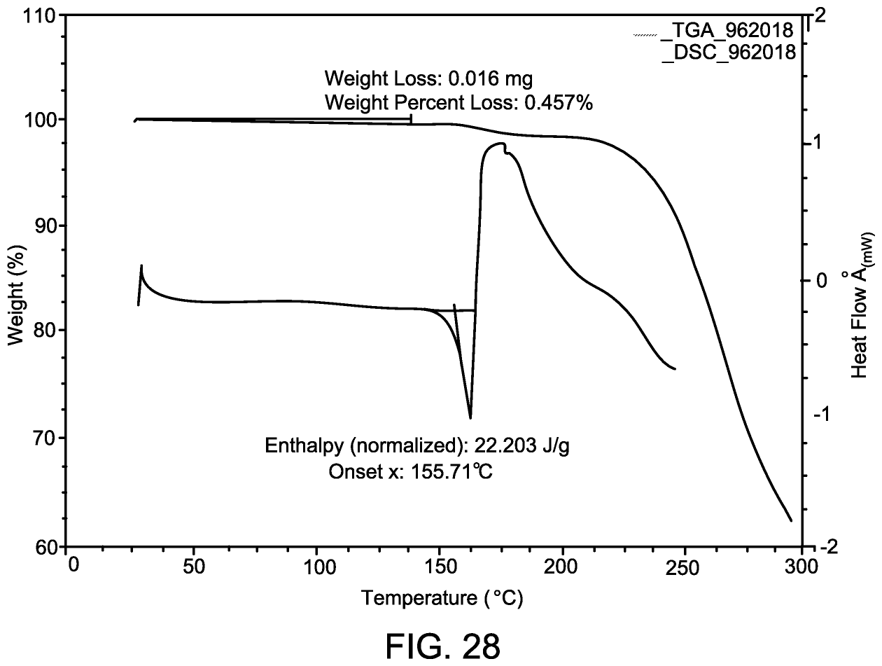

In some embodiments, the p-toluenesulfonic acid crystalline salt has an X-ray powder diffraction pattern as substantially shown in FIG. 27. In some embodiments, the p-toluenesulfonic acid crystalline salt has a DSC thermogram substantially as depicted in FIG. 28. In some embodiments, the p-toluenesulfonic acid crystalline salt has a thermogravimetric analysis (TGA) thermogram substantially as depicted in FIG. 28.

In some embodiments, the p-toluenesulfonic acid crystalline salt has at least one X-ray powder diffraction (XRPD) peak, in terms of 2-theta (±0.2 degrees), selected 9.1, 11.3, 13.2, 16.3 and 21.1 degrees. In some embodiments, the p-toluenesulfonic acid crystalline salt has at least two X-ray powder diffraction (XRPD) peaks, in terms of 2-theta (±0.2 degrees), selected from 9.1, 11.3, 13.2, 16.3 and 21.1 degrees. In some embodiments, the p-toluenesulfonic acid crystalline salt has at least three X-ray powder diffraction (XRPD) peaks, in terms of 2-theta (±0.2 degrees), selected from 9.1, 11.3, 13.2, 16.3 and 21.1 degrees. In some embodiments, the p-toluenesulfonic acid crystalline salt has at least four X-ray powder diffraction (XRPD) peaks, in terms of 2-theta (±0.2 degrees), selected from 9.1, 11.3, 13.2, 16.3 and 21.1 degrees. In some embodiments, the p-toluenesulfonic acid crystalline salt has characteristic X-ray powder diffraction (XRPD) peaks, in terms of 2-theta (±0.2 degrees), at 9.1, 11.3, 13.2, 16.3 and 21.1 degrees. In some embodiments, the p-toluenesulfonic acid crystalline salt has an endothermic peak having an onset of melt at about 156° C. (22.2 J/g) in a differential scanning calorimetry (DSC) thermogram.

### Lipidic Semi-Solid Formulation

Provided herein (for use in any of the methods disclosed herein) is a lipidic semi-solid formulation, which is a pharmaceutical composition comprising:

(a) a compound of Formula (I):

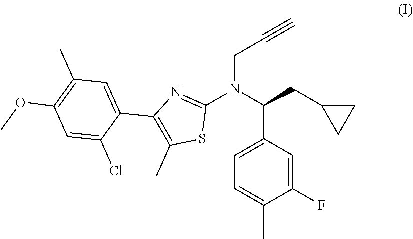 [SMILES: C#CCN(c1nc(-c2cc(C)c(OC)cc2Cl)c(C)s1)[C@@H](CC1CC1)c1ccc(C)c(F)c1]

or a pharmaceutically acceptable salt thereof; and

(b) one or more of an oily phase vehicle, an emulsifying agent, a nonionic surfactant, and a solubilizing agent.

In some embodiments, the pharmaceutical composition comprises about 1 wt % to about 20 wt % of the compound of Formula (I), or a pharmaceutically acceptable salt thereof, based on the weight of the free base. In some embodiments, the pharmaceutical composition comprises about 5 wt % to about 15 wt % of the compound of Formula (I), or a pharmaceutically acceptable salt thereof, based on the weight of the free base. In some embodiments, the pharmaceutical composition comprises about 10 wt % of the compound of Formula (I), or a pharmaceutically acceptable salt thereof, based on the weight of the free base. In some embodiments, the pharmaceutical composition comprises about 1, 2, 3, 4, 5, 6, 7, 8, 9, 10, 11, 12, 13, 14, 15, 16, 17, 18, 19, or 20 wt % of the compound of Formula (I), or a pharmaceutically acceptable salt thereof, based on the weight of the free base, or within a range of any of the preceding values.

In some embodiments, the pharmaceutical composition comprises an oily phase vehicle. An oily phase vehicle is a solvent that is poorly miscible with water. In some embodiments, the pharmaceutical composition comprises about 1 wt % to about 50 wt % of the oily phase vehicle. In some embodiments, the pharmaceutical composition comprises about 20 wt % to about 50 wt % of the oily phase vehicle. In some embodiments, the pharmaceutical composition comprises about 35 wt % to about 45 wt % of the oily phase vehicle. In some embodiments, the pharmaceutical composition comprises about 39 wt % of the oily phase vehicle. In some embodiments, the pharmaceutical composition comprises about 35, 36, 37, 38, 39, 40, 41, 42, 43, 44, or 45 wt % of the oily phase vehicle, or within a range of any of the preceding values.

In some embodiments, the oily phase vehicle is selected from medium-chain triglycerides, glycerin, propylene glycol, polyethylene glycol, olive oil, soybean oil, corn oil, and transcutol. In some embodiments, the oily phase vehicle is medium-chain triglycerides. In some embodiments, the medium-chain triglycerides are Labrafac™ Lipophile WL1349. In some embodiments, the medium-chain triglycerides are Miglyol 812N.

In some embodiments, the pharmaceutical composition comprises an emulsifying agent. An emulsifying agent is a compound or substance that acts as a stabilizer for emulsions. In some embodiments, the pharmaceutical composition comprises about 5 wt % to about 50 wt % of the emulsifying agent. In some embodiments, the pharmaceutical composition comprises about 10 wt % to about 30 wt % of the emulsifying agent. In some embodiments, the pharmaceutical composition comprises about 15 wt % to about 25 wt % of the emulsifying agent. In some embodiments, the pharmaceutical composition comprises about 20 wt % of the emulsifying agent. In some embodiments, the pharmaceutical composition comprises about 10, 11, 12, 13, 14, 15, 16, 17, 18, 19, 20, 21, 22, 23, 24, 25, 26, 27, 28, 29 or 30 wt % of the emulsifying agent, or within a range of any of the preceding values.

In some embodiments, the emulsifying agent is selected from medium-chain triglycerides, propylene glycol dicaprylate/dicaprate, glycerin, propylene glycol, polyethylene glycol, olive oil, soybean oil, corn oil, and transcutol. In some embodiments, the emulsifying agent is propylene glycol dicaprylate/dicaprate. In some embodiments, the propylene glycol dicaprylate/dicaprate is Labrafac™ PG.

In some embodiments, the pharmaceutical composition comprises a nonionic surfactant. A nonionic surfactant is a substance with a hydrophilic head and a hydrophobic tail that has no charge that is a formulation component added to improve solubility or emulsion properties. In some embodiments, the pharmaceutical composition comprises about 5 wt % to about 50 wt % of the nonionic surfactant. In some embodiments, the pharmaceutical composition comprises about 10 wt % to about 30 wt % of the nonionic surfactant. In some embodiments, the pharmaceutical composition comprises about 15 wt % to about 25 wt % of the nonionic surfactant. In some embodiments, the pharmaceutical composition comprises about 19 wt % of the nonionic surfactant. In some embodiments, the pharmaceutical composition comprises about 10, 11, 12, 13, 14, 15, 16, 17, 18, 19, 20, 21, 22, 23, 24, 25, 26, 27, 28, 29 or 30 wt % of the nonionic surfactant, or within a range of any of the preceding values.

In some embodiments, the nonionic surfactant is selected from oleoyl polyoxyl-6 glycerides, linoleoyl polyoxyl-6 glycerides, Polysorbate 80, Polysorbate 20, Gelucire, lauroyl polyoxyl-32 glycerides, Poloxamer, PEG-32 stearate, and PEG-32 hydrogenated palm glycerides. In some embodiments, the nonionic surfactant is lauroyl polyoxyl-32 glycerides. In some embodiments, the lauroyl polyoxyl-32 glycerides are Gelucire® 44/14.

In some embodiments, the pharmaceutical composition comprises a solubilizing agent. A solubilizing agent is a solvent that assists with solubilizing the compound of Formula (I), or a pharmaceutically acceptable salt thereof. In some embodiments, the pharmaceutical composition comprises about 1 wt % to about 50 wt % of the solubilizing agent. In some embodiments, the pharmaceutical composition comprises about 1 wt % to about 20 wt % of the solubilizing agent. In some embodiments, the pharmaceutical composition comprises about 5 wt % to about 15 wt % of the solubilizing agent. In some embodiments, the pharmaceutical composition comprises about 11 wt % of the solubilizing agent. In some embodiments, the pharmaceutical composition comprises about 1, 2, 3, 4, 5, 6, 7, 8, 9, 10, 11, 12, 13, 14, 15, 16, 17, 18, 19, or 20 wt % of the solubilizing agent, or within a range of any of the preceding values.

In some embodiments, the solubilizing agent is selected from oleoyl polyoxyl-6 glycerides, linoleoyl polyoxyl-6 glycerides, Polysorbate 80, Polysorbate 20, vitamin E polyethylene glycol succinate, Gelucire, lauroyl polyoxyl-32 glycerides, and Poloxamer. In some embodiments, the solubilizing agent is vitamin E polyethylene glycol succinate. In some embodiments, the vitamin E polyethylene glycol succinate is Kolliphor® TPGS. In some embodiments, the vitamin E polyethylene glycol succinate is Vitamin E/TPGS 260.

In some embodiments, the pharmaceutical composition comprises:

(a) the compound of Formula (I), or a pharmaceutically acceptable salt thereof;

(b) an oily phase vehicle;

(c) an emulsifying agent;

(d) a nonionic surfactant; and

(e) a solubilizing agent.

In some embodiments, the pharmaceutical composition comprises:

(a) about 5 wt % to about 15 wt % of the compound of Formula (I), or a pharmaceutically acceptable salt thereof, based on the weight of the free base;

(b) about 35 wt % to about 45 wt % of an oily phase vehicle;

(c) about 15 wt % to about 25 wt % of an emulsifying agent;

(d) about 15 wt % to about 25 wt % of a nonionic surfactant; and

(e) about 5 wt % to about 15 wt % of a solubilizing agent.

In some embodiments, the pharmaceutical composition comprises:

(a) about 10 wt % of the compound of Formula (I), or a pharmaceutically acceptable salt thereof, based on the weight of the free base;

(b) about 39 wt % of an oily phase vehicle;

(c) about 20 wt % of an emulsifying agent;

(d) about 19 wt % of a nonionic surfactant; and

(e) about 11 wt % of a solubilizing agent.

In some embodiments, the pharmaceutical composition comprises:

(a) the compound of Formula (I);

(b) a medium-chain triglycerides component;

(c) a propylene glycol dicaprylate/dicaprate component;

(d) a lauroyl polyoxyl-32 glycerides component; and

(e) a vitamin E polyethylene glycol succinate component.

In some embodiments, the pharmaceutical composition comprises:

(a) about 5 wt % to about 15 wt % of the compound of Formula (I);

(b) about 35 wt % to about 45 wt % of medium-chain triglycerides;

(c) about 15 wt % to about 25 wt % of propylene glycol dicaprylate/dicaprate;

(d) about 15 wt % to about 25 wt % of lauroyl polyoxyl-32 glycerides; and

(e) about 5 wt % to about 15 wt % of vitamin E polyethylene glycol succinate.

In some embodiments, the pharmaceutical composition comprises:

(a) about 10 wt % of the compound of Formula (I);

(b) about 39 wt % of medium-chain triglycerides;

(c) about 20 wt % of propylene glycol dicaprylate/dicaprate;

(d) about 19 wt % of lauroyl polyoxyl-32 glycerides; and

(e) about 11 wt % of vitamin E polyethylene glycol succinate.

In some embodiments, the lipidic semi-solid pharmaceutical composition has a viscosity between about 15 to about 40 centipoise at about 45° C. In some embodiments, the lipidic semi-solid pharmaceutical composition has a viscosity between about 26 to about 30 centipoise at about 45° C. In some embodiments, the lipidic semi-solid pharmaceutical composition has a viscosity between about 5 to about 25 centipoise at about 60° C. In some embodiments, the lipidic semi-solid pharmaceutical composition has a viscosity between about 14 to about 18 centipoise at about 60° C.

In some embodiments, the pharmaceutical composition does not comprise a combination of mannitol, croscarmellose sodium, maize starch, hydroxypropyl methylcellulose, and magnesium stearate.

In some embodiments, the pharmaceutical composition does not comprise at least one of mannitol, croscarmellose sodium, maize starch, hydroxypropyl methylcellulose, and magnesium stearate.

In some embodiments, the pharmaceutical composition comprises a compound of Formula (I), or pharmaceutically acceptable salt thereof, in crystalline form. In some embodiments, the pharmaceutical composition comprises a compound of Formula (I), or pharmaceutically acceptable salt thereof, in amorphous form. In some embodiments, the pharmaceutical composition comprises a compound of Formula (I) as a free base. In some embodiments, the crystalline form of the compound of Formula (I) is of Form I.

In some embodiments, the pharmaceutical composition is formulated in unit dosage form, wherein the compound of Formula (I), or a pharmaceutically acceptable salt thereof, is present in an amount of about 5 mg to about 200 mg, based on the weight of the free base. In some embodiments, the compound of Formula (I), or a pharmaceutically acceptable salt thereof, is present in the unit dosage form in an amount of about 75 mg to about 150 mg, based on the weight of the free base. In some embodiments, the compound of Formula (I), or a pharmaceutically acceptable salt thereof, is present in the unit dosage form in an amount of about 50 mg, based on the weight of the free base. In some embodiments, the compound of Formula (I), or a pharmaceutically acceptable salt thereof, is present in the unit dosage form in an amount of about 100 mg, based on the weight of the free base. In some embodiments, the compound of Formula (I), or a pharmaceutically acceptable salt thereof, is present in the unit dosage form in an amount of about 25 mg, based on the weight of the free base.

In some embodiments, the pharmaceutical composition is in the form of a tablet, capsule, sachet, powder, granules, coated particle, coated tablet, enterocoated tablet, enterocoated capsule, melting strip, or melting film. In some embodiments, the pharmaceutical composition is in tablet form. In some embodiments, the pharmaceutical composition is in capsule form. In some embodiments, the dosage form is coated.

### Liquid Formulations

Provided herein (for use in any of the methods disclosed herein) is a pharmaceutical composition in oral solution dosage form comprising:

(a) a compound of Formula (I):

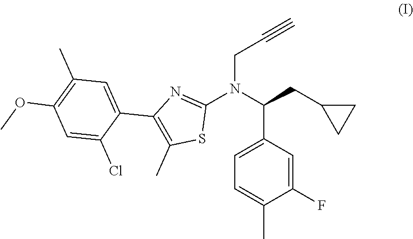 [SMILES: C#CCN(c1nc(-c2cc(C)c(OC)cc2Cl)c(C)s1)[C@@H](CC1CC1)c1ccc(C)c(F)c1]

or a pharmaceutically acceptable salt thereof;

(b) one or more of a sweetener, an anti-oxidant, and a flavor; and

(c) a liquid vehicle.

In some embodiments, the pharmaceutical composition comprises about 1 w/v % to about 50 w/v % of the compound of Formula (I), or a pharmaceutically acceptable salt thereof, based on the weight of the free base. In some embodiments, the pharmaceutical composition comprises about 1 w/v % to about 10 w/v % of the compound of Formula (I), or a pharmaceutically acceptable salt thereof, based on the weight of the free base. In some embodiments, the pharmaceutical composition comprises about 5 w/v % of the compound of Formula (I), or a pharmaceutically acceptable salt thereof, based on the weight of the free base. In some embodiments, the pharmaceutical composition comprises about 1, 2, 3, 4, 5, 6, 7, 8, 9, or 10 w/v % of the compound of Formula (I), or a pharmaceutically acceptable salt thereof, based on the weight of the free base, or within a range of any of the preceding values.

In some embodiments, the pharmaceutical composition comprises a sweetener. A sweetener is a formulation component added to improve taste. In some embodiments, the pharmaceutical composition comprises about 0.01 w/v % to about 1.5 w/v % of the sweetener. In some embodiments, the pharmaceutical composition comprises about 0.1 w/v % to about 0.5 w/v % of the sweetener. In some embodiments, the pharmaceutical composition comprises about 0.15 w/v % of the sweetener. In some embodiments, the pharmaceutical composition comprises about 0.1, 0.2, 0.3, 0.4, or 0.5 w/v % of the sweetener, or within a range of any of the preceding values.

In some embodiments, the sweetener is selected from saccharin, sucrose, sucralose, aspartame, dextrose, fructose, maltitol, mannitol, sorbitol, and avantame. In some embodiments, the sweetener is saccharin.

In some embodiments, the pharmaceutical composition comprises an anti-oxidant. An anti-oxidant is a formulation component included to improve stability by preventing oxidation. In some embodiments, the pharmaceutical composition comprises about 0.01 w/v % to about 1.5 w/v % of the anti-oxidant. In some embodiments, the pharmaceutical composition comprises about 0.1 w/v % to about 0.5 w/v % of the anti-oxidant. In some embodiments, the pharmaceutical composition comprises about 0.17 w/v % of the anti-oxidant. In some embodiments, the pharmaceutical composition comprises about 0.1, 0.2, 0.3, 0.4, or 0.5 w/v % of the anti-oxidant, or within a range of any of the preceding values.

In some embodiments, the anti-oxidant is selected from butylated hydroxytoluene, vitamin E TPGS, butylated hydroxyanisole, ascorbic acid, lecithin, tert-butylhydroquinone, and citric acid. In some embodiments, the anti-oxidant is butylated hydroxytoluene.

In some embodiments, the pharmaceutical composition comprises a flavor. A flavor is a formulation component added to mask taste through aromatics. In some embodiments, the pharmaceutical composition comprises about 0.01 w/v % to about 0.5 w/v % of the flavor. In some embodiments, the pharmaceutical composition comprises about 0.05 w/v % to about 0.2 w/v % of the flavor. In some embodiments, the pharmaceutical composition comprises about 0.10 w/v % of the flavor. In some embodiments, the pharmaceutical composition comprises about 0.05, 0.06, 0.07, 0.08, 0.09, 0.10, 0.11, 0.12, 0.13, 0.14, 0.15, 0.16, 0.17, 0.18, 0.19 or 0.2 w/v % of the flavor, or within a range of any of the preceding values.

In some embodiments, the flavor is selected from FONA orange flavor, FONA Juicy Flavor, FONA Grape Flavor, Firmenich SA Lemon Flavor, Firmenich Tetrarome Orange Flavor, IFF Cherry Flavor, and IFF Grape Flavor. In some embodiments, the flavor is FONA orange flavor.

A liquid vehicle is a solvent capable of dissolving or partially dissolving the compound of Formula (I), or a pharmaceutically acceptable salt thereof, for the purposes of delivery as an oral dosing solution. In some embodiments, the pharmaceutical composition comprises about 50 w/v % to about 99.9 w/v % of the liquid vehicle. In some embodiments, the pharmaceutical composition comprises about 90 w/v % to about 99 w/v % of the liquid vehicle. In some embodiments, the pharmaceutical composition comprises about 92 w/v % to about 97 w/v % of the liquid vehicle. In some embodiments, the pharmaceutical composition comprises about 94.6 w/v % of the liquid vehicle. In some embodiments, the pharmaceutical composition comprises about 90, 91, 92, 93, 94, 95, 96, 97, 98 or 99 w/v % of the liquid vehicle, or within a range of any of the preceding values.

In some embodiments, the liquid vehicle is selected from medium-chain triglycerides, propylene glycol dicaprylate/dicaprate, glycerin, propylene glycol, polyethylene glycol, olive oil, soybean oil, corn oil, and transcutol. In some embodiments, the liquid vehicle is medium-chain triglycerides. In some embodiments, the medium-chain triglycerides is Labrafac Lipophile WL1349.

In some embodiments, the pharmaceutical composition further comprises a surfactant. A surfactant is a formulation component added to improve solubility or emulsion properties. In some embodiments, the pharmaceutical composition comprises about 1 w/v % to about 50 w/v % of the surfactant. In some embodiments, the pharmaceutical composition comprises about 10 w/v % to about 30 w/v % of the surfactant. In some embodiments, the pharmaceutical composition comprises about 20 w/v % of the surfactant. In some embodiments, the pharmaceutical composition comprises about 10, 11, 12, 13, 14, 15, 16, 17, 18, 19, 20, 21, 22, 23, 24, 25, 26, 27, 28, 29 or 30 w/v % of the surfactant, or within a range of any of the preceding values.

In some embodiments, the surfactant is selected from oleoyl polyoxyl-6 glycerides, linoleoyl polyoxyl-6 glycerides, Polysorbate 80, Polysorbate 20, vitamin E polyethylene glycol succinate, Gelucire, lauroyl polyoxyl-32 glycerides, sodium lauryl sulfate, Poloxamer, corn oil PEG-6 esters, and hydrogenated palm/palm kernel oil PEG-6 esters. In some embodiments, the surfactant is oleoyl polyoxyl-6 glycerides. In some embodiments, the oleoyl polyoxyl-6 glycerides is LABRAFIL M 1944 CS.

In some embodiments, the pharmaceutical composition comprises about 50 w/v % to about 90 w/v % of the liquid vehicle. In some embodiments, the pharmaceutical composition comprises about 70 w/v % to about 80 w/v % of the liquid vehicle. In some embodiments, the pharmaceutical composition comprises about 75 w/v % of the liquid vehicle. In some embodiments, the pharmaceutical composition comprises about 74.6 w/v % of the liquid vehicle. In some embodiments, the pharmaceutical composition comprises about 70, 71, 72, 73, 74, 75, 76, 77, 78, 79, or 80 w/v % of the liquid vehicle, or within a range of any of the preceding values.

In some embodiments, the liquid vehicle is selected from medium-chain triglycerides, propylene glycol dicaprylate/dicaprate, glycerin, propylene glycol, polyethylene glycol, olive oil, soybean oil, corn oil, and transcutol. In some embodiments, the liquid vehicle is medium-chain triglycerides. In some embodiments, the medium-chain triglycerides is Labrafac Lipophile WL1349.

In some embodiments, the pharmaceutical composition comprises:

(a) the compound of Formula (I), or a pharmaceutically acceptable salt thereof;

(b) a sweetener;

(c) an anti-oxidant;

(d) a flavor; and

(e) a liquid vehicle.

In some embodiments, the pharmaceutical composition further comprises a surfactant.

In some embodiments, the pharmaceutical composition comprises:

(a) about 4 w/v % to about 6 w/v % of the compound of Formula (I), or a pharmaceutically acceptable salt thereof, based on the weight of the free base;

(b) about 0.1 w/v % to about 0.2 w/v % of a sweetener;

(c) about 0.1 w/v % to about 0.2 w/v % of an anti-oxidant;

(d) about 0.05 w/v % to about 0.2 w/v % of a flavor; and

(e) about 92 w/v % to about 97 w/v % of a liquid vehicle.

In some embodiments, the pharmaceutical composition comprises:

(a) about 5 w/v % of the compound of Formula (I), or a pharmaceutically acceptable salt thereof, based on the weight of the free base;

(b) about 0.15 w/v % of a sweetener;

(c) about 0.17 w/v % of an anti-oxidant;

(d) about 0.1 w/v % of a flavor; and

(e) about 94.6 w/v % of a liquid vehicle.

In some embodiments, the pharmaceutical composition comprises:

(a) about 4 w/v % to about 6 w/v % of the compound of Formula (I), or a pharmaceutically acceptable salt thereof, based on the weight of the free base;

(b) about 0.1 w/v % to about 0.2 w/v % of a sweetener;

(c) about 0.1 w/v % to about 0.2 w/v % of an anti-oxidant;

(d) about 0.05 w/v % to about 0.2 w/v % of a flavor;

(e) about 15 w/v % to about 25 w/v % of a surfactant; and

(f) about 70 w/v % to about 80 w/v % of a liquid vehicle.

In some embodiments, the pharmaceutical composition comprises:

(a) about 5 w/v % of the compound of Formula (I), or a pharmaceutically acceptable salt thereof, based on the weight of the free base;

(b) about 0.15 w/v % of a sweetener;

(c) about 0.17 w/v % of an anti-oxidant;

(d) about 0.1 w/v % of a flavor;

(e) about 20 w/v % of a surfactant; and

(f) about 75 w/v % of a liquid vehicle.

In some embodiments, the pharmaceutical composition comprises:

(a) the compound of Formula (I), or a pharmaceutically acceptable salt thereof;

(b) saccharin;

(c) butylated hydroxytoluene;

(d) FONA orange flavor; and

(e) medium-chain triglycerides.

In some embodiments, the pharmaceutical composition further comprises oleoyl polyoxyl-6 glycerides.

In some embodiments, the pharmaceutical composition comprises:

(a) about 4 w/v % to about 6 w/v % of the compound of Formula (I), or a pharmaceutically acceptable salt thereof, based on the weight of the free base;

(b) about 0.1 w/v % to about 0.2 w/v % of saccharin;

(c) about 0.1 w/v % to about 0.2 w/v % of butylated hydroxytoluene;

(d) about 0.05 w/v % to about 0.2 w/v % of FONA orange flavor; and

(e) about 92 w/v % to about 97 w/v % of medium-chain triglycerides.

In some embodiments, the pharmaceutical composition comprises:

(a) about 5 w/v % of the compound of Formula (I), or a pharmaceutically acceptable salt thereof, based on the weight of the free base;

(b) about 0.15 w/v % of saccharin;

(c) about 0.17 w/v % of butylated hydroxytoluene;

(d) about 0.1 w/v % of FONA orange flavor; and

(e) about 94.6 w/v % of medium-chain triglycerides.

In some embodiments, the pharmaceutical composition comprises:

(a) about 4 w/v % to about 6 w/v % of the compound of Formula (I), or a pharmaceutically acceptable salt thereof, based on the weight of the free base;

(b) about 0.1 w/v % to about 0.2 w/v % of saccharin;

(c) about 0.1 w/v % to about 0.2 w/v % of butylated hydroxytoluene;

(d) about 0.05 w/v % to about 0.2 w/v % of FONA orange flavor;

(e) about 15 w/v % to about 25 w/v % of oleoyl polyoxyl-6 glycerides; and

(f) about 70 w/v % to about 80 w/v % of medium-chain triglycerides.

In some embodiments, the pharmaceutical composition comprises:

(a) about 5 w/v % of the compound of Formula (I), or a pharmaceutically acceptable salt thereof, based on the weight of the free base;

(b) about 0.15 w/v % of saccharin;

(c) about 0.17 w/v % of butylated hydroxytoluene;

(d) about 0.1 w/v % of FONA orange flavor;

(e) about 20 w/v % of oleoyl polyoxyl-6 glycerides; and

(f) about 75 w/v % of medium-chain triglycerides.

In some embodiments, the pharmaceutical composition comprises the compound of Formula (I) as a free base.

In some embodiments, the pharmaceutical composition is formulated in unit dosage form, wherein the compound of Formula (I), or a pharmaceutically acceptable salt thereof, is present in an amount of about 5 mg/mL to about 200 mg/mL, based on the weight of the free base. In some embodiments, the compound of Formula (I), or a pharmaceutically acceptable salt thereof, is present in the unit dosage form in an amount of about 75 mg/mL to about 150 mg/mL, based on the weight of the free base. In some embodiments, the compound of Formula (I), or a pharmaceutically acceptable salt thereof, is present in the unit dosage form in an amount of about 50 mg/mL, based on the weight of the free base. In some embodiments, the compound of Formula (I), or a pharmaceutically acceptable salt thereof, is present in the unit dosage form in an amount of about 100 mg/mL, based on the weight of the free base.

In some embodiments, the liquid pharmaceutical composition has a viscosity between about 1 to about 50 centipoise at about 25° C.

### Spray-Dried Dispersions

The methods and uses of the present disclosure may comprise administering a spray-dried dispersion (SDD) of the compound of Formula (I), or a pharmaceutically acceptable salt thereof, and to the use of the SDDs in the treatment of congenital adrenal hyperplasia (CAH).

In some embodiments, concentration and bioavailability enhancement in an aqueous environment of a low-solubility drug in a spray-dried dispersion is achieved if the SDD exhibits one or more properties, including, for example: (1) the solid dispersion is substantially homogeneous; (2) the drug is substantially amorphous; (3) the SDD has a relatively high drug loading; and (4) the SDD has a low residual solvent content. In some embodiments, the dispersion, when administered to an aqueous environment, provides at least a temporary dissolved drug concentration in the aqueous environment that is greater than the solubility of the crystalline form of the drug in the same environment. The aqueous environment can be, for example, an in vitro environment, such as a dissolution test media (e.g., phosphate buffered saline (PBS) solution), or an in vivo environment, such as the gastrointestinal (GI) tract of an animal, for example, a human. In some embodiments, the aqueous environment is the lower GI tract, such as the small intestine and large intestine.

In some embodiments, the compound of Formula (I), or a pharmaceutically acceptable salt thereof, in the spray-dried dispersion is substantially amorphous. As used herein, “substantially amorphous” means that the amount of the compound of Formula (I), or a pharmaceutically acceptable salt thereof, in amorphous form is at least 60 wt % and that the amount of crystalline form present does not exceed 20 wt %. In some embodiments, the compound of Formula (I), or a pharmaceutically acceptable salt thereof, in the dispersion is “almost completely amorphous,” meaning that at least 90 wt % of the drug is amorphous, or that the amount of the compound of Formula (I), or a pharmaceutically acceptable salt thereof, in the crystalline form does not exceed 10 wt %. Amounts of crystalline drug can be measured by powder X-ray diffraction (PXRD), scanning electron microscope (SEM) analysis, differential scanning calorimetry (DSC), polarized light microscopy (PLM), or any other standard quantitative or qualitative measurement used to detect crystalline material. Without wishing to be bound by any theory, it is believed that the amorphous, or non-crystalline form, in combination with the polymer, leads to greater ease of dissolution and absorption in the desired location, for example, the intestines, resulting in enhanced bioavailability as compared to a crystalline form of the compound of Formula (I) without polymer.

### Deuterated Compounds

The methods and uses disclosed herein encompass compounds having the structure of the following formula (II):

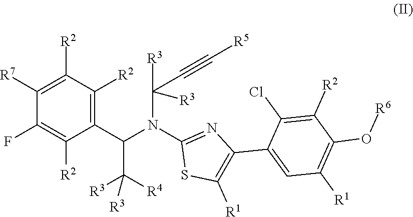

or a pharmaceutically acceptable salt thereof, wherein:

each R¹ is independently C(R^{A})₃;

each R^{A} is independently hydrogen or deuterium;

each R² is independently hydrogen or deuterium;

each R³ is independently hydrogen or deuterium;

R⁴ is

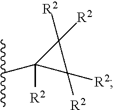

R⁵ is hydrogen or deuterium;

R⁶ is C(R^{A})₃; and

R⁷ is C(R^{B})₃, wherein at least one of R^{A}, R^{B}, R², R³ and R⁵ is deuterium.

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

In some embodiments, the compound of Formula (II) may be one of the following, or a pharmaceutically acceptable salt thereof:

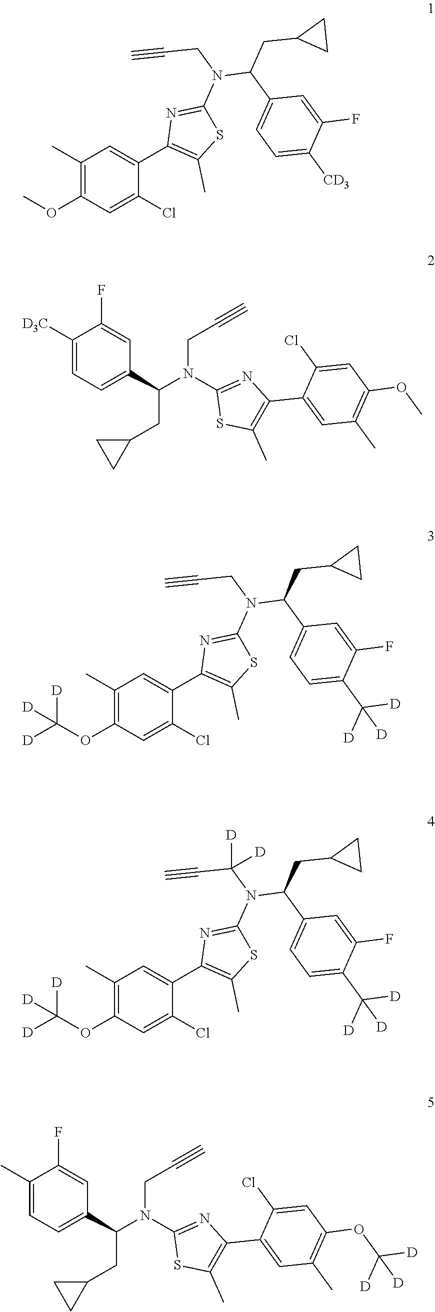

### Pharmaceutical Compositions

The methods and uses disclosed herein can comprise administering the compound of Formula (I) as a pharmaceutical composition.

In some embodiments of the methods described herein, the compound of Formula (I), or a pharmaceutically acceptable salt thereof is administered in a pharmaceutical composition further comprising one or more pharmaceutically acceptable excipients.

Also provided herein is a pharmaceutical composition comprising a compound of Formula (I), or a pharmaceutically acceptable salt thereof, for use in any of the methods described herein.

In some embodiments, the methods and uses described herein comprise administering a pharmaceutical composition that does not comprise a spray-dried dispersion of the compound of Formula (I), as specified, e.g., in Example 1. Accordingly, in some embodiments, the pharmaceutical composition does not comprise any of the following polymers: hydroxypropylmethylcellulose acetate succinate-L (HPMCAS-L); polyvinyl pyrrolidone vinyl acetate 64 (PVP/VA 64); HPMCAS-M; and methyl methacrylate copolymer (1:1) (Eudragit® L100).

In some embodiments, the methods and uses described herein comprise administering a pharmaceutical composition that is not the reference formulation described in Example 9. Accordingly, in some embodiments, the pharmaceutical composition does not comprise at least three of the excipients selected from caprylic/capric triglyceride (Labrafac® Lipophile, Gattefossé, France); propylene glycol dicrapolate/dicaprate (Labrafac® PG, Gattefossé, France); oleoyl polyoxyl-6 glycerides (Labrafil® M 1944 CS, Gattefossé, France); polysorbate 20; polyoxyl castor oil (Kolliphor® RH 40, BASF, Germany); polyoxyl 15 hydroxystearate (Kolliphor® HS 15, BASF, Germany); lauroyl polyoxyl-32 glycerides (Gelucire® 44/14, Gattefossé, France); d-α-tocopheryl polyethylene glycol 1000 succinate (TPGS); and diethylene glycol monoethyl ether (Transcutol®, Gattefossé, France).

In some embodiments, the methods and uses described herein comprise administering a pharmaceutical composition that is the formulation described in Example 9. In some embodiments, the methods and uses described herein comprise administering a pharmaceutical composition that is the formulation described in Example 11. In some embodiments, the methods and uses described herein comprise administering a pharmaceutical composition that is the formulation described in Example 12. In some embodiments, the methods and uses described herein comprise administering a pharmaceutical composition that is the formulation described in Example 13.

In some embodiments, the pharmaceutical compositions include a spray-dried dispersion containing a polymer and the compound of Formula (I), or a pharmaceutically acceptable salt thereof.

In some embodiments, the spray-dried dispersion comprises:

a compound of Formula (I), or a pharmaceutically acceptable salt thereof; and

a polymer that is a copolymer of 1-vinyl-2-pyrrolidone and vinyl acetate having the structure:

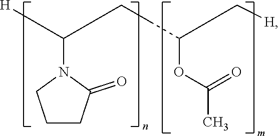

wherein the value of n is about 1 to about 2 times the value of m and the copolymer comprises 1-vinyl-2-pyrrolidone and vinyl acetate at a ratio of about 60:40 by weight; and

wherein the weight ratio of the compound of Formula (I) to the copolymer is from about 1:1 to about 1:9.

In some embodiments, the pharmaceutical composition includes the SDD comprising a compound of Formula (I) and one or more pharmaceutically acceptable excipients. In some embodiments, the SDD is present in the pharmaceutical composition in an amount of about 20% to about 90% w/w of the composition, such as about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 90%, about 50% to about 85%, about 50% to about 80%, about 50% to about 75%, about 50% to about 70%, about 50% to about 65%, about 50% to about 60%, about 50% to about 55%, about 55% to about 90%, about 55% to about 85%, about 55% to about 80%, about 55% to about 75%, about 55% to about 70%, about 55% to about 65%, about 55% to about 60%, about 60% to about 90%, about 60% to about 85%, about 60% to about 80%, about 60% to about 75%, about 60% to about 70%, about 60% to about 65%, about 65% to about 90%, about 65% to about 85%, about 65% to about 80%, about 65% to about 75%, about 65% to about 70%, about 70% to about 90%, about 70% to about 85%, about 70% to about 80%, about 70% to about 75%, about 75% to about 90%, about 75% to about 85%, about 75% to about 80%, about 80% to about 90%, about 80% to about 85%, or about 85% to about 90% w/w of the composition. In some embodiments, the SDD is present in an amount of about 40% to about 90% w/w of the composition. In some embodiments, the SDD is present in an amount of about 40% to about 80% w/w of the composition. In some embodiments, the SDD is present in the pharmaceutical composition in an amount of about 60% to about 80% w/w of the composition. In some embodiments, the SDD is present in an amount of about 80% w/w of the composition. In some embodiments, the SDD is present in the pharmaceutical composition in an amount of about 1% to about 20% w/w of the composition, such as about 13% w/w of the composition.

In some embodiments, the pharmaceutical composition includes the SDD comprising a compound of Formula (I) and one or more pharmaceutically acceptable excipients. In some embodiments, the SDD is present in the pharmaceutical composition in an amount of 20% to 90% w/w of the composition, such as 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 90%, 35% to 85%, 35% to 80%, 35% to 75%, 35% to 70%, 35% to 65%, 35% to about 60%, 35% to 55%, 35% to 50%, 35% to 45%, 35% to 40%, 40% to 90%, 40% to 85%, 40% to 80%, 40% to 75%, 40% to 70%, 40% to 65%, 40% to 60%, 40% to 55%, 40% to 50%, 40% to 45%, 45% to 90%, 45% to 85%, 45% to 80%, 45% to 75%, 45% to 70%, 45% to 65%, 45% to 60%, 45% to 55%, 45% to 50%, 50% to 90%, 50% to 85%, 50% to 80%, 50% to 75%, 50% to 70%, 50% to 65%, 50% to 60%, 50% to 55%, 55% to 90%, 55% to 85%, 55% to 80%, 55% to 75%, 55% to 70%, 55% to 65%, 55% to 60%, 60% to 90%, 60% to 85%, 60% to 80%, 60% to 75%, 60% to 70%, 60% to 65%, 65% to 90%, 65% to 85%, 65% to 80%, 65% to 75%, 65% to 70%, 70% to 90%, 70% to 85%, 70% to 80%, 70% to 75%, 75% to 90%, 75% to 85%, 75% to 80%, 80% to 90%, 80% to 85%, or about 85% to 90% w/w of the composition. In some embodiments, the SDD is present in an amount of 40% to 90% w/w of the composition. In some embodiments, the SDD is present in an amount of 40% to 80% w/w of the composition. In some embodiments, the SDD is present in the pharmaceutical composition in an amount of 60% to 80% w/w of the composition. In some embodiments, the SDD is present in an amount of 80% w/w of the composition. In some embodiments, the SDD is present in the pharmaceutical composition in an amount of about 1% to about 20% w/w of the composition, such as about 13% w/w of the composition.

In some embodiments of the pharmaceutical compositions disclosed herein (e.g., a composition including an SDD), the pharmaceutically acceptable excipient is selected from the group consisting of a filler, a lubricant, and combinations thereof. In some embodiments, the pharmaceutical excipients are selected from the group consisting of a glidant, a filler, a disintegrant, a lubricant, and a combination thereof.

In some embodiments, the pharmaceutical composition includes a filler. In some embodiments, the filler is selected from among binders, diluents, disintegrants, glidants, surfactants, and combinations thereof.

In some embodiments, the filler include saccharides (e.g., sugars, starch, and cellulose), gelatin, calcium carbonate, and synthetic polymers (e.g., polyvinylpyrrolidone, polyethylene glycol, and poloxamers (e.g., Poloxamer 188, a copolymer of polyoxyethylene and polyoxypropylene)). Exemplary fillers include, but are not limited to, glucose, sucrose, lactose, a starch, including modified starches such as sodium starch glycolate (e.g., Explotab®), xylitol, dextrin, saccharose, sorbitol, mannitol (e.g., Parteck® M 200 (mannitol with an average particle size of about 50 μm to about 500 μm) or Parteck® M 100 (mannitol with an average particle size of less than 212 μm)), a cellulose, a polyvinylpyrrolidone, a polyethylene glycol, a polyvinyl alcohol, a polymethacrylate, dibasic calcium phosphate, magnesium stearate, calcium stearate, sodium stearate, stearic acid, hydrogenated vegetable oils, a mineral oil, sodium lauryl sulfate, magnesium lauryl sulfate, glyceryl palmitostearate, sodium benzoate, sodium stearyl fumarate, colloidal silicon dioxide, sodium benzoate, sodium oleate, sodium acetate, aliginic acid, alginates (e.g., sodium alginate), calcium silicate, and ion exchange resins. Exemplary cellulose fillers include microcrystalline cellulose (e.g., Avicel® PH-101 (microcrystalline cellulose with an average particle size of approximately 50 μm) or Avicel® PH 200 (microcrystalline cellulose with an average particle size of approximately 180 μm)), methyl cellulose, ethyl cellulose, hydroxypropyl cellulose, and hydroxypropylmethylcellulose. Exemplary fillers include cross-linked polyvinylpyrrolidone such as with an average particle size of 90 μm to 130 μm) or with an average particle size of 10 μm to 30 μm). Other fillers known to those of skill in the art are also contemplated as being useful when formulated in the pharmaceutical compositions described herein.

In some embodiments, the filler is a binder. Binders include agents that hold the active pharmaceutical ingredient (e.g., spray-dried dispersion containing a polymer and the compound of Formula (I), or a pharmaceutically acceptable salt thereof) and inactive ingredients together in a cohesive mix. Exemplary binders include, but are not limited to, glucose, sucrose, lactose, a starch, including modified starches such as sodium starch glycolate (Explotab®), xylitol, dextrin, saccharose, sorbitol, mannitol (e.g., Parteck® M 200 (mannitol with an average particle size of about 50 μm to about 500 μm), Parteck® M 100 (mannitol with an average particle size of less than 212 μm)), gelatin, gum tragacanth, acacia mucilage, a cellulose, a polyvinylpyrrolidone, a polyethylene glycol, a polyvinyl alcohol, a polymethacrylate, and sodium starch glycolate. Exemplary cellulose fillers include microcrystalline cellulose (e.g., Avicel® PH-101 (microcrystalline cellulose with an average particle size of approximately 50 μm) or Avicel® PH 200 (microcrystalline cellulose with an average particle size of approximately 180 μm)), cellulose ethers, methyl cellulose, ethyl cellulose, croscarmellose sodium, sodium carboxy methyl cellulose starches, hydroxypropyl cellulose, and hydroxypropyl methyl cellulose. Exemplary polyvinylpyrrolidone fillers include cross-linked polyvinylpyrrolidone such as Kollidon® CL (crospovidone with an average particle size of 90 μm to 130 μm) or Kollidon® CL-SF (crospovidone with an average particle size of 10 μm to 30 μm). Other binders known to those of skill in the art are also contemplated as being useful when formulated in the compositions described herein.

In some embodiments, the filler is a diluent. Suitable diluents include, but are not limited to, lactose, mannitol, isomalt, sucrose, dextrose, and sorbitol.

In some embodiments, the filler is a disintegrant. Disintegrants include any agent that promotes breakup of the formulation in an aqueous environment, for example, to promote more rapid release of the active pharmaceutical ingredient (e.g., the compound of Formula (I), or a pharmaceutically acceptable salt thereof). Exemplary disintegrants include, but are not limited to, starch and modified starches, such as corn starch, potato starch, sodium starch glycolate or croscarmellose sodium, alginic acid, alginates, such as sodium alginate, polyvinylpyrrolidone, bentonite, methylcellulose, agar, carboxymethylcellulose, crospovidone, acid-carbonate effervescent systems, such as citric acid with bicarbonate salts, and ion exchange resins. Other disintegrants known to those of skill in the art are also contemplated as being useful when formulated in the compositions described herein.

In some embodiments, the pharmaceutical composition comprises a disintegrant. In some embodiments, the pharmaceutical composition comprises about 1 w/w % to about 30 w/w % of the disintegrant. In some embodiments, the pharmaceutical composition comprises about 5 w/w % to about 15 w/w % of the disintegrant. In some embodiments, the pharmaceutical composition comprises about 10 w/w % of the disintegrant. In some embodiments, the disintegrant is selected from croscarmellose sodium, sodium starch glycolate, crospovidone, and sodium bicarbonate. In some embodiments, the disintegrant is croscarmellose sodium.

In some embodiments, the filler is a glidant. Glidants can be used to improve the flowability of a powder or granules or both. Glidants include, but are not limited to, silicone dioxide, such as colloidal silicon dioxide or hydrated silicon dioxide, magnesium silicate, magnesium aluminometasilicate, talc, starch, calcium silicate, light anhydrous silicic acid, and silicon dioxide aerogels.

In some embodiments, the pharmaceutical composition comprises a glidant. In some embodiments, the pharmaceutical composition comprises about 0.1 w/w % to about 5 w/w % of the glidant. In some embodiments, the pharmaceutical composition comprises about 0.1 w/w % to about 1 w/w % of the glidant. In some embodiments, the pharmaceutical composition comprises about 0.67 w/w % of the glidant. In some embodiments, the glidant is selected from calcium silicate, silicon dioxide, and talc. In some embodiments, the glidant is calcium silicate.

In some embodiments, the filler is a surfactant, wetting agent, solubilizer, or combination thereof. Examples include, but are not limited to, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., Tween®), polyoxyethylene stearates, sodium dodecylsulfate, tyloxapol (a nonionic liquid polymer of the alkyl aryl polyether alcohol type, also known as superinone or triton). Other examples include, but are not limited to, poloxamers such as Pluronic® F68, F127, and F108, which are block copolymers of ethylene oxide and propylene oxide, and polyxamines such as Tetronic® 908 (also known as Poloxamine® 908), which is a tetrafunctional block copolymer derived from sequential addition of propylene oxide and ethylene oxide to ethylenediamine (available from BASF), dextran, lecithin, dialkylesters of sodium sulfosuccinic acid, such as Aerosol® OT, which is a dioctyl ester of sodium sulfosuccinic acid (available from American Cyanimid), Duponol® P, which is a sodium lauryl sulfate (available from DuPont), Triton® X-200, which is an alkyl aryl polyether sulfonate (available from Rohm and Haas), Tween® 20 and Tween® 80, which are polyoxyethylene sorbitan fatty acid esters (available from ICI Specialty Chemicals), Carbowax™ 3550 and 934, which are polyethylene glycols (available from Union Carbide), Crodesta™ F-110, which is a mixture of sucrose stearate and sucrose distearate, and Crodesta™ SL-40 (both available from Croda Inc.), and SA90HCO, which has the chemical formula C₁₈H₃₇—CH₂(CON(CH₃)CH₂(CHOH)₄CH₂OH)₂.

In some embodiments, the pharmaceutical composition comprises a filler. In some embodiments, the pharmaceutical composition comprises about 30 w/w % to about 99 w/w % of the filler. In some embodiments, the pharmaceutical composition comprises about 50 w/w % to about 90 w/w % of the filler. In some embodiments, the pharmaceutical composition comprises about 75.5 w/w % of the filler. In some embodiments, the filler is selected from mannitol, microcrystalline cellulose, lactose, starch, isomalt, silicified microcrystalline cellulose, Dicalcium Phosphate, maltodextrin, and a combination thereof. In some embodiments, the filler is a combination of mannitol and microcrystalline cellulose. In some embodiments, the pharmaceutical composition comprises about 30 w/w % to about 80 w/w % of mannitol. In some embodiments, the pharmaceutical composition comprises about 50 w/w % to about 60 w/w % of mannitol. In some embodiments, the pharmaceutical composition comprises about 56 w/w % of mannitol. In some embodiments, the pharmaceutical composition comprises about 1 w/w % to about 50 w/w % of microcrystalline cellulose. In some embodiments, the pharmaceutical composition comprises about 10 w/w % to about 30 w/w % of microcrystalline cellulose. In some embodiments, the pharmaceutical composition comprises about 20 w/w % of microcrystalline cellulose. In some embodiments, the pharmaceutical composition comprises about 56 w/w % of mannitol and about 20 w/w % of microcrystalline cellulose.

In some embodiments, the pharmaceutical composition includes a lubricant. Lubricants are agents added to pharmaceutical formulations to reduce friction during processing and prevent ingredients from clumping together. Exemplary lubricants include, but are not limited to, talc, starch, magnesium stearate, calcium stearate, sodium stearate, zinc stearate, stearic acid, vegetable stearin, adipic acid, waxy fatty acids, such as glyceryl behenate, a hydrogenated vegetable oil, a mineral oil, a polyethylene glycol, lycopodium, sodium lauryl sulfate, magnesium lauryl sulfate, glyceryl palmitostearate, sodium benzoate, sodium chloride, sterotex, glycerol monostearate, sodium stearyl fumarate, colloidal silicon dioxide, sodium benzoate, sodium oleate, and sodium acetate. Other lubricants known to those of skill in the art are also contemplated as being useful when formulated in the compositions described herein.

In some embodiments, the pharmaceutical composition comprises a lubricant. In some embodiments, the pharmaceutical composition comprises about 0.1 w/w % to about 10 w/w % of the lubricant. In some embodiments, the pharmaceutical composition comprises about 0.1 w/w % to about 1 w/w % of the lubricant. In some embodiments, the pharmaceutical composition comprises about 0.5 w/w % of the lubricant. In some embodiments, the pharmaceutical lubricant is selected from sodium stearyl fumarate, magnesium stearate, stearic acid sodium lauryl sulfate, sodium oleate, glyceryl behenate, and talc. In some embodiments, the lubricant is sodium stearyl fumarate.

In some embodiments, the pharmaceutical composition comprises:

(a) the spray-dried dispersion comprising the compound of Formula (I), or a pharmaceutically acceptable salt thereof, and a polymer;

(b) a glidant;

(c) a filler; and

(d) a disintegrant.

In some embodiments, the pharmaceutical composition comprises:

(a) about 1 w/w % to about 20 w/w % of the spray-dried dispersion comprising the compound of Formula (I), or a pharmaceutically acceptable salt thereof, and a polymer;

(b) about 0.1 w/w % to about 1 w/w % of a glidant;

(c) about 50 w/w % to about 90 w/w % of a filler; and

(d) about 5 w/w % to about 0.2 w/w % of a disintegrant.

In some embodiments, the pharmaceutical composition comprises:

(a) about 13 w/w % of the spray-dried dispersion comprising the compound of Formula (I), or a pharmaceutically acceptable salt thereof, and a polymer;

(b) about 0.67 w/w % of a glidant;

(c) about 75.5 w/w % of a filler; and

(d) about 10 w/w % of a disintegrant.

In some embodiments, the pharmaceutical composition comprises:

(a) the spray-dried dispersion of Example 3;

(b) calcium silicate;

(c) a combination of mannitol and microcrystalline cellulose; and

(d) croscarmellose sodium.

In some embodiments, the pharmaceutical composition comprises:

(a) about 1 w/w % to about 20 w/w % of the spray-dried dispersion of Example 3;

(b) about 0.1 w/w % to about 1 w/w % of calcium silicate;

(c) about 50 w/w % to about 60 w/w % of mannitol and about 10 w/w % to about 30 w/w % of microcrystalline cellulose; and

(d) about 5 w/w % to about 0.2 w/w % of croscarmellose sodium.

In some embodiments, the pharmaceutical composition comprises:

(a) about 13 w/w % of the spray-dried dispersion of Example 3;

(b) about 0.67 w/w % of calcium silicate;

(c) about 56 w/w % of mannitol and about 20 w/w % of microcrystalline cellulose; and

(d) about 10 w/w % of croscarmellose sodium.

Additional excipients can be included in the pharmaceutical formulations of the present disclosure. Further examples of excipients include, but are not limited to, pigments, colorants, flavoring agents, preservatives, and sweeteners. Flavors and colors can be added to improve the taste or appearance of a formulation. Examples of preservatives used in pharmaceutical compositions are aromatic alcohols, such as benzyl or phenol alcohol, antioxidants such as vitamin A, vitamin E, vitamin C, and selenium, amino acids such as cysteine and methionine, citric acid and sodium citrate, or synthetic preservatives such as methyl paraben and propyl paraben. Sweeteners can be added to make the ingredients more palatable, especially in chewable tablets or liquids like syrups.

In some embodiments, the spray-dried dispersion is the spray-dried dispersion described in Example 3.

### Dosage Forms

The pharmaceutical compositions of the present disclosure are formulated for oral administration. In preparing the compositions in oral dosage form, any of the usual pharmaceutical media can be employed. For solid oral preparations such as, for example, powders, capsules, caplets, gelcaps, and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Oral pharmaceutical dosage forms can be solid, gel, or liquid. In some embodiments, the dosage form is a solid dosage form. In some embodiments, the solid dosage form is a pill, tablet, capsule, caplet, gelcaps, granules, powder, sachet, melting strip, or melting film. In some embodiments, the solid dosage form is coated. In some embodiments, the coating is an enteric coating, a sugar coating, or a film coating. In some embodiments, the solid dosage form is a coated particle, coated tablet, enterocoated tablet, or enterocoated capsule. In some embodiments, the solid dosage form is a pill or tablet. Types of oral tablets include compressed, chewable lozenges and tablets which may be enteric coated, sugar coated or film coated. In some embodiments, the pharmaceutical composition is formulated as a capsule. In some embodiments, the pharmaceutical composition is formulated as a powder, solution, or suspension (e.g., in propylene carbonate, vegetable oils, PEG's, poloxamer 124 or triglycerides), or is encapsulated in a capsule (gelatin or cellulose base capsule). Capsules can be hard or soft gelatin capsules, while granules and powders can be provided in non-effervescent or effervescent form with a combination of other ingredients known to those skilled in the art.

The pharmaceutical compositions of the present disclosure can contain, per dosage unit, e.g., tablet, capsule, powder, and the like, an amount of the active ingredient necessary to deliver an effective dose as described above.

In some embodiments, the pharmaceutical compositions of the present disclosure are formulated in unit dosage form. In some embodiments, the compound of Formula (I), or pharmaceutically acceptable salt thereof, is present in an amount of about 5 mg to about 200 mg in the unit dosage form. For example, about 5 mg to about 175 mg, about 5 mg to about 150 mg, about 5 mg to about 125 mg, about 5 mg to about 100 mg, about 5 mg to about 75 mg, about 5 mg to about 50 mg, about 5 mg to about 25 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 200 mg, about 75 mg to about 175 mg, about 75 mg to about 150 mg, about 75 mg to about 125 mg, about 75 mg to about 100 mg, about 100 mg to about 200 mg, about 100 mg to about 175 mg, about 100 mg to about 150 mg, about 100 mg to about 125 mg, about 125 mg to about 200 mg, about 125 mg to about 175 mg, about 125 mg to about 150 mg, about 150 mg to about 200 mg, about 150 mg to about 175 mg, or about 175 mg to about 200 mg in the unit dosage form. In some embodiments, the compound of Formula (I), or pharmaceutically acceptable salt thereof, is present in an amount of about 25 mg to about 125 mg in the unit dosage form. In some embodiments, the compound of Formula (I), or pharmaceutically acceptable salt thereof, is present in an amount of about 75 mg to about 150 mg in the unit dosage form. In some embodiments, the compound of Formula (I), or pharmaceutically acceptable salt thereof, is present in an amount of about 5 mg, about 10 mg, about 25 mg, about 35 mg, about 50 mg, about 65 mg, about 75 mg, about 90 mg, about 100 mg, about 125 mg, about 150 mg, about 175 mg, or about 200 mg in the unit dosage form, or within a range defined by any of the preceding values. In some embodiments, the compound of Formula (I), or pharmaceutically acceptable salt thereof, is present in an amount of about 50 mg in the unit dosage form. In some embodiments, the compound of Formula (I), or pharmaceutically acceptable salt thereof, is present in an amount of about 100 mg in the unit dosage form. In some embodiments, the compound of Formula (I), or pharmaceutically acceptable salt thereof, is present in an amount of about 25 mg in the unit dosage form. In some embodiments, the compound of Formula (I), or pharmaceutically acceptable salt thereof, is present in an amount of 5 mg to 250 mg in the unit dosage form. For example, 5 mg to 175 mg, 5 mg to 150 mg, 5 mg to 125 mg, 5 mg to 100 mg, 5 mg to 75 mg, 5 mg to 50 mg, 5 mg to 25 mg, 25 mg to 200 mg, 25 mg to 175 mg, 25 mg to 150 mg, 25 mg to 125 mg, 25 mg to 100 mg, 25 mg to 75 mg, 25 mg to 50 mg, 50 mg to 200 mg, 50 mg to 175 mg, 50 mg to 150 mg, 50 mg to 125 mg, 50 mg to 100 mg, 50 mg to 75 mg, 75 mg to 200 mg, 75 mg to 175 mg, 75 mg to 150 mg, 75 mg to 125 mg, 75 mg to 100 mg, 100 mg to 200 mg, 100 mg to 175 mg, 100 mg to 150 mg, 100 mg to 125 mg, 125 mg to 200 mg, 125 mg to 175 mg, 125 mg to 150 mg, 150 mg to 200 mg, 150 mg to 175 mg, or 175 mg to 200 mg in the unit dosage form. In some embodiments, the compound of Formula (I), or pharmaceutically acceptable salt thereof, is present in an amount of 25 mg to 125 mg in the unit dosage form. In some embodiments, the compound of Formula (I), or pharmaceutically acceptable salt thereof, is present in an amount of 75 mg to 150 mg in the unit dosage form. In some embodiments, the compound of Formula (I), or pharmaceutically acceptable salt thereof, is present in an amount of 5 mg, 10 mg, 25 mg, 35 mg, 50 mg, 65 mg, 75 mg, 90 mg, 100 mg, 125 mg, 150 mg, 175 mg, or 200 mg in the unit dosage form, or within a range defined by any of the preceding values. In some embodiments, the compound of Formula (I), or pharmaceutically acceptable salt thereof, is present in an amount of 50 mg in the unit dosage form. In some embodiments, the compound of Formula (I), or pharmaceutically acceptable salt thereof, is present in an amount of 100 mg in the unit dosage form. In some embodiments, the pharmaceutical compositions of the present disclosure are formulated as a tablet. In some embodiments, the tablet is coated. In some embodiments, the pharmaceutical compositions of the present disclosure are formulated as capsules. In some embodiments, the pharmaceutical compositions are in sachet form. In some embodiments, the pharmaceutical compositions are in granule form.

### Dosing and Administration

In some embodiments of any of the methods disclosed herein, the compound of Formula (I), or a pharmaceutically acceptable salt thereof, is administered at a frequency of not less than twice daily; and the amount of the compound of Formula (I), or a pharmaceutically acceptable salt thereof, in the first administration is less than the amount of the compound of Formula (I), or a pharmaceutically acceptable salt thereof, in the second and any subsequent administrations.

In some embodiments, the compound of Formula (I), or a pharmaceutically acceptable salt thereof, is administered at a frequency of twice daily (i.e., comprising a first administration and a second administration).

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100, 1:1.1 to 1:95, 1:1.1 to 1:90, 1:1.1 to 1:85, about 1:1.1 to 1:80, about 1:1.1 to 1:75, 1:1.1 to 1:70, 1:1.1 to 1:65, 1:1.1 to 1:60, 1:1.1 to 1:55, 1:1.1 to 1:50, 1:1.1 to 1:45, 1:1.1 to 1:40, 1:1.1 to 1:35, 1:1.1 to 1:30, 1:1.1 to 1:25, 1:1.1 to 1:20, about 1:1.1 to 1:15, 1:1.1 to 1:10, 1:1.1 to 1:9, 1:1.1 to 1:8, 1:1.1 to 1:7, 1:1.1 to 1:6, 1:1.1 to 1:5, 1:1.1 to 1:4, 1:1.1 to 1:3.5, 1:1.1 to 1:3, 1:1.1 to 1:2.5, 1:1.1 to 1:2, 1:1.1 to 1:1.5, or 1:1.1 to 1.25.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:100, about 1:1 to about 1:95, about 1:1 to about 1:90, about 1:1 to about 1:85, about 1:1 to about 1:80, about 1:1 to about 1:75, about 1:1 to about 1:70, about 1:1 to about 1:65, about 1:1 to about 1:60, about 1:1 to about 1:55, about 1:1 to about 1:50, about 1:1 to about 1:45, about 1:1 to about 1:40, about 1:1 to about 1:35, about 1:1 to about 1:30, about 1:1 to about 1:25, about 1:1 to about 1:20, about 1:1 to about 1:15, about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3.5, about 1:1 to about 1:3, about 1:1 to about 1:2.5, about 1:1 to about 1:2, about 1:1 to about 1:1.5 or about 1:1 to about 1.25.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:100.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:50.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:10.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:3.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:2.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:2.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:2.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:2.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration about 1:3.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:3.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:4.

In some embodiments, the amount of the compound of Formula (I), or a pharmaceutically acceptable salt thereof, administered daily is less than or equal to about 1000 mg based on the weight of the free base.

In some embodiments, the amount of the compound of Formula (I), or a pharmaceutically acceptable salt thereof, administered daily is about 25 mg to about 1000 mg, about 50 mg to about 1000 mg, about 50 mg to about 950 mg, about 50 mg to about 900 mg, about 50 mg to about 850 mg, about 50 mg to about 800 mg, about 50 mg to about 750 mg, about 50 mg to about 700 mg, about 50 mg to about 650 mg, about 50 mg to about 600 mg, about 50 mg to about 550 mg, about 50 mg to about 500 mg, about 50 mg to about 450 mg, about 50 mg to about 400 mg, about 50 mg to about 350 mg, about 50 mg to about 300 mg, about 75 mg to about 350 mg, or about 75 mg to about 300 mg, wherein the daily amounts are based on the weight of the free base of the compound of Formula (I).

In some embodiments, the amount of the compound of Formula (I), or a pharmaceutically acceptable salt thereof, administered daily is about 100 mg to about 1000 mg, about 100 mg to about 950 mg, about 100 mg to about 900 mg, about 100 mg to about 850 mg, about 100 mg to about 800 mg, about 100 mg to about 750 mg, about 100 mg to about 700 mg, about 100 mg to about 650 mg, about 100 mg to about 600 mg, about 100 mg to about 550 mg, about 100 mg to about 500 mg, about 100 mg to about 450 mg, about 100 mg to about 400 mg, about 100 mg to about 350 mg, about 100 mg to about 300 mg, or about 100 mg to about 250, wherein the daily amounts are based on the weight of the free base of the compound of Formula (I).

In some embodiments, the amount of the compound of Formula (I), or a pharmaceutically acceptable salt thereof, administered daily is from about 50 mg to about 1000 mg based on the weight of the free base.

In some embodiments, the amount of the compound of Formula (I), or a pharmaceutically acceptable salt thereof, administered daily is from about 100 mg to about 1000 mg based on the weight of the free base.

In some embodiments, the amount of the compound of Formula (I), or a pharmaceutically acceptable salt thereof, administered daily is from about 100 mg to about 500 mg based on the weight of the free base.

In some embodiments, the amount of the compound of Formula (I), or a pharmaceutically acceptable salt thereof, administered daily is from about 100 mg to about 400 mg based on the weight of the free base.

In some embodiments, the amount of the compound of Formula (I), or a pharmaceutically acceptable salt thereof, administered daily is from about 100 mg to about 300 mg based on the weight of the free base.

In some embodiments, the amount of the compound of Formula (I), or a pharmaceutically acceptable salt thereof, administered daily is about 200 mg based on the weight of the free base. In a further embodiment, the first administration of the compound of Formula (I), or a pharmaceutically acceptable salt thereof is about 50 mg based on the weight of the free base and the second administration of the compound of Formula (I), or a pharmaceutically acceptable salt thereof is about 150 mg based on the weight of the free base.

In some embodiments, the amount of the compound of Formula (I), or a pharmaceutically acceptable salt thereof, administered daily is about 250 mg based on the weight of the free base. In further embodiments, the first administration of the compound of Formula (I), or a pharmaceutically acceptable salt thereof is about 100 mg and the second administration of the compound of Formula (I), or a pharmaceutically acceptable salt thereof is about 150 mg based on the weight of the free base.

In some embodiments, the amount of the compound of Formula (I), or a pharmaceutically acceptable salt thereof, administered daily is about 300 mg based on the weight of the free base. In a further embodiments, the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base.

In some embodiments, the subject weighs greater than or equal to about 55 kg. In some embodiments, the amount of the compound of Formula (I), or a pharmaceutically acceptable salt thereof, administered daily is about 200 mg or above and the subject weighs greater than or equal to about 55 kg.

In some embodiments, the amount of the compound of Formula (I), or a pharmaceutically acceptable salt thereof, administered daily is about 50 mg based on the weight of the free base.

In some embodiments, the subject weighs from about 10 kg to about 20 kg.

In some embodiments, the amount of the compound of Formula (I), or a pharmaceutically acceptable salt thereof, administered daily is about 50 mg based on the weight of the free base and the subject weighs from about 10 kg to about 20 kg.

In some embodiments, the amount of the compound of Formula (I), or a pharmaceutically acceptable salt thereof, administered daily is about 100 mg based on the weight of the free base. In some embodiments, the first administration of the compound of Formula (I), or a pharmaceutically acceptable salt thereof is about 25 mg based on the weight of the free base and the second administration of the compound of Formula (I), or a pharmaceutically acceptable salt thereof is about 75 mg based on the weight of the free base.

In some embodiments, the subject weighs from about 20 kg to about 55 kg and the amount of the compound of Formula (I), or a pharmaceutically acceptable salt thereof, administered daily is about 100 mg based on the weight of the free base.

In some embodiments, the compound of Formula (I), or a pharmaceutically acceptable salt thereof, is administered at a frequency of not less than twice daily, wherein the amount of the compound of Formula (I), or a pharmaceutically acceptable salt thereof, administered daily is greater than 200 mg based on the weight of the free base.

In some embodiments, the amount of the compound of Formula (I), or a pharmaceutically acceptable salt thereof, is administered at a frequency of twice daily.

In some embodiments, the amount of the compound of Formula (I), or a pharmaceutically acceptable salt thereof, administered daily is about 200 mg to about 1000 mg, about 200 mg to about 950 mg, about 200 mg to about 900 mg, about 200 mg to about 850 mg, about 200 mg to about 800 mg, about 200 mg to about 750 mg, about 200 mg to about 700 mg, about 200 mg to about 650 mg, about 200 mg to about 600 mg, about 200 mg to about 550 mg, about 200 mg to about 500 mg, about 200 mg to about 450 mg, about 200 mg to about 400 mg, about 200 mg to about 350 mg, about 200 mg to about 300 mg, or about 200 mg to about 250 mg, wherein the daily amounts are based on the weight of the free base of the compound of Formula (I).

In some embodiments, the amount of the compound of Formula (I), or a pharmaceutically acceptable salt thereof, administered daily is about 225 mg to about 1000 mg, about 225 mg to about 950 mg, about 225 mg to about 900 mg, about 225 mg to about 850 mg, about 225 mg to about 800 mg, about 225 mg to about 750 mg, about 225 mg to about 700 mg, about 225 mg to about 650 mg, about 225 mg to about 600 mg, about 225 mg to about 550 mg, about 225 mg to about 500 mg, about 225 mg to about 450 mg, about 225 mg to about 400 mg, about 225 mg to about 350 mg, about 225 mg to about 300 mg, or about 225 mg to about 250 mg, wherein the daily amounts are based on the weight of the free base of the compound of Formula (I).

In some embodiments, the amount of the compound of Formula (I), or a pharmaceutically acceptable salt thereof, administered daily is from about 200 mg to about 1000 mg based on the weight of the free base.

In some embodiments, the amount of the compound of Formula (I), or a pharmaceutically acceptable salt thereof, administered daily is from about 225 mg to about 1000 mg based on the weight of the free base.

In some embodiments, the amount of the compound of Formula (I), or a pharmaceutically acceptable salt thereof, administered daily is from about 225 mg to about 500 mg based on the weight of the free base.

In some embodiments, the amount of the compound of Formula (I), or a pharmaceutically acceptable salt thereof, administered daily is from about 225 mg to about 400 mg based on the weight of the free base.

In some embodiments, the amount of the compound of Formula (I), or a pharmaceutically acceptable salt thereof, administered daily is from about 225 mg to about 300 mg based on the weight of the free base.

In some embodiments, the amount of the compound of Formula (I), or a pharmaceutically acceptable salt thereof, administered daily is about 250 mg based on the weight of the free base. In a further embodiment, the first administration of the compound of Formula (I), or a pharmaceutically acceptable salt thereof is about 125 mg based on the weight of the free base and the second administration of the compound of Formula (I), or a pharmaceutically acceptable salt thereof is about 125 mg based on the weight of the free base.

In some embodiments, the amount of the compound of Formula (I), or a pharmaceutically acceptable salt thereof, administered daily is about 300 mg based on the weight of the free base. In a further embodiment, the first administration of the compound of Formula (I), or a pharmaceutically acceptable salt thereof is about 150 mg based on the weight of the free base and the second administration of the compound of Formula (I), or a pharmaceutically acceptable salt thereof is about 150 mg based on the weight of the free base.

In some embodiments of the methods disclosed herein (e.g., when the compound of Formula (I) is administered at a frequency of twice daily), there are about 6 to about 14 hours between the first and second administrations of the compound of Formula (I), or a pharmaceutically acceptable salt thereof. In some embodiments, there are about 8 to about 14 hours between the first and second administrations of the compound of Formula (I), or a pharmaceutically acceptable salt thereof. In some embodiments, there are about 11 to about 13 hours between the first and second administrations of the compound of Formula (I), or a pharmaceutically acceptable salt thereof. In some embodiments, there are about 12 hours between the first and second administrations of the compound of Formula (I), or a pharmaceutically acceptable salt thereof.

In some embodiments, the compound of Formula (I), or a pharmaceutically acceptable salt thereof, is administered in a dose of about 25 mg, based on the weight of the free base. In some embodiments, the compound of Formula (I), or a pharmaceutically acceptable salt thereof, is administered in a dose of about 50 mg, based on the weight of the free base. In some embodiments, the compound of Formula (I), or a pharmaceutically acceptable salt thereof, is administered in a dose of about 75 mg, based on the weight of the free base. In some embodiments, the compound of Formula (I), or a pharmaceutically acceptable salt thereof, is administered in a dose of about 100 mg, based on the weight of the free base.

In some embodiments, the pharmaceutical composition is administered in a dose of about 25 mg of the compound of Formula (I), or a pharmaceutically acceptable salt thereof, based on the weight of the free base. In some embodiments, the pharmaceutical composition is administered in a dose of about 50 mg of the compound of Formula (I), or a pharmaceutically acceptable salt thereof, based on the weight of the free base. In some embodiments, the pharmaceutical composition is administered in a dose of about 75 mg of the compound of Formula (I), or a pharmaceutically acceptable salt thereof, based on the weight of the free base. In some embodiments, the pharmaceutical composition is administered in a dose of about 100 mg of the compound of Formula (I), or a pharmaceutically acceptable salt thereof, based on the weight of the free base.

In some embodiments, the pharmaceutical composition comprises about 25 mg of the compound of Formula (I), or a pharmaceutically acceptable salt thereof, based on the weight of the free base. In some embodiments, the pharmaceutical composition comprises about 50 mg of the compound of Formula (I), or a pharmaceutically acceptable salt thereof, based on the weight of the free base. In some embodiments, the pharmaceutical composition comprises about 75 mg of the compound of Formula (I), or a pharmaceutically acceptable salt thereof, based on the weight of the free base. In some embodiments, the pharmaceutical composition comprises about 100 mg of the compound of Formula (I), or a pharmaceutically acceptable salt thereof, based on the weight of the free base.

The daily dosage of the compound of Formula (I), or a pharmaceutically acceptable salt thereof, in a pharmaceutical composition as described in the present disclosure can be varied over a wide range from about 1.0 mg to about 10,000 mg per adult human per day, or higher, or any range therein. For oral administration, the compositions can be provided in the form of tablets containing, for example, about 0.01 mg, about 0.05 mg, about 0.1 mg, about 0.5 mg, about 1.0 mg, about 2.5 mg, about 5.0 mg, about 10.0 mg, about 15.0 mg, about 25.0 mg, about 50.0 mg, about 75.0 mg, about 100 mg, about 150 mg, about 200 mg, about 250 or about 500 milligrams of the compound of Formula (I), or pharmaceutically acceptable salt thereof, for the symptomatic adjustment of the dosage to the subject to be treated. In some embodiments, an effective amount of the compound of Formula (I), or pharmaceutically acceptable salt thereof, can be supplied at a dosage level of from about 0.1 mg/kg to about 1000 mg/kg of body weight per day, or any range therein, for example, the range can be from about 0.5 mg/kg to about 500 mg/kg, about 1.0 mg/kg to about 250 mg/kg, about 0.1 mg/kg to about 100 mg/kg, about 0.1 mg/kg to about 50.0 mg/kg of body weight per day, about 0.1 mg/kg to about 15.0 mg/kg of body weight per day, about 0.5 mg/kg to about 7.5 mg/kg of body weight per day, or any amount to range therein. In some embodiments, an effective amount of the compound of Formula (I), or pharmaceutically acceptable salt thereof, can be supplied at a dosage level of from 0.1 mg/kg to 1000 mg/kg of body weight per day, or any range therein, for example, the range can be from 0.5 mg/kg to 500 mg/kg, 1.0 mg/kg to 250 mg/kg, 0.1 mg/kg to 100 mg/kg, 0.1 mg/kg to 50.0 mg/kg of body weight per day, 0.1 mg/kg to 15.0 mg/kg of body weight per day, 0.5 mg/kg to 7.5 mg/kg of body weight per day, or any amount to range therein. A pharmaceutical composition as provided herein can be administered on a regimen of 1 to 4 times per day or in a single daily dose.

In some embodiments, the daily dose of the compound of Formula (I), or a pharmaceutically acceptable salt thereof, is about 25 mg, based on the weight of the free base. In some embodiments, the daily dose of the compound of Formula (I), or a pharmaceutically acceptable salt thereof, is about 50 mg, based on the weight of the free base. In some embodiments, the daily dose of the compound of Formula (I), or a pharmaceutically acceptable salt thereof, is about 75 mg, based on the weight of the free base. In some embodiments, the daily dose of the compound of Formula (I), or a pharmaceutically acceptable salt thereof, is about 100 mg, based on the weight of the free base. In some embodiments, the daily dose of the compound of Formula (I), or a pharmaceutically acceptable salt thereof, is about 150 mg, based on the weight of the free base. In some embodiments, the daily dose of the compound of Formula (I), or a pharmaceutically acceptable salt thereof, is about 200 mg, based on the weight of the free base.

In some embodiments, the compound of Formula (I), or a pharmaceutically acceptable salt thereof, is administered twice daily in a dose of about 25 mg, based on the weight of the free base. In some embodiments, the compound of Formula (I), or a pharmaceutically acceptable salt thereof, is administered twice daily in a dose of about 50 mg, based on the weight of the free base. In some embodiments, the compound of Formula (I), or a pharmaceutically acceptable salt thereof, is administered twice daily in a dose of about 75 mg, based on the weight of the free base. In some embodiments, the compound of Formula (I), or a pharmaceutically acceptable salt thereof, is administered twice daily in a dose of about 100 mg, based on the weight of the free base.

In some embodiments, the daily dose of the pharmaceutical composition is about 25 mg of the compound of Formula (I), or a pharmaceutically acceptable salt thereof, based on the weight of the free base. In some embodiments, the daily dose of the pharmaceutical composition is about 50 mg of the compound of Formula (I), or a pharmaceutically acceptable salt thereof, based on the weight of the free base. In some embodiments, the daily dose of the pharmaceutical composition is about 75 mg of the compound of Formula (I), or a pharmaceutically acceptable salt thereof, based on the weight of the free base. In some embodiments, the daily dose of the pharmaceutical composition is about 100 mg of the compound of Formula (I), or a pharmaceutically acceptable salt thereof, based on the weight of the free base. In some embodiments, the daily dose of the pharmaceutical composition is about 150 mg of the compound of Formula (I), or a pharmaceutically acceptable salt thereof, based on the weight of the free base. In some embodiments, the daily dose of the pharmaceutical composition is about 200 mg of the compound of Formula (I), or a pharmaceutically acceptable salt thereof, based on the weight of the free base.

In some embodiments, the pharmaceutical composition is administered twice daily in a dose of about 25 mg of the compound of Formula (I), or a pharmaceutically acceptable salt thereof, based on the weight of the free base. In some embodiments, the pharmaceutical composition is administered twice daily in a dose of about 50 mg of the compound of Formula (I), or a pharmaceutically acceptable salt thereof, based on the weight of the free base. In some embodiments, the compound of Formula (I), or a pharmaceutically acceptable salt thereof, is administered twice daily in a dose of about 75 mg of the compound of Formula (I), or a pharmaceutically acceptable salt thereof, based on the weight of the free base. In some embodiments, the pharmaceutical composition is administered twice daily in a dose of about 100 mg of the compound of Formula (I), or a pharmaceutically acceptable salt thereof, based on the weight of the free base.

In some embodiments, the method comprises administering a daily dose of the pharmaceutical composition comprising about 25 mg of the compound of Formula (I), or a pharmaceutically acceptable salt thereof, based on the weight of the free base. In some embodiments, the method comprises administering a daily dose of the pharmaceutical composition comprising about 50 mg of the compound of Formula (I), or a pharmaceutically acceptable salt thereof, based on the weight of the free base. In some embodiments, the method comprises administering a daily dose of the pharmaceutical composition comprising about 75 mg of the compound of Formula (I), or a pharmaceutically acceptable salt thereof, based on the weight of the free base. In some embodiments, the method comprises administering a daily dose of the pharmaceutical composition comprising about 100 mg of the compound of Formula (I), or a pharmaceutically acceptable salt thereof, based on the weight of the free base. In some embodiments, the method comprises administering a daily dose of the pharmaceutical composition comprising about 150 mg of the compound of Formula (I), or a pharmaceutically acceptable salt thereof, based on the weight of the free base. In some embodiments, the method comprises administering a daily dose of the pharmaceutical composition comprising about 200 mg of the compound of Formula (I), or a pharmaceutically acceptable salt thereof, based on the weight of the free base.

In some embodiments, the method comprises administering the pharmaceutical composition twice daily in a dose of about 25 mg of the compound of Formula (I), or a pharmaceutically acceptable salt thereof, based on the weight of the free base. In some embodiments, the method comprises administering the pharmaceutical composition twice daily in a dose of about 50 mg of the compound of Formula (I), or a pharmaceutically acceptable salt thereof, based on the weight of the free base. In some embodiments, the method comprises administering the compound of Formula (I), or a pharmaceutically acceptable salt thereof, twice daily in a dose of about 75 mg of the compound of Formula (I), or a pharmaceutically acceptable salt thereof, based on the weight of the free base. In some embodiments, the method comprises administering the pharmaceutical composition twice daily in a dose of about 100 mg of the compound of Formula (I), or a pharmaceutically acceptable salt thereof, based on the weight of the free base.

Factors associated with the particular subject being treated, including subject age, weight, diet, and time of administration, can result in the need to adjust dosages. In some embodiments, the subject is a human adult. In some embodiments, the subject is a pediatric subject.

One skilled in the art will recognize that both in vivo and in vitro trials using suitable, known, and generally accepted cell and/or animal models are predictive of the ability of a test compound to treat or prevent a given disorder. One skilled in the art will further recognize that human clinical trials including first-in-human, dose ranging and efficacy trials, in healthy subjects and/or those suffering from a given disorder, can be completed according to methods well known in the clinical and medical arts. For example, determining proper dosages for pediatric subjects can be determined using known methods, including weight, age, and models such as Simcyp® Pediatric Simulation modeling (CERTARA, Princeton, N.J.) which can be used to establish a pharmacokinetic approach for dosing that takes into account subject age, ontogeny of the clearance pathways that a compound of Formula (I), or a pharmaceutically acceptable salt thereof, and body surface area (BSA).

In some embodiments, the pharmaceutical compositions of the present disclosure are stable for at least 3 months. In some embodiments, the pharmaceutical compositions are stable for at least 6 months. In some embodiments, the pharmaceutical compositions are stable for at least 9 months. In some embodiments, the pharmaceutical compositions are stable for at least 12 months. For example, the compositions do not exhibit a change (e.g., greater than 5%) in appearance, pH, percent impurities, activity (as measured by in vitro assays), or osmolarity over time, e.g., at least 3 months, 6 months, 9 months, or at least 12 months as compared to the original composition after manufacturing. In some embodiments, the pharmaceutical compositions do not exhibit a significant change, as defined by the International Conference on Harmonisation of Technical Requirements for Registration of Pharmaceuticals for Human Use (ICH), in one or more of appearance, pH, percent impurities, activity (as measured by in vitro assays), or osmolarity over time, e.g., at least 12 months as compared to the original pharmaceutical composition after manufacturing.

### Kits

Also provided are kits. Typically, a kit includes one or more pharmaceutical compositions as described herein, e.g., a pharmaceutical composition containing, e.g., a spray-dried dispersion as described in Examples 1-4, or the formulation described in Example 9. In certain embodiments, a kit can include one or more delivery systems, e.g., for delivering or administering the pharmaceutical composition as provided herein, and directions for use of the kit (e.g., instructions for treating a subject). In some embodiments, the kit can include a pharmaceutical composition as described herein and a label that indicates that the contents are to be administered to a subject with congenital adrenal hyperplasia. The actual dose of the compound of Formula (I), or pharmaceutically acceptable salt thereof, provided herein depends on the specific formulation, the weight of the patient, and on the condition to be treated.

### EXAMPLES

### Example 1: Spray-Dried Dispersion Formulations Containing the Compound of Formula (I) and Various Polymers

Spray-Dried Dispersion Formulations

A series of spray-dried dispersion (SDD) formulations containing the compound of Formula (I) and a polymer were prepared. The SDD formulations included: (1) 10% compound of Formula (I)/90% hydroxypropylmethylcellulose acetate succinate-L (HPMCAS-L); (2) 25% compound of Formula (I)/75% HPMCAS-L; (3) 40% compound of Formula (I)/60% HPMCAS-L; (4) 25% compound of Formula (I)/75% polyvinyl pyrrolidone vinyl acetate 64 (PVP/VA 64); (5) 25% compound of Formula (I)/60% Cabosil (fumed silica)/15% HPMCAS-L; (6) 25% compound of Formula (I)/75% HPMCAS-M; and (7) 25% compound of Formula (I)/75% methyl methacrylate copolymer (1:1) (Eudragit® L100).

The PVP/VA polymer was a copolymer of 1-vinyl-2-pyrrolidone and vinyl acetate with a ratio of 60:40 by weight 1-vinyl-2-pyrrolidone:vinyl acetate with an average molecular weight of 45,000-70,000 (copovidone, sold as Kollidon® VA 64, BASF, Florham Park, N.J.). The HPMCAS was a mixture of acetic acid and monosuccinic acid esters of hydroxypropylmethyl cellulose that was either grade L (HPMCAS-L), with an acetyl content of 5-9%, a succinoyl content of 14-18%, a methoxyl content of 20-24%, and a hydroxypropoxy content of 5-9% (sold by Shin-Etsu, Japan); or grade M (HPMCAS-M), with an acetyl content of 7-11%, a succinoyl content of 10-14%, a methoxyl content of 21-25%, and a hydroxypropoxy content of 5-9% (sold by Shin-Etsu, Japan).

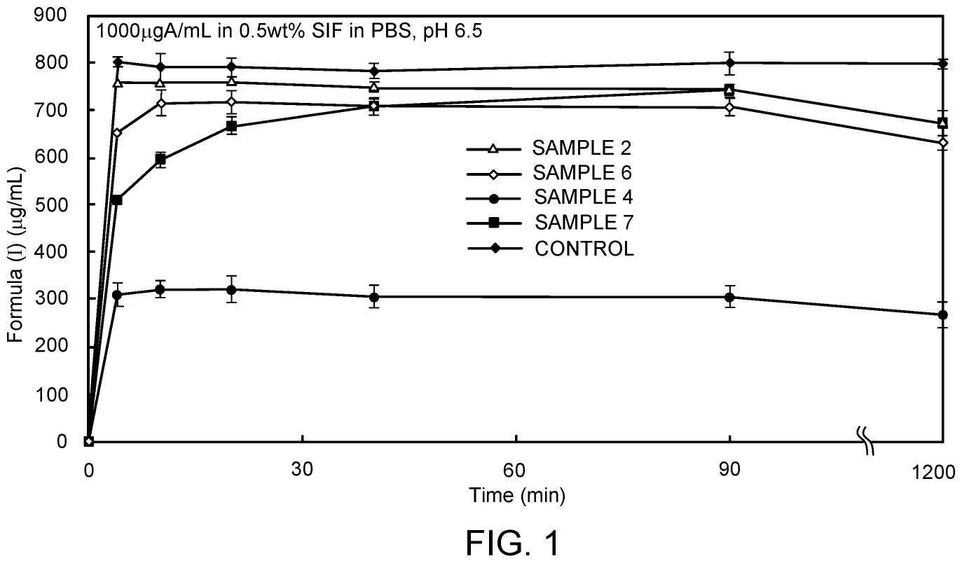

Dissolution performance Dissolution performance of several of the SDD formulations described above was tested (see FIG. 1). 1000 μgA/mL of each SDD was tested in 0.5 wt % simulated intestinal fluid (SIF) in PBS, pH 6.5. Samples were tested at 5, 10, 20, 45, 90, and 1200 minutes. A lipid formulation containing 10% of the compound of Formula (I) was used as a control. The results are shown in Table 4, below.

TABLE 4
Dissolution data of various SDDs

| | Cₘₐₓ₉₀ | AUC₉₀ | Cₘₐₓ₉₀ | Ultra₉₀ | C₁₂₀₀ | Ultra₁₂₀₀ |
|---|---|---|---|---|---|---|
| Sample | (μg/mL) | (min*μg/mL) | (μg/mL) | (μg/mL) | (μg/mL) | (μg/mL) |
| 2 | 762 | 66,080 | 743 | 210 | 671 | 166 |
| 4 | 322 | 27,330 | 306 | 109* | 268 | 199 |
| 6 | 718 | 62,240 | 708 | 202 | 632 | 217 |
| 7 | 742 | 60,600 | 742 | 113* | 674 | 194 |
| Control | 802 | 69,580 | 800 | 253 | 799 | 270 |

*Large variability between replicates, high value discarded

Non-Sink Dissolution

A membrane flux assay was performed (see, e.g., Stewart et al., Mol. Pharm. (2017) 14:2032-2046) and non-sink dissolution data was collected for several of the SDD formulations described above and compared to the compound of Formula (I) and several reference formulations, including a semi-solid lipidic formulation (Reference Formulation 1) and two self-emulsifying drug delivery system (SEDDS) formulations (Reference Formulations 2 and 3). The components of the Reference Formulations are shown in Table 5, below, and include, in addition to the compound of Formula (I), caprylic/capric triglyceride (Labrafac® Lipophile, Gattefossé, France); propylene glycol dicrapolate/dicaprate (Labrafac® PG, Gattefossé, France); oleoyl polyoxyl-6 glycerides (Labrafil® M 1944 CS, Gattefossé, France); polysorbate 20; polyoxyl castor oil (Kolliphor® RH 40, BASF, Germany); polyoxyl 15 hydroxystearate (Kolliphor® HS 15, BASF, Germany); lauroyl polyoxyl-32 glycerides (Gelucire® 44/14, Gattefossé, France); d-α-tocopheryl polyethylene glycol 1000 succinate (TPGS); and diethylene glycol monoethyl ether (Transcutol®, Gattefossé, France).

TABLE 5
Reference formulations (capsules)

| Formulation | Ref. | Ref. | Ref. |
|---|---|---|---|
| (mg/caps) | Formulation 1 | Formulation 2 | Formulation 3 |
| Formula (I) | 50.0 | 50.0 | 50.0 |
| Labrafac ® Lipophile | 196.0 | 100.0 | 100.0 |
| Labrafac ® PG | 102.0 | — | — |
| Labrafil ® M 1944 | | 135.0 | 46.0 |
| CS | | | |
| Polysorbate 20 | — | — | 89.9 |
| Kolliphor ® RH 40 | — | — | 100.0 |
| Kolliphor ® HS 15 | — | 165.0 | — |
| Gelucire ® 44/14 | 95.0 | — | — |
| TPGS | 57.0 | — | 65.0 |
| Transcutol ® | — | 50.0 | 50.0 |
| Total | 500.0 | 500.0 | 500.0 |

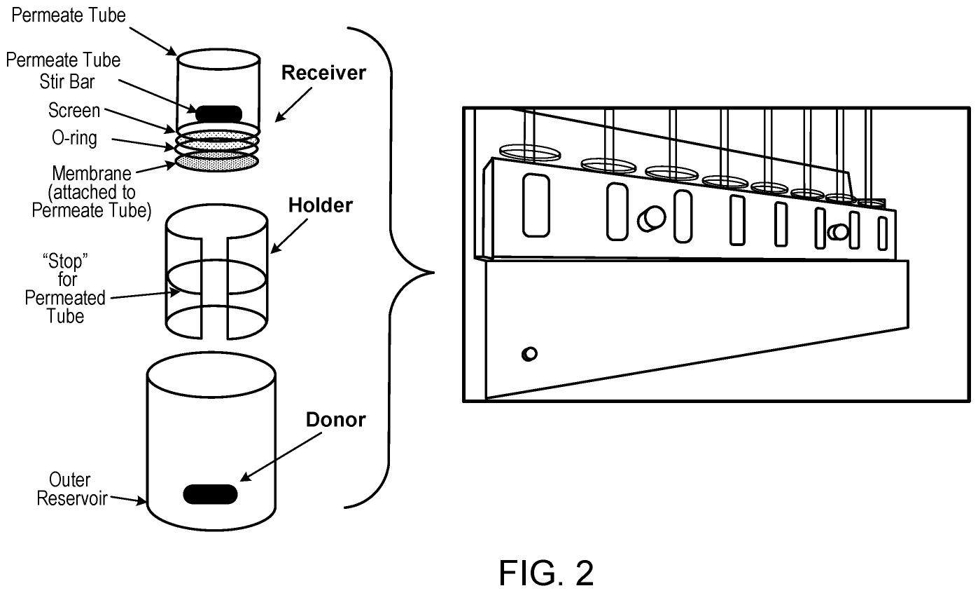

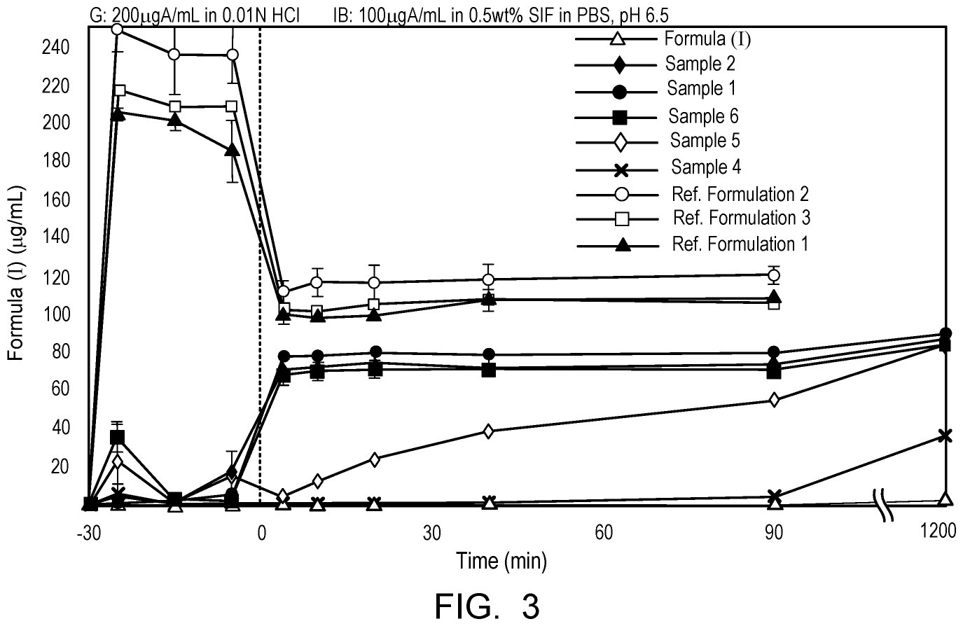

The assay measured the flux across simulated gastric and intestinal walls via UV spectroscopy (μDiss Profiler™, Pion Inc., Billerica, Mass.). Briefly, the assay was performed as follows. A vertical membrane flux cell consisting of a donor compartment and a receiver compartment, and separated by an Accurel PP 1E (55% porous, 100 μm thickness) polypropylene membrane (3M, Maplewood, Minn.) (FIG. 2), was impregnated with 50 μL of Pion GIT-0 lipid solution consisting of 2000 w/w phospholipid dissolved into dodecane (Pion Inc., Billerica, Mass.) and attached to the receiver vessel. Both the donor and receiver compartments were agitated by magnetic stirring. The receiver compartment contained a plastic spacer and grating to elevate the stir bar above the membrane. Samples were introduced to the donor vessel by pre-weighing directly into the donor vessel and subsequently adding dissolution medium. Once the dissolution medium was added to the donor vessel, the receiver vessel was inserted into the donor vessel and suspended vertically 5 mm above the donor compartment by a plastic sleeve. For this assay, the simulated gastric (feed) media was 0.1 N HCl, pH 2 and included 200 μgA/mL of each SDD, and the simulated intestinal (receiver) media was 0.5 wt % SIF in PBS, pH 6.5 and included 100 μgA/mL of each SDD. The temperature for the assay was maintained at 44.5° C. UV probes (10 mm path length) connected to a Rainbow UV spectrometer (Pion Inc.) system were used to determine the apparent drug concentration in the receiver vessels. Samples of the donor compartment were removed with a disposable pipet for centrifugation followed by HPLC and DLS analysis of the supernatant. The results are shown in FIG. 3 and Table 6, below.

TABLE 6
Non-sink dissolution data

| | C_{maxGB} | C_{max90 IB} | AUC_{4-90IB} | C₉₀ | Ultra₉₀ | C₁₂₀₀ |
|---|---|---|---|---|---|---|
| Sample | (μg/mL) | (μg/mL) | (min*μg/mL) | (μg/mL) | (μg/mL) | (μg/mL) |
| Formula (I) | 0 | 1 | 10 | 0 | 0 | 3 |
| 1 | 6 | 80 | 6,800 | 80 | 79 | 90 |
| 2 | 17 | 74 | 6,240 | 73 | 73 | 86 |
| 4 | 6 | 4 | 200 | 4 | 5 | 36 |
| 5 | 23 | 55 | 3,180 | 55 | 54 | 83 |
| 6 | 35 | 71 | 6,070 | 71 | 77 | 83 |
| Ref. Formulation 1 | 205 | 109 | 9,050 | 109 | — | — |
| Ref. Formulation 2 | 249 | 120 | 10,160 | 120 | — | — |
| Ref. Formulation 3 | 218 | 107 | 9,100 | 107 | — | — |

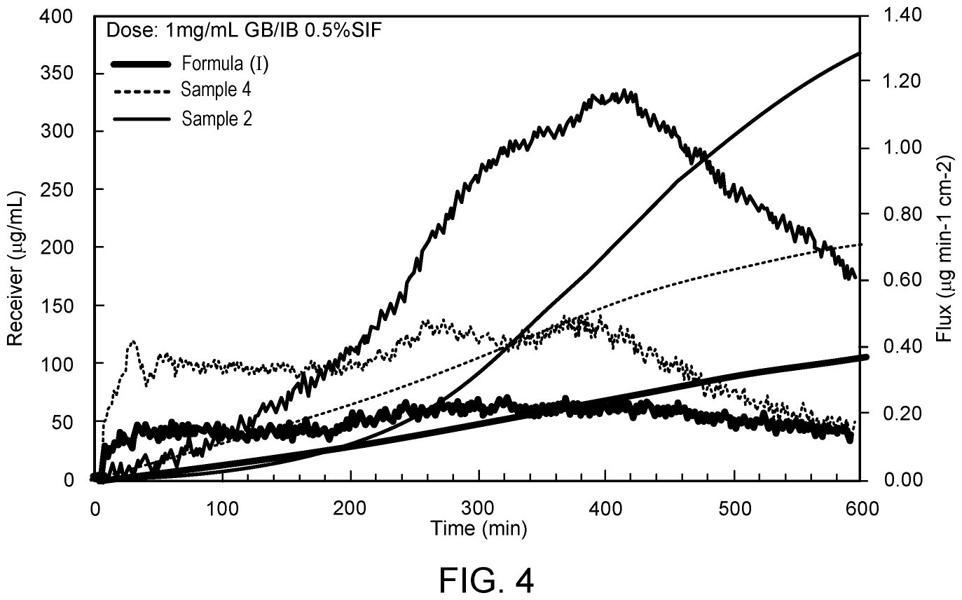

The membrane flux of 1 mg/mL gastric barrier/intestinal barrier (GB/IB) 0.5 wt % SIF doses of the compound of Formula (I) and spray-dried dispersions (2) 25% compound of Formula (I)/75% HPMCAS-L and (4) 25% compound of Formula (I)/75% PVP/VA 64 were also determined. The results are shown in FIG. 4 as receiver concentration vs. time and flux vs. time (smoothed derivative of receiver concentration x volume/surface area).

### Example 2: Characterization of a Spray-Dried Dispersion Containing 25% of the Compound of Formula (I) and 75% of a Polyvinyl Pyrrolidone Vinyl Acetate (PVP/VA) Polymer

### SDD Stability Screening

Several of the SDDs described in Example 1 were tested for chemical and physical stability. Wet SDD stability studies were performed, with samples stored at both 5° C. and 25° C. Measurements were taken after 1 week and 2 weeks of storage. The results are shown in Table 7 below. The column with a retention time of 32.36 min correlates with the compound of Formula (I).

TABLE 7
Wet SDD stability data

| Retention | | | | | 11.04 | 16.79 | 17.26 | 30.94 | 32.26 | z | z | z |
|---|---|---|---|---|---|---|---|---|---|---|---|---|
| time (min) | | | | | | | | | | | | |
| Relative | | | | | 0.34 | 0.52 | 0.53 | 0.96 | 1.00 | | | |
| retention time | | | | | | | | | | | | |
| | Storage | | | | | | | | | Total | Potency | Std |
| | temp | | Timepoint | | | | | | | impurities | (mgA/g) | Dev |
| Ref. Std. | | | | | | | | 0.37 | 99.63 | 0.37 | | |
| Formats | | | | | | | | 0.26 | 99.74 | 0.26 | | |
| (I) | | | | | | | | | | | | |
| Sample 1 | | | initial | | | 0.13 | 0.16 | 0.25 | 99.46 | 0.54 | 100 | 1.3 |
| | 5° | C. | 1 | week | | 0.03 | 0.03 | 0.26 | 99.69 | 0.31 | 99 | 0.0 |
| | | | 2 | weeks | | 0.12 | 0.16 | 0.28 | 99.45 | 0.55 | 99 | 0.5 |
| | 25° | C. | 1 | week | | 0.03 | 0.04 | 0.26 | 99.67 | 0.33 | 99 | 0.3 |
| | | | 2 | weeks | | 0.21 | 0.27 | 0.28 | 99.24 | 0.76 | 98 | 0.8 |
| Sample 2 | | | initial | | <LOQ | 0.07 | 0.08 | 0.26 | 99.59 | 0.41 | 247 | 0.3 |
| | 5° | C. | 1 | week | <LOQ | 0.02 | 0.03 | 0.26 | 99.70 | 0.30 | 248 | 1.3 |
| | | | 2 | weeks | <LOQ | 0.22 | 0.27 | 0.27 | 99.24 | 0.76 | 246 | 0.3 |
| | 25° | C. | 1 | week | <LOQ | 0.02 | 0.03 | 0.26 | 99.69 | 0.31 | 248 | 0.9 |
| | | | 2 | weeks | <LOQ | 0.23 | 0.28 | 0.26 | 99.23 | 0.77 | 246 | 1.4 |

LOQ = limit of quantification

Solution stability studies were also performed, with samples stored at both 5° C. and 25° C. Measurements were taken after 1 week and 2 weeks of storage. The results are shown in Table 8 below. The column with a retention time of 32.36 min correlates with the compound of Formula (I).

TABLE 8
SDD solution stability data

| Retention time | | | | 31.51 | 32.26 | z |
|---|---|---|---|---|---|---|
| (min) | | | | | | |
| Relative | | | | 0.97 | 1.00 | |
| retention time | | | | | | |
| | Storage | | | | | Total |
| | temp | | Timepoint | | | impurities |
| Ref. Std. | | | | 0.74 | 99.26 | 0.74 |
| Formula (I) | | | | 0.26 | 99.74 | 0.26 |
| Sample 1 | | | initial | 0.33 | 99.67 | 0.33 |
| | 5° | C. | 2 weeks | 0.29 | 99.71 | 0.29 |
| | 25° | C. | 2 weeks | 0.38 | 99.62 | 0.38 |
| Sample 2 | | | initial | 0.25 | 99.75 | 0.25 |
| | 5° | C. | 2 weeks | 0.26 | 99.74 | 0.26 |
| | 25° | C. | 2 weeks | 0.33 | 99.67 | 0.33 |

Stability studies were also performed for the SDD containing 25% of the compound of Formula (I) and 75% PVP/VA 64, with samples stored at both 5° C. (closed with desiccant), 25° C. (60% RH, closed with desiccant), and 30° C. (65% RH, closed with desiccant). Measurements were taken after storage for 1 month, 2 months, 3 months, 6 months, and 12 months. No change in purity was observed after 12 months of storage. The results are shown in Table 9 below. The column with a retention time of 30.2 min correlates with the compound of Formula (I).

TABLE 9
SDD stability data

| Retention | | | | 28.7 | 30.2 | | |
|---|---|---|---|---|---|---|---|
| time (min) | | | | | | | |
| Relative | | | | 0.95 | 1.00 | | |
| retention time | | | | | | | |
| | Storage | | | | | Total | Potency |
| | conditions | Timepoint | | | | impurities | (mgA/g) |
| Crystalline | | | | 0.26 | 99.74 | 0.26 | 1001 |
| Formula (I) | | | | | | | |
| Sample 4 | | initial | | 0.26 | 99.74 | 0.26 | 245 |
| (25% | 5° C. | 1 | month | 0.25 | 99.75 | 0.25 | 247 |
| Formula | (closed w/ | 2 | months | 0.25 | 99.75 | 0.25 | 244 |
| (I):75% | desiccant) | 3 | months | 0.26 | 99.74 | 0.26 | 246 |
| PVP/VA 64) | | 6 | months | 0.25 | 99.75 | 0.25 | 245 |
| | | 12 | months | 0.25 | 99.75 | 0.25 | 248 |
| | 25° C./60% RH | 1 | month | 0.25 | 99.75 | 0.25 | 245 |
| | (closed with | 2 | months | 0.25 | 99.75 | 0.25 | 247 |
| | desiccant) | 3 | months | 0.25 | 99.75 | 0.25 | 246 |
| | | 6 | months | 0.25 | 99.75 | 0.25 | 242 |
| | | 12 | months | 0.25 | 99.75 | 0.25 | 245 |
| | 30° C./65% RH | 1 | month | 0.25 | 99.75 | 0.25 | 249 |
| | (closed with | 2 | months | 0.25 | 99.75 | 0.25 | 242 |
| | desiccant) | 3 | months | 0.25 | 99.75 | 0.25 | 246 |
| | | 6 | months | 0.25 | 99.73 | 0.25 | 243 |
| | | 12 | months | 0.25 | 99.75 | 0.25 | 242 |

While Samples 1 and 2 showed degradation after about 2 weeks of storage, the SDD containing 25% of the compound of Formula (I) and 7500 PVP/VA 64 (Sample 4) was found to be both chemically and physically stable and was further screened and characterized as described below.

25% Formula (I)/75% PVP/VA 64 SDD Process Parameter Screening Manufacture Round 1

The 25% Formula (I)/75%0 PVP/VA 64 SDD was prepared on a Pharmaceutical Spray Dryer with 100 kg/hr drying gas capacity (PSD-1). The manufacturing summary is shown in Table 10, below.

TABLE 10
Manufacturing summary of process parameters

| Formulation | 25% Formula (I):75% PVP/VA 64 |
|---|---|
| Solids Loading (wt %) | 10 |
| Batch Size (kg) | 1.5 |
| Solvent | Acetone |
| Atomizer (Pressure Swirl) | SK 80-16 |
| Solution Flow-rate (g/min) | 160 |
| Atomization Pressure (psig) | 480 |
| Inlet Temperature (° C.) | 94 |
| Outlet Temperature (° C.) | 40 |
| Calculated Outlet | 6.2 |
| Acetone Saturation (% RS) | |
| Dry Yield (%) | 73 |

Based on the 73% yield observed in the first round of process screening, three sprays were performed to investigate the effect of reducing solution throughput and outlet temperature on product yield. All sprays were conducted at a reduced flow-rate of 110 g/min. The outlet temperature was varied at 40° C. (Lot A), 35° C. (Lot B), and 30° C. (Lot C). The outlet temperature was decreased while maintaining a low outlet acetone saturation to increase the difference between the chamber outlet temperature and the wet SDD T_{g}, thus improving product yields. The spray dryer chamber and outlet ductwork were cleaned between all manufactures. A manufacturing summary is shown in Table 11.

TABLE 11
Manufacturing summary for process parameters (1.5 kg batch size)

| | | Low Flow- | Low Flow- |
|---|---|---|---|
| | | Rate/Low | Rate/Lower |
| | Low Flow- | Outlet | Outlet |
| Description | Rate | Temperature | Temperature |
| Lot | A | B | C |
| Solids Loading (wt %) | 10 | 10 | 10 |
| Batch Size (kg) | 1.5 | 1.5 | 1.5 |
| Solvent | Acetone | Acetone | Acetone |
| Atomizer (Pressure Swirl) | Steinen A75 | Steinen A75 | Steinen A75 |
| Solution Flow-Rate | no | 110 | 110 |
| (g/min) | | | |
| Atomization Pressure | 275 | 285 | 285 |
| (psig) | | | |
| Inlet Temperature (° C.) | 79 | 79 | 63 |
| Outlet Temperature (° C.) | 40 | 35 | 30 |
| Calculated Outlet | 4.3 | 5.2 | 6.4 |
| Acetone Saturation (% | | | |
| RS) | | | |
| Calculated wet SDD T_{g} | 72 | 71 | 69 |
| (° C.) | | | |
| Dry Yield (%) | 55 | 80 | 43 |

The conditions used for Lot B were found to give the highest yield. One additional spray was then performed at the same processing conditions as Lot B while increasing the batch size from 1.5 kg to 3.5 kg to evaluate process consistency and to determine if product yield would continue to improve over time. The averaged process conditions for this lot are shown in Table 12.

TABLE 12
Manufacturing summary of process parameters
(1.5 kg and 3.5 kg batch sizes)

| | | Low Flow- |
|---|---|---|
| | Low Flow- | Rate/Low Outlet |
| | Rate/Low Outlet | Temperature/Larger |
| Description | Temperature | Batch Size |
| Lot | B | D |
| Solids Loading (wt %) | 10 | 10 |
| Batch Size (kg) | 1.5 | 3.5 |
| Solvent | Acetone | Acetone |
| Atomizer (Pressure Swirl) | Steinen A75 | Steinen A75 |
| Solution Flow-Rate (g/min) | 110 | 110 |
| Atomization Pressure (psig) | 285 | 2.85 |
| Inlet Temperature (° C.) | 72 | 72 |
| Outlet Temperature (° C.) | 35 | 35 |
| Calculated Outlet Acetone | 5.2 | 5.2 |
| Saturation (% RS) | | |
| Calculated wet SDD T_{g} (° C.) | 71 | 71 |
| Dry Yield (%) | 80 | 84 |

The 1.5 kg batch size (Lot D) was sprayed with an 8400 yield compared to the 80% yield of the 3.5 kg batch (Lot B).

25% Formula (I)/75% PVP/VA 64 SDD Process Parameter Screening Characterization

The 25% Formula (I)/75%0 PVP/VA 64 SDDs manufactured to evaluate processing parameters were characterized for powder properties, performance, and physical and chemical properties. Testing included particle size distribution by Malvern, determination of bulk and tapped density, microcentrifuge dissolution, modulated differential scanning calorimetry (mDSC), powder x-ray diffraction (PXRD), scanning electron microscope (SEM), and assay and related substances. The results did not show any significant differences between the lots.

The particle size distribution (PSD) and tabulated powder properties data of the 25% Formula (I)/75% PVP/VA 64 SDDs are shown in Table 13. All 25% Formula (I)/75% PVP/VA 64 SDDs were observed to have a very similar PSD with a D₅₀ of approximately 16 am. All 2500 Formula (I)/75% PVP/VA 64 SDDs were observed to have low bulk and tapped densities.

TABLE 13
Powder properties of process parameter screening PVP/VA-64 SDDs

| | | | | | | | | Bulk | Tapped |
|---|---|---|---|---|---|---|---|---|---|
| | | D₁₀ | D₅₀ | D₉₀ | D_{(3, 2)} | D_{(4, 3)} | | density | density |
| Sample | Lot | (μm) | (μm) | (μm) | (μm) | (μm) | Span | (g/mL) | (g/mL) |
| 40° C. | A | 5 | 15 | 34 | 8 | 17 | 1.93 | 0.12 | 0.25 |
| Outlet | | | | | | | | | |
| 35° C. | B | 5 | 16 | 36 | 9 | 19 | 1.97 | 0.11 | 0.23 |
| Outlet | | | | | | | | | |
| 30° C. | C | 5 | 15 | 32 | 7 | 17 | 1.86 | 0.12 | 0.27 |
| Outlet | | | | | | | | | |
| 35° C. | D | 5 | 16 | 38 | 9 | 19 | 1.98 | 0.12 | 0.24 |
| Outlet, | | | | | | | | | |
| 3.5 kg | | | | | | | | | |
| batch | | | | | | | | | |

The 3.5 kg batch size lot was analyzed and compared to process parameter Lot A. Dissolution performance was similar for each of these lots. Dissolution was rapid to Cₘₐₓ and high free drug was sustained through 90 minutes. These data are shown in Table 14.

TABLE 14
Dissolution performance of Lot A (1.5 kg
batch size) vs. Lot D (3.5 kg batch size)

| | Cₘₐₓ₉₀ | AUC₉₀ | C₉₀ | Ultra₉₀ |
|---|---|---|---|---|
| Sample | (μg/mL) | (min*μg/mL) | (μg/mL) | (μg/mL) |
| Lot A | 447 | 37,740 | 437 | 319 |
| Lot D | 437 | 37,120 | 433 | 301 |

The 25% Formula (I)/75% PVP/VA 64 SDDs were also evaluated by DSC, PXRD, and SEM. The DSC thermograms showed a single T_{g} at 84° C., indicating homogeneous dispersions. PXRD diffractograms showed no evidence of crystals in the SDDs. SEM images showed inflated sphere morphology with some broken particles and some very small particles.

Additional testing on Lot B was carried out, which included assessing the chemical/physical stability of both spray solution and SDD prior to secondary drying (wet SDD) to establish maximum in-process hold times. Residual acetone concentration as a function of secondary drying time in a convection tray dryer was also evaluated to nominate tray drying conditions to ensure the SDD is dried below International Council for Harmonization of Technical Requirements for Pharmaceuticals for Human Use (ICH) guidelines for acetone.

Residual acetone content as a function of drying time was assessed by drying wet SDD in a tray dryer and collecting samples over a 24-hour period. Wet SDD was dried at 40° C./15% relative humidity (RH) and was observed to dry below ICH acetone guidelines (0.5 wt %, 5000 ppm) by four hours.

Spray solution hold time was determined by making up a representative solution that contained 2.5 wt % compound of Formula (I), 7.5 wt % PVP/VA 64, and 90 wt % acetone. These solutions were analyzed initially for related substances. and then aged at 5° C. and 25° C. Aliquots were taken and analyzed for related substances periodically for 14 days. Results showed no change in impurity profile at either condition through 14 days.

Wet SDD was analyzed for impurities after storage at 5° C. and 25° C. for 1 and 2 weeks and compared to the impurity profiles of the ingoing compound of Formula (I) and the SDD that was secondarily dried immediately after spray drying. The impurity profiles were similar to that of the initial dried sample and the ingoing compound of Formula (I) through 2 weeks of storage.

The wet SDD stability samples were characterized for physical stability by DSC, PXRD, and SEM. DSC thermograms showed a single T_{g} at 81° C., indicative of a homogeneous dispersion with no phase separation. The PXRD diffractograms did not show any evidence of crystals after storage at either condition. SEM images showed a typical morphology of mostly inflated spheres with some broken particles.

### Example 3: Preparation of a 1000 g Batch of a Spray-Dried Dispersion Containing 25% of the Compound of Formula (I) and 75% PVP/VA 64

A 1000 g batch of the spray-dried dispersion containing 25% of the compound of Formula (I) and 75% PVP/VA 64 was prepared as described in Example 2 for the 1.5 kg and 3.5 kg batches. Briefly, acetone (90% (w/w) of the total mixture) was added to the mixing tank followed by the addition of 250.0 g of the compound of Formula (I) (2.5% (w/w) of the total mixture). The mixture was mixed for 30 minutes in the dark at a temperature range of 15° C. to 27° C. At the end of the mixing period, the solution was clear and free of undissolved solids. The PVP/VA 64 (750.0 g, 7.5% (w/w) of the total mixture) was then added and the mixture was stirred for an additional 30 minutes in the dark at a temperature range of 15° C. to 27° C. At the end of the mixing period, the solution was clear and free of undissolved solids.

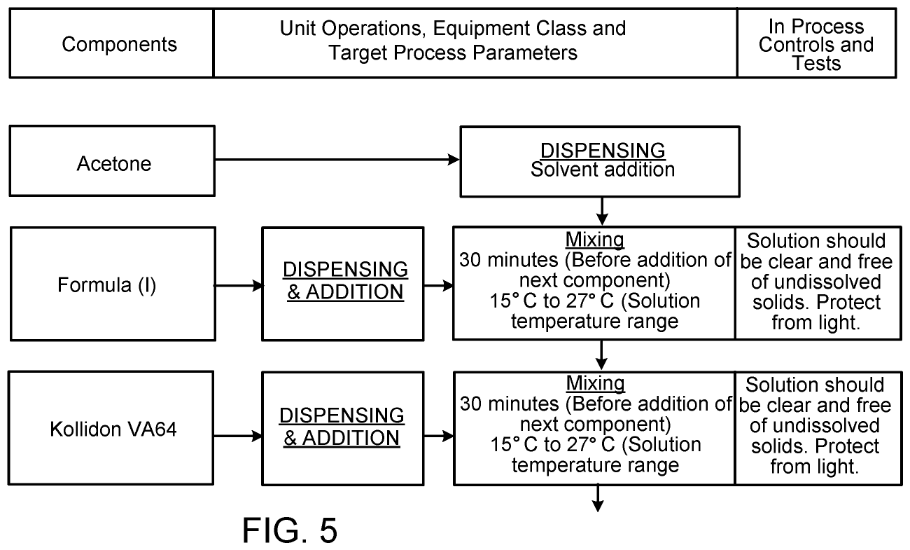

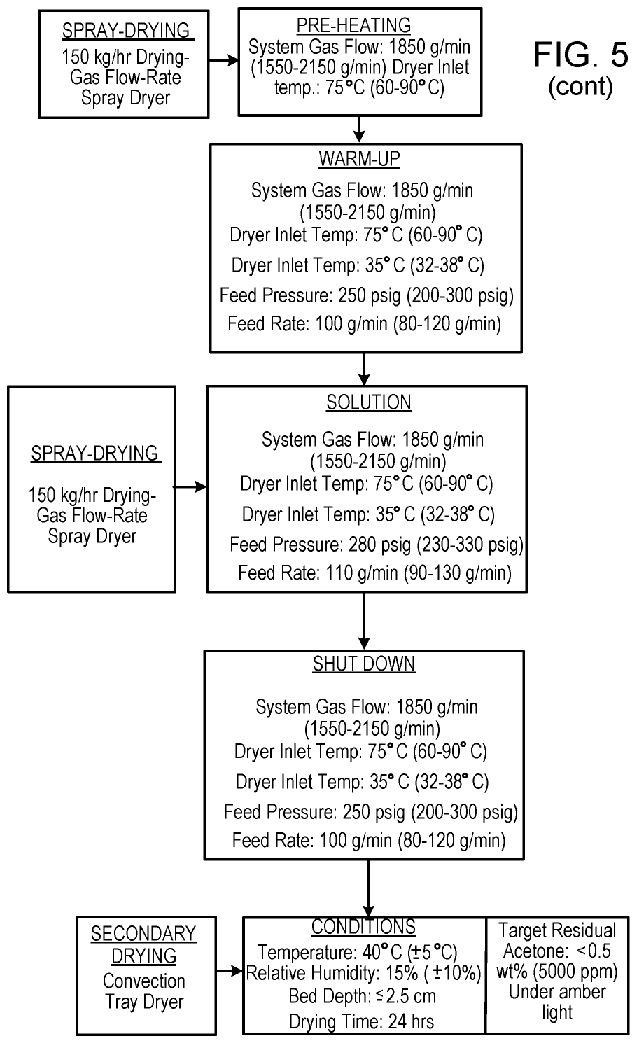

The solution was pumped and atomized in a drying chamber. The spray-dried dispersions were prepared in a Pharmaceutical Spray Dryer with 100 kg/hr drying gas capacity (PSD-1). The inlet temperature was set at 75° C. (varied between 60° C.-90° C.). The outlet temperature was set at 35° C. (varied between 32° C.-38° C.). The feed pressure was set at 280 psig (varied between 230-330 psig). The feed rate was set at 110 g/min (varied between 90-130 g/min). The spray dried powder was then dried in a convection tray dryer with a bed depth of ≤2.5 cm at 40° C. (±5° C.) and 15% relative humidity (±10%) for 24 hours under amber light. The residual acetone after drying was <0.5 wt % (5000 ppm). FIG. 5 is a flow diagram of the manufacturing process.

### Example 4: Preparation of Spray-Dried Dispersion Formulations of the Compound of Formula (I) for Clinical Use

The spray-dried dispersion (SDD) containing 25% compound of Formula (I) and 75% PVP/VA 64, prepared as described above, was formulated as a suspension or a capsule for clinical use.

Suspension Preparation

A suspension that contained 50 mg of the SDD was prepared as follows. A 30 mL amber dosing bottle was tared on a balance. 200.0 mg SDD (50 mgA)±5% was then weighed into the dosing bottle. Using a 10-mL syringe, 5.0 mL of water (purified, USP) was added to the dosing bottle and the bottle was capped and shaken moderately for 30 seconds. The SDD suspension was stored in an amber vial at 2-8° C. prior to use, and dosed within 24 hours of preparation.

Capsule Preparation

An empty hard gelatin capsule, size 0 (Capsugel, Morristown, N.J.), was placed on a balance and the weight was recorded. 200.0 mg SDD (50 mgA)±5% was then weighed onto weigh paper or an equivalent. All contents were transferred to the capsule using a ProFunnel device for Size 0 capsules. The filled capsule was placed on the balance and the weight was recorded. The weight of the empty capsule was subtracted from the filled weight, ensuring that the weight of the SDD within the capsule was 200.0 mg SDD±5%, or from 190.0 mg to 210.0 mg. The capsule was securely closed with the head, assuring it clicked into place. The capsules were stored in an amber vial at 2-8° C. prior to use, and were dosed within 24 hours of preparation.

### Example 5: Dog Relative Bioavailability and Food Effect Study

Four spray-dried dispersions (SDDs), formulated in 0.25% methylcellulose as a suspension, were prepared: (1) 25% compound of Formula (I)/75% HPMCAS-L; (2) 10% compound of Formula (I)/90% HPMCAS-L; (3) 25% compound of Formula (I)/75% methyl methacrylate copolymer (1:1) (Eudragit® L100); and (4) 25% compound of Formula (I)/75% PVP/VA 64. A clinical capsule formulation was prepared as a reference formulation (Reference Formulation 1 from Table 5, above).

Dogs (two cohorts, six dogs in each) were dosed in six sessions, including fasted state sessions and fed sessions (high fat diet), with 50 mg dose of one of the SDDs or reference per dog in a 3-way crossover design. Each session had a 3-day washout in between. All formulations were well tolerated. The study design is shown in Table 15, below.

TABLE 15
Study design

| | Session 1 | Session 2 | Session 3 | Session 4 | Session 5 | Session 6 |
|---|---|---|---|---|---|---|
| | (Fasted) | (Fed) | (Fasted) | (Fed) | (Fasted) | (Fed) |
| Cohort 1 | | | | | | |
| Dog 1001 | Reference | Reference | SDD 1 | SDD 1 | SDD 2 | SDD 2 |
| Dog 1002 | Reference | Reference | SDD 1 | SDD 1 | SDD 2 | SDD 2 |
| Dog 2001 | SDD 1 | SDD 1 | SDD 2 | SDD 2 | Reference | Reference |
| Dog 2002 | SDD 1 | SDD 1 | SDD 2 | SDD 2 | Reference | Reference |
| Dog 3001 | SDD 2 | SDD 2 | Reference | Reference | SDD 1 | SDD 1 |
| Dog 3002 | SDD 2 | SDD 2 | Reference | Reference | SDD 1 | SDD 1 |
| Cohort 2 | | | | | | |
| Dog 4001 | Reference | Reference | SDD 3 | SDD 3 | SDD 4 | SDD 4 |
| Dog 4002 | Reference | Reference | SDD 3 | SDD 3 | SDD 4 | SDD 4 |
| Dog 5001 | SDD 3 | SDD 3 | SDD 4 | SDD 4 | Reference | Reference |
| Dog 5002 | SDD 3 | SDD 3 | SDD 4 | SDD 4 | Reference | Reference |
| Dog 6001 | SDD 4 | SDD 4 | Reference | Reference | SDD 3 | SDD 3 |
| Dog 6002 | SDD 4 | SDD 4 | Reference | Reference | SDD 3 | SDD 3 |

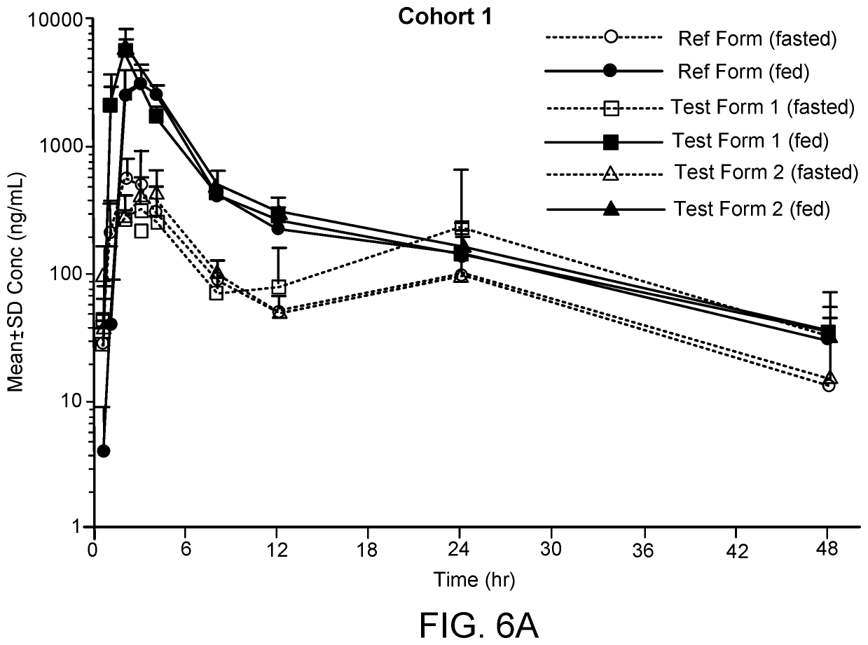

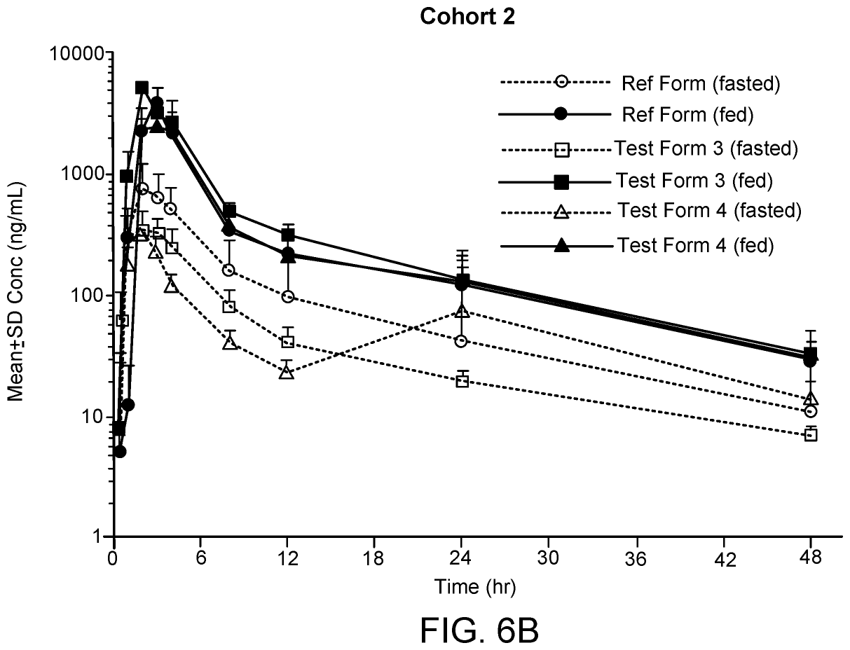

The area under the plasma concentration versus time curve from 0 hours extrapolated to infinity (AUC_{0-∞}), maximum plasma concentration (Cₘₐₓ), the apparent terminal half-life (t_{1/2}), and the time to achieve maximum plasma concentration (tₘₐₓ) were calculated. Results are shown in Table 16, below, and in FIGS. 6A and 6B.

TABLE 16
Pharmacokinetic results

| | | Fasted | | Fed | | | | |
|---|---|---|---|---|---|---|---|---|
| | | [Mean (% CV)] | | [Mean (% CV)] | | Ratio of Fed/Fasted | | |
| | | Cₘₐₓ | AUC_{inf} | Cₘₐₓ | AUC_{inf} | [Mean (% CV)] | | |
| Cohort | Form | (ng/mL) | (hr*ng/mL) | (ng/mL) | (hr*ng/mL) | Cₘₐₓ | | AUC |
| 1 | Ref | 652 (55.5) | 5170 (69.4) | 3650 (19.5) | 20100 (22) | 6.9 | (48) | 5.5 (69.2) |
| (N = 4)* | SDD 1 | 524 (62.7) | 2870 (62.9) | 5760 (25.6) | 22800 (14.6) | 15.4 | (66.9) | 8 (89.6) |
| | SDD 2 | 487 (45.7) | 3950 (26.4) | 4220 (27.4) | 18100 (26.8) | 16.5 | (40.7) | 6.3 (34.6) |
| 2 | Ref | 854 (40.2) | 5520 (59.1) | 4220 (27.4) | 18100 (26.8) | 5.8 | (46.5) | 4.1 (49.2) |
| (N = 6) | SDD 3 | 453 (28.3) | 2830 (21.6) | 5320 (13.5) | 23800 (26.1) | 12.6 | (30.3) | 8.6 (22.2) |
| | SDD 4 | 353 (20.1) | 1840 (21.9) | 3060 (20.7) | 17200 (20.5) | 8.9 | (29.3) | 8.4 (46.1) |

*Animals 2001 and 2002 were excluded from summary statistics due to emesis in ail 3 fed sessions, which resulted in notably lower exposures

The results showed that t_{1/2} and tₘₐₓ were similar among the formulations, and comparable between the fed and fasted states. Under the fed state with a high fat meal, exposures increased and inter-animal variability decreased. The food effect was more notable with the spray-dried dispersion formulations, especially for peak exposure (Cₘₐₓ).

Compared to the reference form, SDD 4 (25% compound of Formula (I)/75% PVP/VA 64) appeared to have lower inter-animal variability, lower exposures under the fasted state, and slightly lower Cₘₐₓ but relatively comparable AUC in the fed state.

### Example 6: Phase 1 Study to Evaluate the Pharmacokinetics, Effect of Food on Pharmacokinetics, and Safety of the Compound of Formula (I) in Healthy Adult Subjects

The present study was designed to evaluate the pharmacokinetics (PK) of the compound of Formula (I) as well as to evaluate the effect of a fed condition on the PK of the compound of Formula (I). The 50 mg dose was chosen for this study because it was within the tested dose range in completed Phase 1 and Phase 2 trials and was well tolerated in those studies. The objectives of the study were: to evaluate the PK of the compound of Formula (I) 50 mg in healthy adult subjects; to evaluate the effect of food on the PK of the compound of Formula (I) 50 mg; and to evaluate the safety and tolerability of the compound of Formula (I) 50 mg.

### Study Design

This was a Phase 1, open-label, randomized, 2-period crossover study of the PK and the effect of food on the PK of the compound of Formula (I) in 16 healthy male and female adult subjects, 18 to 55 years of age.

After providing informed consent, subjects were screened for eligibility to participate in the study up to 28 days prior to Day 1 of treatment period 1. Eligible subjects were admitted to the clinical unit on Day −1 and randomized to 1 of the 2 treatment sequences (16 subjects [8 males and 8 females]; see Table 17, below). On Day 1 of each treatment period, subjects received a single dose of the compound of Formula (I) 50 mg under fasted or fed conditions. There were 21 days between doses.

TABLE 17
Treatment sequences

| Treatment sequence | Treatment period I | Treatment period 2 |
|---|---|---|
| 1 | Formula (I) - fasted | Formula (I) - fed |
| 2 | Formula (I) - fed | Formula (I) - fasted |

Subjects were required to fast for at least 4 hours before check-in on Day −1. In the fasted condition, subjects were required to fast overnight for at least 10 hours prior to dosing and continued to fast for an additional 4 hours after dosing. In the fed condition, subjects were required to fast overnight for at least 10 hours and then ingest a liquid dietary supplement with study drug (liquid dietary supplement was consumed within 30 minutes) and not consume any other food for 4 hours after dosing. During both treatment periods, water was not permitted for 1 hour before dosing until 2 hours after dosing except for the water/liquid dietary supplement provided for study drug dosing. Vanilla-flavored Ensure Plus® was used as the liquid dietary supplement.

On Day 1 of each treatment period, subjects were dosed with the compound of Formula (I) 50 mg. Blood samples were collected for PK analysis over a period of 36 hours during the in-house stay. Subjects remained in the unit on the day of dosing and were discharged on Day 2 of each treatment period, following completion of all required procedures. On the mornings of Days 8 and 15 of each treatment period, subjects returned to the clinical unit on an outpatient basis for PK blood sample collection and safety assessments. On Day 21 of treatment period 1, subjects arrived at the site and had Day 21 assessments completed in the evening, and they stayed overnight at the site and began Day 1 of treatment period 2 the following day. A final follow-up study visit was conducted on Day 22 of treatment period 2 (21±2 days after treatment period 2 dosing) or upon early termination.

During each treatment period, blood samples for PK analysis were collected within 45 minutes before dosing, and at approximately 30 minutes, and 1, 2, 3, 4, 5, 6, 7, 8, 9, 10, 12, 16, 24, 36, 168, 336, and 504 hours after dosing.

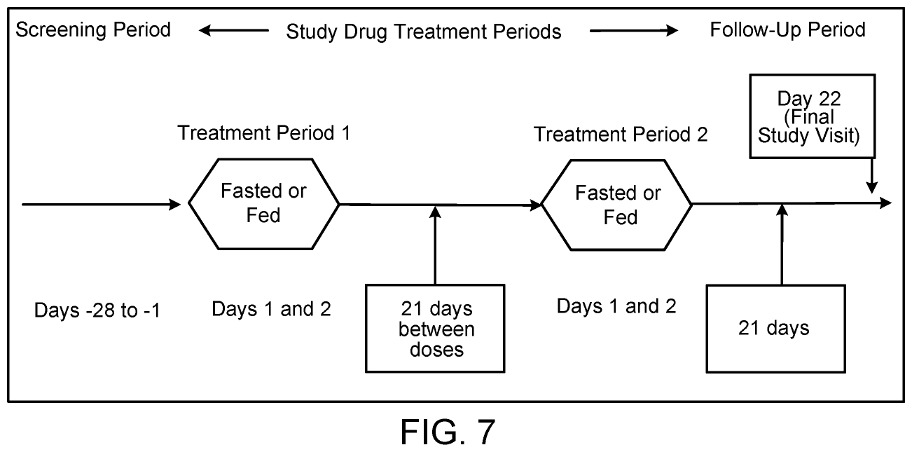

Safety assessments (including clinical safety laboratory tests, vital sign measurements, physical examinations, and electrocardiograms (ECGs)) were conducted at scheduled times throughout the study. Adverse events (AEs) and the use of concomitant medications were monitored throughout the study. FIG. 7 illustrates the study design.

Test Product, Dose and Mode of Administration

The compound of Formula (I) was supplied as an encapsulated, lipidic semi-solid containing 50 mg of the compound of Formula (I) as free base equivalent for oral administration. Subjects swallowed a single capsule with approximately 240 mL of water in the fasted condition. Subjects swallowed a single capsule with a liquid dietary supplement (Ensure Plus® [237 mL container]) with up to an additional 120 mL of water in the fed condition.

Duration of Treatment

The duration of study participation for each adult subject was approximately 10 weeks, including up to 28 days of screening, 2 days of dosing separated by 21 days, and a final follow-up study visit 21 days after receiving the last dose of study drug during treatment period 2.

### Criteria for Evaluation

Pharmacokinetics

The following plasma PK parameters were calculated for the compound of Formula (I):
Area under the plasma concentration versus time curve from 0 hours to last measurable concentration (AUC₀₋ₜₗₐₛₜ)
Area under the plasma concentration versus time curve from 0 to 24 hours (AUC₀₋₂₄)
Area under the plasma concentration versus time curve from 0 hours extrapolated to infinity (AUC_{0-∞})
Maximum plasma concentration (Cₘₐₓ)
Time to achieve maximum plasma concentration (tₘₐₓ)
Delay time between time of dosing and time of appearance of measurable test article (T_{lag})
Apparent terminal half-life (t_{1/2})
Apparent terminal rate constant (λz)
Apparent mean residence time (MRT)
Molar AUC ratio of the hydroxylated metabolite of the compound of Formula (I) to the parent drug the compound of Formula (I)

The following plasma PK parameters were calculated only for the compound of Formula (I):
Apparent systemic clearance after oral administration (CL/F)
Apparent volume of distribution during terminal phase after oral administration (Vz/F)

Safety

Safety was monitored throughout the study and included the following assessments:
Adverse events (AEs)
Clinical laboratory tests (hematology, coagulation, clinical chemistry, and urinalysis)
Vital sign measurements (including orthostatic blood pressure and pulse rate)
Physical examinations
12-lead electrocardiograms (ECGs)

Statistical Methods

Pharmacokinetic parameters were calculated using noncompartmental methods and summarized by condition (fed or fasted) using descriptive statistics. Two-sided 90% confidence intervals were calculated for the ratio under the fed condition vs. under the fasted condition for AUC_{0-∞}, AUC₀₋ₜₗₐₛₜ, and Cₘₐₓ for the compound of Formula (I) and the hydroxylated metabolite of the compound of Formula (I).

Safety data were summarized with descriptive statistics.

### Pharmacokinetics

Pharmacokinetic Assessments

PK plasma samples for analyses of the compound of Formula (I) and the hydroxylated metabolite of the compound of Formula (I) were collected at the following times during each treatment period:
Day 1: within 45 minutes before dosing, and at approximately 30 minutes, and 1, 2, 3, 4, 5, 6, 7, 8, 9, 10, 12, and 16 hours after dosing.
Day 2: approximately 24 and 36 hours after dosing.
Day 8: approximately 168 hours after dosing.
Day 15: approximately 336 hours after dosing.
Approximately 504 hours after dosing (for treatment period 1, this sample was collected in the morning at least 30 minutes prior to the predose sample on Day 1 of treatment period 2).
Final study visit for subjects who terminate early: 1 sample.

Blood samples on Day 1 were collected within 5 minutes of the scheduled sampling times (other than the predose sample). Blood samples on Days 2, 8, and 15 were collected within 2 hours of the scheduled sampling time. The 504 hour blood sample for treatment period 2 had a ±2-day window. A PK sample was to be collected from subjects who terminated early. The exact time of sampling in hour and minutes was recorded.

Bioanalytical Methods

Plasma samples were analyzed for the compound of Formula (I) and for its hydroxylated metabolite by inVentiv Health, Princeton N.J., in compliance with Good Laboratory Practice (GLP) and relevant Standard Operating Procedures (SOPs).

The concentrations of the compound of Formula (I) and the hydroxylated metabolite of the compound of Formula (I) were quantified in plasma samples according to validated methods using tandem mass spectrometry in positive ion mode. This method was validated for the analysis of the compound of Formula (I) and the hydroxylated metabolite of the compound of Formula (I) in 25.0 μL dipotassium ethylenediaminetetraacetic acid (K₂-EDTA) human plasma samples over concentration ranges of 5.00 to 2500 ng/mL and 0.500 and 250 ng/mL, respectively. All analytical results were within acceptable limits. Incurred sample reanalysis (ISR) was successfully conducted in this study for both analytes.

### Results

Pharmacokinetic Results

Eight male and eight female subjects were enrolled. The mean age was 37.1 years (range, 21 to 55 years). The majority of subjects were White (93.8%) and of Hispanic ethnicity (81.3%). The mean weight at screening was 160.28 lbs (range, 102.0 to 222.2 lbs) and mean BMI was 25.50 kg/m² (range, 20.7 to 30.5 kg/m²). The randomization was well balanced with respect to demographics and baseline characteristics.

All 16 subjects were included in the safety analysis set. No subjects were excluded from the safety analysis set and no subject had his or her PK data excluded from analysis.

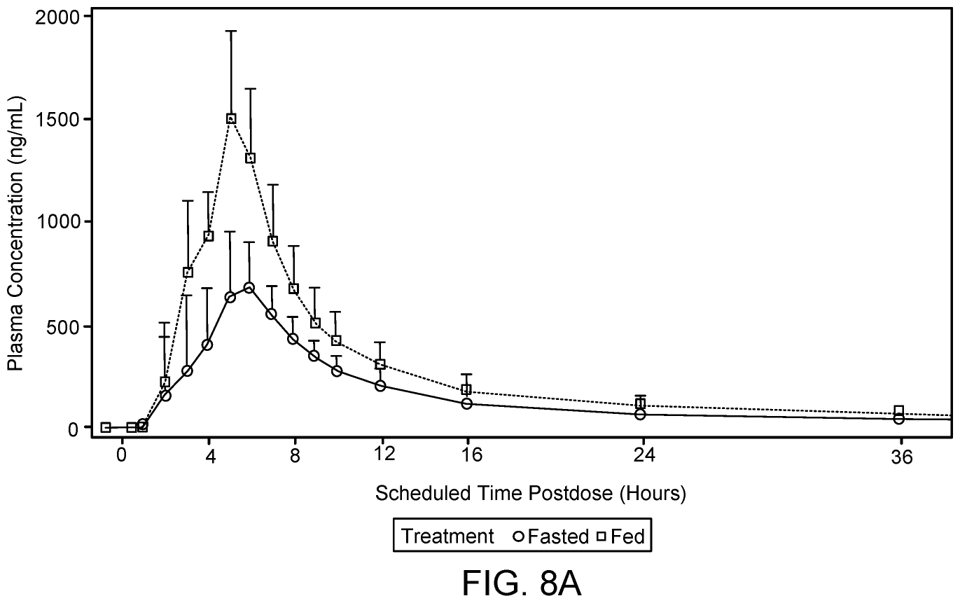

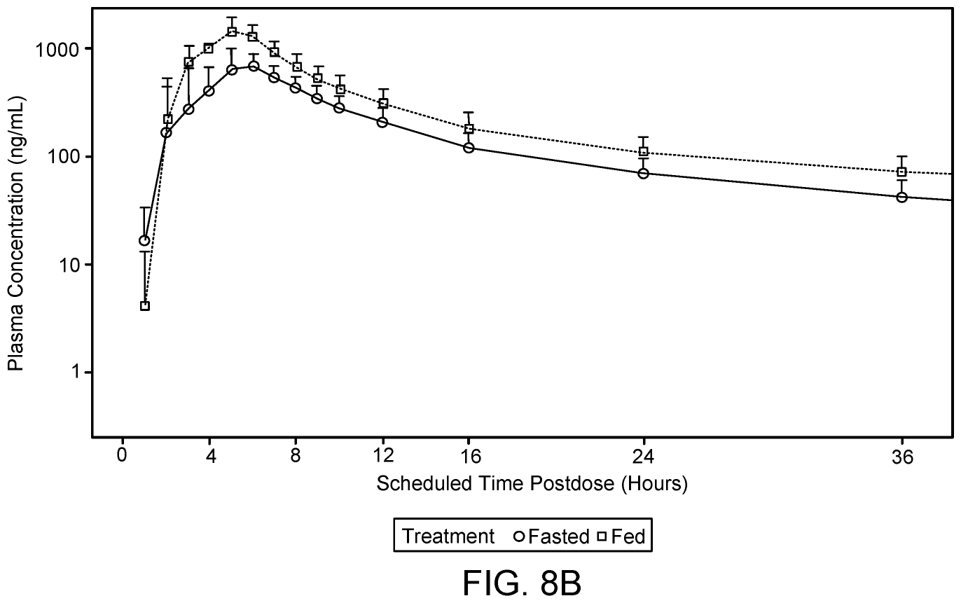

The mean plasma concentration versus time profiles for the compound of Formula (I) under fasted and fed conditions are presented in FIGS. 8A and 8B, respectively. The compound of Formula (I) was slowly absorbed after oral administration in both fasted and fed conditions. Mean plasma concentrations were lower in the fasted than in the fed condition.

PK parameters for the compound of Formula (I) after treatment with the compound of Formula (I) under fasted and fed conditions are summarized in Table 18, below, where AUC₀₋₂₄=area under the plasma concentration vs. time curve from 0 to 24 hours, AUC₀₋ₜₗₐₛₜ=AUC from 0 hours to last measurable concentration, AUC_{0-∞}=AUC from 0 hours extrapolated to infinity, CL/F=apparent systemic clearance after oral administration, CV=coefficient of variation, Cₘₐₓ=maximum plasma concentration, CV (%)=coefficient of variation, max=maximum, min=minimum, MRT=apparent mean residence time, PK=pharmacokinetic, SD=standard deviation, t_{1/2}=apparent terminal half-life, T_{lag}=delay time between time of dosing and time of appearance of measurable test article, tₘₐₓ=time to maximum plasma concentration, VZ/F=apparent volume of distribution during the terminal phase after oral administration.

The PK data for tₘₐₓ, T_{lag}, t_{1/2}, MRT, and Vz/F were rounded to 2 significant figures and all other parameters (AUC₀₋₂₄, AUC₀₋ₜₗₐₛₜ, AUC_{0-∞}, Cₘₐₓ, and CL/F) were rounded to 3 significant figures. The last significant figure was rounded up if the digit to the right of it was ≥5, and was rounded down if the digit to the right of it was ≤4.

TABLE 18
Summary of Formula (I) PK parameters (safety analysis set)

| | Fasted | | Fed | |
|---|---|---|---|---|
| Parameter | Formula (I) (50 mg) | | Formula (I) (50 mg) | |
| Statistic | (N = 16) | | (N=15) | |
| AUC₀₋₂₄ (ng × hr/mL) | | | | |
| Mean (SD) | 5590 | (2230) | 9950 | (2540) |
| Geometric CV % | 32.7 | | 26.2 | |
| AUC₀₋ₜₗₐₛₜ (ng × | | | | |
| hr/mL) | | | | |
| Mean (SD) | 8020 | (5110) | 16200 | (5450) |
| Geometric CV % | 53.6 | | 39.7 | |
| AUC_{0-∞} (ng × hr/mL) | | | | |
| Mean (SD) | 9440 | (2990) [n = 7] | 17800 | (4990) [n = 12] |
| Geometric CV % | 39.7 | | 28.1 | |
| Cₘₐₓ (ng/mL) | | | | |
| Mean (SD) | 731 | (301) | 1550 | (392) |
| Geometric CV % | 36.6 | | 24.2 | |
| tₘₐₓ (hours) | | | | |
| Median (min, max) | 6.0 | (3.0, 6.0) | 5.0 | (3.0, 6.0) |
| T_{lag} (hours) | | | | |
| Mean (SD) | 0.63 | (0.22) | 0.94 | (0.37) |
| t_{1/2} (hours) | | | | |
| Mean (SD) | 33 | (17) [n = 7] | 42 | (6.8) [n = 12] |
| Geometric CV % | 99 | | 15 | |
| MRT (hours) | | | | |
| Mean (SD) | 28 | (11) [n = 7] | 30 | (5.0) [n = 12] |
| Geometric CV % | 49 | | 16 | |
| CL/F (L/hr) | | | | |
| Mean (SD) | 6.0 | (2.7) [n = 7] | 3.0 | (0.82) [n = 12] |
| Geometric CV % | 40 | | 28 | |
| VZ/F (L) | | | | |
| Mean (SD) | 240 | (120) [n = 7] | 180 | (57) [n = 12] |

| | | |
|---|---|---|
| Geometric CV % | 67 | 30 |

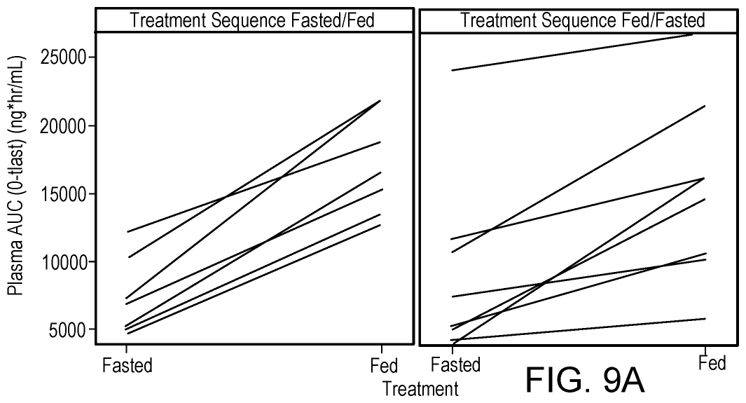

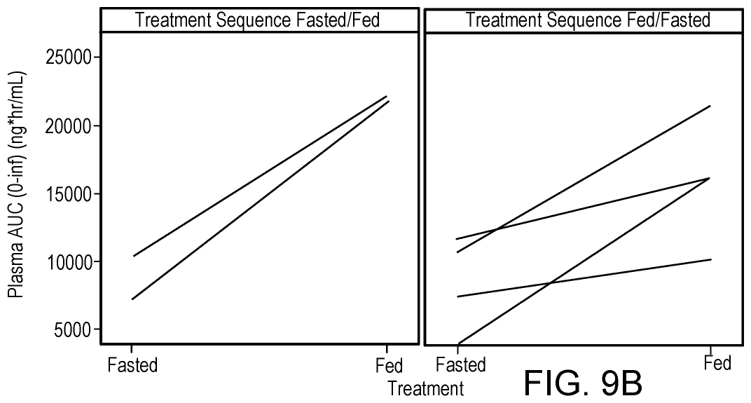

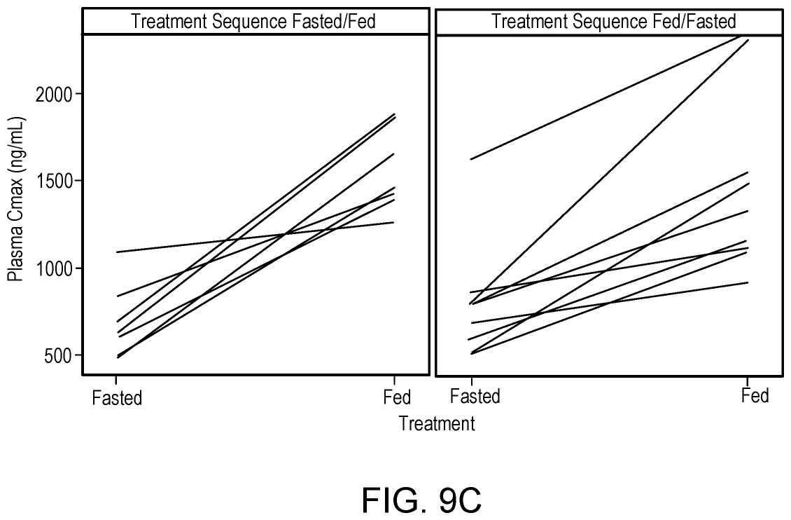

As seen in Table 18, above, administration of the compound of Formula (I) 50 mg under fed compared with fasted conditions resulted in a higher mean Cₘₐₓ of the compound of Formula (I) (approximately 2-fold higher; 1550 vs. 731 ng/mL), a longer t_{1/2} (42 vs. 33 hours), a slightly shorter median tₘₐₓ (5.0 vs. 6.0 hours), and a higher mean AUC_{0-∞} (approximately 2-fold higher; 17800 vs. 9440 ng×hr/mL). The compound of Formula (I) geometric mean ratios for Cₘₐₓ and AUC₀₋ₜₗₐₛₜ for fed vs. fasted conditions were 218.6% and 215.2%, respectively, indicating that the compound of Formula (I) absorption was approximately 2-fold greater when administered with food. The upper and lower 9000 confidence interval (CI) bounds for both Cₘₐₓ (187.4% and 255.1%, respectively) and AUC₀₋ₜₗₐₛₜ (182.9% and 253.1%, respectively) were outside of the “no-effect” range of 80.00% to 125.0000, indicating that there was a food effect on the compound of Formula (I) exposure. Frequency distribution of T_{lag} and tₘₐₓ values are presented in Table 19 and Table 20, respectively. Spaghetti plots for the compound of Formula (I) AUC₀₋ₜₗₐₛₜ, AUC_{0-∞} and Cₘₐₓ are shown in FIGS. 9A, 9B, and 9C, respectively.

TABLE 19
Frequency distribution of plasma Formula (I)
T_{lag} values by treatment (safety analysis set)

| | | Fasted | | Fed | |
|---|---|---|---|---|---|
| T_{lag} | | (Formula (I) 50 mg) | | (Formula (I) 50 mg) | |
| (hr) | Statistic | (N = 16) | | (N = 15) | |
| 0.50 | n (%) | 11 | (68.8%) | 3 | (20.0%) |
| 0.53 | n (%) | 1 | (6.3%) | 0 | (0.0%) |
| 0.55 | n (%) | 0 | (0.0%) | 1 | (6.7%) |
| 1.00 | n (%) | 4 | (25.0%) | 8 | (53.3%) |
| 1.02 | n (%) | 0 | (0.0%) | 1 | (6.7%) |
| 1.03 | n (%) | 0 | (0.0%) | 1 | (6.7%) |
| 2.00 | n (%) | 0 | (0.0%) | 1 | (6.7%) |

TABLE 20
Frequency distribution of plasma Formula (I)
Tₘₐₓ values by treatment (safety analysis set)

| | | Fasted | | Fed | |
|---|---|---|---|---|---|
| Tₘₐₓ | | (Formula (I) 50 mg) | | (Formula (I) 50 mg) | |
| (hr) | Statistic | (N = 16) | | (N = 15) | |
| 3.00 | n (%) | 1 | (6.3%) | 2 | (13.3%) |
| 4.02 | n (%) | 1 | (6.3%) | 0 | (0.0%) |
| 5.00 | n (%) | 4 | (25.0%) | 8 | (53.3%) |
| 5.03 | n (%) | 0 | (0.0%) | 1 | (6.7%) |
| 5.05 | n (%) | 0 | (0.0%) | 1 | (6.7%) |
| 6.00 | n (%) | 10 | (62.5%) | 2 | (13.3%) |
| 6.02 | n (%) | 0 | (0.0%) | 1 | (6.7%) |

The compound of Formula (I) was slowly absorbed after oral administration in the fasted and fed conditions. In the fasted condition, the mean compound of Formula (I) Cₘₐₓ was approximately 53% lower than in the fed condition (731 vs. 1550 ng/mL). Due to a prolonged elimination phase, t_{1/2} values and therefore, AUC_{0-∞} values could not be determined for some the compound of Formula (I) concentration-time profiles. Mean AUC_{0-∞} was approximately 47% lower in the fasted condition than in the fed condition (9440 vs. 17800 ng×hr/mL) for those subjects for whom AUC_{0-∞} could be determined. Mean AUC₀₋ₜₗₐₛₜ was approximately 50% lower in the fasted condition than in the fed condition (8020 vs. 16200 ng×hr/mL). Median tₘₐₓ was slightly longer in the fasted condition than in the fed condition (6.0 vs. 5.0 hours) and mean t_{1/2} was shorter in the fasted condition than in the fed condition (33 vs. 42 hours) for those subjects for whom a t_{1/2} could be determined. Variability in the compound of Formula (I) PK (geometric CV %) for AUC, Cₘₐₓ, t_{1/2}, and MRT was lower in the fed condition compared with the fasted condition.

The geometric mean ratios and associated 90% CIs for AUC₀₋ₜₗₐₛₜ and Cₘₐₓ for the compound of Formula (I) after treatment with the compound of Formula (I) for the fed vs. fasted condition are provided in Table 21, below, where AUC₀₋ₜₗₐₛₜ=AUC from 0 hours to last measurable concentration, Cₘₐₓ=maximum plasma concentration, and PK=pharmacokinetic.

TABLE 21
Formula (I) geometric mean ratios for PK exposure parameters
under fed vs. fasted conditions (safety analysis set)

| | Ratio^{a} (%) | 90% Confidence |
|---|---|---|
| Parameter | (Fed vs. Fasted Condition) | Interval^{b} |
| AUC₀₋ₜₗₐₛₜ (ng × hr/mL) | 215.2 | 182.9, 253.1 |
| Cₘₐₓ (ng/mL) | 218.6 | 187.4, 255.1 |

^{a}Ratio of geometric least-squares means was based on a mixed model using log-transformed (base 10) data.
^{b}The 90% confidence interval for geometric mean ratio was based on least-squares means using log-transformed (base 10) data.

The compound of Formula (I) geometric mean ratios for Cₘₐₓ and AUC₀₋ₜₗₐₛₜ for the fed vs. fasted conditions were 218.6% and 215.2%, respectively, indicating that the compound of Formula (I) absorption was approximately 2-fold greater when administered with food. The upper and lower 90% CI bounds for both Cₘₐₓ (187.4%, 255.1%) and AUC₀₋ₜₗₐₛₜ (182.9%, 253.1%) were outside of the “no-effect” range of 80.00% to 125.00%, indicating that there was a food effect on the compound of Formula (I) exposure. Due to the missing AUC_{0-∞} values, the food effect on overall exposure was not assessed using AUC_{0-∞} values.

Conclusion

Administration of the compound of Formula (I) 50 mg under fed compared with fasted conditions resulted in a higher mean Cₘₐₓ of the compound of Formula (I) (approximately 2-fold higher; 1550 vs. 731 ng/mL), a longer t_{1/2} (42 vs. 33 hours), a slightly shorter median tₘₐₓ (5.0 vs. 6.0 hours), and a higher mean AUC_{0-∞} (approximately 2-fold higher; 17800 vs. 9440 ng×hr/mL). The compound of Formula (I) geometric mean ratios for Cₘₐₓ and AUC₀₋ₜₗₐₛₜ for fed vs. fasted conditions were 218.6% and 215.2%, respectively, indicating that the compound of Formula (I) absorption was approximately 2-fold greater when administered with food. The upper and lower 90% CI bounds for both Cₘₐₓ (187.4%, 255.1%) and AUC₀₋ₜₗₐₛₜ (182.9%, 253.1%) were outside of the “no-effect” range of 80.00% to 125.00%, indicating that there was a food effect on the compound of Formula (I) exposure. Similar results were observed with the hydroxylated metabolite of the compound of Formula (I). Overall, these results indicate that the compound of Formula (I) 50 mg was well tolerated in healthy subjects when administered under fasted or fed conditions and that the total AUC and Cₘₐₓ were increased when the compound of Formula (I) was taken with food.

### Example 7: Phase 1 Study to Evaluate the Relative Bioavailability, Effect of Food on Pharmacokinetics, and Safety of Formulations of the Compound of Formula (I) in Healthy Adult Subjects

A Phase 1 study to compare the relative bioavailability of a 50 mg dose of different formulations of the compound of Formula (I) as well as to evaluate the effect of fasting and fed conditions on the pharmacokinetics (PK) of the compound of Formula (I) was designed. The 50 mg dose was chosen for this study because it is within the tested dose range in completed Phase 1 and Phase 2 trials and was well tolerated in those studies. The objectives of the study are: to evaluate the PK and compare the relative bioavailability of the compound of Formula (I) 50 mg formulations in healthy adult subjects; to evaluate the effect of food on the PK of the compound of Formula (I) 50 mg formulations; and to evaluate the safety and tolerability of the compound of Formula (I) 50 mg formulations.

### Study Design

This is a Phase 1, open-label, randomized, three-period crossover study of the relative bioavailability and the effect of food on the PK of the compound of Formula (I) 50 mg in healthy adult subjects. During treatment period 1 and treatment period 2, subjects will receive a single dose of the compound of Formula (I) 50 mg administered as an encapsulated, lipidic semi-solid (reference) and 1 of 2 different spray-dried dispersion (SDD) test formulations (suspension or capsule) under fed conditions, and during treatment period, three subjects will receive the same SDD test formulation under fasted conditions.

A total of 36 healthy adult subjects will be randomized to 1 of 4 treatment sequences (9 subjects per sequence; approximately equal distribution of males and females per sequence; see Table 22, below). There will be 21 days between each dose.

TABLE 22
Treatment sequences

| | Treatment | Treatment | Treatment |
|---|---|---|---|
| Treatment | period 1 | period 2 | period 3 |
| sequence | (Fed) | (Fed) | (Fasted) |
| 1 | Reference | SDD suspension | SDD suspension |
| 2 | SDD suspension | Reference | SDD suspension |
| 3 | Reference | SDD capsule | SDD capsule |
| 4 | SDD capsule | Reference | SDD capsule |

After providing informed consent, subjects will be screened for eligibility to participate in the study. Screening will begin up to 28 days prior to Day 1 of treatment period 1. Eligible subjects will be admitted to the clinical unit on Day −1, and randomized to 1 of the 4 treatment sequences on Day 1 of treatment period 1. During treatment periods 1 and 2, subjects will fast overnight for at least 10 hours and then ingest a liquid dietary supplement (vanilla-flavored Ensure Plus®, 237 mL container) with the study drug and not consume any other food for 4 hours after dosing. During treatment period 3, subjects will fast overnight for at least 10 hours prior to dosing and continue to fast for an additional 4 hours after dosing. During all treatment periods, water will not be permitted for 1 hour before dosing until 2 hours after dosing except for the water/liquid dietary supplement provided for study drug dosing.

On Day 1 of each treatment period, subjects will be dosed with the compound of Formula (I) 50 mg and have blood samples collected for PK analysis. Subjects will complete a taste satisfaction questionnaire after study drug ingestion on Day 1 of treatment period 3 (only for subjects who receive the SDD suspension under the fasted condition). Subjects will remain in the unit on the day of dosing and will be discharged on Day 2 of each treatment period, following completion of all required procedures, including collection of the 36-hour PK sample. On the mornings of Days 8 and 15 of each treatment period, subjects will return to the clinical unit on an outpatient basis for PK blood sample collection and safety assessments. On Day 21 of treatment period 1 and treatment period 2, subjects will arrive at the site and have Day 21 assessments completed, and they will stay overnight at the site and begin Day 1 of treatment period 2 or treatment period 3 the following day. A final follow-up study visit will be conducted on Day 22 of treatment period 3 (21±2 days after treatment period 3 dosing) or upon early termination.

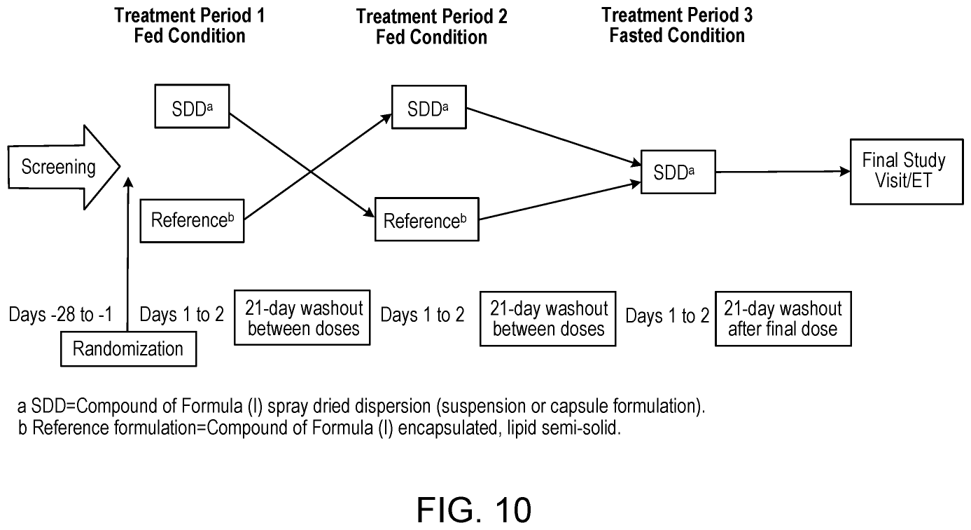

Blood samples for PK analysis and safety assessments will be collected/conducted at scheduled times throughout the study. The study design schematic is shown in FIG. 10.

Duration of Treatment

The expected duration of study participation for each healthy adult subject will be approximately 13 weeks, including up to 28 days of screening, 3 doses each separated by 21 days, and a final follow-up study visit 21 days after receiving the last dose of study drug during treatment period 3.

Test Product, Dose, and Mode of Administration

The compound of Formula (I) will be supplied as two different test formulations for oral administration: as powder in bottles for constitution into a suspension (20 mL) and as powder-filled capsules. The compound of Formula (I) test formulations will contain 50 mg of the compound of Formula (I) as free base equivalent. Subjects must swallow the study drug with a liquid dietary supplement (Ensure Plus® [237 mL container]) with an additional 100 mL of water (SDD capsule formulation) or with an additional 80 mL of water (SDD suspension formulation) during treatment period 1 or 2. Subjects must swallow the study drug with 330 mL of water (SDD capsule formulation) or 310 mL of water (SDD suspension formulation) during treatment period 3.

Reference Therapy, Dose, and Mode of Administration

The compound of Formula (I) reference formulation (encapsulated, lipidic semi-solid formulation) will be supplied as capsules for oral administration. The compound of Formula (I) reference capsules will contain 50 mg of the compound of Formula (I) as free base equivalent. Subjects must swallow a single capsule with a liquid dietary supplement (Ensure Plus [237 mL container]) with an additional 100 mL of water during treatment period 1 or 2.

### Criteria for Evaluation

Pharmacokinetics

Blood samples for assessment of plasma concentrations of the compound of Formula (I) and metabolites will be collected within 45 minutes before dosing, and at approximately 30 minutes, and 1, 2, 3, 4, 5, 6, 7, 8, 9, 10, 12, 16, 24, 36, 168, 336, and 504 hours after dosing.

The following plasma PK parameters will be calculated for the compound of Formula (I) and metabolites:
Area under the plasma concentration versus time curve from 0 hours to the time of the last measurable concentration (AUC₀₋ₜₗₐₛₜ)
Area under the plasma concentration curve extrapolated from 0 hours to infinity (AUC_{0-∞})
Maximum plasma concentration (Cₘₐₓ)
Time to achieve maximum plasma concentration (tₘₐₓ)
Delay time between time of dosing and time of appearance of measurable test article (T_{lag})
Apparent terminal half-life (t_{1/2})
Apparent terminal rate constant (λz)
Apparent mean residence time (MRT)
Molar AUC ratio of primary metabolite(s) to the parent drug the compound of Formula (I)

The following plasma PK parameters will be calculated only for the compound of Formula (I):
Apparent systemic clearance after oral administration (CL/F)
Apparent volume of distribution during terminal phase after oral administration (Vz/F)

Other Assessment

A taste satisfaction questionnaire will be administered.

Safety Assessments

Safety will be monitored throughout the study and will include the following assessments:
AEs
Clinical laboratory tests (hematology, coagulation, clinical chemistry, and urinalysis)
Vital sign measurements (including orthostatic blood pressure and pulse rate)
Physical examinations
12-lead electrocardiogram (ECG)

Statistical Methods

Pharmacokinetic parameters will be calculated using noncompartmental methods and summarized by formulation using descriptive statistics. Two-sided 90% confidence intervals will be calculated for the ratio of each test formulation (SDD suspension and SDD capsule) vs. the reference formulation for AUC_{0-∞}, AUC₀₋ₜₗₐₛₜ, and Cₘₐₓ for the compound of Formula (I) and metabolites under fed conditions. Further, two-sided 90% confidence intervals will be calculated for the ratio of each test formulation under fasted conditions vs. under fed conditions for AUC_{0-∞}, AUC₀₋ₜₗₐₛₜ, and Cₘₐₓ for the compound of Formula (I) and metabolites.

Safety and taste satisfaction questionnaire data will be summarized with descriptive statistics.

### Results

Pharmacokinetic Results

Pharmacokinetic results are shown in Tables 23-26, below.

TABLE 23
Summary of Plasma Pharmacokinetic Parameters
(Safety Analysis Set - SDD Suspension Group)

| | | | Compound of Formula (I) | | | |
|---|---|---|---|---|---|---|
| | | | Plasma Concentration | | | |
| | Reference Capsule | | SDD Suspension | | SDD Suspension | |
| Parameter (units) | (Fed) | | (Fed) | | (Fasted) | |
| Statistic | (N = 18) | | (N = 18) | | (N = 18) | |
| AUC₀₋ₜₗₐₛₜ | | | | | | |
| (ng × hr/mL) | | | | | | |
| Mean (SD) | 16600 | (7880) | 5980 | (3960) | 737 | (417) |
| Geom CV(%) | 46.8 | | 72.8 | | 68.7 | |
| AUC_{0-∞} | | | | | | |
| (ng × hr/mL) | | | | | | |
| Mean | 17400 | (8010) | 6500 | (4180) | 893 | (480) |
| Geom CV(%) | 45.4 | | 71.7 | | 60.1 | |
| Cₘₐₓ (ng/mL) | | | | | | |
| Mean (SD) | 1480 | (506) | 672 | (323) | 72.3 | (44.9) |
| Geom CV(%) | 32.1 | | 57.9 | | 57.5 | |
| tₘₐₓ (hr) | | | | | | |
| Median (min, max) | 5.0 | (3.0, 6.0) | 5.0 | (5.0, 10) | 7.0 | (5.0, 12) |
| T_{lag} (hr) | | | | | | |
| Mean (SD) | 1.0 | (0.31) | 0.85 | (0.29) | 1.2 | (0.65) |
| t_{1/2} (hr) | | | | | | |
| Mean (SD) | 50 | (20) | 19 | (6.1) | 9.0 | (0.46) |
| Geom CV(%) | 140 | | 93 | | 23 | |
| MRT (hr) | | | | | | |
| Mean (SD) | 36 | (43) | 23 | (24) | 17 | (2.9) |
| Geom CV(%) | 84 | | 62 | | 18 | |
| CL/F (L/hr) | | | | | | |
| Mean (SD) | 3.4 | (1.4) | 11 | (8.0) | 80 | (54) |
| Geom CV(%) | 45 | | 72 | | 63 | |
| Vz/F (L) | | | | | | |
| Mean (SD) | 180 | (260) | 210 | (140) | 970 | (560) |
| Geom CV(%) | 93 | | 77 | | 53 | |
| Tₗₐₛₜ (hr) | | | | | | |
| Mean (SD) | 170 | (140) | 77 | (110) | 31 | (7.6) |

| | | | |
|---|---|---|---|
| Geom CV(%) | 120 | 92 | 30 |

TABLE 24
Geometric Mean Ratios for Pharmacokinetic Exposure Parameters by formulation
and fed vs fasted (Safety Analysis Set - SDD Suspension Group)

| Analyte | | | |
|---|---|---|---|
| Parameter (units) | | | |
| Compound of | | Ratio | |
| Formula (I) | Treatment Comparison | (%) | 90% CI |
| AUC₀₋ₜₗₐₛₜ | SDD Suspension (Fed) vs. Reference | 32.2 | (26.4%, 39.4%) |
| (ng × hr/mL) | Capsule (Fed) | | |
| AUC_{0-∞} | SDD Suspension (Fed) vs. Reference | 33.5 | (27.7%, 40.6%) |
| (ng × hr/mL) | Capsule (Fed) | | |
| Cₘₐₓ (ng/mL) | SDD Suspension (Fed) vs. Reference | 42.2 | (34.8%, 51.2%) |
| | Capsule (Fed) | | |
| AUC₀₋ₜₗₐₛₜ | SDD Suspension (Fasted) vs. Reference | 4.4 | (3.4%, 5.7%) |
| (ng × hr/mL) | Capsule (Fed) | | |
| AUC_{0-∞} | SDD Suspension (Fasted) vs. Reference | 5.0 | (3.8%, 6.5%) |
| (ng × hr/mL) | Capsule (Fed) | | |
| Cₘₐₓ (ng/mL) | SDD Suspension (Fasted) vs. Reference | 4.5 | (3.8%, 5.4%) |
| | Capsule (Fed) | | |

TABLE 25
Summary of Plasma Pharmacokinetic Parameters
(Safety Analysis Set - SDD Capsule Group)

| | | | Compound of Formula (I) | | | |
|---|---|---|---|---|---|---|
| | | | Plasma Concentration | | | |
| | Reference Capsule | | SDD Capsule | | SDD Capsule | |
| Parameter (units) | (Fed) | | (Fed) | | (Fasted) | |
| Statistic | (N = 18) | | (N = 18) | | (N = 17) | |
| AUC₀₋ₜₗₐₛₜ | | | | | | |
| (ng × hr/mL) | | | | | | |
| Mean (SD) | 19500 | (7960) | 9470 | (4670) | 1840 | (1650) |
| Geom CV(%) | 50.4 | | 48.0 | | 78.7 | |
| AUC_{0-∞} | | | | | | |
| (ng × hr/mL) | | | | | | |
| Mean | 20100 | (7850) | 10000 | (4610) | 2120 | (1830) |
| Geom CV(%) | 47.1 | | 44.9 | | 73.7 | |
| Cₘₐₓ (ng/mL) | | | | | | |
| Mean (SD) | 1770 | (494) | 1110 | (449) | 143 | (110) |
| Geom CV(%) | 25.7 | | 44.5 | | 74.1 | |
| tₘₐₓ (hr) | | | | | | |
| Median (min, max) | 5.0 | (5.0, 8.0) | 5.0 | (3.0, 6.0) | 7.0 | (6.0, 12) |
| T_{lag} (hr) | | | | | | |
| Mean (SD) | 1.0 | (0.38) | 1.3 | (0.49) | 1.2 | (0.88) |
| t_{1/2} (hr) | | | | | | |
| Mean (SD) | 35 | (18) | 26 | (20) | 14 | (11) |
| Geom CV(%) | 69 | | 98 | | 49 | |
| MRT (hr) | | | | | | |
| Mean (SD) | 28 | (12) | 25 | (14) | 21 | (9.2) |
| Geom CV(%) | 45 | | 55 | | 31 | |
| CL/F (L/hr) | | | | | | |
| Mean (SD) | 3.0 | (1.5) | 5.9 | (2.4) | 36 | (18) |
| Geom CV(%) | 47 | | 45 | | 74 | |
| Vz/F (L) | | | | | | |
| Mean (SD) | 130 | (61) | 190 | (140) | 630 | (380) |
| Geom CV(%) | 47 | | 75 | | 72 | |
| Tₗₐₛₜ (hr) | | | | | | |
| Mean (SD) | 170 | (96) | 100 | (89) | 44 | (32) |

| | | | |
|---|---|---|---|
| Geom CV(%) | 91 | 100 | 39 |

TABLE 26
Geometric Mean Ratios for Pharmacokinetic Exposure Parameters by formulation
and fed vs fasted (Safety Analysis Set - SDD Suspension Group)

| | | | |
|---|---|---|---|
| Analyte Parameter (units) | Treatment Comparison | Ratio (%) | 90% CI |

Compound of Formula (I)

| | | | |
|---|---|---|---|
| AUC₀₋ₜₗₐₛₜ | SDD Capsule (Fed) vs. Reference | 48.2 | (41.4%, 56.1%) |
| (ng × hr/mL) | Capsule (Fed) | | |
| AUC_{0-∞} | SDD Capsule (Fed) vs. Reference | 49.7 | (43.1%, 57.2%) |
| (ng × hr/mL) | Capsule (Fed) | | |
| Cₘₐₓ (ng/mL) | SDD Capsule (Fed) vs. Reference | 59.8 | (51.7%, 69.2%) |
| | Capsule (Fed) | | |
| AUC₀₋ₜₗₐₛₜ | SDD Capsule (Fasted) vs. Reference | 8.0 | (5.7%, 11.2%) |
| (ng × hr/mL) | Capsule (Fed) | | |
| AUC_{0-∞} | SDD Capsule (Fasted) vs. Reference | 9.0 | (6.6%, 12.4%) |
| (ng × hr/mL) | Capsule (Fed) | | |
| Cₘₐₓ (ng/mL) | SDD Capsule (Fasted) vs. Reference | 6.7 | (4.9%, 9.2%) |
| | Capsule (Fed) | | |

### Example 8: Phase 2 Study of the Compound of Formula (I) in Adult Subjects with Congenital Adrenal Hyperplasia

A Phase 2 study to assess the safety, tolerability, pharmacokinetics (PK), and pharmacodynamics (PD) of the compound of Formula (I) in adult subjects with classic congenital adrenal hyperplasia (CAH) was designed. The objectives of the study are: to assess the safety and tolerability of two ascending doses of the compound of Formula (I) in adult subjects with CAH; to evaluate the effect of repeated doses of the compound of Formula (I) on endogenous levels of PD biomarkers in adult subjects with CAH; and to evaluate plasma exposures following repeated doses of the compound of Formula (I) administered nightly.

The lower dose strength selected for this study, the compound of Formula (I) 50 mg, was well tolerated in both single and repeat-dose safety and PK studies in healthy volunteers. Doses up to 100 mg were also well tolerated in both single-dose and repeat-dose Phase 1 studies in healthy volunteers, and importantly, in a large Phase 2 study in non-elderly female and male subjects with major depressive disorder receiving the compound of Formula (I) during an 8-week, double-blind treatment period. Furthermore, the anticipated steady state exposures with the selected the compound of Formula (I) doses, using the predicted Cₘₐₓ and AUC, are within the acceptable safety margins defined by the nonclinical toxicology studies that have been conducted to date.

### Study Design

A Phase 2, open-label, multiple-dose, dose-escalation study to assess the safety, tolerability, PK, and PD of the compound of Formula (I) in approximately 30 adult female and male subjects (18 to 50 years of age) with a documented medical diagnosis of classic 21-hydroxylase deficiency CAH was designed. The study will include a sequential-cohort design with four compound of Formula (I) dose cohorts: 50 mg and 100 mg, with each dose administered for 14 consecutive days:
Cohort 1: compound of Formula (I) 50 mg once daily with a bottle of vanilla-flavored Ensure Plus® (˜237 mL) at approximately 2200 hours.
Cohort 2: compound of Formula (I) 100 mg once daily with a bottle of vanilla-flavored Ensure Plus® (˜237 mL) at approximately 2200 hours.
Cohort 3: compound of Formula (I) 100 mg once daily with the evening meal at approximately 1900 hours.
Cohort 4: compound of Formula (I) 100 mg twice daily with breakfast at approximately 0700 hours and with the evening meal at approximately 1900 hours.

There will be an approximate 2-week period to evaluate safety and tolerability data before proceeding from Cohort 1 to Cohort 2. Subjects who previously completed the current study in Cohort 1 or Cohort 2 may reenroll to participate in Cohorts 3 or 4 (in addition to new subjects). Table 27 below depicts the dose cohorts, doses, and number of subjects per cohort.

TABLE 27
Dose cohorts, doses, and number of subjects

| | Compound of | Approximate | |
|---|---|---|---|
| | Formula (I) | Dosing | Number of |
| Cohort | Dose | Time(s) | Subjects |
| 1 | 50 mg | 2200 hours | 8-10 |
| 2 | 100 mg | 2200 hours | 8-10 |
| 3 | 100 mg | 1900 hours | 8-10 |
| 4 | 100 mg | 0700 and 1900 hours | Up to 8 |

Subjects will be screened for eligibility to participate in the study for up to approximately 3 weeks (Days −28 to −8). Subjects who reenroll and have had a stable medication regimen for CAH since their last visit in this study do not have to undergo screening; those who reenroll and have had a change to their medication regimen for CAH must undergo a second screening visit. During screening, subjects will provide a single blood sample in the morning between 0700 and 1000 hours (prior to first morning dose of hydrocortisone) to determine their 17-hydroxyprogesterone (17-OHP) levels for study entry.

Eligible subjects who have a screening 17-OHP level ≥1,000 ng/dL will be admitted to the study center for 1 night and have baseline serial PD samples collected over a 24-hour period beginning in the evening of Day −7. Baseline serial PD samples will be collected at approximately 2145, 2300, 2400, 0100, 0200, 0400, 0600, 0800, 1000, 1200, 1400, 1600, and 2200 hours. The subjects' usual morning dose of steroidal treatments will be administered after the 1000 hours PD sample is collected on Day −6. Subjects will be discharged on Day −6 after the last PD sample is collected.

Subjects within each dose cohort will be admitted to the study center on Days 1 and 14 (first and last day of dosing). Subjects will have a blood sample collected on Day 1 for CYP21A2 genotyping. Baseline safety assessments will be collected on Day 1 prior to the first dose of study drug. Study drug (the compound of Formula (I) 50 or 100 mg) will be administered at approximately 2200 hours for Cohorts 1 and 2 and at approximately 1845, 2000, 2100, 2200, 2300, 2400, 0100, 0200, 0400, 0600, 0800, 1000, 1200, 1400, 1800, 1900, and 2200 hours for Cohorts 3 and 4. The subjects' usual morning dose of steroidal treatments will be administered after the 1000 hours PD sample is collected on Day −6. Subjects will be discharged on Day −6 after the last PD sample is collected.

Subjects in Cohorts 1 and 2 will be admitted to the study center on Days 1 and 14 (first and last day of dosing). Subjects will have a blood sample collected on Day 1 for cytochrome P450 (CYP) 21A2 genotyping. Baseline safety assessments will be collected on Day 1 prior to the first dose of study drug. Study drug (compound of Formula (I) 50 mg or 100 mg) will be administered at approximately 2200 hours. The subjects' usual morning dose of concurrent steroidal treatments will be administered after the 12-hour post-dose PK/PD samples are collected (at approximately 1000 hours) on Day 2 and after the 16-hour post-dose PK/PD samples are collected (at approximately 1400 hours) on Day 15. Subjects will be discharged from the study center the evening on Days 2 and 15 following completion of all study-related procedures for those days. Prior to this discharge on Day 2, study drug will be administered at the study center at approximately 2200 hours. Study drug will then be self-administered nightly at home at approximately 2200 hours on Days 3 to 13. Subjects will take their usual morning dose of concurrent steroidal treatments at approximately 1000 hours on Days 3 to 14. On Day 7 during the treatment period, PK, PD, and safety assessments will be conducted in an outpatient setting at the study center.

Subjects in Cohorts 3 and 4 will have a blood sample collected on Day 1 for CYP21A2 genotyping (only for subjects who have not previously participated in Cohorts 1 or 2). Baseline safety assessments will be collected on Day 1 prior to the first dose of study drug. For Cohort 3, study drug (compound of Formula (I) 100 mg) will be administered at home on Days 1 to 13 at approximately 1900 hours with each subject's evening meal. For Cohort 4, study drug compound of Formula (I) 100 mg) will be administered at home on Days 2 to 14 at approximately 0700 hours with each subject's breakfast and on Days 1 to 13 at approximately 1900 hours with each subjects' evening meal. For both cohorts, the Day 14 evening dose will be administered at the study site. Subjects will take their usual morning dose of concurrent steroidal treatments at approximately 1000 hours on Days 1 to 14. On Day 7 during the treatment period, PK, PD, and safety assessments will be conducted in an outpatient setting at the study center. Subjects will be admitted to the study center on Day 14 (last day of dosing). On Day 14, subjects will receive study drug in the study center at approximately 1900 hours with a standard (moderate fat/moderate calorie) evening meal. The subjects' usual morning dose of concurrent steroidal treatments will be administered after PK/PD samples are collected at approximately 1400 hours on Day 15. Subjects will be discharged from the study center the evening on Day 15 following completion of all study-related procedures.

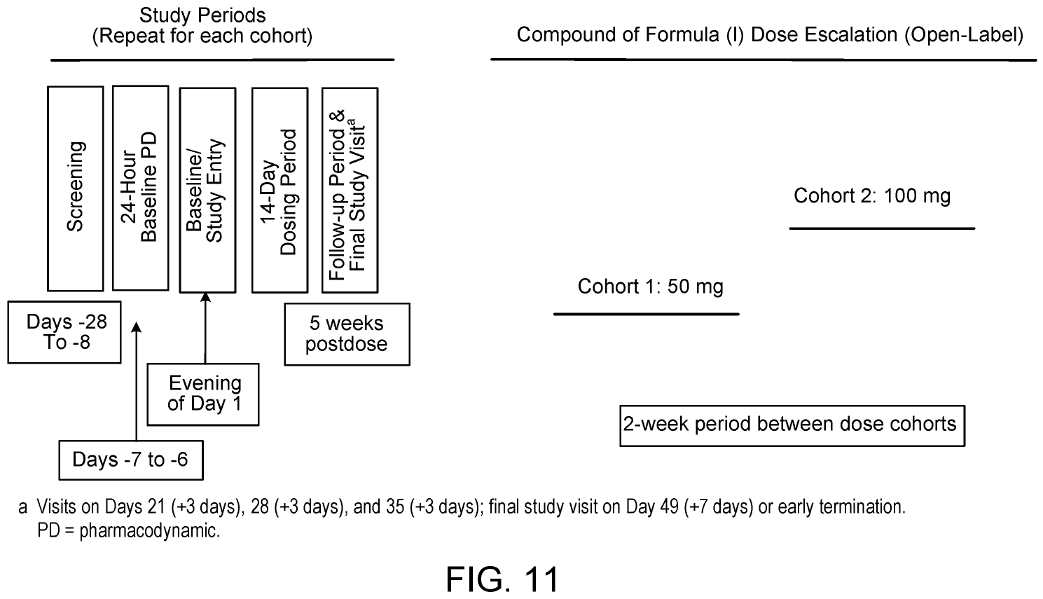

For all cohorts, follow-up visits on Days 21, 28, and 35 will be conducted at the study center or the subject's home by a qualified home healthcare provider (based on the subject's preference). A final study visit will be conducted at the study site approximately 5 weeks after the last dose of study drug (on Day 49 or early termination). There will be a visit window of −8 hours for Day 7, −8 hours/+3 days for Days 21, 28, and 35, and +7 days for the final study visit. Safety, tolerability, PK, and PD will be assessed at scheduled times throughout the study. The study design schematic is shown in FIG. 11.

Dose Escalation Procedure

Cohort 1 will consist of approximately 8 to 10 subjects who will receive a daily dose of the compound of Formula (I) 50 mg at approximately 2200 hours for 14 days (subjects will receive study drug at the site on Days 1, 2, and 14, and self-administer study drug at home on Days 3 to 13). Following the completion of Day 15 assessments for all subjects in the Cohort 1, a medical monitor will review the accumulated safety and tolerability results to ensure there are no safety concerns with proceeding to the 100 mg dose (Cohorts 2 and 3), and to determine if a maximum tolerated dose (MTD) has been reached. If the MTD is reached, no dose escalation will occur. There will be an approximate 2-week period between Cohorts 1 and 2 to accommodate this safety review. A similar procedure will be used prior to proceeding to the 100 mg twice daily dose (Cohort 4).

If the medical monitor determines that it is safe to proceed to the compound of Formula (I) 100 mg, subjects in Cohort 2 will be administered the compound of Formula (I) 100 mg daily for 14 days. Dosing for Cohorts 3 and 4 may begin simultaneously with Cohort 2.

During the 14-day dosing period for any cohort, dosing may be postponed or halted if one or more subjects experience a severe or serious adverse event (AE), or if the type, frequency, or severity of AEs becomes unacceptable. If dosing is postponed, the medical monitor will review all available safety, tolerability, and PK data before allowing any further subjects to receive study drug.

Study Population

Approximately 30 adult female and male subjects (18 to 50 years of age) with a documented medical diagnosis of classic 21-hydroxylase deficiency CAH, who meet all protocol eligibility criteria, will be enrolled. Subjects who previously completed the current study in Cohort 1 or Cohort 2 may reenroll to participate in Cohorts 3 or 4 (in addition to new subjects).

Duration of Treatment

The expected duration of study participation for each subject is approximately 11 weeks, including up to approximately 3 weeks for screening, a 24-hour PD baseline period (approximately 7 days prior to the first day of dosing), 14 days of dosing, and a follow-up period of approximately 5 weeks. The total duration of the study will be an additional 8 to 11 weeks for subjects who reenroll.

Test Product, Dose, and Mode of Administration

The compound of Formula (I) will be supplied as capsules containing 50 mg of the compound of Formula (I) free base for oral administration (see, e.g., Reference 1 formulation as described in Example 9). Doses of the compound of Formula (I) are 50 mg and 100 mg, administered in oral capsule form. Each dose of study drug for Cohorts 1 and 2 is to be administered with a bottle of vanilla-flavored Ensure Plus® (˜237 mL). Each dose of study drug for Cohort 3 is to be administered with each subject's evening meal at approximately 1900 hours. Each dose of study drug for Cohort 4 is to be administered with each subject's breakfast at approximately 1900 hours (i.e., a total daily dose of 200 mg).

### Criteria for Evaluation

Cohorts 1 and 2

Blood samples to evaluate 24-hour PD baseline will be collected on Days −7 to −6 at approximately 2145, 2300, 2400, 0100, 0200, 0400, 0600, 0800, 1000, 1200, 1400, 1600, and 2200 hours. Blood samples to evaluate PK and PD parameters of the compound of Formula (I) will be collected on Days 1 to 2 and Days 14 to 15 at: 15 minutes pre-dose and at 1, 2, 3, 4, 6, 8, 10, 12, 14, 16, 20, and 24 hours post-dose; Day 7 (at approximately 24 hours post-dose); Days 21, 28, and 35 (at approximately 168, 336, and 504 hours post-dose); and at the final study visit (Day 49 or early termination).

Cohorts 3 and 4

Blood samples to evaluate 24-hour PD baseline will be collected on Days −7 to −6 at approximately 1845, 2000, 2100, 2200, 2300, 2400, 0100, 0200, 0400, 0600, 0800, 1000, 1200, 1400, 1800, 1900, and 2200 hours. Blood samples to evaluate PK and PD parameters of the compound of Formula (I) will be collected on Days 14 to 15 at the following times (for Cohort 4, all times are relative to evening dosing unless otherwise indicated): 15 minutes predose and at 1, 2, 3, 4, 5, 6, 7, 9, 11, 13, 15, 17, 19, 23, 24, and 27 hours postdose; Day 7 (at 24 hours postdose for Cohort 3 or at 12 hours post morning dose but prior to the evening dose for Cohort 4); Days 21, 28, and 35 (at approximately 168, 336, and 504 hours postdose); and at the final study visit (Day 49 or early termination).

Pharmacokinetics

The following plasma PK parameters will be calculated for the compound of Formula (I) and metabolites:
Area under the plasma concentration versus time curve from 0 hours to last measurable concentration (AUC₀₋ₜₗₐₛₜ)
Area under the plasma concentration versus time curve from 0 to 24 hours (AUC₀₋₂₄)
Maximum plasma concentration (Cₘₐₓ)
Time to maximum plasma concentration (tₘₐₓ)
Delay time between time of dosing and time of appearance of measurable test article (T_{lag})
Terminal half-life (t_{1/2})
Apparent terminal rate constant (λz)
Apparent mean residence time (MRT)

Additional PK parameters for Day 14 only:

Average plasma concentration at steady state (C_{avg})
Percent fluctuation at steady state (% fluctuation)
Accumulation index at steady state
Apparent systemic clearance after oral administration (CL/F) (the compound of Formula (I) only)

Pharmacodynamics

Primary: Morning 17-OHP (serum; ng/dL) from the 0600, 0800, and 1000 hour samples (8-, 10-, and 12-hour post-dose samples from Cohorts 1 and 2 and 11-, 13-, and 15-hour postdose samples from Cohorts 3 and 4.

Secondary: 17-OHP at all other times, androstenedione (serum; ng/dL), testosterone (serum; ng/dL), cortisol (serum; g/dL), and adrenocorticotropic hormone (plasma ACTH; pg/mL).

Safety

Safety and tolerability will be monitored throughout the study and will include the following assessments:
Adverse events
Clinical laboratory tests—clinical chemistry (including creatine kinase, myoglobin, total and conjugated bilirubin), hematology, coagulation (prothrombin time, aPTT, d-dimer, fibrinogen), and urinalysis (including quantitative myoglobin, casts and crystals)
Vital signs
Physical examinations (including musculoskeletal exam)
12-lead electrocardiograms (ECGs)
Columbia-Suicide Severity Rating Scale (C-SSRS)
Brief Psychiatric Rating Scale (BPRS)

Statistical Methods

Safety, PK, and PD variables will be summarized within each dose cohort (the compound of Formula (I) 50 mg and 100 mg) using descriptive statistics. Summaries of PD measures will include both observed values and changes from pre-dose.

### Results

Pharmacokinetic Results

Eight subjects (four female and four male) were enrolled in this study and completed Cohort 1. Age, sex, and BMI information for the study participants is shown in Table 28, below.

TABLE 28
Cohort 1 Subjects

| Subject | Age | Sex | BMI |
|---|---|---|---|
| ID | (years) | (Male/Female) | (kg/m²) |
| 001 | 37 | Female | 24.9 |
| 002 | 25 | Female | 32.0 |
| 003 | 49 | Male | 37.2 |
| 004 | 36 | Male | 25.0 |
| 005 | 27 | Female | 25.5 |
| 006 | 19 | Male | 21.7 |
| 007 | 25 | Male | 27.5 |
| 008 | 31 | Female | 34.4 |

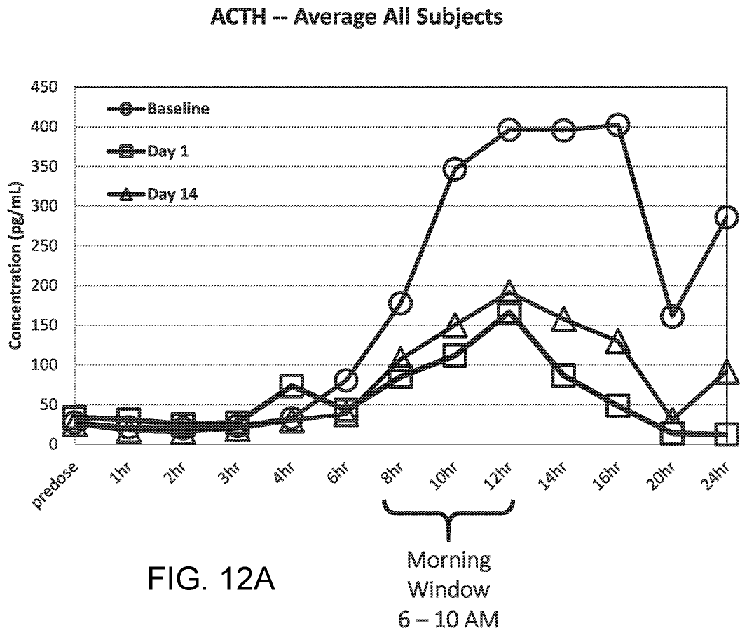

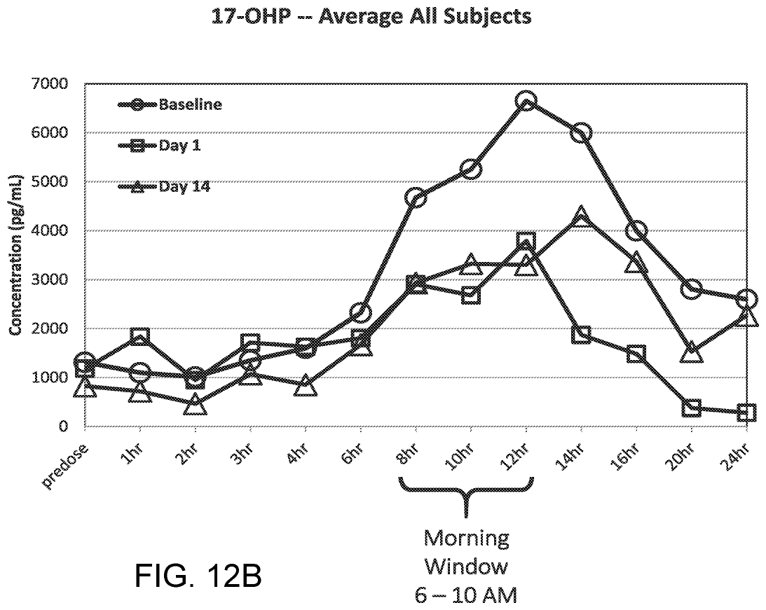

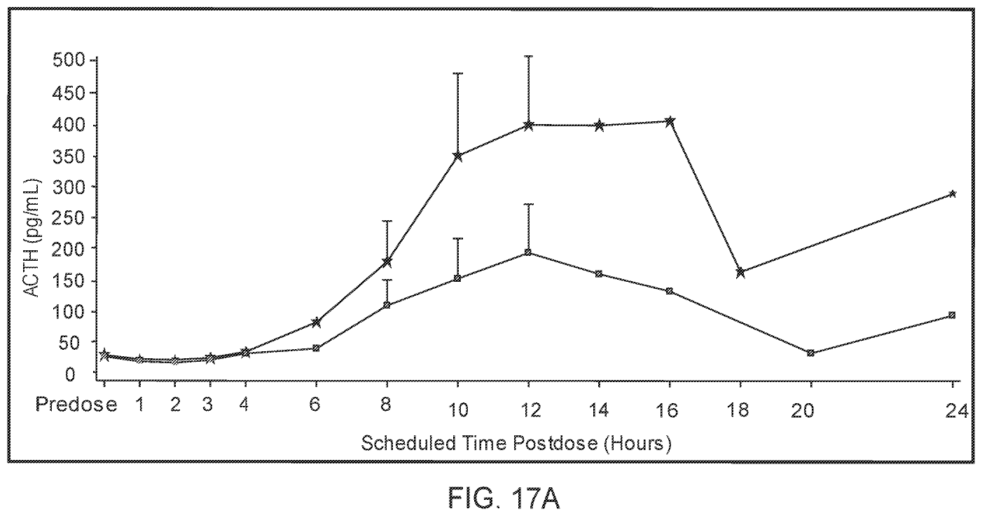

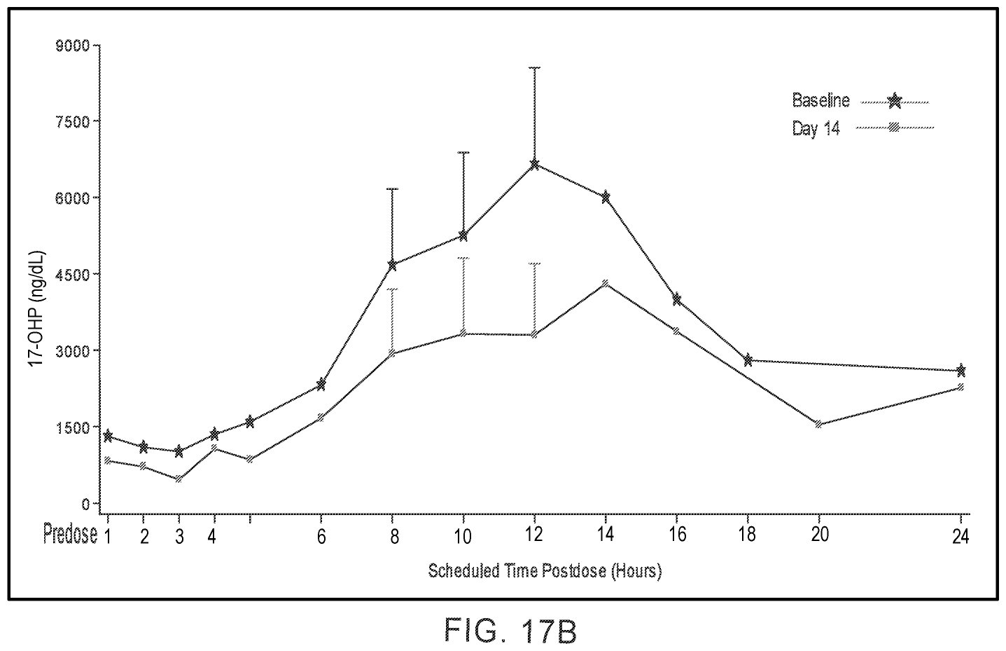

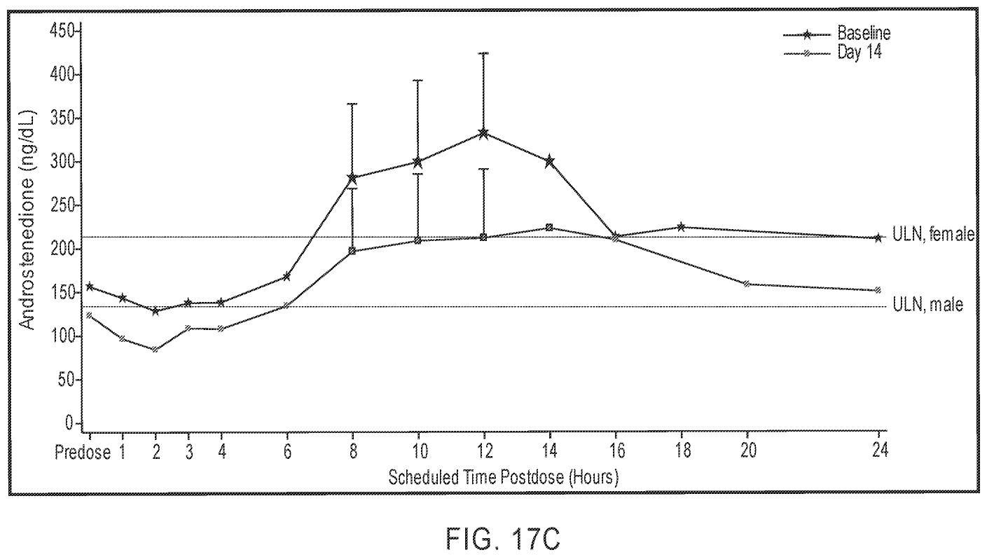

The subjects received a daily dose of the compound of Formula (I) at 50 mg at approximately 2200 hours (˜10 p.m. or bedtime) for 14 days. Arithmetic mean values for ACTH (FIG. 12A) and 17-OHP (FIG. 12B) for all 8 Cohort 1 subjects were plotted at each timepoint for Pre-treatment Baseline, Day 1, and Day 14. Both ACTH and 17OHP concentration profiles at day 1 and day 14 show clear reductions from the baseline mean profiles. Cohort 1 mean PK parameters of Tₘₐₓ, Cₘₐₓ, and AUC₂₄ for the compound of Formula (I) are shown in Table 29 below. These PK parameters are consistent with observations from Phase 1 studies in healthy volunteers.

TABLE 29
Cohort 1 Mean PK parameters of Tₘₐₓ, Cₘₐₓ, and AUC₂₄

| | Day 1 | | | Day 14 | | |
|---|---|---|---|---|---|---|
| | Tₘₐₓ | Cₘₐₓ | AUC₂₄ | Tₘₐₓ | Cₘₐₓ | AUC₂₄ |
| Mean | 5.4 | 1305 | 10,292 | 4.4 | 1349 | 14,297 |
| CV % | 30 | 25 | 24 | 32 | 15 | 24 |

Additional measurements of cohort 1 and cohort 2 mean PK parameters of Tₘₐₓ, Cₘₐₓ, and AUC₂₄ for the compound of Formula (I) on Day 1 of dosing are shown in Table 30 below.

TABLE 30
Mean PK parameters of Tₘₐₓ,
Cₘₐₓ, and AUC₂₄ on Day 1 of dosing

| | Cohort 1 50 mg- Ensure | | | Cohort 2 100 mg-Ensure | | |
|---|---|---|---|---|---|---|
| | Tₘₐₓ* | Cₘₐₓ | AUC₂₄ | Tₘₐₓ | Cₘₐₓ | AUC₂₄ |
| Day 1 | (h) | (ng/ml) | (h*ng/ml) | (h) | (ng/ml) | (h*ng/ml) |
| G.Mean | 6 | 1,270 | 10,411 | 4 | 2,370 | 24,725 |
| CV % | — | 25 | 24 | — | 21 | 42 |
| N | 8 | 8 | 8 | 8 | 4 | 4 |

*Median

Additional measurements of cohort 1, cohort 2 and cohort 3 mean PK parameters of Tₘₐₓ, Cₘₐₓ, and AUC₂₄ for the compound of Formula (I) on Day 14 of dosing are shown in Table 31 below.

TABLE 31
Mean PK parameters of Tₘₐₓ, Cₘₐₓ, and AUC₂₄ on Day 14 of dosing

| | Cohort 1 50 mg- | | | Cohort 2 100 mg- | | | Cohort 3 100 mg | | |
|---|---|---|---|---|---|---|---|---|---|
| | Ensure | | | Ensure | | | w/evening Meal | | |
| | Tmax* | Cmax | AUC24 | Tmax | Cmax | AUC24 | Tmax | Cmax | AUC24 |
| Day 14 | (h) | (ng/ml) | (h*ng/ml) | (h) | (ng/ml) | (h*ng/ml) | (h) | (ng/ml) | (h*ng/ml) |
| G. Mean | 4 | 1,335 | 14,070 | 4 | 3,379 | 35,416 | 3 | 3,650 | 34,706 |
| CV % | | 15 | 24 | | 31 | 37 | | 32 | 15 |
| N | 8 | 8 | 8 | 6 | 6 | 6 | 8 | 8 | 8 |

*Median

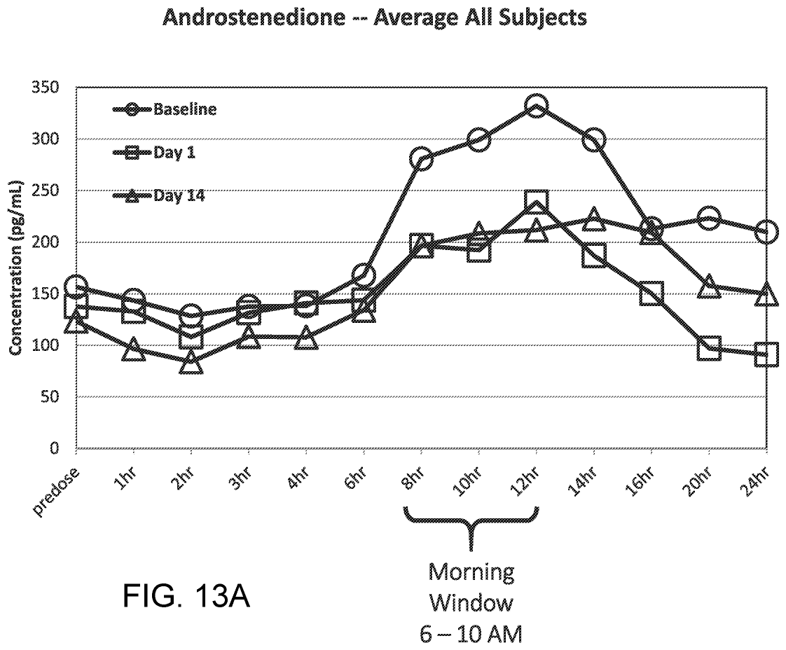

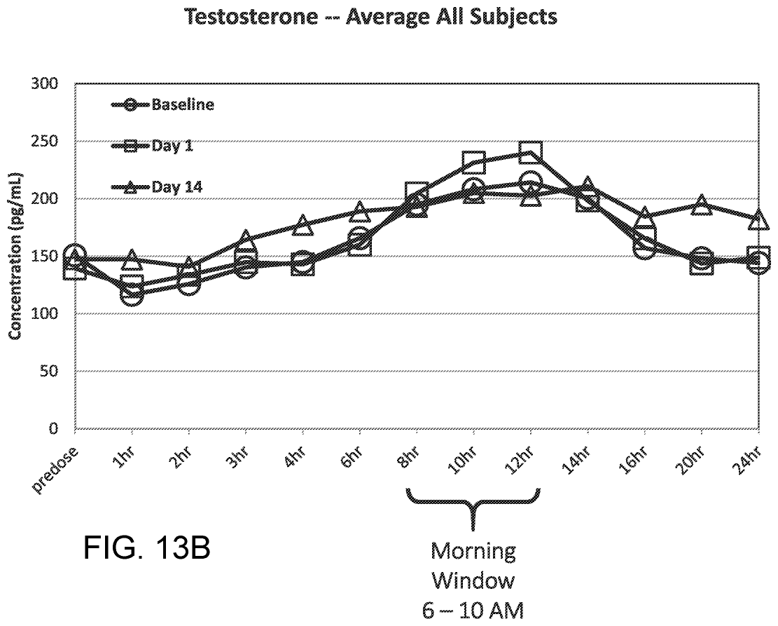

Arithmetic mean values for androstenedione (FIG. 13A) and testosterone (FIG. 13B) for all 8 Cohort 1 subjects were plotted at each timepoint for Pre-treatment Baseline, day 1, and day 14. The androstenedione concentration profile on Day 1 and Day 14 shows a clear reduction from the baseline mean profile.

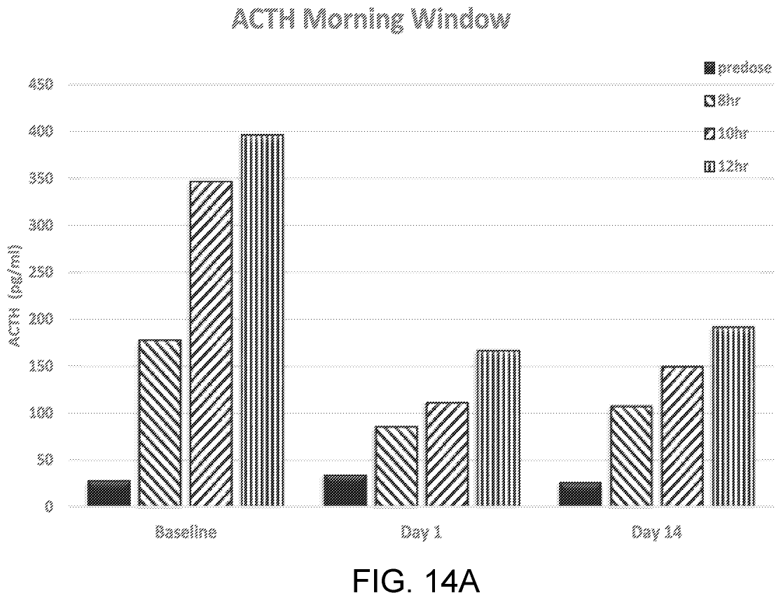

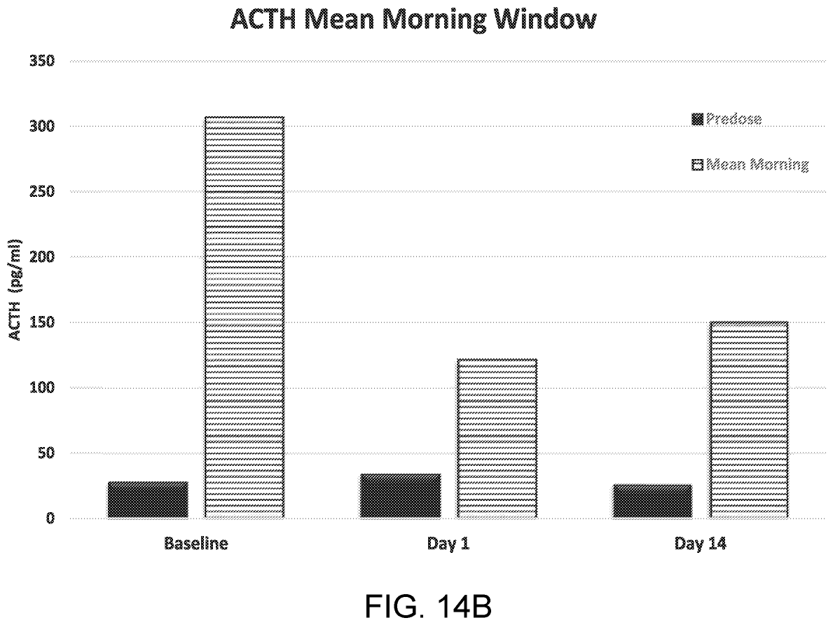

When focusing exclusively on the critical morning window period (timepoints at 8-, 10-, and 12-hours postdose) from 6:00 a.m. to 10:00 a.m., the levels of ACTH show marked reductions from baseline at each of the 3 timepoints on Days 1 and 14 (FIG. 14A). Arithmetic mean values across all three timepoints show >=50% reductions from baseline at Day 1 and Day 14 (FIG. 14B).

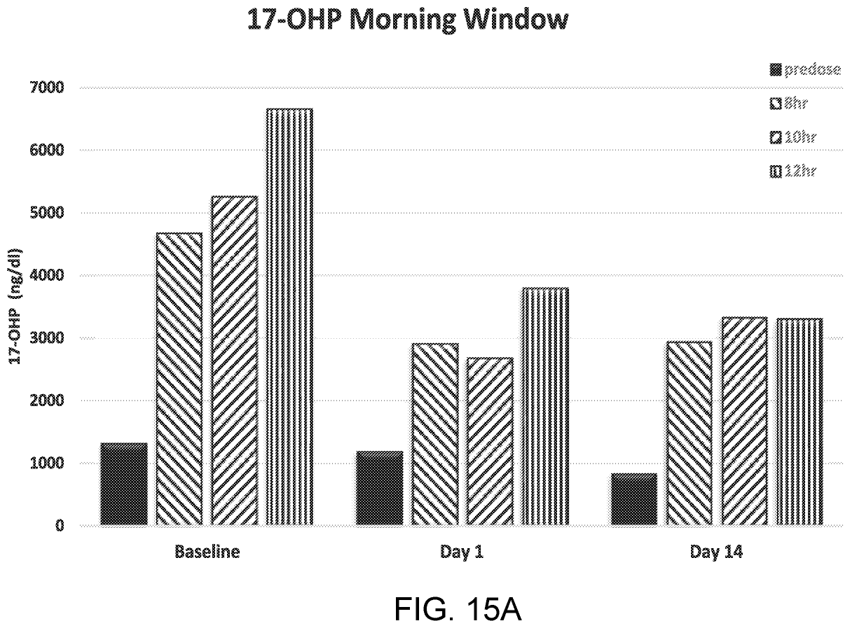

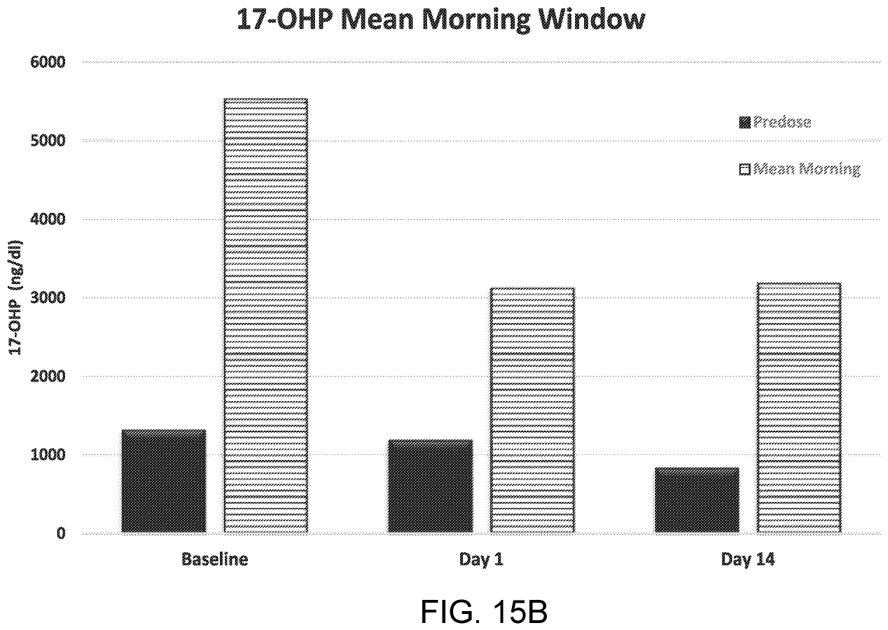

When focusing exclusively on the critical morning window period (timepoints at 8-, 10-, and 12-hours postdose) from 6:00 a.m. to 10:00 a.m., the levels of 17-OHP show marked reductions from baseline at each of the 3 timepoints on Days 1 and 14 (FIG. 15A). Arithmetic mean values across all three timepoints show >=40% reductions from baseline at Day 1 and Day 14 (FIG. 15B).

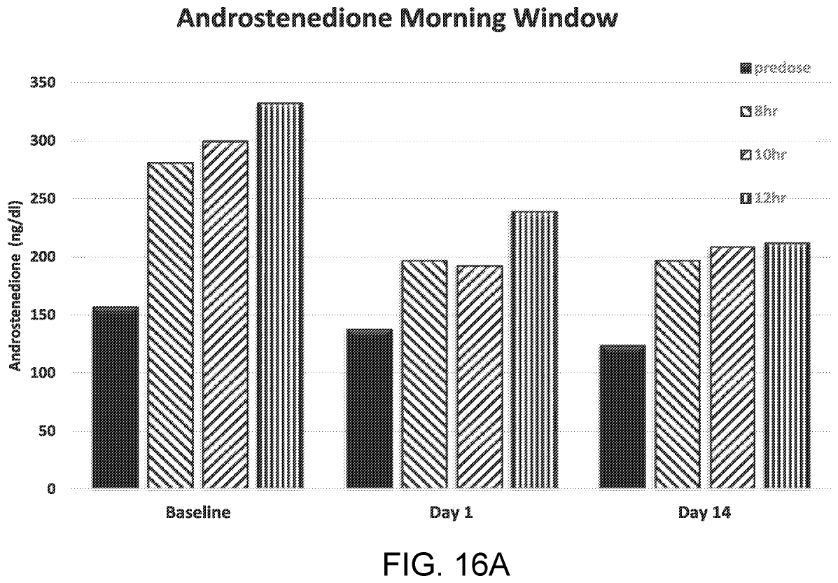

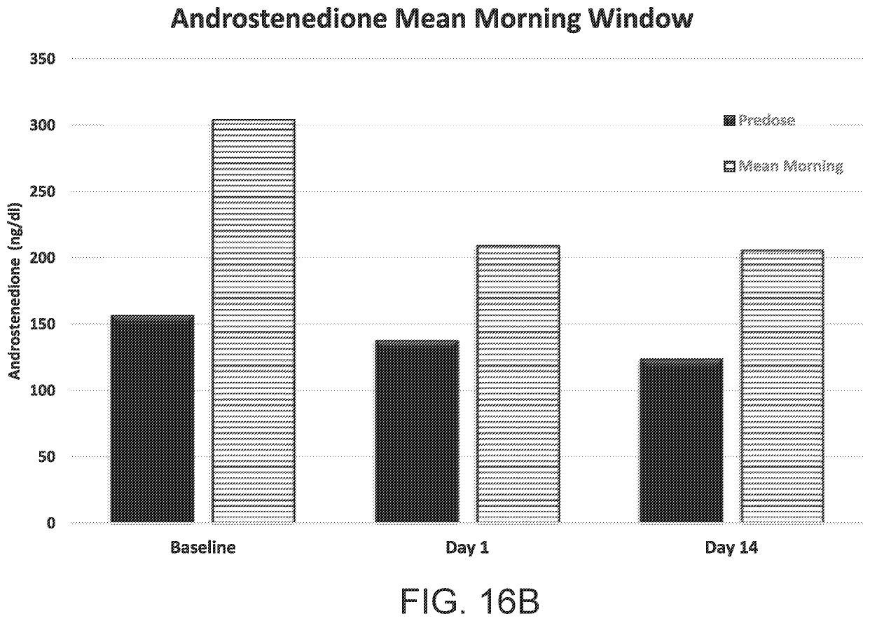

When focusing exclusively on the critical morning window period (timepoints at 8-, 10-, and 12-hours postdose) from 6:00 a.m. to 10:00 a.m., the levels of androstenedione show marked reductions from baseline at each of the 3 timepoints on Days 1 and 14 (FIG. 16A). Arithmetic mean values across all three timepoints show >=30% reductions from baseline at Day 1 and Day 14 (FIG. 16B).

A summary of reduction in 17-OHP and androstenedione in cohort 1 is shown in Table 32. Further, the androstenedione levels of three subjects was normalized by the treatment for the three subjects with Subject ID Nos. 001, 002, and 006).

TABLE 32
Cohort 1 Summary of Reduction in 17-OHP and Androstenedione
at each timepoint in the morning window (6 a.m. to 10 a.m.)

| | | | 17-OHP | | | Androstenedione | | |
|---|---|---|---|---|---|---|---|---|
| | | | % change from | | | % change from | | |
| Subject | Sex/ | Dosing | baseline | | | baseline | | |
| ID | Age | Day | 6 AM | 8 AM | 10 AM | 6 AM | 8 AM | 10 AM |
| 001 | F/37 | D 1 | −61.4 | 14.7 | −24.1 | −42.0 | 2.0 | −19.5 |
| | | D 14 | −90.3 | −67.2 | −37.2 | −84.6 | −58.6 | −55.1 |
| 002 | F/25 | D 1 | −96.0 | −95.5 | 45.4 | −68.0 | −66.7 | −21.7 |
| | | D 14 | −37.7 | −58.6 | −24.9 | −34.2 | −47.8 | −38.7 |
| 003 | M/49 | D 1 | −11.8 | −46.6 | −34.0 | −13.4 | −47.1 | −22.7 |
| | | D 14 | −24.5 | −5.2 | −22.9 | −7.5 | −10.8 | −16.0 |
| 004 | M/36 | D 1 | −13.7 | 17.1 | 10.1 | 4.5 | 15.9 | 3.0 |
| | | D 14 | −53.6 | −46.8 | −35.8 | −13.8 | −15.2 | −21.2 |
| 005 | F/27 | D 1 | −24.4 | −83.6 | −52.2 | −35.6 | −50.3 | −33.5 |
| | | D 14 | −78.8 | −95.1 | −86.3 | 26.7 | −13.7 | −10.9 |
| 006 | M/19 | D 1 | −5.1 | −1.1 | −97.8 | −10.0 | −18.5 | −78.9 |
| | | D 14 | −25.4 | −20.4 | −98.3 | −67.8 | −62.7 | −92.1 |
| 007 | M/25 | D 1 | −50.0 | −28.0 | −27.4 | −24.8 | −30.0 | −15.8 |
| | | D 14 | 14.9 | 4.0 | −7.1 | −4.8 | −36.8 | −30.2 |
| 008 | F/31 | D 1 | −64.5 | −80.7 | −92.8 | −12.2 | 7.0 | −9.3 |
| | | D 14 | −59.0 | −65.2 | −89.1 | 74.5 | 99.4 | 59.2 |

TABLE 33
Cohort 1 Summary of PK parameters of Tₘₐₓ,
Cₘₐₓ, and AUC₂₄ for each subject at days 1 and 14

| | Day 1 | | | Day 14 | | |
|---|---|---|---|---|---|---|
| | Tₘₐₓ | Cₘₐₓ | AUC₂₄ | Tₘₐₓ | Cₘₐₓ | AUC₂₄ |
| Subject ID | (h) | (ng/ml) | (h*ng/ml) | (h) | (ng/ml) | (h*ng/ml) |
| 001 | 6 | 1830 | 14,487 | 6 | 1570 | 20,863 |
| 002 | 6 | 1210 | 11,829 | 4 | 1610 | 15,494 |
| 003 | 6 | 1060 | 11,068 | 3 | 1310 | 15,599 |
| 004 | 4 | 1670 | 9,669 | 3 | 1160 | 10,180 |
| 005 | 8 | 961 | 9,266 | 6 | 1550 | 15,130 |
| 006 | 4 | 1030 | 6,574 | 3 | 1090 | 10,288 |
| 007 | 6 | 1550 | 8,239 | 6 | 1170 | 12,880 |
| 008 | 3 | 1130 | 11,209 | 4 | 1330 | 13,944 |
| Mean | 5.4 | 1305 | 10,292 | 4.4 | 1349 | 14,297 |
| CV % | 30 | 25 | 24 | 32 | 15 | 24 |
| N | 8 | 8 | 8 | 8 | 8 | 8 |

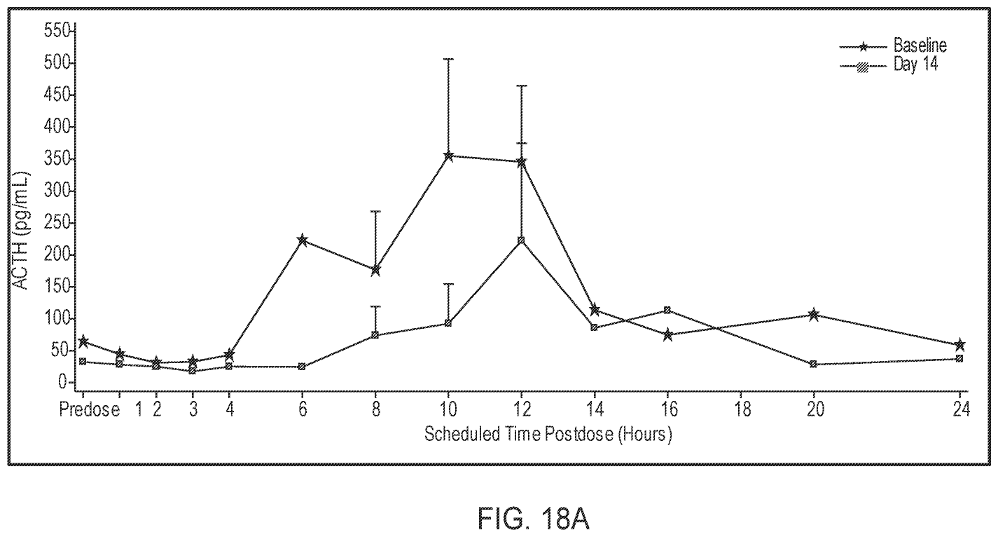

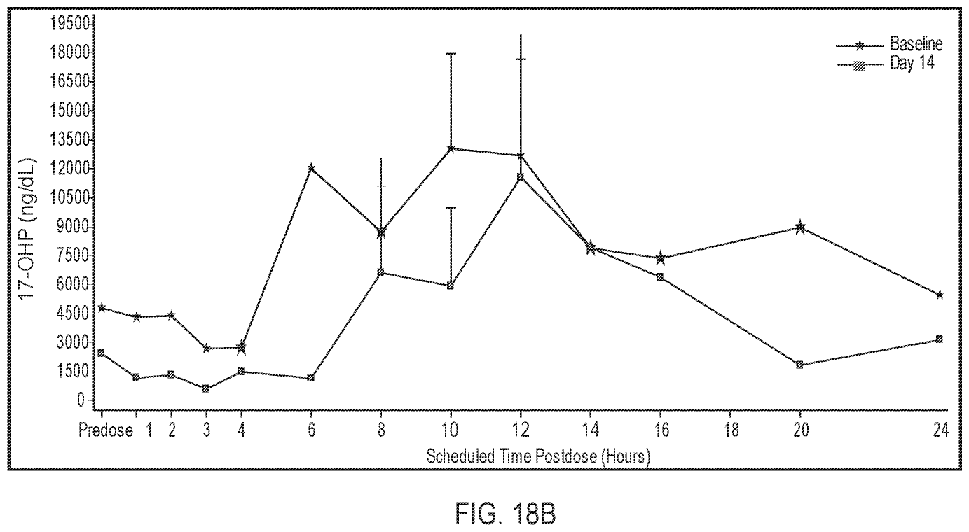

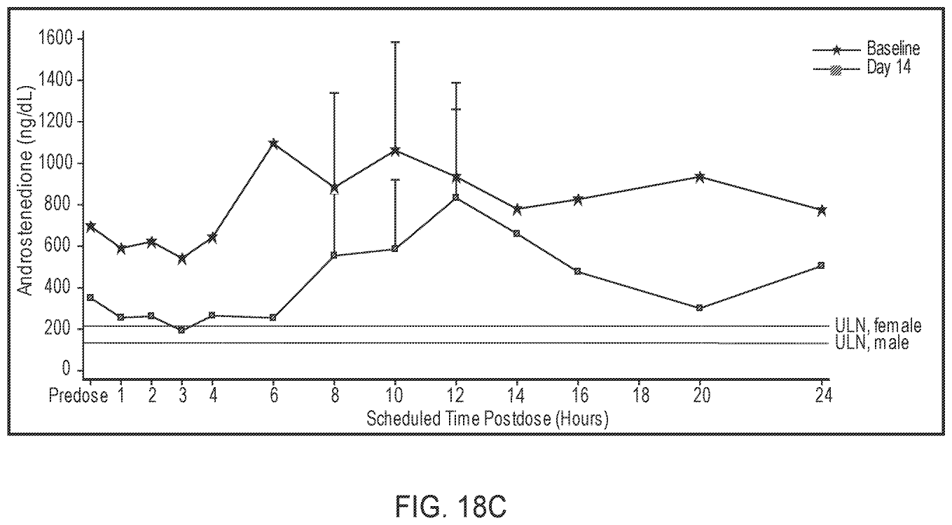

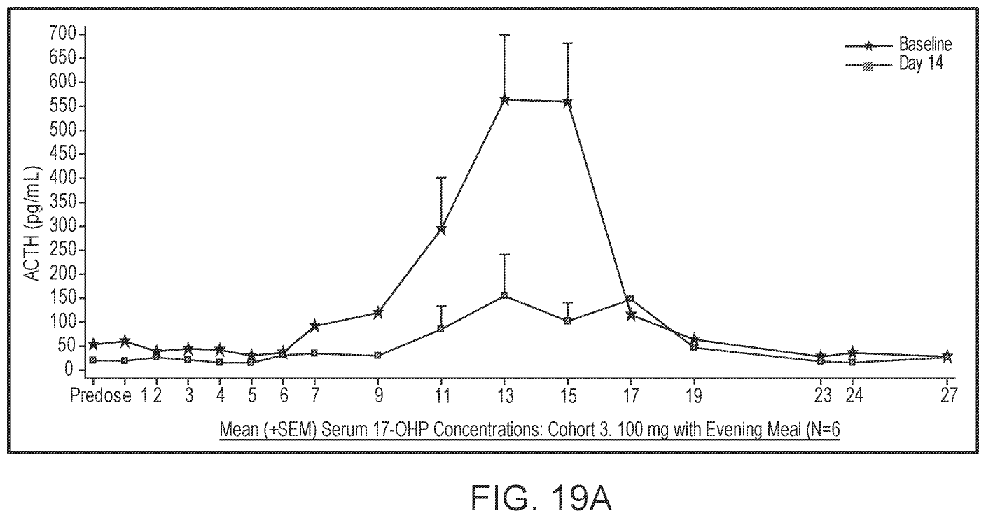

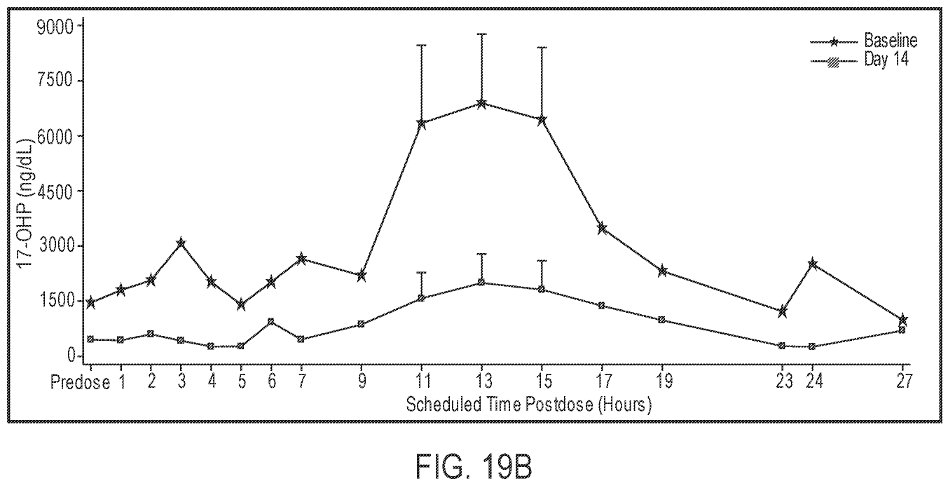

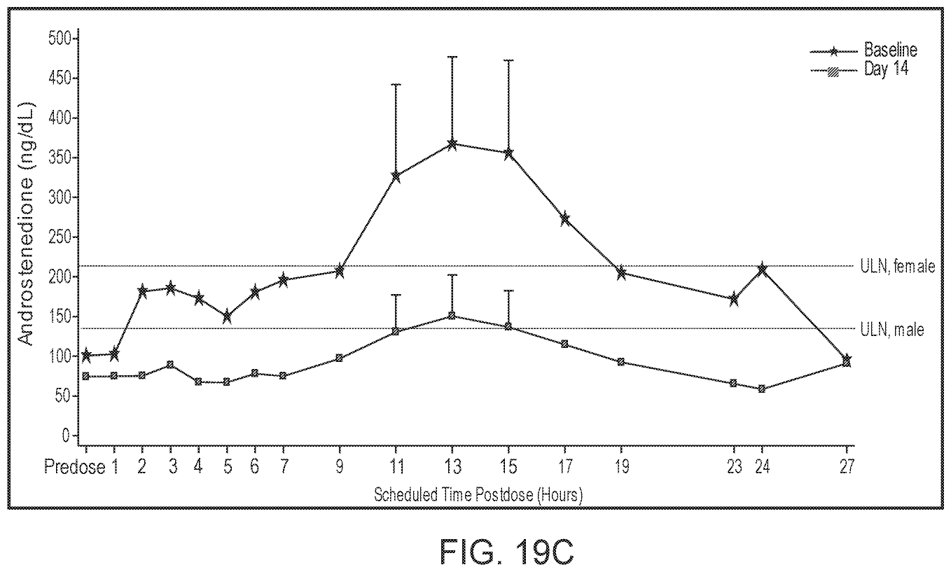

After 14 days of once-daily compound of Formula (I) administration, a majority of participants in Cohorts 1-3 showed reduced serum concentrations of adrenal androgens and precursors. Mean changes from baseline (±standard deviation) in Cohort 1 were as follows: 17-OHP, −2341.0±1535.0 ng/dL; androstenedione, −98.4±98.7 ng/dL; and ACTH, −157.0±194.9 pg/mL. Mean reductions were larger in Cohort 2 (17-OHP, −4406.0±5516.1; androstenedione, −362.8±354.0; ACTH, −180.9±155.2) and Cohort 3 (17-OHP, −4760.1±4018.2; androstenedione, −210.9±188.6; ACTH, −358.9±177.6), suggesting a possible dose response. FIGS. 17A-17C, 18A-18C, and 19A-19C depict results from Cohorts 1, 2, and 3, respectively.

### Summary

The results from this ongoing Phase II open-label study demonstrated a reduction of at least 50 percent from baseline in 17-hydroxyprogesterone (17-OHP) and adrenocorticotropic hormone (ACTH) levels in more than 50 percent of CAH patients in cohort 1 treated with the compound of Formula (I) for 14 days (i.e., 6 of 8 patients in cohort 1 had a reduction of ≥50% from baseline levels of 17-OHP during at least one morning window timepoint, see, e.g., Table 32). Meaningful reductions were also observed in other biomarkers, including androstenedione (i.e., 4 of these patients also had a reduction of ≥50% from baseline levels of androstenedione during at least one morning window timepoint, see, e.g., Table 32). The greater reductions in biomarkers in cohorts 2 and 3, treated with double the dose of the compound of Formula (I) compared with cohort 1, suggest a possible dose response. Further, the compound of Formula (I) was well-tolerated with a relatively small number of mild adverse events (AEs) reported (e.g., headache, ovulation pain, fatigue, localized infection (toe), dizziness, nausea, URI, contusion with the most common being headache). No clinically significant findings from routine labs, vital signs, or electrocardiograms were found.

### Example 9: Reference Formulation 1 of the Compound of Formula (I)

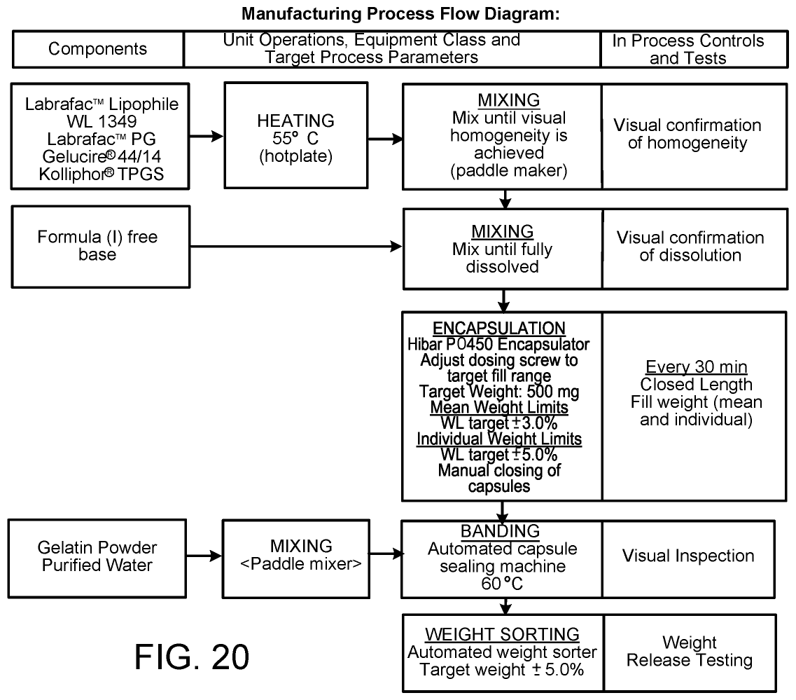

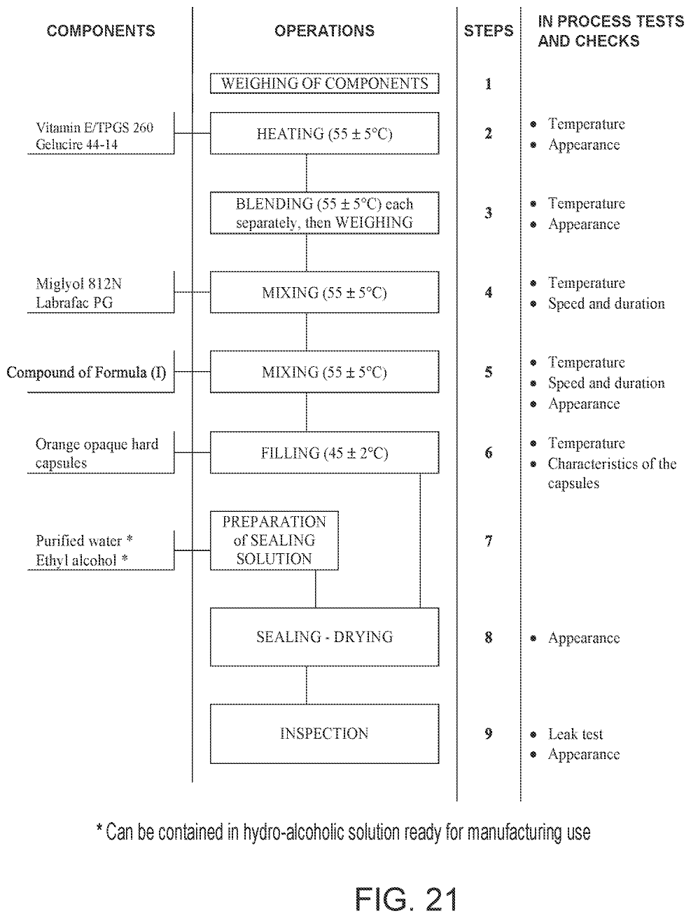

Tables 34A and 34B show Reference formulation 1 of the compound of Formula (I) as used in the clinical studies described in Examples 6 and 8, above. An example manufacturing process is shown in FIG. 20. Another example manufacturing process is shown in FIG. 21.

TABLE 34A

| | | | 50 mg Capsule | |
|---|---|---|---|---|
| | Quality | | Weight | % |
| Component | Standard | Function | (mg/unit) | (w/w) |
| Compound of Formula (I), free | In-house | Active Ingredient | 50.0 | 10.0 |
| base | | | | |
| Medium-Chain Triglycerides | NF | Oily Phase Vehicle | 196.0 | 39.2 |
| (Labrafac TM | | | | |
| Lipophile WL1349) | | | | |
| Propylene Glycol | NF | Emulsifying Agent | 102.0 | 20.4 |
| Dicaprylate/Dicaprate, | | | | |
| (LabrafacTM PG) | | | | |
| Lauroyl Polyoxyl-32 Glycerides | NF | Nonionic Surfactant | 95.0 | 19.0 |
| (Gelucire ® 44/14) | | & Solubilizing | | |
| | | Agent | | |
| Vitamin E Polyethylene Glycol | USP/NF | Solubilizing Agent | 57.0 | 11.4 |
| Succinate (Kolliphor ® TPGS) | | | | |
| Total Emulsion Weight | | | 500.0 | 100.0 |
| Gelatin Capsule Shell. Size #00, | Non | Capsule Shell | — | — |
| Swedish Orange cap/body; (Coni- | Pharmacopoeial | | | |
| Snap ®) | | | | |
| Gelatin Powder, 220 Bloom | USP | Capsule shell | — | — |
| | | banding agent | | |
| Purified Water | USP | Capsule shell | — | — |
| | | banding solvent | | |

TABLE 34B

| | | | 50 mg Capsule | |
|---|---|---|---|---|
| | Quality | | Weight | % |
| Component | Standard | Function | (mg/unit) | (w/w) |
| Compound of Formula (I), free | In-house | Active Ingredient | 50.0 | 10.0 |
| base | | | | |
| Medium-Chain Triglycerides | Ph. Eur./NF | Vehicle | 195.85 | 39.2 |
| (caprylic:capric acid 60:40; | | | | |
| Miglyol 812N) | | | | |
| Propylene Glycol | Ph. Eur. | Vehicle | 102.15 | 20.4 |
| Dicaprylocaprate, (LabrafacTM | | | | |
| PG) | | | | |
| Lauroyl macrogolglycerides type | Ph. Eur./NF | Surfactant | 95.0 | 19.0 |
| 1500-Lauroyl polyoxylglycerides | | | | |
| type 1500 (Gelucire ® 44/14) | | | | |
| Vitamin E Polyethylene Glycol | NF | Surfactant | 57.0 | 11.4 |
| Succinate, 260 mg/g d-alpha | | | | |
| tocopherol (Vitamin E/TPGS | | | | |
| 260) | | | | |
| Total Emulsion Weight | | | 500.0 | 100.0 |
| Orange opaque hard capsule, size | Non | Capsule Shell | — | — |
| 0, composed of gelatin, titanium | Pharmacopoeial | | | |
| dioxide and red ferric oxide | | | | |
| (Swedish Orange ®) | | | | |
| Ethanol (96%) and Purified | USP | Capsule shell | — | — |
| Water | | banding solvent | | |

### Example 10: Study Evaluating Effect of Ensure Plus, Ensure Pudding, Milk and High Fat Meal on Reference Capsule

### Study Design

This is a Phase 1, open-label, randomized, four-period crossover study to evaluate the effect of food with different levels of fat and caloric content on the PK, safety, and tolerability of the compound of Formula (I) in healthy adult subjects.

A total of 16 healthy adult subjects (8 males and 8 females) will be randomly assigned to 1 of 4 treatment sequences (4 subjects per sequence [2 males and 2 females per sequence]; see Table 35 below). During each treatment period, subjects will receive a single dose of the compound of Formula (I) 100 mg administered with the appropriate meal, according to the randomization scheme. There will be a washout of at least 21 days between each dose.

TABLE 35

| Treatment | Treatment | Treatment | Treatment | Treatment |
|---|---|---|---|---|
| Sequence | Period 1 | Period 2 | Period 3 | Period 4 |
| 1 | Reference | Test meal 1 | Test meal 2 | Test meal 3 |
| | meal | | | |
| 2 | Test meal 1 | Test meal 3 | Reference | Test meal 2 |
| | | | meal | |
| 3 | Test meal 2 | Reference | Test meal 3 | Test meal 1 |
| | | meal | | |
| 4 | Test meal 3 | Test meal 2 | Test meal 1 | Reference |
| | | | | meal |

Reference meal: vanilla-flavored Ensure Plus ®
Test meal 1: Low fat, low caloric content meal 1
Test meal 2: Low fat, low caloric content meal 2
Test meal 3: standard high fat, high caloric content meal

After providing informed consent, subjects will be screened for eligibility to participate in the study. Screening will begin up to 28 days before Day 1 of treatment period 1. Eligible subjects will be admitted to the clinical unit on Day −1 and randomized to 1 of the 4 treatment sequences on Day 1 of treatment period 1. During each treatment period, subjects will fast overnight for at least 10 hours until the start of the assigned meal, according to the randomization scheme, and ingest the study drug at approximately 0800 hours. Subjects must complete the entire meal within the specified time period and should not consume any other food for 4 hours after dosing. For all treatment periods, water will not be permitted for 1 hour before dosing until 2 hours after dosing except for the water provided with study drug dosing and planned meals.

On Day 1 of each treatment period, subjects will be dosed with the compound of Formula (I) 100 mg. Blood samples will be collected for PK analysis over a period of 36 hours during the in-house stay. During each treatment period, subjects will remain in the clinic on the day of dosing and will be discharged on Day 2 after completing all required procedures. On the mornings of Days 8 and 15 of each treatment period, subjects will return for an outpatient visit to the clinic for PK blood sample collection and safety assessments. On Day 21 of treatment periods 1 to 3, subjects will arrive at the site and have Day 21 assessments completed and they will stay overnight at the site and begin Day 1 of the subsequent treatment period the following day. A final follow-up study visit will be conducted on Day 22 of treatment period 4 (21±2 days after treatment period 4 dosing) or upon early termination.

Blood samples for PK d/conducted at scheduled times throughout the study.

### Study Population

Sixteen healthy adult subjects (8 males and 8 females) between 18 and 55 years of age, inclusive, who meet all protocol eligibility criteria, will be enrolled.

### Duration of Treatment

The expected duration of study participation for each healthy adult subject will be approximately 16 weeks, including up to 28 days of screening, 4 days of dosing with at least 21 days between consecutive doses, and a final follow-up study visit 21 days (±2 days) after receiving the last dose of study drug during treatment period 4.

### Test Product, Dose, and Mode of Administration

The compound of Formula (I) will be supplied as capsules for oral administration (encapsulated, lipidic semi-solid formulation, e.g., Example 9). The compound of Formula (I) capsules will contain 50 mg of the compound of Formula (I) as free base equivalent. During each treatment period, subjects will receive two 50 mg capsules (100 mg) of the study drug along with a meal and water as defined by the randomization scheme. The food, water, and study drug administration are as follows:
Reference meal: Two capsules of study drug will be administered approximately 5 minutes after the start of a liquid dietary supplement (i.e., Ensure Plus® [237 mL container]) and an additional 120 mL of water for study drug dosing.
Test meal 1: Two capsules of study drug will be administered approximately 5 minutes after the start of a low fat, low caloric meal 1 with 120 mL of water for study drug dosing.
Test meal 2: Two capsules of study drug will be administered approximately 5 minutes after the start of a low fat, low caloric meal 2 with 120 mL of water for study drug dosing.
Test meal 3: Two capsules of study drug will be administered approximately 30 minutes after the start of a high fat, high caloric meal with 120 mL of water for study drug dosing.

### Criteria for Evaluation

### Pharmacokinetics

Blood samples for assessment of plasma concentrations of the compound of Formula (I) and metabolites will be collected within 45 minutes before dosing, and at approximately 30 minutes, and 1, 2, 3, 4, 5, 6, 7, 8, 9, 10, 12, 16, 24, 36, 168, 336, and 504 hours after dosing.

The following plasma PK parameters will be calculated for the compound of Formula (I) and metabolites:
Area under the plasma concentration versus time curve from 0 hours to last measurable concentration (AUC₀₋ₜₗₐₛₜ)
Area under the plasma concentration versus time curve from 0 hours extrapolated to infinity (AUC_{0-∞})
Maximum plasma concentration (Cₘₐₓ)
Time to achieve maximum plasma concentration (tₘₐₓ)
Delay time between time of dosing and time of appearance of measurable test article (T_{lag})
Apparent terminal half-life (t_{1/2})
Apparent terminal rate constant (λz)
Apparent mean residence time (MRT)
Molar AUC ratio of primary metabolite(s) to the parent drug compound of Formula (I). The following plasma PK parameters will be calculated only for the compound of Formula (I):
Apparent systemic clearance after oral administration (CL/F)
Apparent volume of distribution during terminal phase after oral administration (Vz/F)

### Safety Assessments

Safety will be monitored throughout the study and will include the following assessments:
Adverse events (AEs)
Clinical laboratory tests (hematology, coagulation, clinical chemistry, and urinalysis)
Vital sign measurements (including orthostatic blood pressure and pulse rate)
Physical examinations
12-lead electrocardiograms (ECGs)

### Statistical Methods

Pharmacokinetic parameters will be calculated using noncompartmental methods and summarized by meal type (test meal/reference meal) using descriptive statistics. Two-sided 90% confidence intervals will be calculated for the ratio of each test meal versus the reference meal for AUC_{0-∞}, AUC₀₋ₜₗₐₛₜ, and Cₘₐₓ for the compound of Formula (I) and metabolites.

Safety data will be summarized with descriptive statistics.

### Results

Pharmacokinetic Results

Pharmacokinetic results are shown in Table 36 below.

TABLE 36
Summary of Plasma Pharmacokinetic Parameters

| | | | Compound of Formula (I) | | | | | |
|---|---|---|---|---|---|---|---|---|
| | | | Plasma Concentration | | | | | |
| | Ensure Plus | | Ensure Pudding | | Whole Milk | | High Fat Meal | |
| Parameter (units) | (Fed) | | (Fed) | | (Fed) | | (Fed) | |
| Statistic | (N = 18) | | (N = 18) | | (N = 17) | | (N = 17) | |
| AUC₀₋ₜₗₐₛₜ | | | | | | | | |
| (ng × hr/mL) | | | | | | | | |
| Mean (SD) | 36703 | (17400) | 34077 | (11597) | 35561 | (15866) | 55487 | (23242) |
| Geom CV(%) | 58 | | 39.7 | | 44.6 | | 41.9 | |
| AUC_{0-∞} | | | | | | | | |
| (ng × hr/mL) | | | | | | | | |
| Mean | 45386 | (19648) | 40037 | (9515) | 46737 | (17395) | 63755 | (19139) |
| Geom CV(%) | 43.3 | | 23.8 | | 38.8 | | 31 | |
| Cₘₐₓ (ng/mL) | | | | | | | | |
| Mean (SD) | 3090 | (1070) | 3038 | (984) | 2835 | (1005) | 4336 | (1938) |
| Geom CV(%) | 39.7 | | 34.9 | | 34.5 | | 53.6 | |
| tₘₐₓ (hr) | | | | | | | | |
| Median (min, max) | 5.0 | (2.0, 10.0) | 5.0 | (4.0, 6.0) | 5.0 | (4.0, 7.0) | 5.0 | (4.0, 7.0) |
| T_{lag} (hr) | | | | | | | | |
| Mean (SD) | 0.38 | (0.46) | 0.20 | (0.25) | 0.16 | (0.31) | 0.29 | (0.37) |
| t_{1/2} (hr) | | | | | | | | |
| Mean (SD) | 361 | (263) | 373 | (196) | 373 | (143) | 326 | (120) |
| Geom CV(%) | 61.1 | | 48.6 | | 37.1 | | 33.8 | |
| CL/F (L/hr) | | | | | | | | |
| Mean (SD) | 2.49 | (0.956) | 2.636 | (0.659) | 2.427 | (0.975) | 1.706 | (0.526) |
| Geom CV(%) | 41.2 | | 24.8 | | 38.8 | | 31.1 | |
| Vz/F (L) | | | | | | | | |
| Mean (SD) | 1254 | (906) | 1414 | (720) | 1308 | (627) | 816 | (378) |

| | | | | |
|---|---|---|---|---|
| Geom CV(%) | 72.7 | 58.4 | 61.3 | 54.9 |

### Example 11: Spray-Dried Dispersion Granule Formulation of the Compound of Formula (I) (SDD-G)

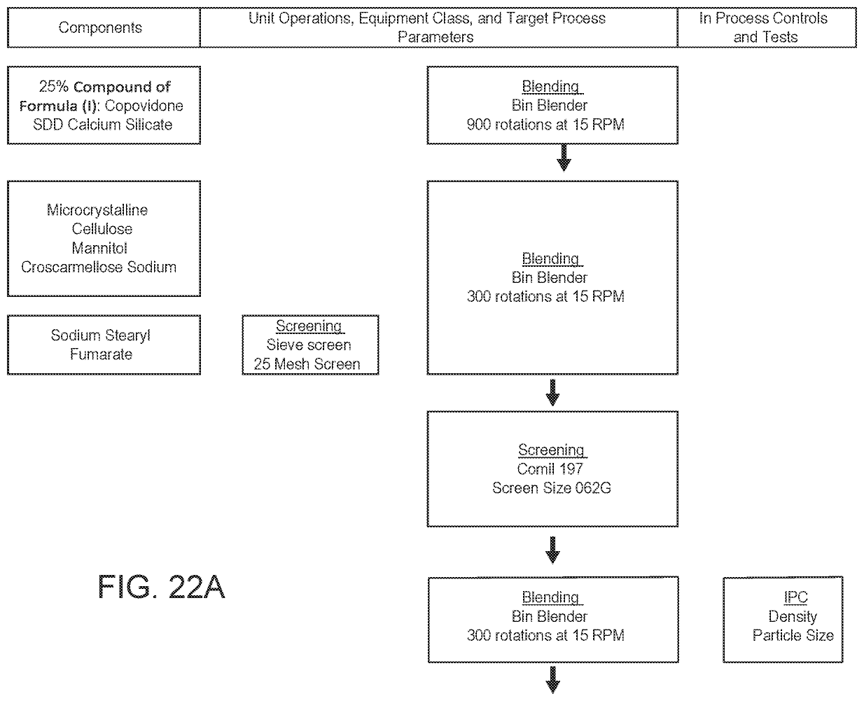

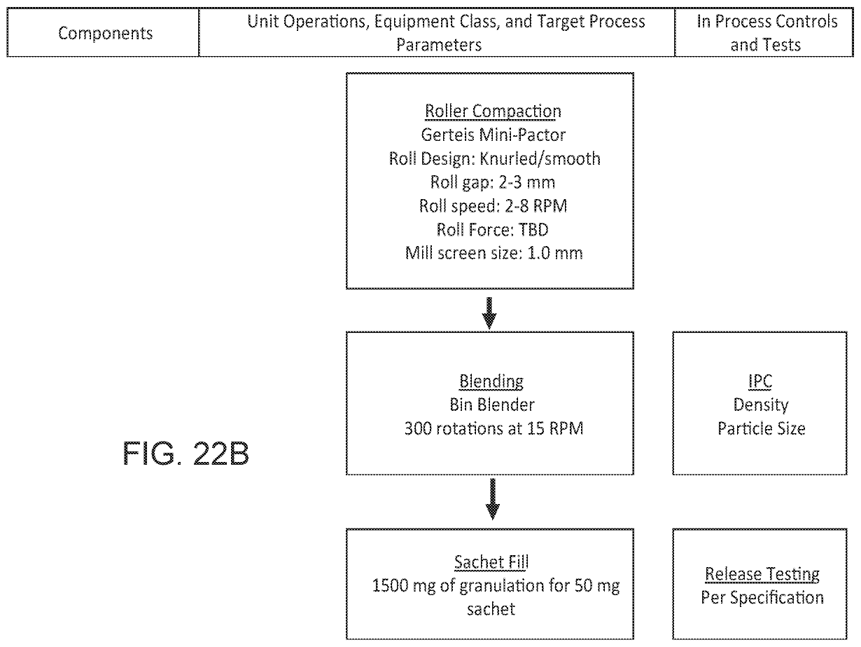

Table 37 shows a granule formulation of the compound of Formula (I) using a SDD prepared according to Example 3, above. An example manufacturing process is shown in FIG. 22A and FIG. 22B.

TABLE 37

| | | | 50 mg Sachet | | Batch |
|---|---|---|---|---|---|
| | Quality | | Weight | % | Weight |
| Component | Standard | Function | (mg/unit) | (w/w) | (g) |
| Example 3, SDD | In-house | Drug Substance | 200.0 | 13.33 | 40 |
| Calcium Silicate (ZEOPHARM | NF | Glidant | 10.0 | 0.67 | 2 |
| 250) | | | | | |
| Mannitol (Pearlitol 200SD) | NF/EP/JP | Filler | 832.5 | 55.5 | 166.5 |
| Microcrystalline Cellulose | NF/EP/JP | Filler | 300.0 | 20.0 | 60 |
| (Avicel PH-102) | | | | | |
| Croscarmellose Sodium (Ac-Di- | NF/EP/JP | Disintegrant | 150.0 | 10.0 | 30 |
| Sol ®) SD711 | | | | | |
| Sodium Stearyl Fumarate | NF/EP/JP | Lubricant | 7.5 | 0.5 | 1.5 |

| | | | |
|---|---|---|---|
| Total | 1500 | 100.0 | 300 |

### Example 12: Liquid Formulation 1 of the Compound of Formula (I)

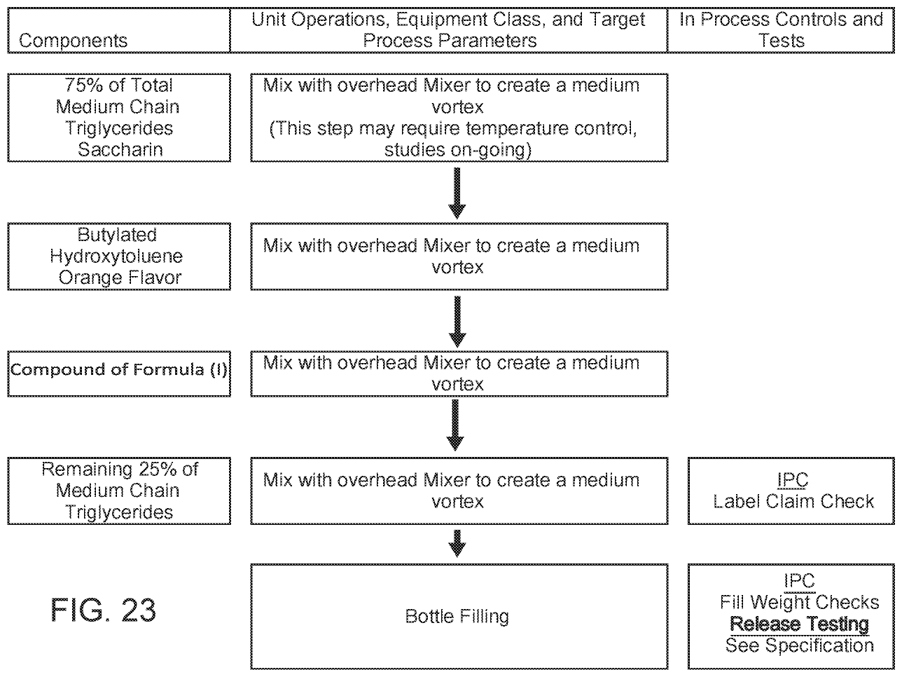

Table 38 shows liquid formulation 1 of the Compound of Formula (I) free base. An example manufacturing process is shown in FIG. 23.

TABLE 38

| | | | 50 mg/mL Oral | | |
|---|---|---|---|---|---|
| | | | Solution | | Batch |
| | Quality | | Weight | % | Weight |
| Component | Standard | Function | (mg/mL) | (w/v) | (g) |
| Compound of Formula (I) FB | In-house | Drug Substance | 50.0 | 5 | 20.03 |
| Saccharin | NF/EP | Sweetener | 1.5 | 0.15 | 0.61 |
| Butylated hydroxytoluene | NF/EP | Anti-oxidant | 1.7 | 0.17 | 0.69 |
| FONA orange flavor | NF | Flavor | 1.0 | 0.1 | 0.41 |
| Labrafac Lipophile WL1349 | NF/EP | Liquid Vehicle | to 1 mL | 94.58 | 358.87 |

| | | | |
|---|---|---|---|
| Total | 1 mL | 100.0 | 380.6 |

### Example 13: Liquid Formulation 2 of the Compound of Formula (I)

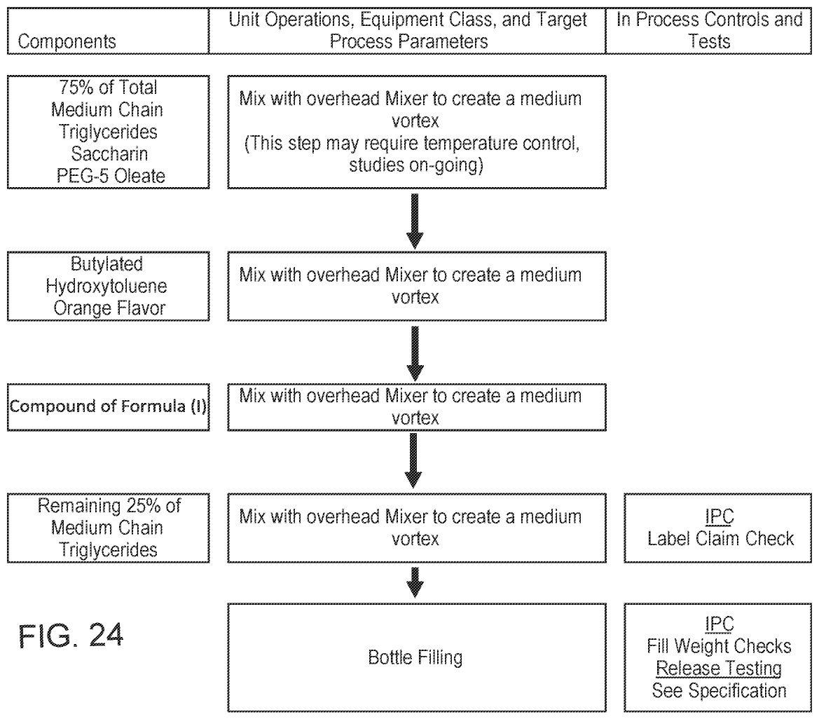

Table 39 shows liquid formulation 2 of the Compound of Formula (I) free base. An example manufacturing process is shown in FIG. 24.

TABLE 39

| | | | 50 mg/mL Oral | | |
|---|---|---|---|---|---|
| | | | Solution | | Batch |
| | Quality | | Weight | % | Weight |
| Component | Standard | Function | (mg/mL) | (w/v) | (g) |
| Compound of Formula (I) FB | In-house | Drug Substance | 50.0 | 5 | 20.17 |
| Saccharin | NF/EP | Sweetener | 1.5 | 0.15 | 0.61 |
| Butylated hydroxytoluene | NF/EP | Anti-oxidant | 1.7 | 0.17 | 0.68 |
| LABRAFIL M 1944 CS | NF/EP | Surfactant | 200.0 | 20 | 80.16 |
| FONA orange flavor | NF | Flavor | 1.0 | 0.1 | 0.40 |
| Labrafac Lipophile WL1349 | NF/EP | Liquid Vehicle | to 1 mL | 74.58 | 278.68 |

| | | | |
|---|---|---|---|
| Total | 1 mL | 100.0 | 380.7 |

### Example 14: A Phase I, Open-Label Study to Evaluate the Pharmacokinetics, Relative Bioavailability, Effect of Food, Safety and Tolerability of Different Compound of Formula (I) Prototype Formulations in Healthy Adult Subjects

### Methodology

This is a single center, open-label, randomized, single dose 4-period crossover study in healthy adult subjects designed to investigate the pharmacokinetic (PK) and safety of up to 4 compound of Formula (I) liquid lipidic prototype formulations (compound of Formula (I) Oral Solution, Prototype Formulations, 50 mg/mL), a compound of Formula (I) spray dried dispersion formulation (compound of Formula (I) Granule for Sprinkle, 25-50 mg) and compound of Formula (I), 50 mg Capsules (Reference). It is planned to enroll 36 subjects to be allocated as 3 cohorts of 12 subjects per cohort, with 6 sub-cohorts of 6 subjects per sub-cohort. In each of these 6 sub-cohorts, 6 subjects will be assigned to one of 3 sub-cohorts where a single oral dose of Investigational Medicinal Product (IMP) is administered in 4 dosing periods (Periods 1 to 4) in multiple fed or fasted states or to one of 3 sub-cohorts where a single oral dose of IMP is administered in 2 dosing periods (Periods 1 and 2) only in the fed state. Within each sub-cohort, subjects will also be randomized before administration of the first dose of IMP in Period 1 to one of the following treatment sequences (Table 40):

TABLE 40

| | | | Total | | | | |
|---|---|---|---|---|---|---|---|
| | | | Number of | | | | |
| Sub- | | Number of | Periods | Regimen | | | |
| Cohort | Sequence | Subjects | Dosed | Period 1 | Period 2 | Period 3 | Period 4 |
| 1A | ABEF | 3 | 4 | Regimen A | Regimen B | Regimen E | Regimen F |
| | BAEF | 3 | 4 | Regimen B | Regimen A | Regimen E | Regimen F |
| 1B | AB | 3 | 2 | Regimen A | Regimen B | N/A | N/A |
| | BA | 3 | 2 | Regimen B | Regimen A | N/A | N/A |
| 2A | ADEF | 3 | 4 | Regimen A | Regimen D | Regimen E | Regimen F |
| | DAEF | 3 | 4 | Regimen D | Regimen A | Regimen E | Regimen F |
| 2B | AD | 3 | 2 | Regimen A | Regimen D | N/A | N/A |
| | DA | 3 | 2 | Regimen D | Regimen A | N/A | N/A |
| 3A | ACEF | 3 | 4 | Regimen A | Regimen C | Regimen E | Regimen F |
| | CAEF | 3 | 4 | Regimen C | Regimen A | Regimen E | Regimen F |
| 3B | AC | 3 | 2 | Regimen A | Regimen C | N/A | N/A |
| | CA | 3 | 2 | Regimen C | Regimen A | N/A | N/A |

At informed consent, subjects will agree either to participate in 2 study periods or 4 study periods. Once placed into a sub-cohort, the order in which subjects receive the study treatments will be randomized based on the schedule above.

Subjects will receive up to 4 regimens in up to 4 periods in an order according to the randomization schedule within each sub-cohort.

The effect of different prandial states on the PK of the compound of Formula (I) may be explored in Periods 3 and 4 by administering in the fasted state or after an alternative meal (e.g., high fat, standard or light breakfast, etc.).

The proposed regimens are presented in Table 41 below:

TABLE 41

| | Investigational | | Route of | |
|---|---|---|---|---|
| Regimen | Medicinal Product | Dose | Administration | Prandial State |
| A | Compound of Formula | 50 mg | Oral | Fed (Ensure Plus) |
| | (I), 50 mg Capsules | | | |
| | (Reference) | | | |
| B | Compound of Formula | 50 mg | Oral | Fed (Ensure Plus) |
| | (I) Oral Solution, | | | |
| | Prototype Formulation | | | |
| | 1, 50 mg/mL | | | |
| C | Compound of Formula | 50 mg | Oral | Fed (Ensure Plus) |
| | (I) Oral Solution, | | | |
| | Prototype Formulation | | | |
| | 2, 50 mg/mL | | | |
| D | Compound of Formula | 50 mg | Oral | Fed (Ensure Plus) |
| | (I) Granule for | | | |
| | Sprinkle, 25-50 mg | | | |
| E | Compound of | 100 mg | Oral | Fed (Ensure Plus) |
| | Formula (I) Oral | | | |
| | Solution, Prototype | | | |
| | Formulation 1 | | | |
| F | Compound of | 50 mg | Oral | Fed (altentative |
| | Formula (I) Oral | | | meal) |
| | Solution, Granule for | | | |
| | Sprinkle | | | |

Study Design: Subjects will be screened for eligibility to participate in the study up to 28 days before the first dose of IMP in Period 1. Each study period will follow the same study design. Subjects will be admitted to the clinical unit on the evening prior to IMP administration (Day −1). For Periods 1 and 2 (Regimen A and one of either Regimens B, C or D), all subjects will receive the compound of Formula (I) formulations in the morning according to the randomization schedule in the fed state with a liquid dietary supplement (Ensure Plus). For Periods 3 and 4 (Regimens E and F), subjects will receive the compound of Formula (I) formulations in the morning according to the randomization schedule in the fed state with a liquid dietary supplement or an alternate prandial state (fasted or alternative meal). IMP administration will be performed on Day 1 with an appropriate interval between subjects based on logistical requirements (approximately 10 min). Meals will be standardized for each treatment regimen across periods.

Subjects will remain in the clinical unit until 36 h post-dose when they will be discharged. Subjects will return to the clinical unit at 168 h (7 days) and 336 h (14 days) post-dose for a PK blood sample and safety assessments. The minimum washout between IMP dosing occasions will be 14 days between Periods 1 and 2 and 21 days or more to accommodate interim data reviews between Periods 2 and 3 and between Periods 3 and 4.

There will be a follow-up phone call 18 to 24 days post-final visit to ensure the ongoing wellbeing of subjects.

Following the completion of Period 2 for all cohorts, there will be an interim data review during which the PK and safety data will be reviewed, plus any relevant emerging Chemistry, Manufacturing and Control (CMC) stability study information, to determine the formulation, dose level and prandial state in which to administer the IMP in Period 3 (Regimen E). There will be a similar interim review following completion of Period 3 (administration of Regimen E) to determine the formulation, dose level and prandial state in which to administer the IMP in Period 4 (Regimen F). The criteria for the interim decisions will be based on available compound of Formula (I) PK data: e.g., Cₘₐₓ, Tₘₐₓ, AUC(0-36), Frei and safety data.

### Number of Subjects Planned:

It is planned to enroll 36 healthy male and female (non-pregnant, non-lactating) subjects in 6 sub-cohorts of 6 subjects per sub-cohort. These sub-cohorts will be combined in sets of 2 to create 3 cohorts of n=12 for Periods 1 and 2 to target data in 10 evaluable subjects in each cohort for the primary objectives per formulation variant. A total of 18 subjects, 6 from each of Sub-Cohorts 1A, 2A and 3A participating in Periods 1 and 2, will additionally participate in Periods 3 and 4 with a target of a minimum of 6 evaluable subjects. A subject will be considered evaluable for a particular regimen if they have received an IMP and has completed sufficient planned PK assessments up to 336 h (14 days) after dosing for that regimen to allow for the assessment of study endpoints. A subject will be considered evaluable for a particular comparison (e.g., food effect, relative bioavailability) if they have received both IMPs under comparison and have sufficient PK data up to 14 days after each regimen to allow for assessment of study endpoints.

Subjects withdrawn due to an IMP-related adverse event (AE) will not be replaced. Subjects who are withdrawn for other reasons may be replaced as required by agreement between the principal investigator (PI) and sponsor to ensure sufficient numbers of evaluable subjects at the end of each study period. Replacement subjects may be required to be dosed with specific formulations from the previous regimens in order to obtain the minimum number of evaluable subjects required for interim decisions and to obtain data in any other regimen that is required to fulfil the study objective comparisons, with the exception that any previously dosed IMP that has been considered sub-optimal will not be dosed. Up to 8 replacement subjects in total may be enrolled into the study. The maximum number of subjects that may be dosed is 44 in total.

If a subject withdraws from Sub-Cohort 1A, 2A or 3A after Period 2, it is acceptable to replace them with a subject from Sub-Cohort 1B, 2B or 3B provided the subject signs an updated consent form agreeing to participate in four treatment periods. At the discretion of the investigator, such a subject may not be required to undergo repeat screening procedures.

### Duration of Study:

For subjects enrolled to receive single dose administration on 4 separate occasions in Periods 1 to 4 (Sub-Cohorts 1A, 2A and 3A), the estimated time from screening until the follow-up phone call is approximately 15 to 16 weeks.

For subjects enrolled to receive single dose administration on 2 separate occasions in Periods 1 to 2 only (Sub-Cohorts 1B, 2B and 3B), the estimated time from screening until the follow-up phone call is approximately 8 to 9 weeks.

### Pharmacokinetic Assessments:

The plasma concentration data for a compound of Formula (I) will be analyzed for final reporting by Quotient Sciences and for interim reviews by Neurocrine Biosciences, Inc. (NBI), using Phoenix WinNonlin v8.0 or a more recent version (Certara USA, Inc., USA). NBI will be responsible for PK analysis for interim review.

PK analysis of the concentration time data obtained will be performed using appropriate non-compartmental techniques to obtain estimates of the following PK parameters (Table 42) where possible and appropriate:

TABLE 42

| | |
|---|---|
| T_{lag} | Time prior to the first measurable (non- |
| | zero) concentration |
| Tₘₐₓ | Time to maximum plasma concentration |
| Cₘₐₓ | Maximum plasma concentration |
| AUC₀₋ₜₗₐₛₜ | Area under the plasma concentration versus |
| | time curve (AUC) from 0 h to last |
| | measurable concentration |
| AUC_{0-inf} | AUC from 0 h extrapolated to infinity |
| AUCₑₓₜᵣₐₚ | Percentage of AUC_{0-inf} extrapolated beyond |
| | the last measurable concentration |
| Lambda-z | Slope of the apparent terminal phase |
| T_{1/2} | Apparent terminal half-life |
| CL/F^{a} | Apparent systemic clearance after oral |
| | administration |
| Vd/F^{a} | Apparent volume of distribution based on |
| | the area after a single oral administration |
| MRT | Mean residence time |
| MPR AUC₀₋ₜₗₐₛₜ | Metabolite to parent ratio based on AUC₀₋ₜₗₐₛₜ |
| MPR AUC_{0-inf} | Metabolite to parent ratio based on AUC_{0-inf} |

### Taste Assessments:

Taste will be assessed for each IMP formulation and vehicle (e.g., Ensure Plus, soft food) using a questionnaire designed for this purpose and adapted for this specific study as required.

The questionnaire will ask subjects to rate the acceptability of smell, sweetness, bitterness, flavor, mouth feel, and aftertaste on a 6-point scale, and overall experience on a 5-point scale for each IMP formulation independently of any previous formulations.

### Statistical Methodology:

Descriptive summaries for all safety data, PK assessments and taste questionnaire data will be provided. No hypothesis testing will be performed for the safety or taste questionnaire data.

### Periods 1 and 2—for Each Cohort Separately

### Relative Bioavailability

Statistical modelling will be performed on the natural log-transformed compound of Formula (I) PK parameters (AUC(0-tlast), AUC(0-inf) and Cmax) to assess relative bioavailability (Frel) using a mixed effects model with terms for regimen, period and sequence as fixed effects and subject within sequence as a random effect. Ratios of geometric means (GMRs) and 90% confidence interval (CI) for the relevant comparison of interest, i.e., between each of the prototype formulations (compound of Formula (I) Oral Solution, Prototype Formulation 1, 50 mg/mL, e.g., Example 12; compound of Formula (I) Oral Solution, Prototype Formulation 2, 50 mg/mL, e.g., Example 13; and compound of Formula (I) Granule for Sprinkle, 25-50 mg, e.g. Example 11 [Regimens B, C and D, respectively]) and compound of Formula (I), 50 mg Capsules, e.g., Example 9 (Reference; Regimen A) will be presented.

### All Periods (Periods 1 to 4)

### Food Effect

Statistical modelling will be performed on the compound of Formula (I) PK parameters AUC(0-tlast), AUC(0-inf) and Cmax to assess for the effects of food, if relevant. The natural log-transformed PK parameters will be analyzed for bioavailability using a mixed effects model with terms for prandial state (and meal type if applicable) as a fixed effect and subject as a random effect. Ratios of geometric means and 90% CI for the relevant comparisons of interest will be presented where the ratio is defined as fasted/fed or test meal/reference meal (if applicable).

### Relative Bioavailability

Statistical modelling will be performed on the natural log-transformed compound of Formula (I) PK parameters (AUC(0-tlast), AUC(0-inf) and Cmax) to assess relative bioavailability using a mixed effects model with terms for regimen as a fixed effect and subject as a random effect. Ratios of geometric means and 90% CI for the relevant comparison of interest i.e., between each of the prototype formulations (Regimens E and F, IMPs to be determined by interim reviews following completion of Periods 2 and 3) and compound of Formula (I), 50 mg Capsules (Reference; Regimen A) will be presented.

### Results

Preliminary PK data is summarized in Table 43 below:

TABLE 43
Preliminary Data from Periods 1 and 2

| | | | B-Oral | | C-Oral | | D- SDD | |
|---|---|---|---|---|---|---|---|---|
| | A- Reference | | Solution 1 | | Solution 2 | | Granule | |
| N | 35 | | 12 | | 12 | | 12 | |
| Tmax * | 5 | (2, 6) | 6 | (5, 7) | 5 | (5, 7) | 5 | (5, 7) |
| (h) | | | | | | | | |
| Cmax | 1361 | (33) | 790 | (31) | 1082 | (46) | 1075 | (35) |
| (ng/ml) | | | | | | | | |
| AUC36 | 9452 | (30) | 5556 | (34) | 7582 | (37) | 6691 | (41) |
| (h*ng/ml) | | | | | | | | |

* Geometric Mean/CV % for AUC and Cmax; Median for Tmax

### Example 15. Compound of Formula (I) Crystalline Free Base Form I

### Example 15A

### Scheme 1: Preparation of 4-(2-chloro-4-methoxy-5-methylphenyl)-N-[(1S)-2-cyclopropyl-1-(3-fluoro-4-methylphenyl)ethyl]-5-methyl-N-(2-propyn-1-yl)-2-thiazolamine (Compound of Formula (I), Form I)

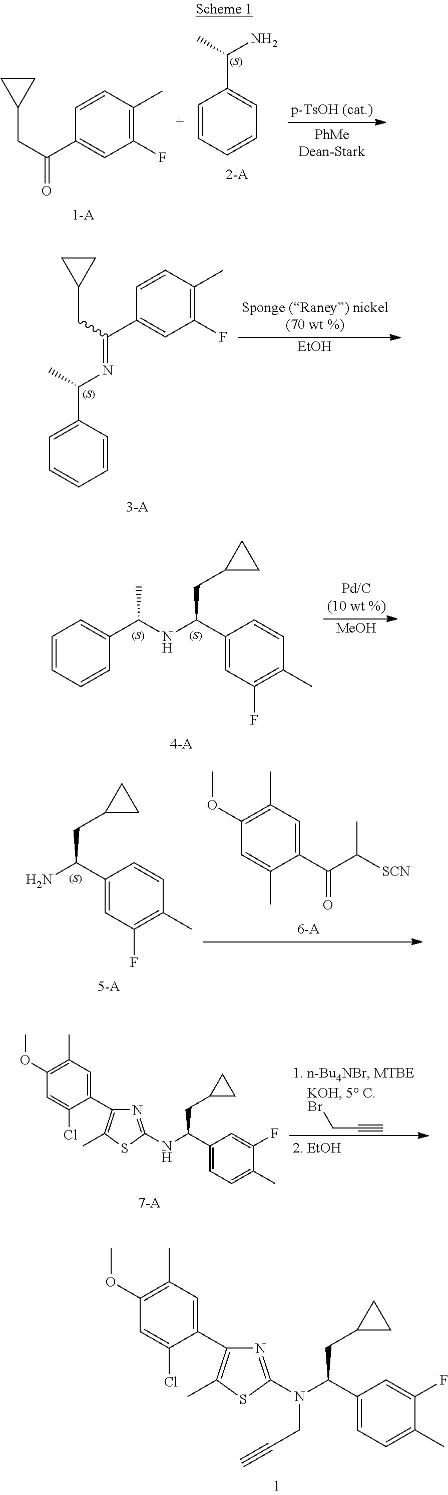

### Step 1: Preparation of (S)-2-cyclopropyl-1-(3-fluoro-4-methylphenyl)-N-(1-phenylethyl)ethan-1-imine (Compound 3-A)

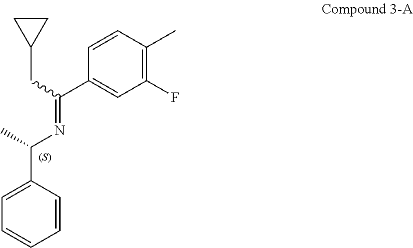

A mixture of 2-cyclopropyl-1-(3-fluoro-4-methylphenyl)ethan-1-one (1-A, 150.7 kg, 1 eq., as a 27.6% w/w solution in toluene, Example 15C), (S)-(−)-1-Phenylethylamine (2-A, 112.9 kg, 1.19 eq.), and p-toluenesulfonic acid (7.4 kg, 0.05 eq.) is refluxed at 110-120° C. for 23-25 h in a reactor set up in a Dean-Stark configuration. The solvent is then removed at 125-135° C. under atmospheric pressure until distillation halts and a portion of toluene (275 kg, 2.24 w/w) is added to afford a suspension. The suspension is refluxed at 110-120° C. for 23-25 h. The mixture is cooled to 22° C. and washed twice with aqueous NH₄Cl (10%, 301.2 kg, 0.72 eq.) and once with aqueous NaHCO₃ (5%, 301.2 kg, 0.23 eq., check pH 8-9). The solvent is removed at 125-135° C. and atmospheric pressure to a target volume of 256 L, the mixture is filtered over celite, the cake is washed with toluene (25 kg). The resulting mixture containing compound 3-A is used directly in the next step without isolation. The yield is determined by correcting for the LOD and GC-FID purity of the sample (208.4 kg, 90.0% corrected, 0.89% Compound 2-A). EI-MS: 294.1 [M−H]⁺, 190.1 [M-C₆H₅CH(CH₃)]⁺, 105.1 [C₆H₅CH(CH₃)]⁺.

### Step 2: Preparation of (S)-2-cyclopropyl-1-(3-fluoro-4-methylphenyl)-N—((S)-1-phenylethyl)ethan-1-amine hydrochloride (Compound 4-A)

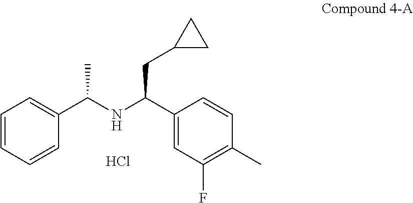 [SMILES: Cc1ccc([C@H](CC2CC2)N[C@@H](C)c2ccccc2)cc1F ; Cl]

Sponge nickel catalyst (144 kg, 0.70 w/w, shipped as a 50% w/w suspension in water) is added to a hydrogenation reactor, equipped with a dip tube capable of removing material from the top of the mass inside, minimizing the amount of water introduced. The supernatant is discarded, ethanol (329.3 kg, 1.58 w/w, anhydrous) is added, the suspension is stirred and then allowed to settle. This process is repeated four more times and the Karl Fisher (KF) of the supernatant is checked (≤1% H₂O w/w). Compound 3-A (208.4 kg, 1 eq., as a 62.6% solution in toluene) is added to the mixture in the hydrogenation reactor and ethanol (387.6 kg, 1.86 w/w) is used to rinse the addition flask into the hydrogenation reactor. The hydrogenation reactor is pressurized/depressurized twice with nitrogen (2 bar) and twice with hydrogen (5 bar) then pressurized with hydrogen (9.8-10.2 bar) and heated to 33-37° C. and stirred for 17-19 h. The system is depressurized/pressurized three times with nitrogen (1 bar) and the suspension is filtered and washed with three times with ethanol (493.8 kg, 2.37 w/w). HCl (concentrated, 83.4 kg, 1.07 eq.) is added and the mixture stirred 25-35 min at 20-24° C. The mixture is concentrated by distillation at 78-80° C. and atmospheric pressure to remove water with a distillate target volume of 1167 L (5.6 L/kg, Compound 3-A) and the KF of the solution is checked (≤1.5% H₂O w/w). The mixture is stirred at 48-52° C. for 55-65 min, then 68-72° C. for 55-65 min, then cooled to 20-24° C. at a rate of 12° C./h and stirred for 25-35 min, then cooled to 0-4° C. at a rate of 10° C./h and stirred for 55-65 min. The suspension is filtered, the cake is washed twice with precooled ethanol (329.2 kg, 1.58 w/w, 0° C.), and the collected solid is dried at 40° C. to afford compound 4-A (156.5 kg, 66.4% uncorrected). ¹H NMR (400 MHz, DMSO-d6) δ ppm −0.33-−0.06 (m, 2H) 0.11-0.31 (m, 3H) 1.57 (d, J=6.57 Hz, 3H) 1.95 (br t, J=7.07 Hz, 2H) 2.26 (d, J=1.26 Hz, 3H) 3.68 (br d, J=7.83 Hz, 1H) 3.92 (br t, J=6.44 Hz, 1H) 6.98 (dd, J=7.71, 1.14 Hz, 1H) 7.28-7.36 (m, 2H) 7.37-7.50 (m, 5H). ESI-MS: 298.2 m/z [M+H]⁺.

### Step 3: Preparation of (S)-2-cyclopropyl-1-(3-fluoro-4-methylphenyl)ethan-1-amine hydrochloride (Compound 5-A)

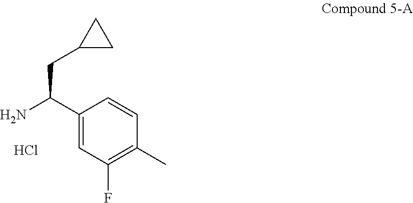

Compound 4-A (156.5 kg, 1.00 eq.) and Pd/C (7.8 kg, 10% Pd basis) are added to an inerted hydrogenation reactor. The reactor is then pressurized/depressurized twice with nitrogen (2 bar) and then methanol (494.5 kg, 3.16 w/w) is added. The reactor is depressurized/pressurized three times with nitrogen (2 bar) then three times with hydrogen (5 bar), pressurized with hydrogen (9.8-10.2 bar), heated to 58-62° C. and stirred for 7-9 h. The reaction mixture is cooled to 20-24° C. The reactor is depressurized/pressurized three times with nitrogen (1 bar) and the suspension is filtered and washed three times with methanol (492.9 kg, 3.15 w/w). The solution is concentrated at 63-67° C. and atmospheric pressure to a distillate target volume of 1408 L (9.0 L/kg Compound 4-A). n-Heptane (1173.8 kg, 7.5 w/w) is added and the mixture is refluxed at 65-80° C. and atmospheric pressure in Dean-Stark configuration to remove methanol. The suspension is cooled to 31-35° C. and filtered, the cake washed with n-heptane (147.1 kg, 0.94 w/w), and the solid dried at 40° C. (101.0 kg, 93.8% uncorrected, 99.2% ee). ¹H NMR (400 MHz, DMSO-d6) δ ppm −0.12-0.14 (m, 2H) 0.26-0.42 (m, 2H) 0.44-0.55 (m, 1H) 1.70-1.83 (m, 2H) 2.23 (d, J=1.52 Hz, 3H) 4.24 (t, J=7.33 Hz, 1H) 7.22-7.29 (m, 1H) 7.29-7.36 (m, 1H) 7.40 (dd, J=10.99, 1.39 Hz, 1H). ESI-MS: 194.2 [M+H]⁺, 177.0 [M-NH₂]⁺.

### Step 4: Preparation of (S)-4-(2-chloro-4-methoxy-5-methylphenyl)-N-(2-cyclopropyl-1-(3-fluoro-4-methylphenyl)ethyl)-5-methylthiazol-2-amine (Compound 7-A)

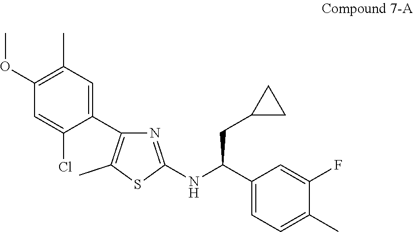 [SMILES: COc1cc(Cl)c(-c2nc(N[C@@H](CC3CC3)c3ccc(C)c(F)c3)sc2C)cc1C]

A mixture of n-heptane (146 kg), water (142 kg), Compound 5-A (57.4 kg), and aqueous sodium hydroxide (30% w/w, 41.0 kg) was stirred together. The layers were partitioned, and the aqueous layer removed. The organic layer was washed with water (170 kg) and the layers partitioned. The organic layer was set aside using n-heptane (40 kg) to rinse and n-heptane (145 kg) and 1-(2-chloro-4-methoxy-5-methylphenyl)-2-thiocyanatopropan-1-one (6-A, 66.1 kg) were added to the reactor and heated to 85° C. The previously set aside organic layer containing the free base of Compound 5-A was added at 84-85° C. to the reactor and rinsed with n-heptane (20 kg). The resulting mixture was stirred for 2 h at 83° C. Subsequently, the solvent was switched to methanol by four put-and-take additions/vacuum distillations of methanol (180 kg) at 55° C. with the target volume being 287 L remaining in the reactor. The suspension was cooled to 5° C. and water (570 kg) was added over 4 h at 5-10° C., with the first 60 kg added very slowly. The suspension was aged 2 h at ° C. and then isolated by filtration and washed with a mixture of methanol/water (91/115 kg) and then a mixture of methanol/water (134/57 kg). The yellow solid was dried at 25° C. and 1 mbar for 17 h then 40° C. and 1 mbar for 22 h to afford Compound 7-A (97.4 kg, 87.5% yield). ¹H NMR (400 MHz, DMSO-d6) δ ppm −0.01-0.14 (m, 2H) 0.29-0.42 (m, 2H) 0.61-0.73 (m, 1H) 1.47 (dt, J=13.83, 6.85 Hz, 1H) 1.76 (dt, J=13.89, 7.20 Hz, 1H) 2.00 (s, 3H) 2.11 (s, 3H) 2.19 (d, J=1.01 Hz, 3H) 3.82 (s, 3H) 4.54 (q, J=7.58 Hz, 1H) 7.00 (s, 1H) 7.06 (d, J=0.76 Hz, 1H) 7.08-7.14 (m, 2H) 7.18-7.23 (m, 1H) 7.89 (d, J=8.08 Hz, 1H). ESI-MS: 445.3 m/z [M+H]⁺.

### Step 5: Preparation of 4-(2-chloro-4-methoxy-5-methylphenyl)-N-[(1S)-2-cyclopropyl-1-(3-fluoro-4-methylphenyl)ethyl]-5-methyl-N-(2-propyn-1-yl)-2-thiazolamine (Compound of Formula (I))

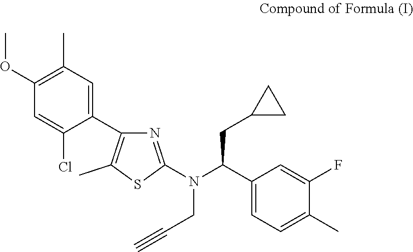 [SMILES: C#CCN(c1nc(-c2cc(C)c(OC)cc2Cl)c(C)s1)[C@@H](CC1CC1)c1ccc(C)c(F)c1]

A mixture of MTBE (279 kg), tetra-n-butylammonium bromide (10.5 kg), and Compound 7-A (95.4 kg) were heated at 60° C. external temperature for 30 min and then cooled to 0° C. Aqueous potassium hydroxide (52.4% w/w, 364 kg) and propargyl bromide (39.4 kg as an 80% w/w solution in toluene, 1.19 eq.) were added at 0-5° C. and the biphasic mixture aged 14.5 h at 4-6° C. with vigorous stirring. Subsequently, water (191 kg) was added and the aqueous layer was discharged. The organic layer was washed twice with water (382 kg) and once with aqueous acetic acid (5.26% w/w, 190 kg) at 20° C. The mixture is polish filtered, rinsed with ethanol (11 kg) and then the solvent switched to ethanol by 3 put-and-take additions/vacuum distillations of ethanol (300 kg) at 25-30° C. for the first cycle and then 35-40° C. with the target volume of each cycle being 250 L remaining in the reactor. Ethanol (164 kg) was added and the mixture heated at 60° C. external for 0.5 h before it was cooled to 25° C. in 1 h and seeded with authentic compound of Formula (I) (0.340 kg) which may be prepared as described below in Example 15B. The suspension was aged for 5 h, cooled to 0° C. in 2 h, aged 12 h, filtered, and washed twice with ethanol (24 kg each) pre-cooled to 0° C. The white solid was dried at 40° C. and 1 mbar for 19 h to yield 80.15 kg of the compound of Formula (I), Form I (77.2% yield). ¹H NMR (400 MHz, DMSO-d6) δ ppm 0.14 (qt, J=8.59, 4.42 Hz, 2H) 0.29-0.48 (m, 2H) 0.61-0.82 (m, 1H) 1.89 (dt, J=14.08, 6.98 Hz, 1H), 2.07 (br d, J=7.83 Hz, 1H) 2.10 (s, 3H) 2.14 (s, 3H) 2.20 (d, J=1.01 Hz, 3H) 3.11 (t, J=2.27 Hz, 1H) 3.83 (s, 3H) 3.94-4.22 (m, 2H) 5.26 (t, J=7.58 Hz, 1H) 7.05 (s, 1H) 7.10-7.36 (m, 4H). ESI-MS: 483.2 m/z [M+H]⁺.

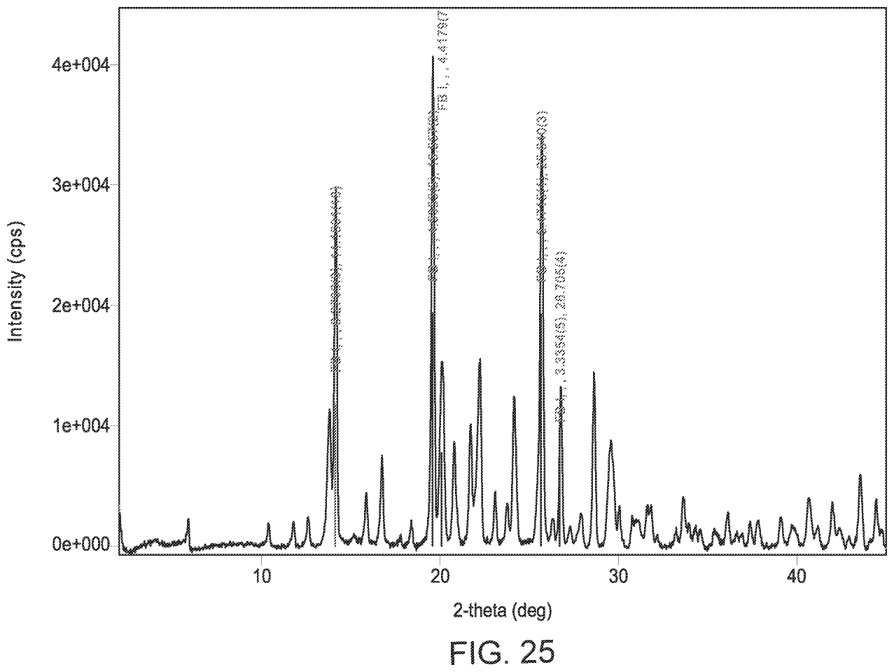

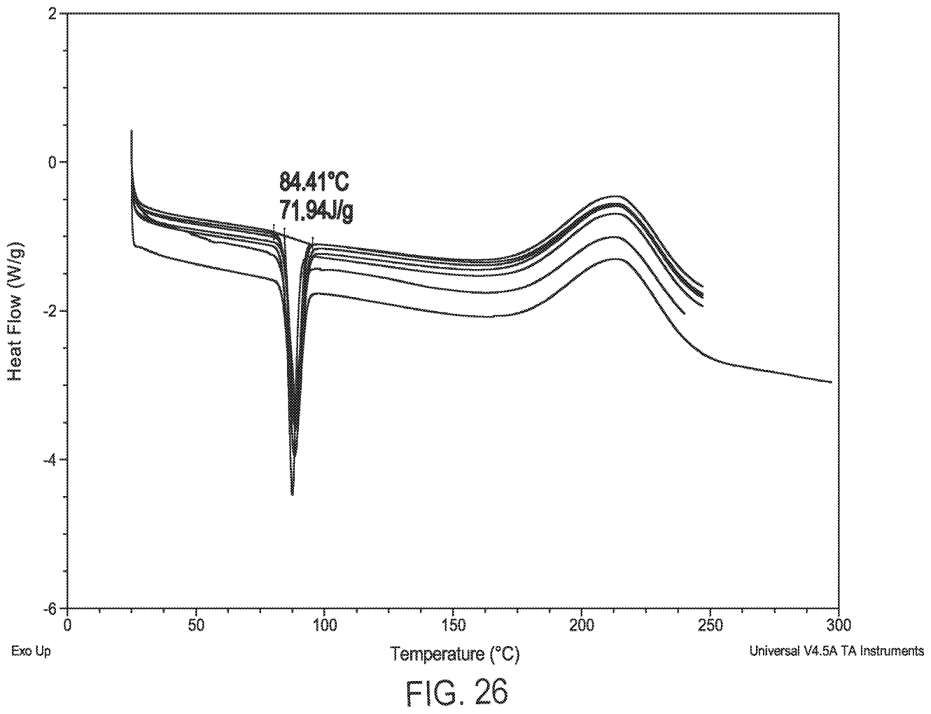

The crystallinity of the crystalline free base Compound of Formula (I), Form I was confirmed by XRPD (FIG. 25, Table 44) and further supported by DSC (FIG. 26), indicating the crystalline compound having an onset of melt at about 84.4° C. (71.9 J/g). TGA of the crystalline free base exhibited about 0.6% of weight loss due to solvent/H₂O.

TABLE 44
XRPD Peak Data for the Compound of Formula
(I) Crystalline Free Base Form I

| 2-Theta (°) | Height (cps) |
|---|---|
| 5.901(15) | 1221(101) |
| 10.367(11) | 1280(103) |
| 11.762(13) | 1377(107) |
| 12.582(11) | 1591(115) |
| 13.802(2) | 7326(247) |
| 14.1541(16) | 20179(410) |
| 15.173(14) | 449(61) |
| 15.854(7) | 2906(156) |
| 16.746(5) | 5113(206) |
| 18.366(7) | 1171(99) |
| 19.586(2) | 27789(481) |
| 20.100(4) | 10759(299) |
| 20.794(4) | 5441(213) |
| 21.730(5) | 7125(244) |
| 22.239(7) | 10370(294) |
| 23.056(11) | 3482(170) |
| 23.714(7) | 2300(138) |
| 24.115(7) | 8402(265) |
| 25.666(4) | 26173(467) |
| 26.296(6) | 1505(112) |
| 26.752(4) | 9919(288) |
| 27.264(7) | 1016(92) |
| 27.874(7) | 2092(132) |
| 28.623(3) | 10560(297) |
| 29.546(6) | 5811(220) |
| 30.025(3) | 2248(137) |
| 30.737(10) | 1333(105) |
| 31.017(19) | 1406(108) |
| 31.588(10) | 2292(138) |
| 31.809(8) | 2212(136) |
| 32.126(13) | 593(70) |
| 33.200(16) | 839(84) |
| 33.613(13) | 2996(158) |
| 33.914(13) | 1156(98) |
| 34.276(16) | 1008(92) |
| 34.564(12) | 1056(94) |
| 35.397(18) | 816(82) |
| 36.073(10) | 1928(127) |
| 36.67(3) | 562(68) |
| 37.347(9) | 1553(114) |
| 37.776(12) | 1573(114) |
| 39.070(7) | 1890(125) |
| 39.743(15) | 1042(93) |
| 40.643(9) | 2808(153) |
| 41.106(8) | 1107(96) |
| 41.984(11) | 2686(150) |
| 42.376(16) | 986(91) |
| 42.901(16) | 492(64) |
| 43.543(10) | 4744(199) |
| 44.419(16) | 2810(153) |

### Example 15B

### Preparation of 4-(2-chloro-4-methoxy-5-methylphenyl)-N-[(1S)-2-cyclopropyl-1-(3-fluoro-4-methylphenyl)ethyl]-5-methyl-N-(2-propyn-1-yl)-2-thiazolamine (Compound of Formula (I), Seed Batch)

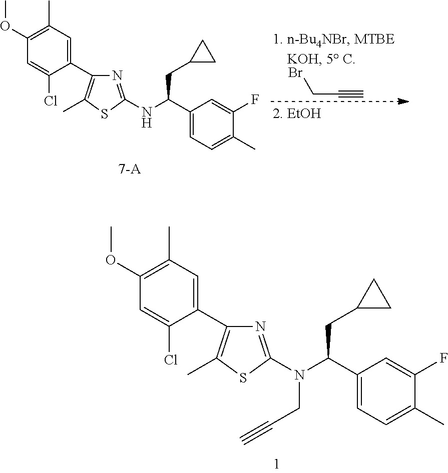

A mixture of MTBE, tetra-n-butylammonium bromide, and Compound 7-A cooled to 0° C. is treated with aqueous potassium hydroxide and propargyl bromide maintaining the temperature at 0-5° C. The resulting biphasic mixture is aged 23 h at 4-6° C. Subsequently, water and MTBE are added and the aqueous layer is discharged. The organic layer is washed twice with water and once with aqueous acetic acid at 20° C. Ethanol is added and then the solvent switched to ethanol by 3 put-and-take additions/vacuum distillations of ethanol at 35-40° C. with a target volume of each cycle remaining in the vessel, except for the third cycle where the mixture is concentrated to dryness. Ethanol is added to the vessel and the mixture heated at 60° C. external for 0.5 h before it is cooled to 20° C. in 1 h and aged 18 h affording a suspension. The suspension is cooled to 0° C., aged 6 h, filtered, and washed twice with ethanol pre-cooled to 0° C. to afford a solid. The solid is dried at 40° C. under vacuum to afford the compound of Formula (I).

### Example 15C

### Scheme 2: Preparation of 2-cyclopropyl-1-(3-fluoro-4-methylphenyl)ethan-1-one (Compound 1-A)

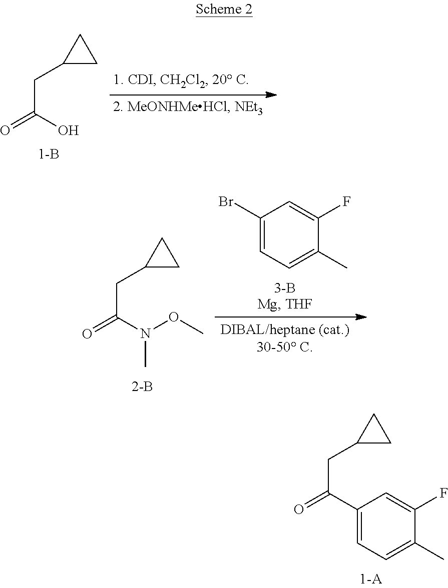

### Step 1: Preparation of 2-cyclopropyl-N-methoxy-N-methylacetamide (Compound 2-B)

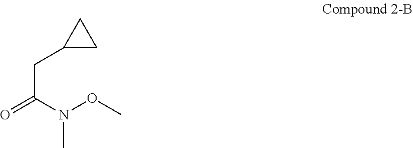

A suspension of 1,1′-Carbonyldiimidazole (152.6 kg, 1.01 eq.) in DCM (682 kg, 513 L, 7.3 w/w relative to 2-cyclopropylacetic acid) was treated with a solution of 2-cyclopropylacetic acid (1-B, 93.6 kg, 1 eq.) in DCM (248 kg, 186 L, 2.65 w/w) over at least 1 h, keeping the temperature ≤25° C. and compensating for significant effervescence. The resulting mixture is stirred for 15 min at 22° C. and then N,O-dimethylhydroxylamine·HCl (93.3 kg, 1.03 eq.) is added in portions, keeping the temperature ≤30° C. Subsequently, triethylamine (46.4 kg, 0.49 eq.) is added to the stirring mixture at 20-25° C. The resulting mixture is stirred at 22° C. at least 1 h. The mixture is washed once with KHSO₄ solution (0.24 M, 357.1 kg, 0.09 eq.), once with KHSO₄ solution (0.40 M, 365.4 kg, 0.15 eq.), once with KHSO₄ solution (0.80 M, 384.5 kg, 0.30 eq.), and once with NaHCO₃ solution (0.60 M, 393.1 kg, 0.24 eq.). Residual DCM is removed by three put-and-takes of THF (166.6 kg, 1.78 w/w) and vacuum distillation (50-60° C., to minimum volume/until distillation stops). THE (333.2 kg. 3.56 w/w) is added and the yield is determined by correcting for the LOD and GC-FID purity of the sample (131.5 kg, 98.2% corrected). ¹H-NMR (400 MHz, DMSO-d6) δ: −0.01-0.03 (m, 2H) 0.32-0.36 (m, 2H) 0.81-0.90 (br m, 1H) 2.18 (d, J=6.80 Hz, 2H) 2.97 (s, 3H) 3.53 (s, 3H). ESI-MS: 144.0 [M+H]⁺.

### Step 2: Preparation of 2-cyclopropyl-1-(3-fluoro-4-methylphenyl)ethan-1-one (Compound 1-A)

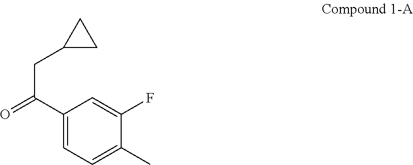

Mg (turnings, 28.6 kg, 1.37 eq.) are suspended in THE (244.7 kg, 2.0 w/w) and DIBAL-H (1 M in n-heptane, 18.9 kg, 0.03 eq.) is added dropwise at 30° C., The resulting mixture is stirred at 30° C. for at least 10 min and then 4-bromo-2-fluoro-1-methylbenzene (3-B, neat, 21.1 kg, 0.13 eq.) is added over at least 30 min at 30-50° C. Subsequently, the mixture is treated with a solution of 4-bromo-2-fluoro-1-methylbenzene (3-B, 191.6 kg, 1.18 eq.) in THF (414.5 kg, 3.37 w/w) at 30-50° C. over 3 h or less. The mixture is stirred at 30° C. for at least 1 h. Subsequently, the mixture is treated with 2-cyclopropyl-N-methoxy-N-methylacetamide (2-B, 123.0 kg, 1 eq., 25.9% w/w solution in THF) over at least 1 h at 15-25° C. The resulting mixture is stirred at 20-25° C. for at least 1 h. The stirring mixture is then treated with aqueous HCl (3 M, 10.3% w/w, 668.9 kg, 2.24 eq.) at 10-25° C. and the resulting mixture is stirred at least 2 h (check pH 3.0-3.5). The layers are separated, and the aqueous layer is combined with heptane (290.3 kg, 2.36 w/w). The layers are separated, and the organic layer is washed once with NaHCO₃ solution (0.63 M, 211.6 kg, 0.15 eq.) and once with NaCl solution (2.57 M, 213.0 kg, 0.55 eq.). The residual solvents are removed by vacuum distillation at 58-62° C. until distillation stops and then one put-and-take of toluene (275.5 kg, 2.24 w/w) at 107-117° C. until distillation stops. Toluene (275.5 kg, 2.24 w/w) is added and the yield is determined by correcting for the LOD and GC-FID purity of the sample (150.7 kg, 91.3% corrected). ¹H NMR (400 MHz, DMSO-d6) δ ppm 0.07-0.21 (m, 2H) 0.40-0.54 (m, 2H) 1.02 (ttt, J=8.16, 8.16, 6.68, 6.68, 4.86, 4.86 Hz, 1H) 2.30 (d, J=1.77 Hz, 3H) 2.91 (d, J=6.57 Hz, 2H) 7.44 (t, J=7.83 Hz, 1H) 7.57-7.78 (m, 2H). ESI-MS: 193.1 [M+H]⁺.

### Example 16: Compound of Formula (I) Crystalline Tosylate Salt Form 1

Approximately 20 mg of the Compound of Formula (I) was weighed into a vial. Using a positive displacement pipette, 250 μL of solvent (IPA) was added to the vial along with a stir bar. The vial was placed in an aluminum block on a Reacti-Therm mixer and heated to 60° C. for ˜1 hour. A molar equivalent of para-toluenesulfonic acid was added to the vial (20 μL of a 2M solution in water) and allowed to stir. The sample was slow cooled back to room temperature along with mild Nitrogen gas for evaporation. Precipitate was collected, left to dry overnight, and then analyzed by XRPD, DSC, and TGA.

The crystallinity of the crystalline tosylate form 1 was confirmed by XRPD (FIG. 27, Table 45) and further supported by DSC (FIG. 28), indicating the crystalline compound having an onset of melt at about 156° C. (22.2 J/g). TGA of the crystalline compound is provided in FIG. 28, and exhibited about 0.5% of weight loss due to solvent/H₂O.

TABLE 45
XRPD Peak Data for the Compound of Formula
(I) Crystalline Tosylate Form 1

| 2-Theta (°) | Height (cps) |
|---|---|
| 8.112(9) | 957(89) |
| 9.124(2) | 12296(320) |
| 9.471(2) | 4519(194) |
| 10.525(3) | 8507(266) |
| 11.316(3) | 10211(292) |
| 13.182(5) | 6158(227) |
| 13.494(6) | 1598(115) |
| 14.249(9) | 2197(135) |
| 15.208(9) | 1746(121) |
| 15.711(7) | 1437(109) |
| 16.252(5) | 5723(218) |
| 16.692(3) | 3848(179) |
| 17.540(4) | 1578(115) |
| 19.031(6) | 6774(238) |
| 19.265(4) | 6491(233) |
| 19.499(10) | 3152(162) |
| 20.401(3) | 8581(267) |
| 20.656(4) | 3040(159) |
| 21.142(3) | 11498(310) |
| 21.703(6) | 4979(204) |
| 21.870(11) | 5331(211) |
| 22.277(5) | 3701(176) |
| 22.769(3) | 10159(291) |
| 23.297(3) | 14954(353) |
| 23.532(4) | 3597(173) |
| 23.810(3) | 9590(283) |
| 24.73(2) | 2325(139) |
| 25.47(4) | 1704(119) |
| 26.087(5) | 2413(142) |
| 26.71(3) | 422(59) |
| 27.210(6) | 1648(117) |
| 27.593(5) | 2825(153) |
| 28.472(4) | 6417(231) |
| 29.51(3) | 1423(109) |
| 29.98(3) | 849(84) |
| 30.63(6) | 1262(103) |
| 30.77(3) | 1121(97) |
| 31.577(3) | 5563(215) |
| 32.74(4) | 874(85) |
| 33.73(4) | 1111(96) |
| 34.415(13) | 2054(131) |
| 34.799(11) | 706(77) |
| 35.139(15) | 1320(105) |
| 35.682(16) | 1028(93) |
| 38.39(4) | 751(79) |
| 39.743(17) | 1790(122) |
| 40.219(16) | 762(80) |
| 41.23(3) | 925(88) |
| 43.16(3) | 1540(113) |
| 44.288(12) | 1142(98) |

### Example 17: Randomized, Double-Blind, Placebo-Controlled Study to Evaluate the Safety and Efficacy of a Compound of Formula (I) in Adult Subjects with Classic Congenital Adrenal Hyperplasia, Followed by Open-Label Treatment

### (I) Objectives

To evaluate the efficacy of crinecerfont, (100 mg twice daily [i.e., BID] based on the free base), compared with placebo, in reducing daily glucocorticoid dosage while maintaining adrenal androgen control.
To evaluate the efficacy of crinecerfont, compared with placebo, in reducing adrenal steroid levels following an initial 4-week treatment period.
To evaluate the effect of crinecerfont compared with placebo, on clinical endpoints associated with supraphysiologic glucocorticoid dosing.
To evaluate plasma concentrations of crinecerfont and metabolites.
To assess the safety and tolerability of crinecerfont.
To evaluate an alternate dosing regimen of crinecerfont in subjects who have not reduced their glucocorticoid dose by Month 12.

### (II) Methodology:

This is a Phase 3, randomized, double-blind, placebo-controlled study to evaluate the efficacy, safety, and tolerability of crinecerfont, versus placebo administered BID with breakfast and the evening meal (doses separated by approximately 12 hours) for 24 weeks in approximately 165 adult subjects with classic CAH due to 21-hydroxylase deficiency. Eligible subjects will be randomly assigned in a 2:1 ratio (active:placebo) to 2 treatment groups: crinecerfont, 100 mg BID or placebo. After the 24-week randomized treatment period, there will be a 6 month, open-label treatment period, during which all subjects will receive crinecerfont at 100 mg BID. At Month 12, subjects who have not reduced their glucocorticoid dose to 11 mg/m²/day will be re-randomized (2:1) to receive 50 mg every morning (qAM) and 150 mg every evening (qPM) or to continue 100 mg BID, in a blinded fashion. Subjects who have reduced their glucocorticoid dose to 11 mg/m²/day will continue to receive 100 mg BID in an open label fashion. A final study visit will be conducted approximately 4 weeks after the Month 18 visit.

(A) Screening Period (Weeks −4 Up to Day −1)

All subjects must provide signed and witnessed informed consent prior to the conduct of any study-related procedures. Subjects will undergo screening for up to 4 weeks (Weeks −4 to Day −1) to determine eligibility. There will be a second visit (optional at home) during the screening period to collect a blood sample (for hormone measurements). Subjects must be on a supraphysiologic glucocorticoid regimen defined as >14 mg/m²/day in hydrocortisone dose equivalents adjusted for body surface area (BSA) that has been stable at least 1 month leading up to screening. The glucocorticoid regimen should be optimized by the treating physician to achieve control of adrenal androgen levels and minimization of glucocorticoid dosage to the extent appropriate for the subject's individual medical needs and treatment goals.

Rescreening is permitted if a subject does not meet all eligibility requirements and returns to be rescreened. A subject that has failed screening twice may not be rescreened again without prior permission.

(B) Randomized, Double-Blind, Placebo-Controlled Treatment Period (Day 1 Up to Week 24)

(a) 4-Week Glucocorticoid Stable Period (Day 1 Up to Week 4)

During the first 4 weeks of the study, subjects should maintain their stable glucocorticoid regimen, except for sick-day guidelines (e.g., based on guidance provided by the investigator or their treating physician).

On Day 1 (baseline), subjects will collect a urine sample (all voids from midnight the night before the study visit to the first morning void after awakening for the day) at home in the morning and bring it to the site for measurement of androgen metabolite levels. They will hold their morning glucocorticoid dose and bring it with them to the study site so that a blood sample can be obtained prior to taking the morning glucocorticoid dose; subjects will then take their morning dose of glucocorticoid at the study site, and another blood sample will be taken approximately 2 hours postdose in order to establish the baseline pre- and post-glucocorticoid hormone levels. Subjects should be fasting from the night before so that fasting blood tests and an oral glucose tolerance test can be performed, but should be encouraged to drink water to avoid any hypovolemic status.

Subjects will be randomized on Day 1 in a 2:1 ratio (active:placebo). Randomization will be stratified by total daily glucocorticoid dose, glucocorticoid type, and sex. Beginning on Day 1 (baseline), the study drug or placebo in the form of one or more capsules will be administered at home with the subject's evening meal; thereafter, the capsule(s) will be administered BID with the subject's breakfast and evening meal (doses separated by approximately 12 hours).

(b) 8-Week Glucocorticoid Reduction Period (Week 4 Up to Week 12)

During this period, subjects will undergo a down-titration (in 4 or fewer steps) of their glucocorticoid dose with the goal to reach a target dose of 8 to 10 mg/m²/day (hydrocortisone equivalents adjusted for body surface area (BSA)) by Week 12, unless the subject has any signs or symptoms suggestive of clinically relevant glucocorticoid insufficiency or unacceptable symptoms of hyperandrogenism.

At the week 4 visit, a similar procedure will be followed as for Day 1 to obtain a more detailed assessment of androgen status, with collection of a urine sample at home and collection of blood samples prior to and approximately 2 hours after dosing of morning glucocorticoid and capsule(s) at the study site. At this visit, the investigator will instruct the subject on the first step of the glucocorticoid dose reduction and arrange to contact the subject by telephone within a week of the study visit to assess how the subject is tolerating the glucocorticoid dose reduction. During the follow-up telephone contact, if the investigator feels that a clinical assessment and/or laboratory tests are needed, these can be performed as an unscheduled visit.

Subjects will have study visits at Weeks 6 (optional at home), 9 (optional at home), and 12 for study assessments, including collection of blood samples to assess hormone levels and routine safety assessments.

At the Week 6 visit, the investigator will instruct the subject on the second step of the glucocorticoid dose reduction and will arrange to contact the subject by telephone within a week of the study visit to assess how the subject is tolerating the glucocorticoid dose reduction. The investigator will contact the subject at approximately Week 8 to advise on the third step of glucocorticoid dose reduction (if applicable).

At the Week 9 study visit, the investigator will assess whether the subject is tolerating the third glucocorticoid dose reduction. The investigator will contact the subject at approximately Week 10 to advise on the fourth step of glucocorticoid dose reduction (if applicable).

If the subject experiences any of the following signs or symptoms at any time during the glucocorticoid dose reduction process, the glucocorticoid dose should NOT be reduced further but returned to the previous dose that was tolerated. However, before the glucocorticoid dose reduction is stopped for symptoms or signs of orthostatic hypotension, volume status should be optimized (e.g., with additional dietary salt, salt tablets, intravenous saline).
Unexplained hyponatremia (serum sodium <135 mmol/L)
Orthostatic hypotension with decrease in systolic blood pressure >20 mmHg or in diastolic blood pressure >10 mmHg after standing (from a seated position) after approximately 2 minutes, or severe symptoms of dizziness or lightheadedness upon standing
Severe nausea, food aversion, vomiting
Unacceptable symptoms of hyperandrogenism (e.g., hirsutism, acne, amenorrhea)

Glucocorticoid dose reductions during Weeks 4 to 12 should proceed even if androstenedione levels increase transiently, provided that the increase is asymptomatic and tolerated by the subject.

At the Week 12 visit, based on review of the subject's hormone levels collected up to that visit as well as based on clinical assessment, the investigator will determine the appropriate dose of glucocorticoid to continue past Week 12 (the reduced dose if tolerated, or a prior [higher] dose) in order to achieve adequate control of androgen levels (i.e., androstenedione ≤120% of the subject's baseline or ≤upper limit of normal [ULN] for age and sex).

(c) 12-Week Glucocorticoid Optimization Period (Week 12 Up to Week 24)

Subjects will continue on the glucocorticoid regimen as instructed by the investigator at Week 12 and return to the study site at Week 16 (optional at home), Week 20 (optional at home), and Week 24 during the glucocorticoid optimization period. At these visits, the investigator will review the laboratory results from the preceding study visit and determine if the glucocorticoid regimen requires adjustment in order to achieve adequate control of androgen levels (i.e., androstenedione ≤120% of the subject's baseline or ≤ULN for age and sex).

At the Week 24 visit, subjects will follow a similar procedure as Day 1 for additional androgen assessments with collection of a urine sample at home and collection of blood samples prior to and approximately 2 hours after dosing of morning glucocorticoid and study drug at the study site. Subjects should be fasting from the night before, but should be encouraged to drink water to avoid any hypovolemic status, and a glucose tolerance test will be performed (with study drug taken with the glucose load rather than a meal).

(C) Open-Label Treatment Period (Week 24 Up to Month 12)

For the purpose of this study, months are defined as 4 week intervals.

Starting the evening of the Week 24 visit (after all Week 24 assessments have been performed), all subjects will receive capsule(s) comprising active study drug (crinecerfont) at 100 mg BID with breakfast and evening meals. Subjects should continue the glucocorticoid regimen specified by the investigator at Week 24. Subjects and investigators will remain blinded to subjects' treatment group assignment from the double-blind period.

(a) 1-Month Glucocorticoid Stable Period (Week 24 Up to Month 7)

During the first month of open-label treatment with crinecerfont, subjects should maintain a stable glucocorticoid regimen (except for sick-day guidelines).

(b) 3-Month Glucocorticoid Reduction Period (Month 7 Up to Month 10)

At Months 7 (optional at home), 8, and 9 (optional at home), investigators will decrease glucocorticoid doses in those subjects whose glucocorticoid dose is still greater than 11 mg/m²/day at Month 7 (unless there is a safety concern with regard to glucocorticoid insufficiency), with the goal to achieve a target physiologic dose of 8 to 10 mg/m²/day by Month 10. The glucocorticoid dose should be reduced by approximately 10% to 20% at each visit (Months 7, 8, and 9), as long as androstenedione levels are within control (i.e., androstenedione ≤120% of the subject's baseline or ≤ULN for age and sex) and the subject is not experiencing any signs or symptoms suggestive of clinically relevant glucocorticoid insufficiency or unacceptable symptoms of hyperandrogenism. The glucocorticoid dose reduction will not require dose reduction below 8 mg/m²/day hydrocortisone equivalents. After each of the glucocorticoid dose reduction steps, the site should contact the subject by telephone (within a week) to assess how the subject is tolerating the glucocorticoid dose reduction. Subjects will have study visits at Months 8, 9, and 10 for study assessments including collection of blood samples for hormone levels.

(c) 2-Month Glucocorticoid Maintenance Period (Month 10 Up to Month 12)

Subjects will return to the study site at Months 10 and 12 for study assessments as outlined in the Schedule of Assessments. During this period, the goal should be to maintain stable glucocorticoid doses; however, the dose can be adjusted according to standard of care (e.g., to achieve the control of androgen levels appropriate to the treatment targets for each subject).

At the Month 12 visit, subjects will have additional androgen assessments with collection of a urine sample at home and blood sample collection before and approximately 2 hours after dosing of morning glucocorticoid and study drug at the study site. Subjects should be fasting from the night before (subjects should be encouraged to drink water to avoid any hypovolemic status). A glucose tolerance test will be performed (with capsule(s) taken with the glucose load rather than a meal) at the Month 12 visit.

(D) Open-Label or Double-Blind Active-Controlled Treatment (Month 12 to Month 18)

(a) 6-Month Glucocorticoid Maintenance Period (Month 12 to Month 18) for Subjects with Month 12 Glucocorticoid Dose ≤11 mg/m²/Day

Subjects with glucocorticoid dose ≤11 mg/m²/day at Month 12 will continue on active study drug at 100 mg BID until Month 18 with study visits at Months 14, 16, and 18. The goal during this period is to maintain stable glucocorticoid doses while androstenedione levels are within control (i.e., androstenedione ≤120% of the subject's baseline or ≤ULN for age and sex), although the dose can be adjusted according to standard of care.

At the Month 18 visit, subjects will have additional androgen assessments with collection of a urine sample at home and blood sample collection before and approximately 2 hours after dosing of morning glucocorticoid and capsule(s) administration at the study site. Subjects should be fasting from the night before (subjects should be encouraged to drink water to avoid any hypovolemic status).

(E) Follow-Up Period (Month 19)

A final post-treatment visit will be conducted at Month 19, 1 month after subjects' final dose of capsule(s).

(F) Study Assessments and Study Visit Scheduling

Efficacy, safety, and PK will be assessed at scheduled times throughout the study. As much as possible, all study visits (including baseline and follow-up) should occur at approximately the same time in the morning to standardize time of day for assessment of efficacy, safety, and drug exposure.

In the double-blind, placebo-controlled portion of the study, all visits during the glucocorticoid stable period and glucocorticoid reduction period have a visit window of ±5 days, and all visits during the glucocorticoid optimization period have a visit window of ±5 days. In the open-label treatment period, visits from Month 7 to Month 10 have a visit window of 5 days and visits from Month 12 to Month 19 will have a visit window of ±7 days. If a subject's glucocorticoid regimen is adjusted due to sick-day guidelines, the subject should resume their glucocorticoid dosing regimen for at least 3 days before their next scheduled hormone panel assessment, and this 3-day window supersedes all other visit windows. An independent Data and Safety Monitoring Board (DSMB) will periodically review ongoing clinical safety data to ensure the safety and well-being of study subjects.

### (III) Study Population

Approximately 165 female and male subjects, at least 18 years of age, with a documented medical diagnosis of classic CAH due to 21-hydroxylase deficiency will be enrolled into this study.

To participate in this study, subjects must meet the following criteria:
1. Subjects must provide written informed consent.
2. Be a female or male at least 18 years of age.
3. Have a medically confirmed diagnosis of classic 21-hydroxylase deficiency CAH based on standard medically accepted criteria such as elevated 17-OHP level, confirmed CYP21A2 genotype, positive newborn screening with confirmatory second-tier testing, or cosyntropin stimulation.
4. Be on a stable, supraphysiologic glucocorticoid dose regimen (defined as >14 mg/m²/day in hydrocortisone dose equivalents) that has been stable for at least 1 month prior to screening, is intended for chronic use, and consists of 1 or more of the following glucocorticoids: hydrocortisone (except sustained release), prednisone, prednisolone, methylprednisolone, or dexamethasone. Subjects who are on dexamethasone alone must be receiving ≥0.5 mg/day.
5. If treated with fludrocortisone, dose should be stable for at least 1 month prior to screening with an upright plasma renin activity (PRA) during screening within the normal range on the subject's usual sodium intake. If PRA is not within the normal range, the subject must have systolic blood pressure >100 mmHg, without orthostatic hypotension, and with serum sodium and potassium in the normal range.
6. Female subjects of childbearing potential must agree to use contraception consistently from screening until the final study visit or 30 days after the last dose of study drug, whichever is longer. A female who is not of childbearing potential must meet 1 of the following:
   Postmenopausal, defined as no menses for 12 months without an alternative medical cause and confirmed by elevated follicle-stimulating hormone (FSH) consistent with a postmenopausal range
   Permanent sterilization procedure, such as hysterectomy, bilateral salpingectomy, or bilateral oophorectomy
7. Male subjects must agree to use contraception consistently from screening until 90 days after the last dose of study drug. The acceptable method of contraception for male subjects is condom with spermicide (cream, spray, foam, gel, suppository, or polymer film).

### (IV) Investigational Product, Dosage and Mode of Administration:

Crinecerfont will be administered at 100 mg BID (200 mg total daily dose), based on the free base, in oral capsule form with subjects' breakfast and evening meal (doses separated by approximately 12 hours). The dose may be adjusted to 50 mg qAM and 150 mg qPM at Month 12. Each administration will comprise one or more capsules containing 50 mg of crinecerfont.

Subjects will take the capsule(s) by mouth beginning with the evening meal on Day 1, and then with breakfast and the evening meal (doses separated by approximately 12 hours) for the remainder of the treatment period. Each meal should be completed within 30 minutes of taking the capsule(s).

If a subject forgets or is unable to take the capsule(s), the subject should take his or her dose of study drug as soon as possible, as long as the subject's next dose will be at least 8 hours later. The subject will need to skip the dose if he or she is unable to take the study drug at least 8 hours prior to the next dose.

### (V) Criteria for Evaluation:

(A) Efficacy:

Daily glucocorticoid regimen expressed in hydrocortisone equivalents adjusted for body surface area (BSA) (mg/m²/day).

Hormone measurements: 17-hydroxyprogesterone (17-OHP) (serum; ng/dL), androstenedione (serum; ng/dL), testosterone (serum; ng/dL), adrenocorticotropic hormone (ACTH) (plasma; pg/mL), cortisol (serum; g/dL), luteinizing hormone (LH) (serum; IU/L), follicle stimulating hormone (FSH; IU/L), progesterone (serum; ng/mL), plasma renin activity (measured upright) (ng/mL/hr).

Urine androgen metabolite levels (androsterone and etiocholanolone).

Metabolic assessments (fasting lipid panel, homeostatic model assessment of insulin resistance [HOMA-IR] based on fasting glucose and insulin levels, glycated hemoglobin [HbA1c], glucose tolerance test).

Dual-energy X-ray absorptiometry (DXA) scan (bone mineral density and body composition). Blood pressure.

Hirsutism and Acne Scales (female subjects only).

Testicular ultrasounds (to detect adrenal rest tissue) (male subjects only).

Menstrual Cycle Questionnaire (only in female subjects of childbearing potential who are not on hormonal or intrauterine device contraceptives).

Bone markers: serum osteocalcin, serum bone-specific alkaline phosphatase, serum C-terminal telopeptide, urine N-terminal telopeptide.

(B) Patient-Reported Outcomes:

36-Item Short Form Health Survey (SF-36), EuroQol 5 Dimensions 5 Levels (EQ-5D-5L), Multidimensional Assessment of Fatigue (MAF), Psychological General Well-Being Index (PGWBI), and Medical Outcomes Study 12-Item Sleep Scale (MOS-12).

(C) Pharmacokinetics:

Blood samples to evaluate plasma concentrations of crinecerfont and metabolites will be collected throughout the study.

(D) Safety:

Safety and tolerability will be monitored throughout the study and will include the following assessments:
Adverse events (including glucocorticoid-related events)
Clinical laboratory tests
Vital signs
Weight/body mass index (BMI), and waist circumference
Physical examinations
12-lead electrocardiograms
Brief Psychiatric Rating Scale (BPRS)
Columbia-Suicide Severity Rating Scale (C-SSRS)

### (VI) Study Endpoints:

The primary endpoint is the percent change from baseline in glucocorticoid daily dose (in hydrocortisone equivalents adjusted for BSA [mg/m²/day]) at Week 24, while Week 24 androstenedione is adequately controlled at ≤120% of baseline or ≤upper limit of normal for age and sex. The primary analysis of the primary endpoint will be performed using an analysis of covariance (ANCOVA) model.

The first key secondary endpoint is the change from baseline in serum androstenedione at Week 4, which will be analyzed using an ANCOVA model.

The second key secondary endpoint is the achievement of a reduction in glucocorticoid daily dose to physiologic levels (≤11 mg/m²/day in hydrocortisone equivalent adjusted for BSA) at Week 24 while maintaining androstenedione levels (as defined above in the primary endpoint), which will be analyzed using a Cochran-Mantel-Haenszel (CMH) test.

Additional key secondary endpoints are the changes from baseline in HOMA-IR, weight, and fat mass at Week 24, which will be analyzed using an ANCOVA model.

### Example 18: A Randomized, Double-Blind, Placebo-Controlled Study to Evaluate the Safety and Efficacy of a Compound of Formula (I) in Pediatric Subjects with Classic Congenital Adrenal Hyperplasia, Followed by Open-Label Treatment

### (I) Objectives

To evaluate the efficacy of crinecerfont compared with placebo, in reducing adrenal androgen and precursor levels during a glucocorticoid-stable period.
To evaluate the efficacy of crinecerfont compared with placebo, in reducing daily glucocorticoid dosage while maintaining adrenal androgen control.
To evaluate plasma concentrations of crinecerfont and metabolites.
To assess the safety and tolerability of crinecerfont.

### (II) Methodology

This is a Phase 3, randomized, double-blind, placebo-controlled study to evaluate the efficacy, safety, and tolerability of crinecerfont versus placebo administered twice daily (BID) with breakfast and evening meals for 28 weeks in approximately 81 pediatric subjects with classic CAH due to 21-hydroxylase deficiency. Eligible subjects will be randomly assigned in a 2:1 ratio (active:placebo) to either crinecerfont (25 mg BID oral liquid for subjects 10 to <20 kg, 50 mg BID oral liquid for subjects 20 to <55 kg, or 100 mg BID oral capsule for subjects ≥55 kg) or matching placebo (oral liquid placebo for subjects <55 kg and oral capsule placebo for subjects ≥55 kg). After the 28-week placebo-controlled treatment period, there will be a 24-week, open-label treatment period, during which all subjects will receive crinecerfont at the same doses as administered during the placebo-controlled treatment period. A final study visit will be conducted approximately 4 weeks after the Week 52 visit.

(A) Screening Period (Weeks −4 Up to Day −1)

Parental or legal guardian informed consent with signed and witnessed study subject assent (as required by the governing institutional review board or ethics committee and according to local laws and regulations) will be obtained prior to any study-related procedures. Subjects will undergo screening for up to 4 weeks (Weeks −4 to Day −1) to determine eligibility. Rescreening is permitted if a subject does not meet all eligibility requirements and returns to be rescreened. A subject that has failed screening twice may not be rescreened again without prior permission.

(B) Randomized, Double-Blind, Placebo-Controlled Treatment Period (Day 1 Up to Week 28)

(a) Glucocorticoid-Stable Period

One Day 1, subjects ≥12 years of age should be fasting after midnight the night before until the first blood sample is collected at the site, after which they will be provided breakfast. They should be encouraged to drink water during the fasting period to avoid any hypovolemic status. Subjects <12 years of age do not need to fast.

On Day 1 (baseline), subjects ≥6 years of age and ≥20 kg body weight will hold their morning glucocorticoid dose and bring it with them to the study site, arriving to the site by approximately 0800 hours. Blood samples will be obtained serially over approximately 3.5 hours (at 0830, 0900, 1000, 1100, and 1200 hours), with the morning glucocorticoid dose administered after the 0900 hour sample is collected. Subjects <6 years of age or <20 kg body weight will take their morning glucocorticoid dose at home and have a single blood sample collected at the site, timed to be approximately 2 hours after the morning glucocorticoid dose. Salivary samples for adrenal androgens and precursors will also be collected.

Subjects will be randomized on Day 1 in a 2:1 ratio (active:placebo). Randomization will be stratified by pubertal stage (Tanner stage 1 or 2 vs. 3, 4 or 5) and sex within each dose group. Beginning on Day 1 (baseline), the oral liquid or capsule(s) will be administered at home with the subject's evening meal; thereafter, the oral liquid or capsule(s) will be administered BID with the subject's breakfast and evening meals (doses separated by approximately 12 hours).

From Day 1 until Week 4, subjects should maintain a stable glucocorticoid regimen to the extent possible, except for sick-day guidelines. Sick-day dosing may follow alternate guidelines or can be based on guidance provided by the investigator or the subject's treating physician.

(b) Glucocorticoid Adjustment Period

At the Week 4 visit, subjects ≥6 years of age and ≥20 kg body weight will hold their morning glucocorticoid and oral liquid or capsule(s) and bring it with them to the study site, arriving to the site by approximately 0800 hours. Blood samples will be obtained serially over approximately 6.5 hours (at 0830, 0900, 1000, 1100, 1200, 1300, and 1500 hours). The morning glucocorticoid dose and oral liquid or capsule(s) will be administered after the 0900 hours sample is collected. Subjects <6 years of age or <20 kg body weight will take their morning glucocorticoid dose at home (at approximately the same time as on Day 1) but hold their morning oral liquid or capsule(s) and have a single blood sample collected at the site, timed to be approximately 2 hours after the morning glucocorticoid dose.

Salivary samples for adrenal androgens and precursors will also be collected.

From Week 4 until Week 28, the subject's glucocorticoid dose should be adjusted according to their androstenedione levels, with the goal to reach a dose of approximately 8 to 10 mg/m²/day at Week 28, if androstenedione can be maintained ≤baseline levels. Glucocorticoid dose adjustments can occur in as few as 1 or up to 4 steps, depending on the starting and target glucocorticoid doses and the amount of dose adjustment at each step. The target glucocorticoid dose should be within the range of 8 to 10 mg/m²/day to the extent possible, but could be lower than this range depending on practical issues considered in clinical practice related to available dosage strengths. Before any glucocorticoid dose reduction is implemented, the investigator will evaluate the subject for any symptoms suggestive of glucocorticoid insufficiency using a standardized checklist and will arrange for follow-up if needed after the dose reduction.

The first glucocorticoid dose adjustment step should be guided by the change in androstenedione (A4) at Week 4 from baseline. A suggested guideline is provided in the table below, but the exact amount adjusted may differ from this guideline based on practical issues considered in clinical practice related to available dosage strengths. The investigator should contact the subject once the Week 4 lab results are available in order to provide guidance on the amount of the first glucocorticoid dose adjustment.

TABLE 46

| Percent Change from Baseline in | GC Dose Adjustment Step #1 |
|---|---|
| Androstenedione at Week 4 | (approximately Week 6) |
| No change or increase | Consider whether GC dose needs to be |
| | increased |
| Decrease of >0 to ≤20% | 1 to 2 mg/m²/day GC dose decrease |
| Decrease of >20% to ≤40% | 2 to 3 mg/m²/day GC dose decrease |
| Decrease of >40% | 3 to 4 mg/m²/day GC dose decrease |

A follow-up blood test should be arranged approximately 2 weeks later at Week 8 (at home or the study site).

For all blood tests after the Week 4 visit, subjects should take their morning glucocorticoid dose at home with blood sample collection timed approximately 2 hours after the glucocorticoid dose.

If needed, subsequent glucocorticoid dose adjustment steps should occur when lab results are available (at approximately Week 10, Week 14, and Week 18) with follow-up blood tests at Week 12 (at home or the study site, and only if the glucocorticoid dose is modified at Week 10), Week 16 (at the study site), and Week 20 (at home or the study site).

The target amount of glucocorticoid dose reduction at each step is approximately 1 to 4 mg/m²/day but should be guided by the androstenedione level at the preceding blood test as well as on practical issues considered in clinical practice related to available dosage strengths.

TABLE 47

| Blood Test | Glucocorticoid Dose Adjustment Step |
|---|---|
| Week 8 (at home or | Step 2 (if needed) at approximately |
| the study site) | Week 10 (or when Week 8 labs available) |
| Week 12 (if Step 2 | Step 3 (if needed) at approximately |
| needed, at home or | Week 14 (or when Week 12 labs available) |
| the study site) | |
| Week 16 (at the study | Step 4 (if needed) at approximately |
| site) | Week 18 (or when Week 16 labs available) |
| Week 20 (at home or | If androstenedione not ≤baseline, further |
| the study site) | glucocorticoid dose adjustment may be needed |

Subjects will return to the study site at Week 16 and Week 28 for assessments as outlined in the Schedule of Assessments. Prior to the Week 16 and Week 28 visits, subjects will hold their morning oral liquid or capsule(s) and bring it with them to the study site, but will take their morning glucocorticoid dose at home, with blood sample collection timed to be approximately 2 hours later.

For the Week 28 visit, subjects ≥12 years of age should be fasting after midnight the night before until the first blood sample is collected at the site, after which they will be provided breakfast. Subjects should be encouraged to drink water during the fasting period to avoid hypovolemic status. Subjects <12 years of age do not need to fast.

(C) Open-Label Treatment Period (Week 28 to Week 52)

Starting the evening of the Week 28 visit (after all Week 28 assessments have been performed), all subjects will receive crinecerfont (crinecerfont; 25 mg BID oral liquid for subjects 10 to <20 kg, 50 mg BID oral liquid for subjects 20 to <55 kg, or 100 mg BID oral capsule for subjects ≥55 kg) with breakfast and evening meals. Subjects and investigators will remain blinded to subjects' treatment group assignment during the placebo-controlled treatment period.

For subjects who are still on greater than 10 mg/m²/day glucocorticoid dose at Week 28, further adjustments in glucocorticoid dose should be made following the guidelines used during the placebo-controlled period, and a blood sample will be collected at Week 32 (at home or the study site).

The first glucocorticoid dose adjustment step (if done) should be guided by the androstenedione change at Week 32 (compared with Week 28), after all subjects have been on open-label active study drug for 4 weeks. A suggested guideline is provided below but the exact amount adjusted may differ from this guideline based on practical issues considered in clinical practice related to available dosage strengths. The investigator should contact the subject once the Week 32 lab results are available in order to provide guidance on the amount of the first glucocorticoid dose adjustment (if needed) during the open-label period.

TABLE 48

| Percent Change from | |
|---|---|
| Week 28 in | |
| Androstenedione at | GC Dose Adjustment Step #1 |
| Week 32 | (approximately Week 34) |
| No change or increase | Consider whether GC dose needs to be increased) |
| Decrease of >0 to ≤20% | 1 to 2 mg/m²/day GC dose reduction |
| Decrease of >20% to ≤40% | 2 to 3 mg/m²/day GC dose reduction |
| Decrease of >40% | 3 to 4 mg/m²/day GC dose reduction |

If the glucocorticoid dose is modified at approximately Week 34, a follow-up blood test should be arranged approximately 2 weeks later at Week 36 (at home or the study site).

If needed, subsequent glucocorticoid dose adjustments should occur at approximately Week 38 and Week 42 (or when lab results are available) with follow-up blood tests at Week 40 (at the study site) and Week 44 (at home or the study site, and only if the glucocorticoid dose is modified at Week 42). The target amount of glucocorticoid dose reduction at each step is approximately 1 to 4 mg/m²/day but should be guided by the androstenedione level at the preceding blood test as well as practical issues considered in clinical practice related to available dosage strengths.

TABLE 49

| Blood Test | GC dose adjustment step |
|---|---|
| Week 36 (at home or at | Step 2 (if needed) at approximately Week 38 |
| the study site) | (or when Week 36 A4 result is available) |
| Week 40 (at the study site) | Step 3 (if needed) at approximately Week 42 |
| | (or when Week 40 A4 resut is available) |
| Week 44 (if Step 3 needed, | If A4 not < baseline, further GC dose |
| at home or the study site) | adjustment may be needed |

Subjects will return to the study site at Week 40 and Week 52 for assessments as outlined in the Schedule of assessments. Prior to the Week 40 and Week 52 visits, subjects will hold their morning oral liquid or capsule(s) and bring it with them to the study site, but will take their morning glucocorticoid dose at home, with blood sample collection timed to be approximately 2 hours later.

For the Week 52 visit, subjects ≥12 years of age should be fasting after midnight the night before until the first blood sample is collected at the site, after which they will be provided breakfast. Subjects should be encouraged to drink water during the fasting period to avoid any hypovolemic status. Subjects <12 years of age do not need to fast.

(D) Study Assessments and Study Visit Scheduling

Efficacy, safety, and PK will be assessed at scheduled times throughout the study. As much as possible, all study visits (including baseline, during the study, and follow-up) should occur at approximately the same time in the morning to standardize time of day for assessment of efficacy, safety, and drug exposure.

The Week 4 visit will have a visit window of 5 days, and subsequent visits will have a visit window of ±7 days. If a subject's glucocorticoid regimen is adjusted due to sick-day guidelines, the subject should resume their glucocorticoid dosing regimen for at least 3 days before their next scheduled lab test, and this 3-day window supersedes all other visit windows.

An independent Data Monitoring Committee will periodically review unblinded study data to ensure the safety and well-being of study subjects and to confirm observed exposures are consistent with expected target exposures.

### (III) Study Population

Approximately 81 female and male subjects, 2 to 17 years of age, with a documented medical diagnosis of classic CAH due to 21-hydroxylase deficiency will be enrolled into this study.

To participate in this study, subjects must meet the following criteria:
1. Have documentation of witnessed written or oral pediatric assent from the subject deemed capable of providing assent, and written informed consent from the subject's parent(s) or legal guardian in accordance with the governing institutional review board or ethics committee and according to local laws and regulations.
2. Be a female or male at least 2 years of age and less than 18 years of age and a body weight of at least 10 kg.
3. Have a medically confirmed diagnosis of classic 21-hydroxylase deficiency CAH based on standard medically accepted criteria such as elevated 17-OHP level, confirmed CYP21A2 genotype, positive newborn screening with confirmatory second-tier testing, or cosyntropin stimulation.
4. Be on a supraphysiologic glucocorticoid dose regimen (defined as >12 mg/m²/day in hydrocortisone dose equivalents) that has been above this threshold for at least 6 months and at a stable dose for at least 1 month prior to screening, is intended for chronic use, and consists of 1 or more of the following glucocorticoids: hydrocortisone (except sustained release), prednisone, prednisolone, methylprednisolone, or dexamethasone. Subjects must be on a morning dose of glucocorticoid.
5. Have an androstenedione level (prior to the morning glucocorticoid dose) greater than upper limit of normal (according to age, sex, and/or pubertal stage).
6. Have a 17-hydroxyprogesterone level (prior to the morning glucocorticoid dose) greater than 800 ng/dL.
7. If treated with fludrocortisone, dose should be stable for at least 1 month prior to screening with an upright plasma renin activity (PRA) during screening within the normal range on the subject's usual sodium intake. If PRA is not within the normal range, the subject must have systolic blood pressure >100 mmHg, without orthostatic hypotension, and with serum sodium and potassium in the normal range.
8. Female subjects of childbearing potential who are sexually active must agree to use contraception consistently from screening until the final study visit or 30 days after the last dose of study drug, whichever is longer. A female subject of childbearing potential is defined as a female capable of becoming pregnant, which includes subjects who have had their first menstrual cycle (i.e., menarche) and are not surgically sterile (i.e., bilateral oophorectomy, hysterectomy or bilateral tubal ligation for at least 3 months prior to screening). A male subject of childbearing potential is defined as a subject who has reached spermarche and has not been vasectomized for at least 3 months prior to screening. Male subjects of childbearing potential who are sexually active must agree to use effective barrier contraception consistently from screening until 90 days after the last dose of study drug. The acceptable method of contraception for male subjects is condom with spermicide (cream, spray, foam, gel, suppository, or polymer film).

### (IV) Investigational Product, Dosage and Mode of Administration

Crinecerfont (25 mg BID oral liquid for subjects 10 to <20 kg, 50 mg BID oral liquid for subjects 20 to <55 kg, or 100 mg BID oral capsule for subjects ≥55 kg) will be administered with subjects' breakfast and evening meals (doses separated by approximately 12 hours). Each oral capsule contains 50 mg crinecerfont (free base). The oral liquid contains 50 mg of crinecerfont (free base) per 1 mL and will be administered via a calibrated oral dosing syringe.

### (V) Criteria for Evaluation

(A) Efficacy
Hormone measurements: Androstenedione (A4; serum and saliva), 17-hydroxyprogesterone (17-OHP; serum and saliva), adrenocorticotropic hormone (ACTH; plasma), luteinizing hormone (LH; serum), testosterone (serum), plasma renin activity (measured upright).
Daily glucocorticoid regimen expressed in hydrocortisone equivalents adjusted for body surface area (BSA) (mg/m²/day).
Body weight and body mass index.
Growth (assessed as height velocity).
Bone age based on X-ray (only for subjects not at adult height and not with fused phalangeal epiphyses on X-ray).
Metabolic assessments (only in subjects ≥12 years of age; fasting lipid panel and homeostatic model assessment of insulin resistance [HOMA-IR] based on fasting glucose and insulin levels).
Menstrual cycle questionnaire (only in female subjects who have undergone menarche and are not on hormonal or intrauterine device contraceptives).
Hirsutism (only for female subjects) and acne scales.
Testicular ultrasounds (to detect adrenal rest tissue; only in male subjects).

(B) Patient and Caregiver Reported Outcomes
EuroQol (European Quality of Life)-5 Dimensions-Youth (EQ-5D-Y)
Pediatric Quality of Life Instrument (Peds-QL)
Peds-QL Family Impact

(C) Pharmacokinetics
Blood samples to evaluate plasma concentrations of crinecerfont and metabolites will be collected throughout the study.

(D) Other
Palatability assessment

(E) Safety
Adverse events (including glucocorticoid-related events)
Clinical laboratory tests (chemistry, hematology, coagulation, urinalysis)
Vital signs
Physical examinations, including height, weight, and Tanner stage
6- or 12-lead electrocardiograms
Brief Psychiatric Rating Scale, Children's Version (BPRS-C)
Columbia-Suicide Severity Rating Scale (C-SSRS) Children's Version (only for subjects ≥6 years of age)

### (VI) Study Endpoints and Statistical Analysis

The primary endpoint is the change from baseline to Week 4 in serum androstenedione (average across all values obtained from 0830 to 1200 hours). The first key secondary endpoint is the change from baseline to Week 4 in serum 17-OHP (average across all values obtained from 0830 to 1200 hours). The second key secondary endpoint is the percent change from baseline to Week 28 in glucocorticoid daily dose (in hydrocortisone equivalents adjusted for BSA [mg/m²/day]), while Week 28 androstenedione is less than or equal to the baseline value.

One secondary endpoint is the achievement of a reduction in glucocorticoid daily dose to physiologic levels (≤11 mg/m²/day in hydrocortisone equivalent adjusted for BSA) at Week 28, while Week 28 androstenedione is less than or equal to the baseline value. Additional secondary endpoints include change from baseline in body mass index standard deviation score (SDS) at Week 28 and change in height velocity at Week 28 (restricted to subset of subjects not at adult height).

The continuous endpoints will be analyzed using an analysis of covariance (ANCOVA) model and will include treatment group (crinecerfont v. placebo), stratification factors used in the randomization and, as appropriate, baseline value. The binary endpoint will be compared by treatment group (crinecerfont vs. placebo) using a Cochran-Mantel-Haenszel (CMH) test stratified by factors used in the randomization. The overall Type I error of 0.05 will be controlled by testing the primary, first key secondary, and second key secondary endpoints sequentially in this order.

### Example 19. A Phase 1 Study to Evaluate Tolerability and Pharmacokinetics of Crinecerfont in Healthy Subjects

### (I) Objectives

To assess the safety and tolerability of crinecerfont (NBI-74788) in healthy subjects at total daily doses of 250 mg or 300 mg.
To assess the pharmacokinetics (PK) of crinecerfont and metabolites in healthy subjects at total daily doses of 250 mg or 300 mg.

### (II) Methodology

This will be a Phase 1, multiple-dose, randomized, double-blind, placebo-controlled study designed to assess the safety, tolerability, and PK of crinecerfont at 2 dose levels in healthy subjects. Approximately 30 subjects (male or female) will be enrolled and randomized 1:1:1 to crinecerfont 300 mg, crinecerfont 250 mg, or placebo taken as described in the table below. Randomization will occur on Day 1, and each subject will receive a 28-day regimen of crinecerfont or placebo in a blinded fashion. Doses will be administered with breakfast and evening meals, approximately 12 hours apart.

TABLE 50
Dosing Regimen for Each Treatment Group:

| | Type/Timing of Dose | |
|---|---|---|
| Treatment Group | Morning (AM) Dose | Evening (PM) Dose |
| A | Crinecerfont 100 mg | Crinecerfont 200 mg |
| (N = 10) | (2 × 50 mg capsules) | (4 × 50 mg capsules) |
| B | Crinecerfont 100 mg | Crinecerfont 150 mg |
| (N = 10) | (2 × 50 mg capsules) | (3 × 50 mg capsules + |
| | | 1 × placebo capsule) |
| C | Placebo | Placebo |
| (N = 10) | (2 × placebo capsules) | (4 × placebo capsules) |

Subjects will be screened for eligibility within 42 days prior to the initiation of dosing on Day 1. Subjects will be admitted to the study site no later than Day −1 at the time indicated by the study site. Subjects will be confined to the study center for the duration of dosing.

Confinement will end after collection of the PM blood sample and completion of scheduled study procedures on Day 28. Follow-up visits will be conducted at Weeks 5, 6, and 8 (i.e., days 35, 42, and 56) during a wash-out period after the final dose.

Blood samples for PK analysis of crinecerfont and metabolites will be collected within 30 minutes prior to the first dose on Day 1, predose for both AM and PM doses daily on Days 7 through 14 (inclusive), Day 21, Day 28, and at follow-up visits at Weeks 5, 6, and 8. Additionally, blood samples for PK analysis of crinecerfont and metabolites will be collected at approximately 0.5, 1, 2, 3, 4, 5, 6, 7, 8, 9, 10, and 12 hours following both the AM and PM doses on Day 1 and Day 14 (i.e., the final sample will be collected prior to the AM dose on the following day, Day 2 or Day 15, respectively).

Safety and tolerability assessments including adverse events (AEs), clinical laboratory tests (including chemistry, hematology, coagulation, and urinalysis), morning cortisol levels, body weight, vital signs measurements, physical examinations, and 12-lead electrocardiogram (ECG) will be monitored during the study.

### (III) Study Population

A total of approximately 30 healthy subjects (male or female), 18 to 55 years old inclusive, will be enrolled.

### (IV) Investigational Product, Dosage and Mode of Administration:

Crinecerfont will be supplied as capsules containing 50 mg of NBI-74788 free base for oral administration, with breakfast and evening meals (approximately 12 hours apart).

### (V) Criteria for Evaluation:

(A) Pharmacokinetics:

Blood samples for plasma concentrations of crinecerfont and metabolites will be collected predose on Day 1 (within 15 minutes prior to first dose), on Days 7, 8, 9, 10, 11, 12, 13, 14, 21, and 28 (within 15 minutes prior to each AM and PM dose), and at follow up visits at Weeks 5, 6, and 8 (Days 35, 42, and 56).

Blood samples to determine plasma concentrations for the PK profile of crinecerfont and metabolites will be collected approximately 0.5, 1, 2, 3, 4, 5, 6, 7, 8, 9, 10, and 12 hours following both the AM and PM doses on Day 1 and Day 14. The following plasma PK parameters will be calculated for crinecerfont and metabolites:
Area under the plasma concentration versus time curve (AUC) over the dosing interval (AUC₀₋ₜₐᵤ)
Area under the plasma concentration versus time curve from 0 to 24 hours (AUC₀₋₂₄)
Maximum plasma concentration (Cₘₐₓ)
Time to maximum plasma concentration (tₘₐₓ)
Molar AUC ratio of metabolites to the parent drug crinecerfont
Apparent systemic clearance after oral administration clearance (CL/F) (crinecerfont only)
Accumulation ratio (R_{ac})

(B) Safety
AEs
Clinical laboratory tests (chemistry, hematology, coagulation, and urinalysis)
Morning cortisol levels
Vital signs (including orthostatic blood pressure and pulse) and body weight
Physical examinations
12-lead ECGs

### Other Embodiments

It is to be understood that the foregoing description is intended to illustrate and not limit the scope of the disclosure, which is defined by the scope of the appended claims. Other aspects, advantages, and modifications are within the scope of the following claims.

What is claimed is:

1. A compound of Formula (I): 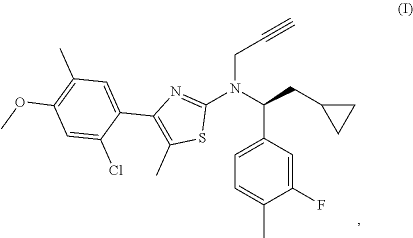 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
for use in a method of treating congenital adrenal hyperplasia in a subject,
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

2. The compound of claim 1, wherein the compound of Formula (I), or a pharmaceutically acceptable salt thereof, is administered at a frequency of twice daily.

3. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100.

4. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:50.

5. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:10.

6. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:5.

7. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:3.

8. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.5.

9. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.

10. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5.

11. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.

12. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.5.

13. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration about 1:3.

14. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:3.5.

15. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:4.

16. The compound of any one of claims 1 to 15, wherein the amount of the compound of Formula (I), or a pharmaceutically acceptable salt thereof, administered daily is less than or equal to about 1000 mg based on the weight of the free base.

17. The compound of any one of claims 1 to 15, wherein the amount of the compound of Formula (I), or a pharmaceutically acceptable salt thereof, administered daily is from about 50 mg to about 1000 mg based on the weight of the free base.

18. The compound of any one of claims 1 to 15, wherein the amount of the compound of Formula (I), or a pharmaceutically acceptable salt thereof, administered daily is from about 100 mg to about 1000 mg based on the weight of the free base.

19. The compound of any one of claims 1 to 15, wherein the amount of the compound of Formula (I), or a pharmaceutically acceptable salt thereof, administered daily is from about 100 mg to about 500 mg based on the weight of the free base.

20. The compound of any one of claims 1 to 15, wherein the amount of the compound of Formula (I), or a pharmaceutically acceptable salt thereof, administered daily is from about 100 mg to about 400 mg based on the weight of the free base.

21. The compound of any one of claims 1 to 15, wherein the amount of the compound of Formula (I), or a pharmaceutically acceptable salt thereof, administered daily is from about 100 mg to about 300 mg based on the weight of the free base.

22. The compound of any one of claims 1 to 15, wherein the amount of the compound of Formula (I), or a pharmaceutically acceptable salt thereof, administered daily is about 200 mg based on the weight of the free base.

23. The compound of claim 22, wherein the first administration of the compound of Formula (I), or a pharmaceutically acceptable salt thereof is about 50 mg based on the weight of the free base and the second administration of the compound of Formula (I), or a pharmaceutically acceptable salt thereof is about 150 mg based on the weight of the free base.

24. The compound of any one of claims 1 to 15, wherein the amount of the compound of Formula (I), or a pharmaceutically acceptable salt thereof, administered daily is about 250 mg based on the weight of the free base.

25. The compound of claim 24, wherein the first administration of the compound of Formula (I), or a pharmaceutically acceptable salt thereof is about 100 mg and the second administration of the compound of Formula (I), or a pharmaceutically acceptable salt thereof is about 150 mg based on the weight of the free base.

26. The compound of any one of claims 1 to 15, wherein the amount of the compound of Formula (I), or a pharmaceutically acceptable salt thereof, administered daily is about 300 mg based on the weight of the free base.

27. The compound of claim 26, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base.

28. The compound of any one of claims 1 to 27, wherein the subject weighs greater than or equal to about 55 kg.

29. The compound of any one of claims 1 to 15, wherein the amount of the compound of Formula (I), or a pharmaceutically acceptable salt thereof, administered daily is about 50 mg based on the weight of the free base.

30. The compound of claim 29, wherein the subject weighs from about 10 kg to about 20 kg.

31. The compound of any one of claims 1 to 15, wherein the amount of the compound of Formula (I), or a pharmaceutically acceptable salt thereof, administered daily is about 100 mg based on the weight of the free base.

32. The compound of claim 31, wherein the wherein the first administration of the compound of Formula (I), or a pharmaceutically acceptable salt thereof is about 25 mg based on the weight of the free base and the second administration of the compound of Formula (I), or a pharmaceutically acceptable salt thereof is about 75 mg based on the weight of the free base.

33. The compound of claim 31 or 32, wherein the subject weighs from about 20 kg to about 55 kg.

34. A compound of Formula (I): 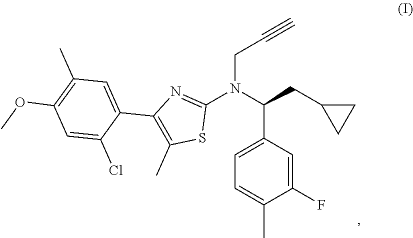 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
for use in a method of treating congenital adrenal hyperplasia in a subject,
wherein the compound of Formula (I), or a pharmaceutically acceptable salt thereof, is administered at a frequency of not less than twice daily; and
wherein the amount of the compound of Formula (I), or a pharmaceutically acceptable salt thereof, administered daily is greater than 200 mg based on the weight of the free base.

35. The compound of claim 34, wherein the amount of the compound of Formula (I), or a pharmaceutically acceptable salt thereof, is administered at a frequency of twice daily.

36. The compound of claim 34 or 35, wherein the amount of the compound of Formula (I), or a pharmaceutically acceptable salt thereof, administered daily is from about 200 mg to about 1000 mg based on the weight of the free base.

37. The compound of claim 34 or 35, wherein the amount of the compound of Formula (I), or a pharmaceutically acceptable salt thereof, administered daily is from about 225 mg to about 1000 mg based on the weight of the free base.

38. The compound of claim 34 or 35, wherein the amount of the compound of Formula (I), or a pharmaceutically acceptable salt thereof, administered daily is from about 225 mg to about 500 mg based on the weight of the free base.

39. The compound of claim 34 or 35, wherein the amount of the compound of Formula (I), or a pharmaceutically acceptable salt thereof, administered daily is from about 225 mg to about 400 mg based on the weight of the free base.

40. The compound of claim 34 or 35, wherein the amount of the compound of Formula (I), or a pharmaceutically acceptable salt thereof, administered daily is from about 225 mg to about 300 mg based on the weight of the free base.

41. The compound of claim 34 or 35, wherein the amount of the compound of Formula (I), or a pharmaceutically acceptable salt thereof, administered daily is about 250 mg based on the weight of the free base.

42. The compound of any one of claims 34 to 41, wherein the first administration of the compound of Formula (I), or a pharmaceutically acceptable salt thereof is about 125 mg based on the weight of the free base and the second administration of the compound of Formula (I), or a pharmaceutically acceptable salt thereof is about 125 mg based on the weight of the free base.

43. The compound of claim 34 or 35, wherein the amount of the compound of Formula (I), or a pharmaceutically acceptable salt thereof, administered daily is about 300 mg based on the weight of the free base.

44. The compound of any one of claims 34 to 40 and 43, wherein the first administration of the compound of Formula (I), or a pharmaceutically acceptable salt thereof is about 150 mg based on the weight of the free base and the second administration of the compound of Formula (I), or a pharmaceutically acceptable salt thereof is about 150 mg based on the weight of the free base.

45. The compound of any one of claims 1 to 44, wherein the compound of Formula (I), or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject.

46. The compound of claim 45, wherein the reduction in level of any of biomarkers is determined by comparing the level of the biomarker as measured during the circadian release during a time period prior to administering the compound of Formula (I), or a pharmaceutically acceptable salt thereof and the level of the biomarker as measured during the circadian release on a day after administering the compound of Formula (I), or a pharmaceutically acceptable salt thereof.

47. The compound of claim 46, wherein the circadian release occurs between the hours of 2 a.m. and 10 a.m.

48. The compound of any one of claims 45 to 47, wherein the level of 17-hydroxyprogesterone is reduced by at least 25%.

49. The compound of any one of claims 45 to 47, wherein the level of 17-hydroxyprogesterone is reduced by at least 50%.

50. The compound of any one of claims 45 to 47, wherein the level of adrenocorticotropic hormone is reduced by at least 25%.

51. The compound of any one of claims 45 to 49, wherein the level of adrenocorticotropic hormone is reduced by at least 40%.

52. The compound of any one of claims 45 to 49, wherein the level of adrenocorticotropic hormone is reduced by at least 50%.

53. The compound of any one of claims 45 to 52, wherein the level of androstenedione is reduced by at least 25%.

54. The compound of any one of claims 45 to 52, wherein the level of androstenedione is reduced by at least 30%.

55. The compound of any one of claims 45 to 52, wherein the level of androstenedione is reduced by at least 50%.

56. The compound of any one of claims 45 to 55, wherein the level of 17-hydroxyprogesterone is reduced by at least 50% and the level of androstenedione is reduced by at least 50%.

57. A compound of Formula (I): 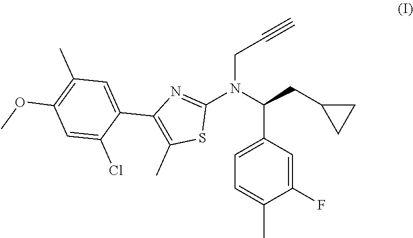 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
for use in a method of reducing the severity of one or more symptoms selected from hirsutism, precocious puberty, fertility problems, acne, and growth impairment in a subject having classic congenital adrenal hyperplasia,
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

58. The compound of claim 57, wherein the total daily amount of the compound of Formula (I) is sufficient to reduce the level of androstenedione in the subject.

59. The compound of claim 57 or 58, wherein the growth impairment is selected from one or more of accelerated height velocity, accelerated weight velocity, or accelerated bone age.

60. The compound of claim 58 or 59, wherein the androstenedione is reduced by at least 25%.

61. The compound of claim 58 or 59, wherein the androstenedione is reduced by at least 30%.

62. The compound of claim 58 or 59, wherein the androstenedione is reduced by at least 50%.

63. A compound of Formula (I): 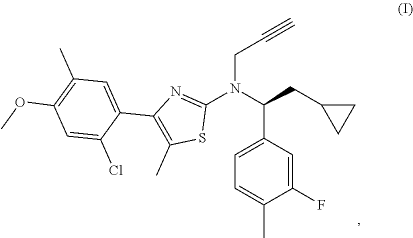 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof,
for use in method of reducing the level of one or more biomarkers of congenital adrenal hyperplasia in a subject having congenital adrenal hyperplasia,
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

64. The compound of claim 63, wherein the one or more biomarkers of congenital adrenal hyperplasia are selected from (a) 17-hydroxyprogesterone (17-OHP); (b) adrenocorticotropic hormone (ACTH); and (c) androstenedione.

65. A compound of Formula (I): 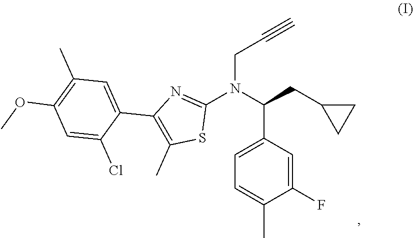 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
for use in a method of reducing the dosage of corticosteroid administered to a subject having congenital adrenal hyperplasia for controlling congenital adrenal hyperplasia,
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

66. The compound of claim 65, wherein the corticosteroid is a glucocorticoid.

67. A compound of Formula (I): 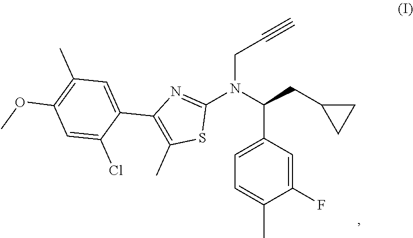 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof,
for use in a method of reducing the severity of one or more side effects of glucocorticoid treatment in a subject having congenital adrenal hyperplasia,
wherein the compound of Formula (I), or a pharmaceutically acceptable salt thereof, is administered at a frequency of not less than twice daily;
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations; and
wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

68. The compound of any one of claims 63 to 67, wherein the compound of Formula (I), or a pharmaceutically acceptable salt thereof, is administered at an amount sufficient to reduce the level of 17-hydroxyprogesterone (17-OHP) by at least 50% as compared to the level prior to administration.

69. The method of any one of claims 63 to 68, wherein the compound of Formula (I), or a pharmaceutically acceptable salt thereof is administered at an amount sufficient to reduce the level of androstenedione by at least 30% as compared to the level prior to administration.

70. The method of any one of claims 63 to 68, wherein the compound of Formula (I), or a pharmaceutically acceptable salt thereof is administered at an amount sufficient to (a) reduce the level of 17-hydroxyprogesterone (17-OHP) by at least 50% as compared to the level prior to administration; and (b) reduce the level of androstenedione by at least 30% as compared to the level prior to administration.

71. The method of any one of claims 57 to 70, wherein the compound of Formula (I), or a pharmaceutically acceptable salt thereof, is administered twice daily at an amount from about 25 mg to about 250 mg based on the weight of the free base.

72. The method of any one of claims 57 to 70, wherein the compound of Formula (I), or a pharmaceutically acceptable salt thereof, is administered twice daily as:
(a) a first administration of about 50 mg based on the weight of the free base and a second administration of about 150 mg based on the weight of the free base; or
(b) a first administration of about 100 mg based on the weight of the free base and a second administration of about 150 mg based on the weight of the free base; or
(c) a first administration of about 100 mg based on the weight of the free base and a second administration of about 200 mg based on the weight of the free base.

73. The compound of any one of claims 1 to 72, wherein the compound of Formula (I) is administered in the free base form.

74. The compound of any one of claims 1 to 73, wherein the subject is in a fed state.

75. The compound of claim 74, wherein the compound of Formula (I), or a pharmaceutically acceptable salt thereof, is administered to the subject with a nutritional composition.

76. The compound of claim 75, wherein the nutritional composition is a liquid dietary supplement comprising about 1000 to about 2000 calories per liter with a fat content greater than about 30%.

77. The compound of claim 75, wherein the nutritional composition is a liquid dietary supplement comprising 1500 calories per liter with a caloric distribution of 14.7% protein, 32% fat and 53.3% carbohydrate.

78. The compound of any one of claims 75 to 77, wherein the nutritional composition is administered in an amount of about 6 to about 12 fluid ounces.

79. The compound of claim 78, wherein the nutritional composition is administered in an amount of about 8 fluid ounces.

80. The compound of any one of claims 75 to 79, wherein the nutritional composition is administered within 30 minutes of each administration of the compound of Formula (I), or a pharmaceutically acceptable salt thereof.

81. The compound of any one of claims 1 to 80, wherein the compound of Formula (I), or a pharmaceutically acceptable salt thereof, is administered to the subject with a meal.

82. The compound of claim 81, wherein the meal is a high fat meal.

83. The compound of claim 81, wherein the meal is a low fat meal.

84. The compound of any one of claims 81 to 83, wherein the meal is completed within about 30 minutes after administration of the compound of Formula (I), or a pharmaceutically acceptable salt thereof.

85. The compound of any one of claims 1 to 74 and 81 to 84, wherein the first administration of the compound of Formula (I), or a pharmaceutically acceptable salt thereof is with a morning meal.

86. The compound of any one of claims 1 to 74 and 81 to 84, wherein the second administration of the compound of Formula (I), or a pharmaceutically acceptable salt thereof is with an evening meal.

87. The compound of any one of claims 1 to 74 and 81 to 84, wherein the first administration of the compound of Formula (I), or a pharmaceutically acceptable salt thereof, is with a morning meal and the second administration of the compound of Formula (I), or a pharmaceutically acceptable salt thereof is with an evening meal.

88. The compound of any one of claims 1 to 87, wherein there are about 6 to about 14 hours between the first and second administrations of the compound of Formula (I), or a pharmaceutically acceptable salt thereof.

89. The compound of any one of claims 1 to 87, wherein there are about 8 to about 14 hours between the first and second administrations of the compound of Formula (I), or a pharmaceutically acceptable salt thereof.

90. The compound of any one of claims 1 to 87, wherein there are about 11 to about 13 hours between the first and second administrations of the compound of Formula (I), or a pharmaceutically acceptable salt thereof.

91. The compound of any one of claims 1 to 87, wherein there are about 12 hours between the first and second administrations of the compound of Formula (I), or a pharmaceutically acceptable salt thereof.

92. The compound of any one of claims 1 to 91, wherein the subject is concurrently receiving a dose of a glucocorticoid.

93. The compound of claim 92, wherein the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.

94. The compound of claim 92 or 93, wherein the glucocorticoid is cortisol (hydrocortisone).

95. The compound of claim 92 or 93, wherein the glucocorticoid is cortisone.

96. The compound of claim 92 or 93, wherein the glucocorticoid is prednisone.

97. The compound of any one of claims 92 to 96, wherein the glucocorticoid dose is measured in hydrocortisone equivalents.

98. The compound of any one of claims 92 to 96, wherein the glucocorticoid dose is measured as a multiple of the upper limit of normal of physiologic dosing in hydrocortisone equivalents.

99. The compound of any one of claims 92 to 96, wherein the glucocorticoid dose is a physiologic dose as measured after a time period of administration of the compound of Formula (I), or a pharmaceutically acceptable salt thereof.

100. The compound of any one of claims 92 to 96, wherein the glucocorticoid dose is a physiologic dose of about 4 to about 12 mg/m²/day as measured after a time period of administration of the compound of Formula (I), or a pharmaceutically acceptable salt thereof.

101. The compound of any one of claims 92 to 96, wherein the glucocorticoid dose is a physiologic dose of about 4 to about 9 mg/m²/day as measured after a time period of administration of the compound of Formula (I), or a pharmaceutically acceptable salt thereof.

102. The compound of any one of claims 92 to 96, wherein the glucocorticoid dose is a physiologic dose that is less than about 8 mg/m²/day as measured after a time period of administration of the compound of Formula (I), or a pharmaceutically acceptable salt thereof.

103. The compound of any one of claims 92 to 102, wherein the glucocorticoid dose of the subject is reduced by about 10% after a time period of administration of the compound of Formula (I), or a pharmaceutically acceptable salt thereof, wherein the reduction of the glucocorticoid dose is relative to the glucocorticoid dose prior to administration of the compound of Formula (I).

104. The compound of any one of claims 92 to 102, wherein the glucocorticoid dose of the subject is reduced by about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

105. The compound of any one of claims 92 to 102, wherein the glucocorticoid dose of the subject is reduced by about 30% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

106. The compound of any one of claims 92 to 102, wherein the glucocorticoid dose of the subject is reduced by about 40% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

107. The compound of any one of claims 92 to 102, wherein the glucocorticoid dose of the subject is reduced by about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

108. The compound of any one of claims 92 to 102, wherein the glucocorticoid dose of the subject is reduced by about 60% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

109. The compound of any one of claims 92 to 102, wherein the glucocorticoid dose of the subject is reduced by about 70% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

110. The compound of any one of claims 92 to 102, wherein the glucocorticoid dose of the subject is reduced by less than about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

111. The compound of any one of claims 92 to 102, wherein the glucocorticoid dose of the subject is reduced by about 20% to about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

112. The compound of any one of claims 92 to 102, wherein the glucocorticoid dose of the subject is reduced by greater than about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

113. The compound of any one of claims 1 to 112, wherein the level of 17-hydroxyprogesterone is less than about 1.5 times the upper limit of normal after a time period of administration of the compound of Formula (I), or a pharmaceutically acceptable salt thereof.

114. The compound of any one of claims 1 to 113, wherein the level of 17-hydroxyprogesterone is within normal limits after a time period of administration of the compound of Formula (I), or a pharmaceutically acceptable salt thereof.

115. The compound of any one of claims 1 to 114, wherein the level of adrenocorticotropic hormone is less than about 1.5 times the upper limit of normal after a time period of administration of the compound of Formula (I), or a pharmaceutically acceptable salt thereof.

116. The compound of any one of claims 1 to 115, wherein the level of adrenocorticotropic hormone is within normal limits after a time period of administration of the compound of Formula (I), or a pharmaceutically acceptable salt thereof.

117. The compound of any one of claims 1 to 116, wherein the level of androstenedione is less than about 1.5 times the upper limit of normal after a time period of administration of the compound of Formula (I), or a pharmaceutically acceptable salt thereof.

118. The compound of any one of claims 1 to 117, wherein the level of androstenedione is within normal limits after a time period of administration of the compound of Formula (I), or a pharmaceutically acceptable salt thereof.

119. The compound of any one of claims 1 to 118, wherein the level of testosterone is reduced by at least about 25% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

120. The compound of any one of claims 1 to 118, wherein the level of testosterone is reduced by at least about 30% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

121. The compound of any one of claims 1 to 118, wherein the level of testosterone is reduced by at least about 50% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

122. The compound of any one of claims 1 to 121, wherein the level of testosterone is less than about 1.5 times the upper limit of normal after a time period of administration of the pharmaceutical composition.

123. The compound of any one of claims 1 to 122, wherein the level of testosterone is within normal limits after a time period of administration of the pharmaceutical composition comprising the compound of Formula (I), or a pharmaceutically acceptable salt thereof.

124. The compound of any one of claims 92 to 123, wherein the subject exhibits a decrease in glucocorticoid burden after a time period of administration of the compound of Formula (I), or a pharmaceutically acceptable salt thereof, wherein the decrease in glucocorticoid burden is relative to the glucocorticoid burden prior to administration of the compound of Formula (I), or a pharmaceutically acceptable salt thereof.

125. The compound of claim 124, wherein one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor, control of ovarian adrenal rest tumor, and fertility, is improved after a time period of administration of the compound of Formula (I), or a pharmaceutically acceptable salt thereof, wherein the improvement in the one or more symptoms is relative to the status of the one or more symptoms prior to administration of the compound of Formula (I), or a pharmaceutically acceptable salt thereof.

126. The compound of any one of claims 99 to 125, wherein the time period of administration is at least about 4 weeks.

127. The compound of any one of claims 99 to 125, wherein the time period of administration is at least about 24 weeks.

128. The compound of any one of claims 99 to 125, wherein the time period of administration is at least about one year.

129. A compound of Formula (I): 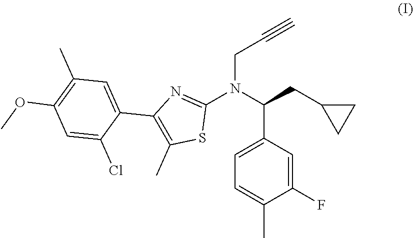 [SMILES: C#CCN(c1nc(-c2cc(C)c(OC)cc2Cl)c(C)s1)[C@@H](CC1CC1)c1ccc(C)c(F)c1]
or a pharmaceutically acceptable thereof,
for use in a method of treating congenital adrenal hyperplasia in a subject, the method comprising:
(a) selecting a subject who has a glucocorticoid dose of greater than 11 mg/m²/day after a time period of being administered a compound of Formula (I), or a pharmaceutically acceptable thereof, at an amount of about 100 mg twice daily based on the weight of the free base;
and
(b) administering to the subject the compound of Formula (I), or a pharmaceutically acceptable salt thereof, at a frequency of twice daily;
wherein the amount of the compound of Formula (I), or a pharmaceutically acceptable salt thereof, in the first administration is less than the amount of the compound of Formula (I), or a pharmaceutically acceptable salt thereof, in the second administration;
and wherein the amount of the compound of Formula (I), or a pharmaceutically acceptable salt thereof, administered daily is greater than or equal to about 200 mg based on the weight of the free base.

130. The compound of claim 129, wherein the time period of administration in step (a) is at least about 4 weeks.

131. The compound of claim 129, wherein the time period of administration in step (a) is at least about 24 weeks.

132. The compound of claim 129, wherein the time period of administration in step (a) is at least about one year.

133. The compound of any one of claims 1 to 132, wherein the subject is female.

134. The compound of any one of claims 1 to 132, wherein the subject is male.

135. The compound of any one of claims 1 to 134, wherein the compound of Formula (I) is administered as a pharmaceutical composition comprising: (a) the compound of Formula (I), or a pharmaceutically acceptable salt thereof; and (b) one or more of an oily phase vehicle, an emulsifying agent, a nonionic surfactant, and a solubilizing agent.

136. The compound of claim 135, wherein the pharmaceutical composition comprises about 1 wt % to about 20 wt % of the compound of Formula (I), or a pharmaceutically acceptable salt thereof, based on the weight of the free base.

137. The compound of claim 135, wherein the pharmaceutical composition comprises about 5 wt % to about 15 wt % of the compound of Formula (I), or a pharmaceutically acceptable salt thereof, based on the weight of the free base.

138. The compound of claim 135, wherein the pharmaceutical composition comprises about 10 wt % of the compound of Formula (I), or a pharmaceutically acceptable salt thereof, based on the weight of the free base.

139. The compound of any one of claims 135 to 138, wherein the pharmaceutical composition comprises an oily phase vehicle.

140. The compound of any one of claims 135 to 139, wherein the pharmaceutical composition comprises about 1 wt % to about 50 wt % of the oily phase vehicle.

141. The compound of any one of claims 135 to 139, wherein the pharmaceutical composition comprises about 20 wt % to about 50 wt % of the oily phase vehicle.

142. The compound of any one of claims 135 to 139, wherein the pharmaceutical composition comprises about 35 wt % to about 45 wt % of the oily phase vehicle.

143. The compound of any one of claims 135 to 139, wherein the pharmaceutical composition comprises about 39 wt % of the oily phase vehicle.

144. The compound of any one of claims 135 to 143, wherein the oily phase vehicle is selected from medium-chain triglycerides, glycerin, propylene glycol, polyethylene glycol, olive oil, soybean oil, corn oil, and transcutol.

145. The compound of any one of claims 135 to 144, wherein the oily phase vehicle is medium-chain triglycerides.

146. The compound of claim 145, wherein the medium-chain triglycerides are Labrafac™ Lipophile WL1349.

147. The compound of claim 145, wherein the medium-chain triglycerides are Miglyol 812N.

148. The compound of any one of claims 135 to 147, wherein the pharmaceutical composition comprises an emulsifying agent.

149. The compound of any one of claims 135 to 148, wherein the pharmaceutical composition comprises about 5 wt % to about 50 wt % of the emulsifying agent.

150. The compound of any one of claims 135 to 148, wherein the pharmaceutical composition comprises about 10 wt % to about 30 wt % of the emulsifying agent.

151. The compound of any one of claims 135 to 148, wherein the pharmaceutical composition comprises about 15 wt % to about 25 wt % of the emulsifying agent.

152. The compound of any one of claims 135 to 148, wherein the pharmaceutical composition comprises about 20 wt % of the emulsifying agent.

153. The compound of any one of claims 135 to 152, wherein the emulsifying agent is selected from medium-chain triglycerides, propylene glycol dicaprylate/dicaprate, glycerin, propylene glycol, polyethylene glycol, olive oil, soybean oil, corn oil, and transcutol.

154. The compound of any one of claims 135 to 153, wherein the emulsifying agent is propylene glycol dicaprylate/dicaprate.

155. The compound of claim 154, wherein the propylene glycol dicaprylate/dicaprate is Labrafac™ PG.

156. The compound of any one of claims 135 to 155, wherein the pharmaceutical composition comprises a nonionic surfactant.

157. The compound of any one of claims 135 to 156, wherein the pharmaceutical composition comprises about 5 wt % to about 50 wt % of the nonionic surfactant.

158. The compound of any one of claims 135 to 156, wherein the pharmaceutical composition comprises about 10 wt % to about 30 wt % of the nonionic surfactant.

159. The compound of any one of claims 135 to 156, wherein the pharmaceutical composition comprises about 15 wt % to about 25 wt % of the nonionic surfactant.

160. The compound of any one of claims 135 to 156, wherein the pharmaceutical composition comprises about 19 wt % of the nonionic surfactant.

161. The compound of any one of claims 135 to 160, wherein the nonionic surfactant is selected from oleoyl polyoxyl-6 glycerides, linoleoyl polyoxyl-6 glycerides, Polysorbate 80, Polysorbate 20, Gelucire, lauroyl polyoxyl-32 glycerides, Poloxamer, PEG-32 stearate, and PEG-32 hydrogenated palm glycerides.

162. The compound of any one of claims 135 to 161, wherein the nonionic surfactant is lauroyl polyoxyl-32 glycerides.

163. The compound of claim 162, wherein the lauroyl polyoxyl-32 glycerides are Gelucire® 44/14.

164. The compound of any one of claims 135 to 163, wherein the pharmaceutical composition comprises a solubilizing agent.

165. The compound of any one of claims 135 to 164, wherein the pharmaceutical composition comprises about 1 wt % to about 50 wt % of the solubilizing agent.

166. The compound of any one of claims 135 to 164, wherein the pharmaceutical composition comprises about 1 wt % to about 20 wt % of the solubilizing agent.

167. The compound of any one of claims 135 to 164, wherein the pharmaceutical composition comprises about 5 wt % to about 15 wt % of the solubilizing agent.

168. The compound of any one of claims 135 to 164, wherein the pharmaceutical composition comprises about 11 wt % of the solubilizing agent.

169. The compound of any one of claims 135 to 168, wherein the solubilizing agent is selected from oleoyl polyoxyl-6 glycerides, linoleoyl polyoxyl-6 glycerides, Polysorbate 80, Polysorbate 20, vitamin E polyethylene glycol succinate, Gelucire, lauroyl polyoxyl-32 glycerides, and Poloxamer.

170. The compound of any one of claims 135 to 169, wherein the solubilizing agent is vitamin E polyethylene glycol succinate.

171. The compound of claim 170, wherein the vitamin E polyethylene glycol succinate is Kolliphor® TPGS.

172. The compound of claim 170, wherein the vitamin E polyethylene glycol succinate is Vitamin E/TPGS 260.

173. The compound of claim 135, wherein the pharmaceutical composition comprises:
(a) the compound of Formula (I), or a pharmaceutically acceptable salt thereof,
(b) an oily phase vehicle;
(c) an emulsifying agent;
(d) a nonionic surfactant; and
(e) a solubilizing agent.

174. The compound of claim 135, wherein the pharmaceutical composition comprises:
(a) about 5 wt % to about 15 wt % of the compound of Formula (I), or a pharmaceutically acceptable salt thereof, based on the weight of the free base;
(b) about 35 wt % to about 45 wt % of an oily phase vehicle;
(c) about 15 wt % to about 25 wt % of an emulsifying agent;
(d) about 15 wt % to about 25 wt % of a nonionic surfactant; and
(e) about 5 wt % to about 15 wt % of a solubilizing agent.

175. The compound of claim 135, wherein the pharmaceutical composition comprises:
(a) about 10 wt % of the compound of Formula (I), or a pharmaceutically acceptable salt thereof, based on the weight of the free base;
(b) about 39 wt % of an oily phase vehicle;
(c) about 20 wt % of an emulsifying agent;
(d) about 19 wt % of a nonionic surfactant; and
(e) about 11 wt % of a solubilizing agent.

176. The compound of claim 135, wherein the pharmaceutical composition comprises:
(a) the compound of Formula (I);
(b) a medium-chain triglycerides component;
(c) a propylene glycol dicaprylate/dicaprate component;
(d) a lauroyl polyoxyl-32 glycerides component; and
(e) a vitamin E polyethylene glycol succinate component.

177. The compound of claim 135, wherein the pharmaceutical composition comprises:
(a) about 5 wt % to about 15 wt % of the compound of Formula (I);
(b) about 35 wt % to about 45 wt % of medium-chain triglycerides;
(c) about 15 wt % to about 25 wt % of propylene glycol dicaprylate/dicaprate;
(d) about 15 wt % to about 25 wt % of lauroyl polyoxyl-32 glycerides; and
(e) about 5 wt % to about 15 wt % of vitamin E polyethylene glycol succinate.

178. The compound of claim 135, wherein the pharmaceutical composition comprises:
(a) about 10 wt % of the compound of Formula (I);
(b) about 39 wt % of medium-chain triglycerides;
(c) about 20 wt % of propylene glycol dicaprylate/dicaprate;
(d) about 19 wt % of lauroyl polyoxyl-32 glycerides; and
(e) about 11 wt % of vitamin E polyethylene glycol succinate.

179. The compound of any one of claims 135 to 178, wherein the pharmaceutical composition comprises the compound of Formula (I), or pharmaceutically acceptable salt thereof, in crystalline form.

180. The compound of any one of claims 135 to 179, wherein the pharmaceutical composition comprises the compound of Formula (I) as a free base.

181. The compound of claim 180, wherein the crystalline form comprises Form I of the compound of Formula (I) as a free base.

182. The compound of any one of claims 135 to 181, wherein the pharmaceutical composition is formulated in unit dosage form, wherein the compound of Formula (I), or a pharmaceutically acceptable salt thereof, is present in an amount of about 5 mg to about 200 mg, based on the weight of the free base.

183. The compound of claim 182, wherein the compound of Formula (I), or a pharmaceutically acceptable salt thereof, is present in the unit dosage form in an amount of about 25 mg to about 150 mg, based on the weight of the free base.

184. The compound of claim 182, wherein the compound of Formula (I), or a pharmaceutically acceptable salt thereof, is present in the unit dosage form in an amount of about 50 mg, based on the weight of the free base.

185. The compound of claim 182, wherein the compound of Formula (I), or a pharmaceutically acceptable salt thereof, is present in the unit dosage form in an amount of about 100 mg, based on the weight of the free base.

186. The compound of claim 182, wherein the compound of Formula (I), or a pharmaceutically acceptable salt thereof, is present in the unit dosage form in an amount of about 25 mg, based on the weight of the free base.

187. The compound of any one of claims 135 to 186, wherein the pharmaceutical composition is in the form of a tablet, capsule, sachet, powder, granules, coated particle, coated tablet, enterocoated tablet, enterocoated capsule, melting strip, or melting film.

188. The compound of claim 187, wherein the pharmaceutical composition is in tablet form.

189. The compound of claim 187, wherein the pharmaceutical composition is in capsule form.

190. The compound of any one of claims 188 to 189, wherein the tablet form or capsule form is coated.

191. The compound of any one of claims 1 to 134, wherein the compound of Formula (I) is administered as a pharmaceutical composition in oral solution dosage form comprising:
(a) the compound of Formula (I), or a pharmaceutically acceptable salt thereof,
(b) one or more of a sweetener, an anti-oxidant, and a flavor; and
(c) a liquid vehicle.

192. The compound of claim 191, wherein the pharmaceutical composition comprises:
(a) the compound of Formula (I), or a pharmaceutically acceptable salt thereof,
(b) a sweetener;
(c) an anti-oxidant;
(d) a flavor; and
(e) a liquid vehicle.

193. The compound of claim 192, wherein the pharmaceutical composition further comprises a surfactant.

194. The compound of claim 191, wherein the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor;
(e) about 15 w/v % to about 25 w/v % of a surfactant; and
(f) about 70 w/v % to about 80 w/v % of a liquid vehicle.

195. The compound of claim 191, wherein the pharmaceutical composition comprises:
(a) about 5 w/v % of the compound of Formula (I), or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor;
(e) about 20 w/v % of a surfactant; and
(f) about 75 w/v % of a liquid vehicle.

196. The compound of claim 191, wherein the pharmaceutical composition comprises:
(a) a compound of Formula (I), or a pharmaceutically acceptable salt thereof;
(b) saccharin;
(c) butylated hydroxytoluene;
(d) FONA orange flavor; and
(e) medium-chain triglycerides.

197. The compound of claim 196, wherein the pharmaceutical composition further comprises oleoyl polyoxyl-6 glycerides.

198. The compound of claim 191, wherein the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor;
(e) about 15 w/v % to about 25 w/v % of oleoyl polyoxyl-6 glycerides; and
(f) about 70 w/v % to about 80 w/v % of medium-chain triglycerides.

199. The compound of claim 191, wherein the pharmaceutical composition comprises:
(a) about 5 w/v % of the compound of Formula (I), or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor;
(e) about 20 w/v % of oleoyl polyoxyl-6 glycerides; and
(f) about 75 w/v % of medium-chain triglycerides.

200. The compound of any one of claims 191 to 199, wherein the pharmaceutical composition comprises the compound of Formula (I) as a free base.

201. The compound of any one of claims 191 to 200, wherein the pharmaceutical composition is formulated in unit dosage form, wherein the compound of Formula (I), or a pharmaceutically acceptable salt thereof, is present in an amount of about 5 mg/mL to about 200 mg/mL, based on the weight of the free base.

202. The compound of claim 201, wherein the unit dosage form comprises the compound of Formula (I), or a pharmaceutically acceptable salt thereof, in an amount of about 50 mg/mL, based on the weight of the free base.

203. The compound of any one of claims 1 to 134, wherein the compound of Formula (I), or a pharmaceutically acceptable salt thereof, is administered in a pharmaceutical composition comprising a spray-dried dispersion comprising:
a compound of Formula (I), or a pharmaceutically acceptable salt thereof; and
a polymer selected from a neutral polymer, an enteric polymer, and a pyrrolidone polymer;
wherein the weight ratio of the compound of Formula (I) to the polymer is from about 1:1 to about 1:9.

204. The compound of claim 203, wherein the spray-dried dispersion comprises:
a compound of Formula (I), or a pharmaceutically acceptable salt thereof; and
a polymer that is a copolymer of 1-vinyl-2-pyrrolidone and vinyl acetate having the structure: 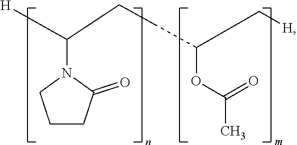
wherein the value of n is about 1 to about 2 times the value of m and the copolymer comprises 1-vinyl-2-pyrrolidone and vinyl acetate at a ratio of about 60:40 by weight; and
wherein the weight ratio of the compound of Formula (I) to the copolymer is from about 1:1 to about 1:9.

205. The compound of claim 203, wherein the pharmaceutical composition comprises:
(a) the spray-dried dispersion of Example 3;
(b) calcium silicate;
(c) a combination of mannitol and microcrystalline cellulose; and
(d) croscarmellose sodium.

206. The compound of claim 203, wherein the pharmaceutical composition comprises:
(a) about 1 w/w % to about 20 w/w % of the spray-dried dispersion of Example 3;
(b) about 0.1 w/w % to about 1 w/w % of calcium silicate;
(c) about 50 w/w % to about 60 w/w % of mannitol and about 10 w/w % to about 30 w/w % of microcrystalline cellulose; and
(d) about 5 w/w % to about 0.2 w/w % of croscarmellose sodium.

207. The compound of claim 203, wherein the pharmaceutical composition comprises:
(a) about 13 w/w % of the spray-dried dispersion of Example 3;
(b) about 0.67 w/w % of calcium silicate;
(c) about 56 w/w % of mannitol and about 20 w/w % of microcrystalline cellulose; and
(d) about 10 w/w % of croscarmellose sodium.

208. The compound of any one of claims 203 to 207, wherein the pharmaceutical composition is formulated as a tablet, capsule, sachet, powder, granules, coated particle, coated tablet, enterocoated tablet, enterocoated capsule, melting strip, or melting film.

209. The compound of claim 208, wherein the pharmaceutical composition is in capsule form.

210. A method of treating congenital adrenal hyperplasia in a subject in need thereof comprising administering to the subject a compound of Formula (I): 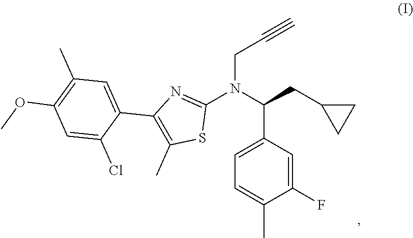 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

211. The method of claim 210, wherein the compound of Formula (I), or a pharmaceutically acceptable salt thereof, is administered at a frequency of twice daily.

212. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100.

213. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:50.

214. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:10.

215. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:5.

216. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:3.

217. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.5.

218. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.

219. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5.

220. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.

221. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.5.

222. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration about 1:3.

223. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:3.5.

224. The method of claim 210 or 211, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:4.

225. The method of any one of claims 210 to 224, wherein the amount of the compound of Formula (I), or a pharmaceutically acceptable salt thereof, administered daily is less than or equal to about 1000 mg based on the weight of the free base.

226. The method of any one of claims 210 to 224, wherein the amount of the compound of Formula (I), or a pharmaceutically acceptable salt thereof, administered daily is from about 50 mg to about 1000 mg based on the weight of the free base.

227. The method of any one of claims 210 to 224, wherein the amount of the compound of Formula (I), or a pharmaceutically acceptable salt thereof, administered daily is from about 100 mg to about 1000 mg based on the weight of the free base.

228. The method of any one of claims 210 to 224, wherein the amount of the compound of Formula (I), or a pharmaceutically acceptable salt thereof, administered daily is from about 100 mg to about 500 mg based on the weight of the free base.

229. The method of any one of claims 210 to 224, wherein the amount of the compound of Formula (I), or a pharmaceutically acceptable salt thereof, administered daily is from about 100 mg to about 400 mg based on the weight of the free base.

230. The method of any one of claims 210 to 224, wherein the amount of the compound of Formula (I), or a pharmaceutically acceptable salt thereof, administered daily is from about 100 mg to about 300 mg based on the weight of the free base.

231. The method of any one of claims 210 to 224, wherein the amount of the compound of Formula (I), or a pharmaceutically acceptable salt thereof, administered daily is about 200 mg based on the weight of the free base.

232. The method of claim 231, wherein the first administration of the compound of Formula (I), or a pharmaceutically acceptable salt thereof is about 50 mg based on the weight of the free base and the second administration of the compound of Formula (I), or a pharmaceutically acceptable salt thereof is about 150 mg based on the weight of the free base.

233. The method of any one of claims 210 to 224, wherein the amount of the compound of Formula (I), or a pharmaceutically acceptable salt thereof, administered daily is about 250 mg based on the weight of the free base.

234. The method of claim 233, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

235. The method of any one of claims 210 to 224, wherein the amount of the compound of Formula (I), or a pharmaceutically acceptable salt thereof, administered daily is about 300 mg based on the weight of the free base.

236. The method of claim 26, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base.

237. The method of any one of claims 210 to 236, wherein the subject weighs greater than or equal to about 55 kg.

238. The method of any one of claims 210 to 236, wherein the amount of the compound of Formula (I), or a pharmaceutically acceptable salt thereof, administered daily is about 50 mg based on the weight of the free base.

239. The method of claim 238, wherein the subject weighs from about 10 kg to about 20 kg.

240. The method of any one of claims 210 to 236, wherein the amount of the compound of Formula (I), or a pharmaceutically acceptable salt thereof, administered daily is about 100 mg based on the weight of the free base.

241. The method of claim 240, wherein the wherein the first administration of the compound of Formula (I), or a pharmaceutically acceptable salt thereof is about 25 mg based on the weight of the free base and the second administration of the compound of Formula (I), or a pharmaceutically acceptable salt thereof is about 75 mg based on the weight of the free base.

242. The method of claim 240 or 241, wherein the subject weighs from about 20 kg to about 55 kg.

243. A method of treating congenital adrenal hyperplasia in a subject in need thereof comprising administering to the subject a compound of Formula (I): 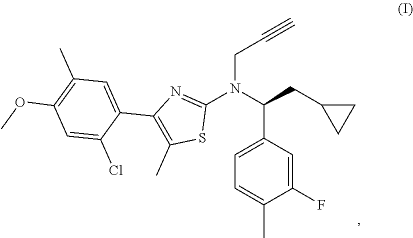 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
wherein the compound of Formula (I), or a pharmaceutically acceptable salt thereof, is administered at a frequency of not less than twice daily; and
wherein the amount of the compound of Formula (I), or a pharmaceutically acceptable salt thereof, administered daily is greater than 200 mg based on the weight of the free base.

244. The method of claim 243, wherein the amount of the compound of Formula (I), or a pharmaceutically acceptable salt thereof, is administered at a frequency of twice daily.

245. The method of claim 243 or 244, wherein the amount of the compound of Formula (I), or a pharmaceutically acceptable salt thereof, administered daily is from about 200 mg to about 1000 mg based on the weight of the free base.

246. The method of claim 243 or 244, wherein the amount of the compound of Formula (I), or a pharmaceutically acceptable salt thereof, administered daily is from about 225 mg to about 1000 mg based on the weight of the free base.

247. The method of claim 243 or 244, wherein the amount of the compound of Formula (I), or a pharmaceutically acceptable salt thereof, administered daily is from about 225 mg to about 500 mg based on the weight of the free base.

248. The method of claim 243 or 244, wherein the amount of the compound of Formula (I), or a pharmaceutically acceptable salt thereof, administered daily is from about 225 mg to about 400 mg based on the weight of the free base.

249. The method of claim 243 or 244, wherein the amount of the compound of Formula (I), or a pharmaceutically acceptable salt thereof, administered daily is from about 225 mg to about 300 mg based on the weight of the free base.

250. The method of claim 243 or 244, wherein the amount of the compound of Formula (I), or a pharmaceutically acceptable salt thereof, administered daily is about 250 mg.

251. The method of claim 243 or 244, wherein the first administration of the compound of Formula (I), or a pharmaceutically acceptable salt thereof is about 125 mg based on the weight of the free base and the second administration of the compound of Formula (I), or a pharmaceutically acceptable salt thereof is about 125 mg based on the weight of the free base.

252. The method of claim 243 or 244, wherein the amount of the compound of Formula (I), or a pharmaceutically acceptable salt thereof, administered daily is about 300 mg based on the weight of the free base.

253. The method of claim 252, wherein the first administration of the compound of Formula (I), or a pharmaceutically acceptable salt thereof is about 150 mg based on the weight of the free base and the second administration of the compound of Formula (I), or a pharmaceutically acceptable salt thereof is about 150 mg based on the weight of the free base.

254. The method of any one of claims 210 to 253, wherein the compound of Formula (I), or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject.

255. The method of claim 254, wherein the reduction in level of any of biomarkers is determined by comparing the level of the biomarker as measured during the circadian release during a time period prior to administering the compound of Formula (I), or a pharmaceutically acceptable salt thereof and the level of the biomarker as measured during the circadian release on a day after administering the compound of Formula (I), or a pharmaceutically acceptable salt thereof.

256. The method of claim 255, wherein the circadian release occurs between the hours of 2 a.m. and 10 a.m.

257. The method of any one of claims 254 to 256, wherein the level of 17-hydroxyprogesterone is reduced by at least 25%.

258. The method of any one of claims 254 to 256, wherein the level of 17-hydroxyprogesterone is reduced by at least 50%.

259. The method of any one of claims 254 to 258, wherein the level of adrenocorticotropic hormone is reduced by at least 25%.

260. The method of any one of claims 254 to 258, wherein the level of adrenocorticotropic hormone is reduced by at least 40%.

261. The method of any one of claims 254 to 258, wherein the level of adrenocorticotropic hormone is reduced by at least 50%.

262. The method of any one of claims 254 to 261, wherein the level of androstenedione is reduced by at least 25%.

263. The method of any one of claims 254 to 261, wherein the level of androstenedione is reduced by at least 30%.

264. The method of any one of claims 254 to 261, wherein the level of androstenedione is reduced by at least 50%.

265. The method of any one of claims 254 to 264, wherein the level of 17-hydroxyprogesterone is reduced by at least 50% and the level of androstenedione is reduced by at least 50%.

266. A method for reducing the severity of one or more symptoms selected from hirsutism, precocious puberty, fertility problems, acne, and growth impairment in a subject having classic congenital adrenal hyperplasia,
comprising administering to the subject a compound of Formula (I): 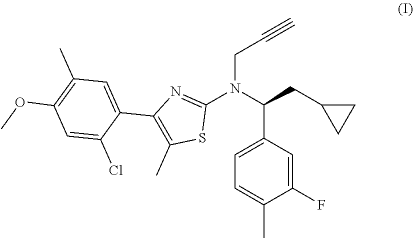 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

267. The method of claim 266, wherein the total daily amount of the compound of Formula (I) is sufficient to reduce the level of androstenedione in the subject.

268. The method of claim 266 or 267, wherein the growth impairment is selected from one or more of accelerated height velocity, accelerated weight velocity, or accelerated bone age.

269. The method of claim 267 or 268, wherein the androstenedione is reduced by at least 25%.

270. The method of claim 267 or 268, wherein the androstenedione is reduced by at least 30%.

271. The method of claim 267 or 268, wherein the androstenedione is reduced by at least 50%.

272. A method of reducing the level of one or more biomarkers of congenital adrenal hyperplasia in a subject having congenital adrenal hyperplasia comprising administering to the subject a compound of Formula (I): 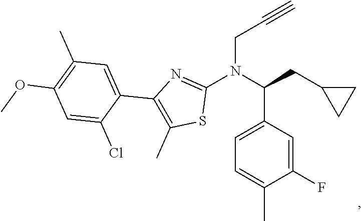 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

273. The method of claim 272, wherein the one or more biomarkers of congenital adrenal hyperplasia are selected from (a) 17-hydroxyprogesterone (17-OHP); (b) adrenocorticotropic hormone (ACTH); and (c) androstenedione.

274. A method of reducing the dosage of corticosteroid administered to a subject having congenital adrenal hyperplasia for controlling congenital adrenal hyperplasia comprising administering to the subject a compound of Formula (I): 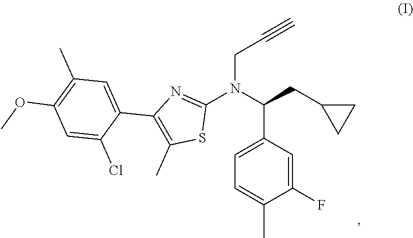 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

275. The method of claim 274, wherein the corticosteroid is a glucocorticoid.

276. A method of reducing the severity of one or more side effects of glucocorticoid treatment in a subject having congenital adrenal hyperplasia comprising administering to the subject a compound of Formula (I): 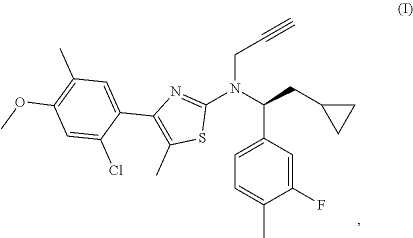 [SMILES: C#CCN(c1nc(-c2cc(C)c(OC)cc2Cl)c(C)s1)[C@@H](CC1CC1)c1ccc(C)c(F)c1]
or a pharmaceutically acceptable salt thereof,
wherein the compound of Formula (I), or a pharmaceutically acceptable salt thereof, is administered at a frequency of not less than twice daily;
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations; and
wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

277. The method of any one of claims 272 to 276, wherein the compound of Formula (I), or a pharmaceutically acceptable salt thereof, is administered at an amount sufficient to reduce the level of 17-hydroxyprogesterone (17-OHP) by at least 50% as compared to the level prior to administration.

278. The method of any one of claims 272 to 277, wherein the compound of Formula (I), or a pharmaceutically acceptable salt thereof is administered at an amount sufficient to reduce the level of androstenedione by at least 30% as compared to the level prior to administration.

279. The method of any one of claims 272 to 277, wherein the compound of Formula (I), or a pharmaceutically acceptable salt thereof is administered at an amount sufficient to (a) reduce the level of 17-hydroxyprogesterone (17-OHP) by at least 50% as compared to the level prior to administration; and (b) reduce the level of androstenedione by at least 30% as compared to the level prior to administration.

280. The method of any one of claims 266 to 279, wherein the compound of Formula (I), or a pharmaceutically acceptable salt thereof, is administered twice daily at an amount from about 25 mg to about 250 mg based on the weight of the free base.

281. The method of any one of claims 266 to 279, wherein the compound of Formula (I), or a pharmaceutically acceptable salt thereof, is administered twice daily as:
(a) a first administration of about 50 mg and a second administration of about 150 mg based on the weight of the free base; or
(b) a first administration of about 100 mg and a second administration of about 150 mg based on the weight of the free base; or
(c) a first administration of about 100 mg and a second administration of about 200 mg based on the weight of the free base.

282. The method of any one of claims 210 to 281, wherein the compound of Formula (I) is administered in the free base form.

283. The method of any one of claims 210 to 282, wherein the subject is in a fed state.

284. The method of claim 283, wherein the compound of Formula (I), or a pharmaceutically acceptable salt thereof, is administered to the subject with a nutritional composition.

285. The method of claim 284, wherein the nutritional composition is a liquid dietary supplement comprising about 1000 to about 2000 calories per liter with a fat content greater than about 30%.

286. The method of claim 284, wherein the nutritional composition is a liquid dietary supplement comprising 1500 calories per liter with a caloric distribution of 14.7% protein, 32% fat and 53.3% carbohydrate.

287. The method of any one of claims 284 to 286, wherein the nutritional composition is administered in an amount of about 6 to about 12 fluid ounces.

288. The method any one of claims 284 to 286, wherein the nutritional composition is administered in an amount of about 8 fluid ounces.

289. The method of any one of claims 284 to 288, wherein the nutritional composition is administered within 30 minutes of each administration of the compound of Formula (I), or a pharmaceutically acceptable salt thereof.

290. The method of any one of claims 210 to 282, wherein the compound of Formula (I), or a pharmaceutically acceptable salt thereof, is administered to the subject with a meal.

291. The method of claim 290, wherein the meal is a high fat meal.

292. The method of claim 290, wherein the meal is a low fat meal.

293. The method of any one of claims 290 to 292, wherein the wherein the meal is completed within about 30 minutes after administration of the compound of Formula (I), or a pharmaceutically acceptable salt thereof.

294. The method of any one of claims 210 to 283 and 290 to 293, wherein the first administration of the compound of Formula (I), or a pharmaceutically acceptable salt thereof is with a morning meal.

295. The method of any one of claims 210 to 283 and 290 to 294, wherein the second administration of the compound of Formula (I), or a pharmaceutically acceptable salt thereof is with an evening meal.

296. The method of any one of claims 210 to 283 and 290 to 295, wherein the first administration of the compound of Formula (I), or a pharmaceutically acceptable salt thereof, is with a morning meal and the second administration of the compound of Formula (I), or a pharmaceutically acceptable salt thereof is with an evening meal.

297. The method of any one of claims 210 to 296, wherein there are about 6 to about 14 hours between the first and second administrations of the compound of Formula (I), or a pharmaceutically acceptable salt thereof.

298. The method of any one of claims 210 to 296, wherein there are about 8 to about 14 hours between the first and second administrations of the compound of Formula (I), or a pharmaceutically acceptable salt thereof.

299. The method of any one of claims 210 to 296, wherein there are about 11 to about 13 hours between the first and second administrations of the compound of Formula (I), or a pharmaceutically acceptable salt thereof.

300. The method of any one of claims 210 to 296, wherein there are about 12 hours between the first and second administrations of the compound of Formula (I), or a pharmaceutically acceptable salt thereof.

301. The method of any one of claims 210 to 300, wherein the subject is concurrently receiving a dose of a glucocorticoid.

302. The method of claim 301, wherein the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.

303. The method of claim 301 or 302, wherein the glucocorticoid is cortisol (hydrocortisone).

304. The method of claim 301 or 302, wherein the glucocorticoid is cortisone.

305. The method of claim 301 or 302, wherein the glucocorticoid is prednisone.

306. The method of any one of claims 301 to 305, wherein the glucocorticoid dose is measured in hydrocortisone equivalents.

307. The method of any one of claims 301 to 305, wherein the glucocorticoid dose is measured as a multiple of the upper limit of normal of physiologic dosing in hydrocortisone equivalents.

308. The method of any one of claims 301 to 305, wherein the glucocorticoid dose is a physiologic dose as measured after a time period of administration of the compound of Formula (I), or a pharmaceutically acceptable salt thereof.

309. The method of any one of claims 301 to 305, wherein the glucocorticoid dose is a physiologic dose of about 4 to about 12 mg/m²/day as measured after a time period of administration of the compound of Formula (I), or a pharmaceutically acceptable salt thereof.

310. The method of any one of claims 301 to 305, wherein the glucocorticoid dose is a physiologic dose of about 4 to about 9 mg/m²/day as measured after a time period of administration of the compound of Formula (I), or a pharmaceutically acceptable salt thereof.

311. The method of any one of claims 301 to 305, wherein the glucocorticoid dose is a physiologic dose that is less than about 8 mg/m²/day as measured after a time period of administration of the compound of Formula (I), or a pharmaceutically acceptable salt thereof.

312. The method of any one of claims 301 to 311, wherein the glucocorticoid dose of the subject is reduced by about 10% after a time period of administration of the compound of Formula (I), or a pharmaceutically acceptable salt thereof, wherein the reduction of the glucocorticoid dose is relative to the glucocorticoid dose prior to administration of the compound of Formula (I).

313. The method of any one of claims 301 to 311, wherein the glucocorticoid dose of the subject is reduced by about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

314. The method of any one of claims 301 to 311, wherein the glucocorticoid dose of the subject is reduced by about 30% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

315. The method of any one of claims 301 to 311, wherein the glucocorticoid dose of the subject is reduced by about 40% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

316. The method of any one of claims 301 to 311, wherein the glucocorticoid dose of the subject is reduced by about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

317. The method of any one of claims 301 to 311, wherein the glucocorticoid dose of the subject is reduced by about 60% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

318. The method of any one of claims 301 to 311, wherein the glucocorticoid dose of the subject is reduced by about 70% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

319. The method of any one of claims 301 to 311, wherein the glucocorticoid dose of the subject is reduced by less than about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

320. The method of any one of claims 301 to 311, wherein the glucocorticoid dose of the subject is reduced by about 20% to about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

321. The method of any one of claims 301 to 311, wherein the glucocorticoid dose of the subject is reduced by greater than about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

322. The method of any one of claims 210 to 321, wherein the level of 17-hydroxyprogesterone is less than about 1.5 times the upper limit of normal after a time period of administration of the compound of Formula (I), or a pharmaceutically acceptable salt thereof.

323. The method of any one of claims 210 to 322, wherein the level of 17-hydroxyprogesterone is within normal limits after a time period of administration of the compound of Formula (I), or a pharmaceutically acceptable salt thereof.

324. The method of any one of claims 210 to 323, wherein the level of adrenocorticotropic hormone is less than about 1.5 times the upper limit of normal after a time period of administration of the compound of Formula (I), or a pharmaceutically acceptable salt thereof.

325. The method of any one of claims 210 to 324, wherein the level of adrenocorticotropic hormone is within normal limits after a time period of administration of the pharmaceutical composition.

326. The method of any one of claims 210 to 325, wherein the level of androstenedione is less than about 1.5 times the upper limit of normal after a time period of administration of the compound of Formula (I), or a pharmaceutically acceptable salt thereof.

327. The method of any one of claims 210 to 326, wherein the level of androstenedione is within normal limits after a time period of administration of the compound of Formula (I), or a pharmaceutically acceptable salt thereof.

328. The method of any one of claims 210 to 327, wherein the level of testosterone is reduced by at least about 25% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

329. The method of any one of claims 210 to 327, wherein the level of testosterone is reduced by at least about 30% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

330. The method of any one of claims 210 to 327, wherein the level of testosterone is reduced by at least about 50% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

331. The method of any one of claims 210 to 330, wherein the level of testosterone is less than about 1.5 times the upper limit of normal after a time period of administration of the pharmaceutical composition.

332. The method of any one of claims 210 to 331, wherein the level of testosterone is within normal limits after a time period of administration of the pharmaceutical composition comprising the compound of Formula (I), or a pharmaceutically acceptable salt thereof.

333. The method of any one of claims 301 to 332, wherein the subject exhibits a decrease in glucocorticoid burden after a time period of administration of the compound of Formula (I), or a pharmaceutically acceptable salt thereof, wherein the decrease in glucocorticoid burden is relative to the glucocorticoid burden prior to administration of the compound of Formula (I), or a pharmaceutically acceptable salt thereof.

334. The method of claim 333, wherein one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor, control of ovarian adrenal rest tumor, and fertility, is improved after a time period of administration of the compound of Formula (I), or a pharmaceutically acceptable salt thereof, wherein the improvement in the one or more symptoms is relative to the status of the one or more symptoms prior to administration of the compound of Formula (I), or a pharmaceutically acceptable salt thereof.

335. The method of any one of claims 308 to 334, wherein the time period of administration is at least about 4 weeks.

336. The method of any one of claims 308 to 334, wherein the time period of administration is at least about 24 weeks.

337. The method of any one of claims 308 to 334, wherein the time period of administration is at least about one year.

338. A method of treating congenital adrenal hyperplasia in a subject, the method comprising:
(a) selecting a subject who has a glucocorticoid dose of greater than 11 mg/m²/day after a time period of being administered a compound of Formula (I), or a pharmaceutically acceptable thereof, at an amount of about 100 mg twice daily based on the weight of the free base; and
(b) administering to the subject the compound of Formula (I), or a pharmaceutically acceptable salt thereof, at a frequency of twice daily;
wherein the amount of the compound of Formula (I), or a pharmaceutically acceptable salt thereof, in the first administration is less than the amount of the compound of Formula (I), or a pharmaceutically acceptable salt thereof, in the second administration;
and wherein the amount of the compound of Formula (I), or a pharmaceutically acceptable salt thereof, administered daily is greater than or equal to about 200 mg based on the weight of the free base.

339. The method of claim 338, wherein the time period of administration in step (a) is at least about 4 weeks.

340. The method of claim 338, wherein the time period of administration in step (a) is at least about 24 weeks.

341. The method of claim 338, wherein the time period of administration in step (a) is at least about one year.

342. The method of any one of claims 210 to 341, wherein the subject is female.

343. The method of any one of claims 210 to 341, wherein the subject is male.

344. The method of any one of claims 210 to 343, wherein the compound of Formula (I) is administered as a pharmaceutical composition comprising: (a) the compound of Formula (I), or a pharmaceutically acceptable salt thereof; and (b) one or more of an oily phase vehicle, an emulsifying agent, a nonionic surfactant, and a solubilizing agent.

345. The method of claim 344, wherein the pharmaceutical composition comprises about 1 wt % to about 20 wt % of the compound of Formula (I), or a pharmaceutically acceptable salt thereof, based on the weight of the free base.

346. The method of claim 344, wherein the pharmaceutical composition comprises about 5 wt % to about 15 wt % of the compound of Formula (I), or a pharmaceutically acceptable salt thereof, based on the weight of the free base.

347. The method of claim 344, wherein the pharmaceutical composition comprises about 10 wt % of the compound of Formula (I), or a pharmaceutically acceptable salt thereof, based on the weight of the free base.

348. The method of any one of claims 344 to 347, wherein the pharmaceutical composition comprises an oily phase vehicle.

349. The method of any one of claims 344 to 348, wherein the pharmaceutical composition comprises about 1 wt % to about 50 wt % of the oily phase vehicle.

350. The method of any one of claims 344 to 348, wherein the pharmaceutical composition comprises about 20 wt % to about 50 wt % of the oily phase vehicle.

351. The method of any one of claims 344 to 348, wherein the pharmaceutical composition comprises about 35 wt % to about 45 wt % of the oily phase vehicle.

352. The method of any one of claims 344 to 348, wherein the pharmaceutical composition comprises about 39 wt % of the oily phase vehicle.

353. The method of any one of claims 344 to 352, wherein the oily phase vehicle is selected from medium-chain triglycerides, glycerin, propylene glycol, polyethylene glycol, olive oil, soybean oil, corn oil, and transcutol.

354. The method of any one of claims 344 to 353, wherein the oily phase vehicle is medium-chain triglycerides.

355. The method of claim 354, wherein the medium-chain triglycerides are Labrafac™ Lipophile WL1349.

356. The method of claim 354, wherein the medium-chain triglycerides are Miglyol 812N.

357. The method of any one of claims 344 to 356, wherein the pharmaceutical composition comprises an emulsifying agent.

358. The method of any one of claims 344 to 357, wherein the pharmaceutical composition comprises about 5 wt % to about 50 wt % of the emulsifying agent.

359. The method of any one of claims 344 to 357, wherein the pharmaceutical composition comprises about 10 wt % to about 30 wt % of the emulsifying agent.

360. The method of any one of claims 344 to 357, wherein the pharmaceutical composition comprises about 15 wt % to about 25 wt % of the emulsifying agent.

361. The method of any one of claims 344 to 357, wherein the pharmaceutical composition comprises about 20 wt % of the emulsifying agent.

362. The method of any one of claims 344 to 361, wherein the emulsifying agent is selected from medium-chain triglycerides, propylene glycol dicaprylate/dicaprate, glycerin, propylene glycol, polyethylene glycol, olive oil, soybean oil, corn oil, and transcutol.

363. The method of any one of claims 344 to 362, wherein the emulsifying agent is propylene glycol dicaprylate/dicaprate.

364. The method of claim 363, wherein the propylene glycol dicaprylate/dicaprate is Labrafac™ PG.

365. The method of any one of claims 344 to 364, wherein the pharmaceutical composition comprises a nonionic surfactant.

366. The method of any one of claims 344 to 365, wherein the pharmaceutical composition comprises about 5 wt % to about 50 wt % of the nonionic surfactant.

367. The method of any one of claims 344 to 365, wherein the pharmaceutical composition comprises about 10 wt % to about 30 wt % of the nonionic surfactant.

368. The method of any one of claims 344 to 365, wherein the pharmaceutical composition comprises about 15 wt % to about 25 wt % of the nonionic surfactant.

369. The method of any one of claims 344 to 365, wherein the pharmaceutical composition comprises about 19 wt % of the nonionic surfactant.

370. The method of any one of claims 344 to 369, wherein the nonionic surfactant is selected from oleoyl polyoxyl-6 glycerides, linoleoyl polyoxyl-6 glycerides, Polysorbate 80, Polysorbate 20, Gelucire, lauroyl polyoxyl-32 glycerides, Poloxamer, PEG-32 stearate, and PEG-32 hydrogenated palm glycerides.

371. The method of any one of claims 344 to 370, wherein the nonionic surfactant is lauroyl polyoxyl-32 glycerides.

372. The method of claim 371, wherein the lauroyl polyoxyl-32 glycerides are Gelucire® 44/14.

373. The method of any one of claims 344 to 372, wherein the pharmaceutical composition comprises a solubilizing agent.

374. The method of any one of claims 344 to 373, wherein the pharmaceutical composition comprises about 1 wt % to about 50 wt % of the solubilizing agent.

375. The method of any one of claims 344 to 373, wherein the pharmaceutical composition comprises about 1 wt % to about 20 wt % of the solubilizing agent.

376. The method of any one of claims 344 to 373, wherein the pharmaceutical composition comprises about 5 wt % to about 15 wt % of the solubilizing agent.

377. The method of any one of claims 344 to 370, wherein the pharmaceutical composition comprises about 11 wt % of the solubilizing agent.

378. The method of any one of claims 344 to 377, wherein the solubilizing agent is selected from oleoyl polyoxyl-6 glycerides, linoleoyl polyoxyl-6 glycerides, Polysorbate 80, Polysorbate 20, vitamin E polyethylene glycol succinate, Gelucire, lauroyl polyoxyl-32 glycerides, and Poloxamer.

379. The method of any one of claims 344 to 378, wherein the solubilizing agent is vitamin E polyethylene glycol succinate.

380. The method of claim 379, wherein the vitamin E polyethylene glycol succinate is Kolliphor® TPGS.

381. The method of claim 379, wherein the vitamin E polyethylene glycol succinate is Vitamin E/TPGS 260.

382. The method of claim 344, wherein the pharmaceutical composition comprises:
(a) the compound of Formula (I), or a pharmaceutically acceptable salt thereof,
(b) an oily phase vehicle;
(c) an emulsifying agent;
(d) a nonionic surfactant; and
(e) a solubilizing agent.

383. The method of claim 344, wherein the pharmaceutical composition comprises:
(a) about 5 wt % to about 15 wt % of the compound of Formula (I), or a pharmaceutically acceptable salt thereof, based on the weight of the free base;
(b) about 35 wt % to about 45 wt % of an oily phase vehicle;
(c) about 15 wt % to about 25 wt % of an emulsifying agent;
(d) about 15 wt % to about 25 wt % of a nonionic surfactant; and
(e) about 5 wt % to about 15 wt % of a solubilizing agent.

384. The method of claim 344, wherein the pharmaceutical composition comprises:
(a) about 10 wt % of the compound of Formula (I), or a pharmaceutically acceptable salt thereof, based on the weight of the free base;
(b) about 39 wt % of an oily phase vehicle;
(c) about 20 wt % of an emulsifying agent;
(d) about 19 wt % of a nonionic surfactant; and
(e) about 11 wt % of a solubilizing agent.

385. The method of claim 344, wherein the pharmaceutical composition comprises:
(a) the compound of Formula (I);
(b) a medium-chain triglycerides component;
(c) a propylene glycol dicaprylate/dicaprate component;
(d) a lauroyl polyoxyl-32 glycerides component; and
(e) a vitamin E polyethylene glycol succinate component.

386. The method of claim 344, wherein the pharmaceutical composition comprises:
(a) about 5 wt % to about 15 wt % of the compound of Formula (I);
(b) about 35 wt % to about 45 wt % of medium-chain triglycerides;
(c) about 15 wt % to about 25 wt % of propylene glycol dicaprylate/dicaprate;
(d) about 15 wt % to about 25 wt % of lauroyl polyoxyl-32 glycerides; and
(e) about 5 wt % to about 15 wt % of vitamin E polyethylene glycol succinate.

387. The method of claim 344, wherein the pharmaceutical composition comprises:
(a) about 10 wt % of the compound of Formula (I);
(b) about 39 wt % of medium-chain triglycerides;
(c) about 20 wt % of propylene glycol dicaprylate/dicaprate;
(d) about 19 wt % of lauroyl polyoxyl-32 glycerides; and
(e) about 11 wt % of vitamin E polyethylene glycol succinate.

388. The method of any one of claims 344-387, wherein the pharmaceutical composition comprises the compound of Formula (I), or pharmaceutically acceptable salt thereof, in crystalline form.

389. The method of any one of claims 344-387, wherein the pharmaceutical composition comprises the compound of Formula (I) as a free base.

390. The method of claim 389, wherein the crystalline form comprises Form I of the compound of Formula (I) as a free base.

391. The method of any one of claims 344 to 390, wherein the pharmaceutical composition is formulated in unit dosage form, wherein the compound of Formula (I), or a pharmaceutically acceptable salt thereof, is present in an amount of about 5 mg to about 200 mg, based on the weight of the free base.

392. The method of claim 391, wherein the compound of Formula (I), or a pharmaceutically acceptable salt thereof, is present in the unit dosage form in an amount of about 25 mg to about 150 mg, based on the weight of the free base.

393. The method of claim 391, wherein the compound of Formula (I), or a pharmaceutically acceptable salt thereof, is present in the unit dosage form in an amount of about 50 mg, based on the weight of the free base.

394. The method of claim 391, wherein the compound of Formula (I), or a pharmaceutically acceptable salt thereof, is present in the unit dosage form in an amount of about 100 mg, based on the weight of the free base.

395. The method of claim 391, wherein the compound of Formula (I), or a pharmaceutically acceptable salt thereof, is present in the unit dosage form in an amount of about 25 mg, based on the weight of the free base.

396. The method of any one of claims 344 to 395, that is in the form of a tablet, capsule, sachet, powder, granules, coated particle, coated tablet, enterocoated tablet, enterocoated capsule, melting strip, or melting film.

397. The method of claim 396, wherein the pharmaceutical composition is in tablet form.

398. The method of claim 396, wherein the pharmaceutical composition is in capsule form.

399. The pharmaceutical composition of claim 397 or 398, wherein the tablet form or capsule form is coated.

400. The method of any one of claims 210 to 343, wherein the compound of Formula (I) is administered as a pharmaceutical composition in oral solution dosage form comprising:
(a) the compound of Formula (I),
or a pharmaceutically acceptable salt thereof;
(b) one or more of a sweetener, an anti-oxidant, and a flavor; and
(c) a liquid vehicle.

401. The method of claim 400, wherein the pharmaceutical composition comprises:
(a) the compound of Formula (I), or a pharmaceutically acceptable salt thereof,
(b) a sweetener;
(c) an anti-oxidant;
(d) a flavor; and
(e) a liquid vehicle.

402. The method of claim 401, wherein the pharmaceutical composition further comprises a surfactant.

403. The method of claim 400, wherein the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor;
(e) about 15 w/v % to about 25 w/v % of a surfactant; and
(f) about 70 w/v % to about 80 w/v % of a liquid vehicle.

404. The method of claim 400, wherein the pharmaceutical composition comprises:
(a) about 5 w/v % of the compound of Formula (I), or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor;
(e) about 20 w/v % of a surfactant; and
(f) about 75 w/v % of a liquid vehicle.

405. The method of claim 400, wherein the pharmaceutical composition comprises:
(a) a compound of Formula (I), or a pharmaceutically acceptable salt thereof;
(b) saccharin;
(c) butylated hydroxytoluene;
(d) FONA orange flavor; and
(e) medium-chain triglycerides.

406. The method of claim 405, wherein the pharmaceutical composition further comprises oleoyl polyoxyl-6 glycerides.

407. The method claim 400, wherein the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor;
(e) about 15 w/v % to about 25 w/v % of oleoyl polyoxyl-6 glycerides; and
(f) about 70 w/v % to about 80 w/v % of medium-chain triglycerides.

408. The method of claim 400, wherein the pharmaceutical composition comprises:
(a) about 5 w/v % of the compound of Formula (I), or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor;
(e) about 20 w/v % of oleoyl polyoxyl-6 glycerides; and
(f) about 75 w/v % of medium-chain triglycerides.

409. The method of any one of claims 400 to 408, wherein the pharmaceutical composition comprises the compound of Formula (I) as a free base.

410. The method of any one of claims 400 to 409, wherein the pharmaceutical composition is formulated in unit dosage form, wherein the compound of Formula (I), or a pharmaceutically acceptable salt thereof, is present in an amount of about 5 mg/mL to about 200 mg/mL, based on the weight of the free base.

411. The method of claim 410, wherein the unit dosage form comprises the compound of Formula (I), or a pharmaceutically acceptable salt thereof, in an amount of about 50 mg/mL, based on the weight of the free base.

412. The method of any one of claims 210 to 343, wherein the compound of Formula (I) is administered in a pharmaceutical composition comprising a spray-dried dispersion comprising:
a compound of Formula (I), or a pharmaceutically acceptable salt thereof, and
a polymer selected from a neutral polymer, an enteric polymer, and a pyrrolidone polymer;
wherein the weight ratio of the compound of Formula (I) to the polymer is from about 1:1 to about 1:9.

413. The method of claim 412, wherein the spray-dried dispersion comprises:
a compound of Formula (I), or a pharmaceutically acceptable salt thereof, and
a polymer that is a copolymer of 1-vinyl-2-pyrrolidone and vinyl acetate having the structure: 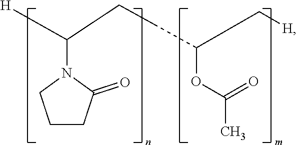
wherein the value of n is about 1 to about 2 times the value of m and the copolymer comprises 1-vinyl-2-pyrrolidone and vinyl acetate at a ratio of about 60:40 by weight; and
wherein the weight ratio of the compound of Formula (I) to the copolymer is from about 1:1 to about 1:9.

414. The method of claim 412, wherein the pharmaceutical composition comprises:
(a) the spray-dried dispersion of Example 3;
(b) calcium silicate;
(c) a combination of mannitol and microcrystalline cellulose; and
(d) croscarmellose sodium.

415. The method of claim 412, wherein the pharmaceutical composition comprises:
(a) about 1 w/w % to about 20 w/w % of the spray-dried dispersion of Example 3;
(b) about 0.1 w/w % to about 1 w/w % of calcium silicate;
(c) about 50 w/w % to about 60 w/w % of mannitol and about 10 w/w % to about 30 w/w % of microcrystalline cellulose; and
(d) about 5 w/w % to about 0.2 w/w % of croscarmellose sodium.

416. The method of claim 412, wherein the pharmaceutical composition comprises:
(a) about 13 w/w % of the spray-dried dispersion of Example 3;
(b) about 0.67 w/w % of calcium silicate;
(c) about 56 w/w % of mannitol and about 20 w/w % of microcrystalline cellulose; and
(d) about 10 w/w % of croscarmellose sodium.

417. The method of any one of claims 412-416, wherein the pharmaceutical composition is formulated as a tablet, capsule, sachet, powder, granules, coated particle, coated tablet, enterocoated tablet, enterocoated capsule, melting strip, or melting film.

418. The method of claim 417, wherein the pharmaceutical composition is in capsule form.

419. The method of claim 282, wherein the free base form of the compound of Formula (I) is a crystalline form.

420. The method of claim 419, wherein the crystalline form comprises Form I.

421. The method of any one claims 210 to 281, wherein the compound of Formula (I), or a pharmaceutically acceptable salt thereof, is a p-toluenesulfonic acid salt of the compound of Formula (I).
